(19) United States
(12) Patent Application Publication
(10) Pub. No.: US 2013/0157876 A1
(43) Pub. Date: Jun. 20, 2013

(54) Systems and Methods for Detecting Antibiotic Resistance

(71) Applicants: THE REGENTS OF THE UNIVERSITY OF CALIFORNIA, Oakland, CA (US); TESSARAE, LLC, Potomac Falls, VA (US)

(72) Inventors: Susan V. Lynch, Piedmont, CA (US); Eoin L. Brodie, Piedmont, CA (US); Ulas Karaoz, Berkeley, CA (US); Ramya Srinivasan, San Francisco, CA (US); Anjan Purkayastha, Vienna, VT (US); Matthew C. Lorence, Alamo, CA (US); Clark J. Tibbetts, Sperryville, VT (US)

(73) Assignees: TESSARAE, LLC, Potomac Falls, VA (US); THE REGENTS OF THE UNIVERSITY OF CALIFORNIA, Oakland, CA (US)

(21) Appl. No.: 13/773,631

(22) Filed: Feb. 21, 2013

Related U.S. Application Data
(63) Continuation of application No. PCT/US2011/048698, filed on Aug. 22, 2011.
(60) Provisional application No. 61/375,816, filed on Aug. 21, 2010.

(51) Int. Cl. C12Q 1/68

(52) U.S. Cl. CPC C12Q 1/689; C12Q 1/6874; C12Q 1/6806
USPC 506/2; 435/6.12; 506/16; 506/30

(57) ABSTRACT

A robust, automated computational pipeline was used to design a system comprising a microarray for the identification of microorganisms and their antibiotic resistance profiles. This system and methods will facilitate the study of the epidemiology and microbial ecology of antibiotic resistance and be an invaluable tool to rapidly and simultaneously identify organisms and their antimicrobial resistance elements in environmental, food and clinical samples.

### CROSS-REFERENCE TO RELATED APPLICATIONS

This application is a non-provisional continuation-in-part application of and claims priority to PCT/US2011/048698 filed on Aug. 22, 2011, and to U.S. Provisional Patent Application No. 61/375,816 filed on Aug. 21, 2010, both of which are herein incorporated by reference in their entirety.

### STATEMENT REGARDING GOVERNMENTAL SUPPORT

This invention was made with Government support under Contract No. DE-AC03-05CH11231 awarded by the Department of Energy and under Grant No. U01 AI075410-01 awarded by the NIH/NIAID. The Government has certain rights in the invention.

### REFERENCE TO TABLES AND SEQUENCE LISTING

The present application includes and incorporates by reference the attached Table 2.

The instant application contains a Sequence Listing which has been submitted in ASCII format via EFS-Web and is hereby incorporated by reference in its entirety. Said ASCII copy, created on Feb. 14, 2013, is named 2927US_SequenceListing_ST25.txt and is 542,906 bytes in size.

### BACKGROUND OF THE INVENTION

1. Field of the Invention

The present embodiments relate to the design and implementation of a microarray-based assay and array system for detecting specific target genes from organisms permitting microbial identification and profiling of antibiotic resistance and methods of use of the assay and array system thereof.

2. Description of the Related Art

A DNA microarray (also commonly known as gene or genome chip, DNA chip, or gene array) is a collection of microscopic DNA spots attached to a solid surface, made of such materials as glass, plastic or silicon and forming an array. The solid surface can be planar or other surface shapes, such as microbeads, etc. The affixed DNA segments are known as probes (although some sources will use different nomenclature), thousands of which can be used in a single DNA microarray. Microarrays may be used to detect specific genes and to measure their expression which is relevant to many areas of biology and medicine, such as studying treatments, disease, and developmental stages. For example, microarrays can be used to identify disease genes by comparing gene expression in diseased and normal cells.

Healthcare-associated infections [HAIs] are a worldwide problem affecting diverse groups of patients. A major challenge in fighting HAIs is the rising tide of antimicrobial resistance. Nonclinical environments are the natural origin of most antibiotic resistance elements; however, the use of broad-spectrum antibiotics in the clinical setting places significant selective pressure on microorganisms, altering the functional role of these elements.

There is a need to provide a sensitive and specific molecular tool for rapid, culture-independent identification of microbial species present in a clinical sample as well as providing information on the antibiotic resistance potential of these strains. This is relevant to research applications, but particularly pertinent for clinical applications. Specifically, there is currently no clinical diagnostic tool available that can simultaneously identify a large panel of pathogenic microorganisms (at least 44 species) and their associated antibiotic resistance genes within a time frame that will permit therapeutic antimicrobial treatment strategies to dramatically impact patient outcomes. Though it has been determined that appropriate (meaning efficacious against the infectious agent) and adequate (meaning sufficient concentrations to effect a >90% bactericidal rate) administration of antibiotics within 24 hours of presentation with infection significantly improves patient outcomes, to date no current technique simultaneously allows organism detection and antibiotic resistance profiling for a multitude of nosocomial pathogenic agents in one assay within 10-24 h. Current clinical laboratory tests are primarily culture-based or, if molecular, rely upon fluorescence in situ hybridization or PCR amplification alone (which are typically single species specific). [(1) PNA-FISH, AdvanDx (2) Amplified MTD, Gen-Probe (3) IDI-MRSA, Cepheid] These detect only a limited number of infecting pathogens and/or specific antibiotic resistance determinants in clinical samples. Their detection sensitivity is not at strain level. Almost all of these tests are each limited to a couple of pathogens and resistance determinants. Each one is laborious and running a battery of these tests make them expensive and time consuming, and in many cases do not satisfy the requirements of clinical timelines to affect treatment decisions.

Early work has already demonstrated the potential of microarray methods for detection of pathogenic bacteria and their associated resistance elements and virulence factors (Wilson W J, Strout C L, DeSantis T Z et al. Sequence-specific identification of 18 pathogenic microorganisms using microarray technology. Mol Cell Probes 2002; 16:119-27). Anthony et al. used universal PCR primers to amplify 23S ribosomal DNA from bacteria in clinical blood cultures, followed by hybridization to a microarray with species-specific sequences for 95 bacterial and fungal pathogens (Anthony R M, Brown T J, French G L. Rapid diagnosis of bacteremia by universal amplification of 23S ribosomal DNA followed by hybridization to an oligonucleotide array. J Clin Microbiol 2000; 38:781-8). Of 158 culture-positive clinical specimens, 125 (80%) were correctly identified using this approach. Discrepancies were due to inadequate target coverage on the microarray, false-negative PCR amplifications and false-negative cultures.

Other groups have developed microarray techniques to accurately characterize the presence of antimicrobial resistance elements in microorganisms. Lee et al. developed a DNA chip to detect a variety of different classes of beta-lactamase genes from Gram-negative bacteria, demonstrating an ability to detect the presence of resistance genes in as little as a single organism (Lee Y, Lee C S, Kim Y J et al. Development of DNA chip for the simultaneous detection of various beta-lactam antibiotic-resistant genes. Mol Cells 2002; 14:192-7). The potential of microarrays to detect single nucleotide polymorphism variants in antibiotic resistance genes was demonstrated by Grimm et al. in their study of the common Gram-negative beta-lactamase TEM-1 (Grimm V, Ezaki S, Susa M et al. Use of DNA microarrays for rapid genotyping of TEM beta-lactamases that confer resistance. J Clin Microbiol 2004; 42:3766-74). This study correctly identified different TEM variants in Escherichia coli, Enterobacter cloacae, and Klebsiella pneumoniae. Frye et al. developed a microarray with probes for 94 antimicrobial resistance genes (Frye J G, Jesse T, Long F et al. DNA microarray detection of antimicrobial resistance genes in diverse bacteria. Int J Antimicrob Agents 2006; 27:138-51). When tested against 51 Gram-negative and Gram-positive bacteria, 61 resistance genes were detected, with good agreement between the presence of genes for resistance and phenotypic susceptibility results.

The most advanced work to date in development of a clinically useful microarray for rapid detection of pathogens and antimicrobial resistance was reported by Cleven et al. (Cleven B E, Palka-Santini M, Gielen J et al. Identification and characterization of bacterial pathogens causing bloodstream infections by DNA microarray. J Clin Microbiol 2006; 44:2389-97.). This group designed a microarray platform to perform pathogen identification and detection of resistance elements in Staphylococcus aureus, Escherichia coli, and Pseudomonas aeruginosa. Identification of all three species from blood culture samples was 100%, and there was excellent agreement between resistance gene probes and results of phenotypic susceptibility testing for Staphylococcus aureus.

One limitation of current methods for detection of antimicrobial resistance genes via DNA microarrays is the inability to predict expression levels among the identified genes. While the presence of antimicrobial resistance genes in many organisms correlates well with phenotypic resistance, some resistance genes may be chromosomally located and have variable effect on resistance based on their level of expression. Chromosomally encoded resistance genes are common among wild-type isolates of Gram-negative bacteria such as P. aeruginosa, Enterobacter, and Acinetobacter (Hancock R E. Resistance mechanisms in Pseudomonas aeruginosa and other nonfermentative Gram-negative bacteria. Clin Infect Dis 1998; 27 Suppl 1:S93-9). These genes usually encode either beta-lactamases or drug efflux pumps, and are expressed at low levels among drug-susceptible isolates. Mutations in the regulatory genes for these elements allows for their high-level expression, with subsequent phenotypic resistance (often to multiple drug classes). In order to characterize accurately the resistance profiles of these organisms, molecular methods such as microarrays must either be able to quantitatively measure gene expression or interrogate regulatory genes to determine the presence of mutations . . . .

### SUMMARY OF THE INVENTION

In one embodiment, a computational pipeline to select both species-level identification and antibiotic resistance determinants for microbial detection, identification and antibiotic resistance phenotype prediction of important nosocomial pathogens. The focus was on 24 pathogenic organisms with clinical relevance and shown in Table 1.

In one embodiment, a robust, automated computational pipeline used to design a microarray of pathogen identification and antibiotic resistance elements. This array will facilitate the detection of organisms, including nosocomial pathogens and their resistance profiles in a clinical setting. In addition, the tool will have applications in epidemiological studies, given its capability to provide virtual real-time information on strain identity and resistance profile, it may be used during infectious outbreaks to track dissemination and evolution of infectious agents.

In one embodiment, the array comprises probes to detect and identify specific nosocomial pathogens and their antibiotic resistance elements corresponding to at least the 24 pathogens in Table 1, wherein the probes are attached to a substrate or solid surface.

In other aspects, the probes which are 25 nucleotides long are used to detect and identify specific organisms and their antibiotic resistance elements.

In some aspects, the sample is an environmental sample. In other aspects, the environmental sample comprises at least one of soil, water, or atmosphere.

In some aspects, the sample is a processed or unprocessed food product. In other aspects, the food sample comprises at least one of meat, turkey, chicken and other poultry, milk, eggs, eggs products, dairy products, fresh or dried fruits and vegetables and their juices, grains, fish, seafood, pet food, baby food and infant formula,

In yet other aspects, the sample is a clinical sample. In still other aspects, the clinical sample comprises at least one of tissue, skin, stool, bodily fluid, or blood.

In another aspect, the sample is a bacterial isolate.

Some embodiments relate to a method of detecting an organism including applying a pool of DNA fragments amplified from genomes extracted from the sample to the array system which includes a microarray that comprises probes that specifically target genomic regions specific to an organism and their antibiotic resistance elements.

In some aspects, the plurality of organisms comprise bacteria.

In some aspects, the specific antibiotic resistance element and the organism (pathogenic or other) are detected in the sample.

Some embodiments relate to a method of fabricating an array system including identifying sequences conferring antibiotic resistance; clustering those sequences that target resistance determinants of clinical relevance; creating variant sequence fragments corresponding to these antibiotic resistance sequences; and fabricating said array system.

In one embodiment, the array system is a microarray system comprising: a resequencing microarray configured to simultaneously detect a plurality of organisms and antibiotic resistance elements in a sample, wherein the microarray comprises resequencing probes for organism identification, antibiotic resistance element detection, and detection of polymorphisms related to said antibiotic resistance, whereby said resequencing probes for organism detection can provide strain-level detection and identification of a pathogen in a sample and whereby said resequencing probes for antibiotic resistance elements and said resequencing probes for antibiotic resistance related polymorphisms provide for detection of emerging antibiotic resistance of pathogens in a sample

In one aspect, the invention provides a method for parallel detection and strain level-identification of a panel of more than 44 organisms, in parallel with antibiotic resistance profiling of said organisms comprising the steps of: (a) extraction of nucleic acids from a patient sample using a rapid optimized protocol; (b) amplifying a target loci in said nucleic acid using multiplex polymerase chain reaction; (c) pooling and labeling said target locus amplified products; (d) contacting the labeled amplified pool of products with a plurality of resequencing probes which target both the sense and anti-sense strands of the target loci represented in SEQ ID NOS:1-1323; (e) determining hybridization signal strength for each of said resequencing probes, wherein said determination identifies the specific sequence of the target locus, providing either strain level organism identification or single nucleotide polymorphism resolution or antibiotic resistance determinant sequence information. In some embodiments, the method further comprises in step (c) fragmenting the target locus amplified products.

In one embodiment, the array comprises probes to detect the presence of regulatory genes related to antibiotic resistance. In another embodiment, it comprises probes that cover promoter regions of genes involved in antibiotic resistance.

In some embodiments, the sample is a pulmonary sample, including but not limited to sputum, endotracheal aspirate, a bronchoalveolar lavage sample, or a swab of the endotrachea.

In some embodiments, the biosignature comprises the presence and antibiotic resistance elements profile of one or more nosocomial pathogens.

In another embodiment, the invention provides a method of diagnosis, prognosis, and/or prediction of an outcome of a subject. In one embodiment, the method comprises: (a) isolating nucleic acid material from a sample from said subject; (b) determining hybridization signal strength distributions across sets of 8 probes (4 based on sense strain, 4 on anti-sense strand) per nucleotide base interrogated in each target locus; (c) determining hybridization signal strengths for a plurality of different interrogation probes, each of which is complementary to a section within said loci; (d) using the hybridization signal strengths of probe sets to determine the sequence of the target locus. (e) using this information to determine the presence and antibiotic resistance repertoire of one or more target nosocomial pathogens; (f) defining therapeutic strategy based on the results of step (d); (g) classifying, diagnosing, prognosing, and/or predicting an outcome of said pulmonary condition based on the results of step (d). In some embodiments, the sample is a pulmonary sample, including but not limited to sputum, endotracheal aspirate, a bronchoalveolar lavage sample, or a swab of the endotrachea. In some embodiments, the method further comprises making a healthcare decision based on the results of step (d).

In one aspect, the invention provides a method of diagnosis, prognosis, and/or prediction of an outcome of a condition in a subject. In one embodiment, the method comprises: (a) obtaining a sample from a patient; (b) isolating nucleic acid material from said sample; (c) amplifying a target locus in said nucleic acid material; (d) contacting said target locus with a set of resequencing probes, wherein said set comprising 8 probes (4 probes based on the sense strand and 4 probes based on the anti-sense strand) per nucleotide base interrogated in each target locus; (e) determining hybridization signal strengths across the set of probes; (f) determining hybridization signal strengths for a plurality of different interrogation probes, each of which is complementary to a section within said target locus; (g) determining the sequence of the target locus by analysis of the hybridization signal strengths of the resequencing probe set; (h) comparing the target locus sequence with a set of known sequences to determine the presence and/or antibiotic resistance repertoire of one or more target organisms; (i) defining therapeutic strategy for said patient based on the results of step (h); (j) classifying, diagnosing, prognosing, and/or predicting an outcome of said condition based on the results of step (h). In some embodiments, the presence and antibiotic resistance profile is detected with a confidence level greater than 95%. Highly conserved target loci targeted with this invention include, but are not limited to, 16S rRNA gene, 23S rRNA gene, 5S rRNA gene, 5.8S rRNA gene, 12S rRNA gene, 18S rRNA gene, 28S rRNA gene, gyrB gene, rpoB gene, fusA gene, recA gene, coxl gene, nif13 gene, ahpC gene, embA gene, kasA gene, katG gene, parC gene, pncA gene and rlmN gene or RNA molecules derived therefrom, or a combination thereof. Single nucleotide polymorphisms present in target gene sequences include, but are not limited to, 16S rRNA, 23S rRNA, ahpC, alr, embA, embB, embC, ethA, fabG1, folP, gyrA, gyrB, kasA, katG, ndh, parC, parE, pncA, rlmN, rpoB, and/or rpsL genes, RNA molecules derived therefrom, or a combination thereof.

In one aspect, the invention provides a system for practicing the methods of the invention. A method is provided for simultaneously detecting an organism in a sample and its antibiotic resistance comprising the steps: applying a sample comprising a plurality of organisms to the array system; and simultaneously identifying at least one organism in the sample and determining its antibiotic resistance. Probes used in methods and systems of the present invention can be used to detect the presence, absence of at least 44 clinically relevant bacterial species and their associated antibiotic resistance determinants in a single assay. In some embodiments, probes are attached to a substrate. Substrates can comprise any suitable material, including but not limited to glass, plastic, or silicon. Substrates can take any suitable shape, such as a flat surface, a bead, or a microsphere.

In another aspect, an optimized pretreatment process for clinical samples.

### BRIEF DESCRIPTION OF THE DRAWINGS

FIG. 1 shows a chart of the breakdown of the seed sequences. The seed sequences of antibiotic resistance relevance consisted of a manually curated set of 268 sequences and sequences from a recent database of antibiotic resistance elements (ARDB, Liu B, Pop M. ARDB-Antibiotic Resistance Genes Database. Nucleic Acids Res. 2009 January; 37 (Database issue):D443-). The total number of seed sequences was 22413 and spanned 642 species and 145 antibiotics.

FIG. 2 is a flowchart of the computational pipeline to select antibiotic resistance determinants for identification and antibiotic resistance phenotype prediction of important nosocomial pathogens.

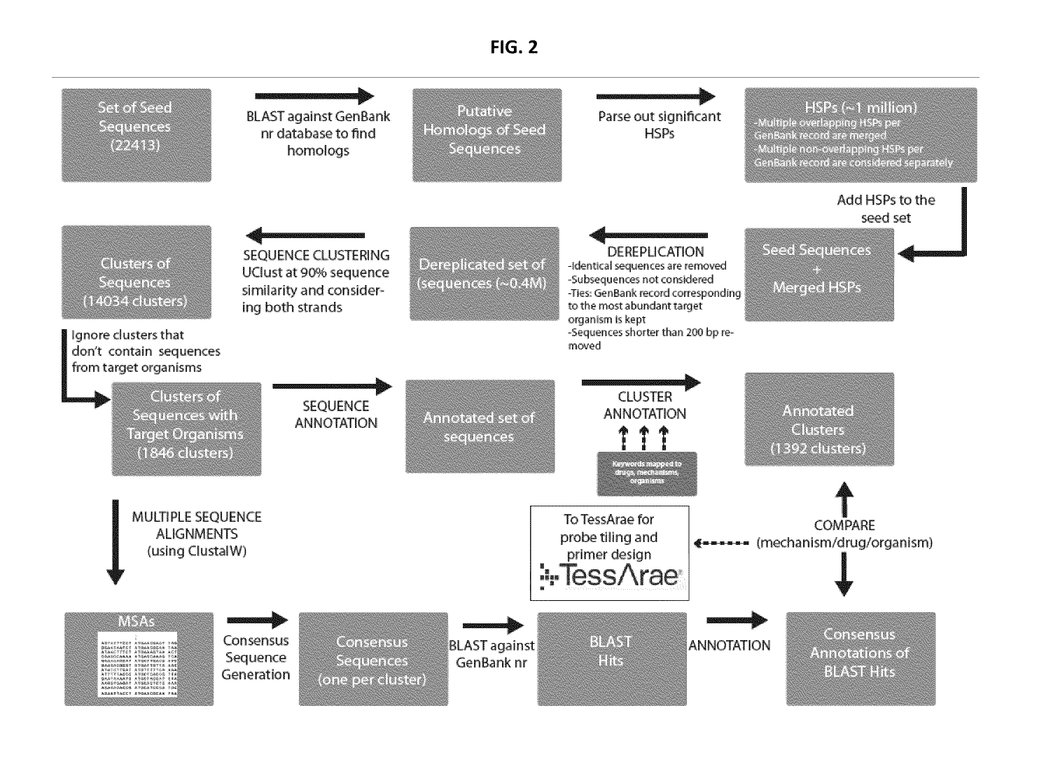

FIG. 3 is a bar graph demonstrating that increasing bead beating time improves detection of Gram-positive organisms (dark gray bars). Addition of BSA, further enhances sensitivity (light gray bars).

### DETAILED DESCRIPTION

The present embodiments are related to system comprising an array and methods for detecting and identifying biomolecules and organisms having antibiotic resistance. More specifically, some embodiments relate to an array system comprising a microarray configured to simultaneously detect a plurality of nosocomial pathogens and clinically relevant antibiotic resistance elements in a sample at a high confidence level.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a target locus” includes a plurality of such substrates and reference to “the antibiotic resistance element” includes reference to one or more recombinant polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The term “microarray,” as used herein, refers to an arrangement of distinct polynucleotides arrayed on a substrate, e.g., paper, nylon or any other type of membrane, filter, chip, glass slide, or any other suitable solid support.

As used herein, the term “oligonucleotide” refers to a polynucleotide, usually single stranded, that is either a synthetic polynucleotide or a naturally occurring polynucleotide. The length of an oligonucleotide is generally governed by the particular role thereof, such as, for example, probe, primer and the like. Various techniques can be employed for preparing an oligonucleotide, for instance, biological synthesis or chemical synthesis.

The nucleic acid may be DNA, RNA, or a hybrid and may contain any combination of deoxyribo- and ribo-nucleotides, and any combination of bases, including uracil, adenine, thymine, cytosine, guanine, inosine, xanthanine, hypoxanthanine, isocytosine, isoguanine, and base analogs such as nitropyrrole and nitroindole, etc. Oligonucleotides can be synthesized by standard methods such as those used in commercial automated nucleic acid synthesizers and later attached to an array, bead or other suitable surface. Alternatively, the oligonucleotides can be synthesized directly on the assay surface using photolithographic or other techniques. In some embodiments, linkers are used to attach the oligonucleotides to an array surface or to beads.

As used herein, the term “nucleic acid molecule” or “polynucleotide” refers to a compound or composition that is a polymeric nucleotide or nucleic acid polymer. The nucleic acid molecule may be a natural compound or a synthetic compound. The nucleic acid molecule can have from about 2 to 10,000,000 or more nucleotides. The larger nucleic acid molecules are generally found in the natural state. In an isolated state, the nucleic acid molecule can have about 10 to 50,000 or more nucleotides, usually about 100 to 20,000 nucleotides. It is thus obvious that isolation of a nucleic acid molecule from the natural state often results in fragmentation. It may be useful to fragment longer target nucleic acid molecules, particularly RNA, prior to hybridization to reduce competing intramolecular structures. Fragmentation can be achieved chemically, enzymatically, or mechanically. Typically, when the sample contains DNA, a nuclease such as deoxyribonuclease (DNase) is employed to cleave the phosphodiester linkages. Nucleic acid molecules, and fragments thereof, include, but are not limited to, purified or unpurified forms of DNA (dsDNA and ssDNA) and RNA, including tRNA, mRNA, rRNA, mitochondrial DNA and RNA, chloroplast DNA and RNA, DNA/RNA hybrids, biological material or mixtures thereof, genes, chromosomes, plasmids, cosmids, the genomes of microorganisms, e.g., bacteria, yeasts, phage, chromosomes, viruses, viroids, molds, fungi, or other higher organisms such as plants, fish, birds, animals, humans, and the like. The polynucleotide can be only a minor fraction of a complex mixture such as a biological sample.

The phrases “nucleic acid” or “nucleic acid sequence,” as used herein, refer to a nucleotide, oligonucleotide, polynucleotide, or any fragment thereof. These phrases also refer to DNA or RNA of genomic or synthetic origin which may be single-stranded or double-stranded and may represent the sense or the antisense strand, to peptide nucleic acid (PNA), or to any DNA-like or RNA-like material. In this context, “fragment” often refers to those shorter nucleic acid sequences containing a specific region or loci of interest.

As used herein, the term “hybridize” refers to the process by which single strands of polynucleotides form a double-stranded structure through hydrogen bonding between the constituent bases. The ability of two polynucleotides to hybridize with each other is based on the degree of complementarity of the two polynucleotides, which in turn is based on the fraction of matched complementary nucleotide pairs. The more nucleotides in a given polynucleotide that are complementary to another polynucleotide, the more stringent the conditions can be for hybridization and the more specific will be the binding between the two polynucleotides. Increased stringency may be achieved by elevating the temperature, increasing the ratio of co-solvents, lowering the salt concentration, and combinations thereof.

As used herein, the terms “complementary,” “complement,” and “complementary nucleic acid sequence” refer to the nucleic acid strand that is related to the base sequence in another nucleic acid strand by the Watson-Crick base-pairing rules. In general, two polynucleotides are complementary when one polynucleotide can bind another polynucleotide in an anti-parallel sense wherein the 3′-end of each polynucleotide binds to the 5′-end of the other polynucleotide and each A, T(U), G, and C of one polynucleotide is then aligned with a T(U), A, C, and G, respectively, of the other polynucleotide. Polynucleotides that comprise RNA bases can also include complementary G/U or U/G basepairs. Two complementary strands may comprise complementary regions comprising all or one or more portions of one or both strands.

As used herein, the term “kmer” refers to a polynucleotide of length k. In some embodiments, k is an integer from 1 to 1000. In some embodiments, k is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 125, 150, 175, 200, 250, 300, 400, 500, 600, 700, 800, 900, or 1000.

As used herein, the term “organism” refers to any prokaryote, eukaryote, archaea, fungi or bacteria.

Given the limited microarray real estate, a computational pipeline was developed that would make maximum use of a microarray while still enabling concurrent detection of as many antibiotic resistance elements as possible in a list of our top 24 pathogens (Table 1) or 44 pathogens (Table 4 or Table 7).

In some embodiments, a system of the invention comprises an array targeting the 24 nosocomial pathogens shown here in Table 1. In other embodiments, the array targeting 24 or more pathogens. And in another embodiment, the array targeting 44 or more organisms, the 44 organisms identified in Table 4 or 7 infra.

TABLE 1
Targeted 24 common nosocomial pathogens

| | |
|---|---|
| Acinetobacter baumannii | Bordetella pertussis |
| Burkholderia cenocepacia | Citrobacter freundii |
| Clostridium difficile | Enterobacter aerogenes |
| Enterobacter cloacae | Enterococcus faecalis |
| Enterococccus faecium | Escherichia coli |
| Haemophilus influenzae | Klebsiella oxytoca |
| Klebsiella pneumoniae | Mycobacterium tuberculosis |
| Neisseria meningitidis | Proteus mirabilis |
| Pseudomonas aeruginosa | Serratia marcescens |
| Stenotrophomonas maltophilia | Staphylococcus aureus |
| Staphylococcus epidermidis | Streptococcus pneumoniae |
| Streptococcus viridans | Streptococcus pyogenes |

The biological mechanisms underlying antibiotic resistance are diverse and complex. Moreover, partly due to varying selective pressures, for specific mechanisms strong biases might exist towards some of these pathogens. An extensive list of genes related to antibiotic resistance was compiled and reviewed.

To represent the most recent sequence information, we mined public sequence databases. Such an approach gives a huge number of sequences. To be useful as a basis for microarray design, the diversity of these sequences was quantified, each sequence annotated, and the sensitivity of different sequence candidates to be put on the microarray was evaluated for sensitivity and specificity.

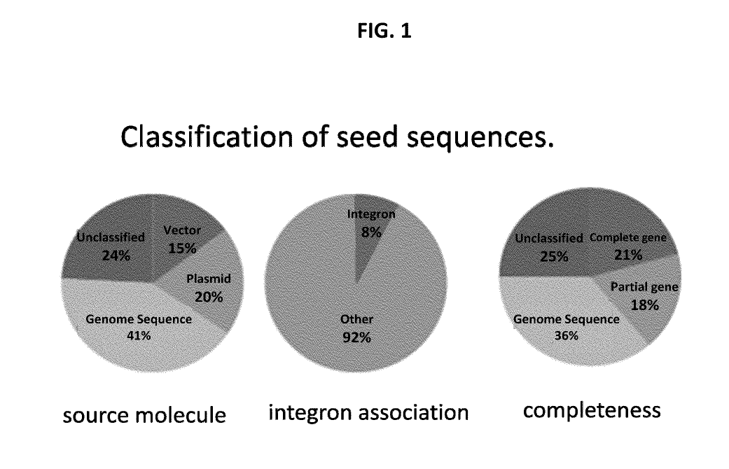

A schematic or flowchart showing the computational pipeline for the present design of the array is shown in FIG. 1. Antibiotic resistance element gene sequences representing current information on antimicrobial resistance were compiled, and a two-step computational pipeline was built. First, Genbank nr database was searched using Blast to obtain orthologs and all known sequence variants, using a lenient BLAST E-value cutoff (10⁻³) to increase sensitivity as well as to assess cross-hybridization potential, followed by sequence clustering.

We initially compiled 22,413 sequences spanning 642 species and 145 antibiotics. Using this sequence set as a “seed”, a total of more than 1 million sequences were harvested from Genbank nr database representing 1392 sequence clusters. Clusters were annotated by antibiotic resistance element, antibiotic resistance mechanism, and host, and cluster sequence representatives were chosen for downstream probe design.

The antibiotic resistance elements clustered generally into the following categories but are not limited to these
Aminoglycoside Resistance: acetylation (aac), phosphorylation (aph), adenylation (ant)
Beta Lactams: Class A, Class B, Class C, Class D
Macrolide Resistance: rRNA methylases (erm), ATP-binding transporters (ABC), major facilitator family transporters, esterases, hydrolases, transferases, phosphorylases
Multidrug Transporters: major facilitator superfamily transporter, ATP-binding cassette transporter, resistance nodulation cell division transporter
Tetracycline Resistance: efflux resistance, ribosome protection resistance
Vancomycin Resistance: VanA, VanB, VanC, VanD, VanE, VAnG type operons
Toxin genes that code for Toxin A and B proteins (Clostridium Difficile)

The resulting antibiotic resistance element sequences clustered into 1392 clusters according to the various biological mechanisms as detailed in Table 2.

In one embodiment, these seed sequences were used to identify similar sequences found in publicly available databases such as GenBank, and probes were made from those identified sequences. Therefore, in some embodiments, an array system can be fabricated for identification and detection of pathogens listed in Table 1, Table 4 and Table 7 and antibiotic resistance (AR) element sequences shown in Table 5 and listed herein.

Other embodiments provide a method for selecting and/or utilizing a set of oligonucleotide probes for use in an analysis system or bead multiplex system for simultaneously detecting a plurality of organisms in a sample and determining the antibiotic resistance element profile of the organisms in a sample. The method targets known diversity within target nucleic acid molecules to determine if a pathogen or a specific strain is present in the microbial community in the sample and what antibiotic resistance element(s) to which it is associated. In some embodiments, the organism-specific oligonucleotide probes may be used to also establish the antibiotic resistance phenotype. In other embodiments, the array comprises a set of resequencing probes covering antibiotic resistance elements and a set of resequencing probes covering gene(s) that are organism-specific. In some embodiments, this latter set of probes can provide strain-level identification of organisms present in a sample.

Examples of genes for strain-level identification include but are not limited to, 16S rRNA gene, CARDS toxin gene, toxic shock syndrome toxin-1 (tst) gene, adhP gene, adk gene, alpha toxin gene, aroE gene, aspartic semialdehyde dehydrogenase (asd) gene, atl gene, atp operon, cdt (cytolethal distending toxin) toxin gene, cep (cell envelope proteinase) gene, copper resistance (cop) gene, chaperonin cpn60 gene, cyaA gene, eae gene, etk gene, etx gene (epsilon-toxin inducer), folP gene, fumarase (fumC) gene, ferric update regulator (fur) gene, gapdh dehydrogenase gene, gdh dehydrogenase gene, glucose kinase gene, citrate synthase (gltA) gene, gro operon genes, glutamine transport protein (gtr) gene, glycosyltransferase (gttB) gene, IMP dehydrogenase gua gene, DNA gyrases, adhesin gene, gnd gene, groEL gene, hsp65 gene, carboxylesterase (lipT) gene, pepB gene, hsp60 and hsp65 genes, isocitrate dehydrogenase gene, icmB gene, immunity factor for SPN gene, infB gene, katG gene, lepC gene, lgt genes, elt operons, lukPVS and lukPVF genes, mcaP gene, malate dehydrogenase gene, macrophage infectivity potentiator (mip) gene, mitilysin gene, major outer membrane protein (mompS) gene, mismatch repair protein genes, MviN gene, NAD+glycohydrolase (spn) gene, (di)nucleoside polyphosphate hydrolase gene, outer membrane protein II (ompA) gene, catalase-peroxidase (hydroperoxidase I) gene, glucose-6-phosphate isomerase (pgi) gene, pheS gene, plr gene, pneumolysin (ply) gene, pyrazinamidase/nicotinamidas pncA gene, pertactin (prn) gene, zinc metalloproteinase precursor (proA) gene, PtxS1 gene, pyruvate kinase (pykF) gene, recA gene, ribonuclease PH gene, rpoB gene, sctC gene, SCO1/SenC family protein gene, smeZ gene, sodA gene, speA gene, STX transposon-like element and pilus assembly region, tcf gene, tkt gene, tonB-dependent receptor gene, toxA gene, trpA gene, tuf gene, ubiquitous surface protein (usp) gene, or Xanthine phosphoribosyltransferase (xpt) gene.

In another embodiment, the elements whose role in antibiotic resistance are probed are promoter regions of genes. These genes include, but are not limited to, genes such as ethA, embA.

In another embodiment, the antibiotic resistance elements are single nucleotide polymorphisms of specific genes. These genes include, but are not limited to, genes such as 16S rRNA, 23S rRNA, ahpC, alr, embA, embB, embC, ethA, fabG1, folP, gyrA, gyrB, kasA, katG, ndh, parC, parE, pncA, rlmN, rpoB, and rpsL. In some embodiments, target probes directed to detection of these SNPs may be also be designed and provided in addition to the target sequences to detect organisms at the species and/or strain levels.

The oligonucleotide probes can each be from about 5 bp to about 100 bp, preferably from about 10 bp to about 50 bp, more preferably from about 15 bp to about 35 bp, even more preferably from about 20 bp to about 30 bp. In some embodiments, the probes may be 5-mers, 6-mers, 7-mers, 8-mers, 9-mers, 10-mers, 11-mers, 12-mers, 13-mers, 14-mers, 15-mers, 16-mers, 17-mers, 18-mers, 19-mers, 20-mers, 21-mers, 22-mers, 23-mers, 24-mers, 25-mers, 26-mers, 27-mers, 28-mers, 29-mers, 30-mers, 31-mers, 32-mers, 33-mers, 34-mers, 35-mers, 36-mers, 37-mers, 38-mers, 39-mers, 40-mers, 41-mers, 42-mers, 43-mers, 44-mers, 45-mers, 46-mers, 47-mers, 48-mers, 49-mers, 50-mers, 51-mers, 52-mers, 53-mers, 54-mers, 55-mers, 56-mers, 57-mers, 58-mers, 59-mers, 60-mers, 61-mers, 62-mers, 63-mers, 64-mers, 65-mers, 66-mers, 67-mers, 68-mers, 69-mers, 70-mers, 71-mers, 72-mers, 73-mers, 74-mers, 75-mers, 76-mers, 77-mers, 78-mers, 79-mers, 80-mers, 81-mers, 82-mers, 83-mers, 84-mers, 85-mers, 86-mers, 87-mers, 88-mers, 89-mers, 90-mers, 91-mers, 92-mers, 93-mers, 94-mers, 95-mers, 96-mers, 97-mers, 98-mers, 99-mers, 100-mers or combinations thereof.

In some embodiments, the chosen oligonucleotide probes can then be synthesized by any available method in the art. Some examples of suitable methods include printing with fine-pointed pins onto glass slides, photolithography using pre-made masks, photolithography using dynamic micromirror devices, ink jet printing or electrochemistry. In one example, a photolithographic method can be used to directly synthesize the chosen oligonucleotide probes onto a surface. Suitable examples for the surface include glass, plastic, silicon and any other surface available in the art. In certain examples, the oligonucleotide probes can be synthesized on a glass surface at an approximate density of from about 1,000 probes per μm² to about 100,000 probes per μm², preferably from about 2000 probes per μm² to about 50,000 probes per μm², more preferably from about 5000 probes per μm² to about 20,000 probes per μm². In one example, the density of the probes is about 10,000 probes per μm². The array can then be arranged in any configuration, such as, for example, a square grid of rows and columns. Some areas of the array can be pathogen or organism identification or classification, and others can be used for image orientation, normalization controls or other analyses. In some embodiments, materials for fabricating the array can be obtained from Affymetrix, GE Healthcare (Little Chalfont, Buckinghamshire, United Kingdom), Agilent Technologies (Palo Alto, Calif.), or TessArae (Potomac Falls, Va.).

In one embodiment, the genomic sequence of each antibiotic resistance element is queried by 8, 25-mer i.e. 25 nucleotides in length, probes per base position in the sequence to be detected. For example, if the element is 100 base pairs long and the element is probed with 8 probes per position, then there will be 800 probes to identify that element. Thus, 8 short probes are designed for each base pair in the sequence for each identification or antibiotic resistance element. In certain examples, oligonucleotide fragments are used as probes.

In another embodiment, the presently provided target loci identified may be used for identification and detection of organisms and antibiotic resistance. In some embodiments, short oligonucleotide probes can be designed to cover the entire sequence of each antibiotic resistance element found in the 1392 clusters in Table 2. In such an embodiment, probes can be designed using the target loci sequences of SEQ ID NOS:1-1323 by one having skill in the art.

The target loci are provided herein as SEQ ID NOS: 1-1323, and consist of seed sequences of antibiotic resistance elements (SEQ ID NOS:1-970), organism-specific genes for organism-identification (SEQ ID NOS:971-1232), and regions in genes having antibiotic resistance relevance where a single nucleotide polymorphism is found (SEQ ID NOS:1233-1323).

In some embodiments, oligonucleotide primers can be designed to amplify fragments, specific regions or the entire sequence of each gene or target loci. The primers can be of varying lengths. Primers can be created using the nucleotide sequences of SEQ ID NOS: 1-1323, or surrounding genomic sequence containing and flanking any organism-specific gene or sequence or antibiotic resistance element, for sequence amplification. As is known in the art, primers or oligonucleotides are generally 15-40 bp in length, and usually flank unique sequence that can be amplified by methods such as polymerase chain reaction (PCR) or reverse transcriptase PCR. For all PCR-based methods, primers may be designed using commercially available software, such as OLIGO 4.06 primer analysis software (National Biosciences Inc., Plymouth, Minn.) or another appropriate program, to be, for example, about 19- to 30, or about 22 to 30 nucleotides in length, and in some embodiments, to have a GC content of about 50% or more, and/or to anneal to the template at temperatures of about 68° C. to 72° C.

Methods for design of detector tiles, selection of primers, and configuration of multiplex amplification protocols for the assay are known in the art and also described in U.S. Pat. Nos. 7,979,446; 7,695,941; 7,668,664; and 7,623,997, all of which are hereby incorporated by reference in their entirety.

In one embodiment, the methods for designing suitable probes and methods of fabricating a system herein are as described in International application publication Nos. WO 2010/151842 and in WO 2011/046614, both hereby incorporated by reference in their entireties.

Some embodiments relate to a method of designing or fabricating an array system including identifying antibiotic resistance element sequences corresponding to a plurality of organisms of interest, selecting fragments of antibiotic resistance element and other sequences unique to each organism and creating variant DNA fragments corresponding to the fragments of antibiotic resistance element and, optionally, the sequences unique to each organism and then fabricating the array system.

Non-limiting examples of arrays include microarrays, bead arrays, through-hole arrays, well arrays, and other arrays known in the art suitable for use in hybridizing probes to targets. Arrays can be arranged in any appropriate configuration, such as, for example, a grid of rows and columns. Some areas of an array comprise the detection probes whereas other areas can be used for image orientation, normalization controls, signal scaling, noise reduction processing, or other analyses. Control probes can be placed in any location in the array, including along the perimeter of the array, diagonally across the array, in alternating sections or randomly. In some embodiments, the control probes on the array comprise probe pairs of PM and MM probes. The number of control probes can vary, but typically the number of control probes on the array range from 1 to about 500,000. In some embodiments, at least 10, 100, 500, 1,000, 5,000, 10,000, 25,000, 50,000, 100,000, 250,000 or 500,000 control probes are present. When control probe pairs are used, the probe pairs will range from 1 to about 250,000 pairs. In some embodiments, at least 5, 50, 250, 500, 2,500, 5,000, 12,500, 25,000, 50,000, 125,000 or 250,000 control probe pairs are present. The arrays can have other components besides the probes, such as linkers attaching the probes to a support. In some embodiments, materials for fabricating the array can be obtained from Affymetrix (Santa Clara, Calif.), GE Healthcare (Little Chalfont, Buckinghamshire, United Kingdom) or Agilent Technologies (Palo Alto, Calif.).

In some embodiments, selected oligonucleotide probes are synthesized by any relevant method known in the art. Some examples of suitable methods include printing with fine-pointed pins onto glass slides, photolithography using pre-made masks, photolithography using dynamic micromirror devices, ink jet printing, or electrochemistry. In one example, a photolithographic method can be used to directly synthesize the chosen oligonucleotide probes onto a surface. Suitable examples for the surface include glass, plastic, silicon and any other surface available in the art. In certain examples, the oligonucleotide probes can be synthesized on a glass surface at an approximate density from about 1,000 probes per μm² to about 100,000 probes per μm², preferably from about 2000 probes per μm² to about 50,000 probes per μm², more preferably from about 5000 probes per μm² to about 20,000 probes per μm². In one example, the density of the probes is about 10,000 probes per μm². The number of probes on the array can be quite large e.g., at least 10⁵, 10⁶, 10⁷, 10⁸ or 10⁹ probes per array.

In one embodiment, the target probes selected and/or utilized by the methodologies of the invention can be organized into tiled sequences that provide an assay with a sensitivity and/or specificity of more than 80%, 85%, 90%, 91%, 92%, 93%, 94%, 95%, 96%, 97%, 98% or 99%. In some embodiments, sensitivity and specificity depends on the hybridization signal strength, number of probes in the tiled sequence, the number of potential cross hybridization reactions, the signal strength of the mismatch probes, if present, background noise, or combinations thereof. In some embodiments, an AR target sequence containing one probe may provide an assay with a sensitivity and specificity of at least 90%, while another target sequence may require at least 20 probes to provide an assay with sensitivity and specificity of at least 90%.

Besides arrays where probes are attached to the array substrate, numerous other technologies may be employed in the disclosed system for the practice of the methods of the invention. In one embodiment, the probes are attached to beads that are then placed on an array as disclosed by Ng et al. (Ng et al. A spatially addressable bead-based biosensor for simple and rapid DNA detection. Biosensors & Bioelectronics, 23:803-810, 2008).

In another embodiment, probes are attached to beads or microspheres, the hybridization reactions are performed in solution, and then the beads are analyzed by flow cytometry, as exemplified by the Luminex multiplexed assay system. In this analysis system, homogeneous bead subsets, each with beads that are tagged or labeled with a plurality of identical probes, are combined to produce a pooled bead set that is hybridized with a sample and then analyzed in real time with flow cytometry, as disclosed in U.S. Pat. No. 6,524,793. Bead subsets can be distinguished from each other by variations in the tags or labels, e.g., using variability in laser excitable dye content.

In a further embodiment, probes are attached to cylindrical glass microbeads as exemplified by the Illumina Veracode multiplexed assay system. Here, subsets of microbeads embedded with identical digital holographic elements are used to create unique subsets of probe-labeled microbeads. After hybridization, the microbeads are excited by laser light and the microbead code and probe label are read in real time multiplex assay.

In another embodiment, a solution based assay system is employed as exemplified by the NanoString nCounter Analysis System (Geiss G et al. Direct multiplexed measurement of gene expression with color-coded probe pairs. Nature Biotech. 26:317-325, 2008). With this methodology, a sample is mixed with a solution of reporter probes that recognize unique sequences and capture probes that allow the complexes formed between the nucleic acids in the sample and the reporter probes to be immobilized on a solid surface for data collection. Each reporter probe is color-coded and is detected through fluorescence.

In a further embodiment, branched DNA technology, as exemplified by Panomics QuantiGene Plex 2.0 assay system, is used. Branched DNA technology comprises a sandwich nucleic acid hybridization assay for RNA detection and quantification that amplifies the reporter signal rather than the sequence. By measuring the RNA at the sample source, the assay avoids variations or errors inherent to extraction and amplification of target polynucleotides. The QuantiGene Plex technology can be combined with multiplex bead based assay system such as the Luminex system described above to enable simultaneous quantification of multiple RNA targets directly from whole cells or purified RNA preparations.

In one embodiment, the microarray is a resequencing microarray. The resequencing microarray design had two main criteria: (1) to cover the maximum number of sequence variants currently known to be associated with antibiotic resistance and (2) to assure that the chosen sequences are suitable for simultaneous detection of a wide range of genes from multiple organisms and their antimicrobial resistance determinants. In some embodiments, probes are tiled to cover each nucleotide position in every locus to be interrogated, thus enabling not only accurate detection of the selected nosocomial pathogens but also any new variants of these organisms that may emerge.

In some embodiments, the array system uses multiple probes for increasing confidence of identification of a particular organism using an antibiotic resistance gene targeted high density microarray. The use of multiple probes can greatly increase the confidence level of a match to a particular pathogen. Also, in some embodiments, mismatch control probes corresponding to each perfect match probe can be used to further increase confidence of sequence-specific hybridization of a target to a probe. Probes with a mismatch at the 13^{th} nucleotide can be used to indicate non-specific binding and a likely non-match to the sequence of that probe at that nucleotide position.

Arrays and methods of making and using phylogenetic arrays, resequencing arrays and preparing samples are known in the art and are also described in U.S. Pat. Nos. 7,623,997; 7,668,664; 7,961,323; 7,979,446; U.S. Application Publication No. 20070212718 and 20110039710, all of which are hereby incorporated by reference in their entireties for all purposes, and also described in Wang, Z., Daum, L. T., Vora, G. J., Metzgar, D., Walter, E. A., Canas, L. C., Malanoski, A. P., Lin, B. and Stenger, D. A. (2006) Identifying Influenza Viruses with Resequencing Microarrays. Emerg Infect Dis, 12, 638-646; Lin, B., Wang, Z., Vora, G. J., Thornton, J. A., Schnur, J. M., Thach, D. C., Blaney, K. M., Ligler, A. G., Malanoski, A. P., Santiago, J. et al. (2006) Broad-spectrum respiratory tract pathogen identification using resequencing DNA microarrays. Genome Res. 16:527-535, and Davignon, L., Walter, E. A., Mueller, K. M., Barrozo, C. P., Stenger, D. A. and Lin, B. (2005) Use of resequencing oligonucleotide microarrays for identification of Streptococcus pyogenes and associated antibiotic resistance determinants. J Clin Microbiol, 43, 5690-5695; Wilson, W. J., Strout, C. L., DeSantis, T. Z., Stilwell, J. L., Carrano, A. V. and Andersen, G. L. (2002) Sequence-specific identification of 18 pathogenic microorganisms using microarray technology. Mol Cell Probes, 16, 119-127; Wilson, K. H., Wilson, W. J., Radosevich, J. L., DeSantis, T. Z., Viswanathan, V. S., Kuczmarski, T. A. and Andersen, G. L. (2002) High-density microarray of small-subunit ribosomal DNA probes. Appl Environ Microbiol, 68, 2535-2541; Zwick, M. E., McAfee, F., Cutler, D. J., Read, T. D., Ravel, J., Bowman, G. R., Galloway, D. R. and Mateczun, A. (2005) Microarray-based resequencing of multiple Bacillus anthracis isolates. Genome Biol, 6, R10; Wong, C. W., Albert, T. J., Vega, V. B., Norton, J. E., Cutler, D. J., Richmond, T. A., Stanton, L. W., Liu, E. T. and Miller, L. D. (2004) Tracking the evolution of the SARS coronavirus using high-throughput, high-density resequencing arrays. Genome Res, 14, 398-405; Sulaiman, I. M., Liu, X., Frace, M., Sulaiman, N., Olsen-Rasmussen, M., Neuhaus, E., Rota, P. A. and Wohlhueter, R. M. (2006) Evaluation of affymetrix severe acute respiratory syndrome resequencing GeneChips in characterization of the genomes of two strains of coronavirus infecting humans. Appl Environ Microbiol, 72, 207-211; and Hacia, J. G. (1999) Resequencing and mutational analysis using oligonucleotide microarrays. Nat Genet, 21, 42-47, all of which are hereby incorporated by reference for all purposes.

As used herein, a “sample” is from any source, including, but not limited to a biological sample, a gas sample, a fluid sample, a solid sample, or any mixture thereof.

Other methods of sample processing and handling to extract DNA from a sample are known in the art. See Nylund L, Heilig H G, Salminen S, de Vos W M, Satokari R, “Semi-automated extraction of microbial DNA from feces for qPCR and phylogenetic microarray analysis”, J Microbiol Methods. 2010 November; 83(2):231-5. Epub 2010 Sep. 16; Yu and Morrison, Improved extraction of PCR-quality community DNA from digesta and fecal samples, Biotechniques 2004 May; 36(5):808-12; Verollet R, A major step towards efficient sample preparation with bead-beating, Biotechniques. 2008 May; 44(6):832-3, all of which are hereby incorporated by reference in their entirety.

In one embodiment, a novel culture-independent resequencing microarray diagnostic and methods capable of strain-level identification across a panel of clinically-relevant nosocomial pathogens and also supporting prediction of their antibiotic resistance phenotype. In some embodiments, the diagnostic and methods can provide strain-level identification within 48, 24, 18, 12, or 8 hours. In one embodiment, the diagnostic and methods provide strain-level-identification in 8 hours.

Another advantage of the present embodiments is that simultaneous detection of a majority of currently known top pathogens and the type of antibiotic resistance element is possible with one sample. While achieving the results from each of these tests in parallel, the present resequencing microarray system should prove to be more cost effective, provide faster results, and greater sensitivity. This allows for much more efficient study and determination of particular organisms within a particular sample. Current microarrays do not have this capability.

In some embodiments, the samples used can be environmental samples from any environmental source, for example, naturally occurring or artificial atmosphere, water systems, soil or any other sample of interest. In some embodiments, the samples may be obtained from, for example, atmospheric pathogen collection systems, manufacturing plants involved in food preparation or handling, hospital or clinic exam rooms and surfaces, etc. In a preferred embodiment, the array system of the present embodiments can be used in any environment.

In other embodiments, the sample used with the array system can be any kind of clinical or medical sample. In one embodiment, the clinical sample comprises at least one of tissue, skin, stool, bodily fluid, or blood. Tissues can include biopsied cells from various organs or systems including but not limited to, lungs, throat, esophagus, stomach, intestinal, bone, spinal, brain, skin, etc. For example, samples from blood, the lungs or the gastrointestinal tract of mammals may be assayed using the array system. Also, the array system of the present embodiments can be used to identify an infection in the blood of an animal. The array system of the present embodiments can also be used to assay medical samples that are directly or indirectly exposed to the outside of the body, such as the lungs, ear, nose, throat, the entirety of the digestive system or the skin of a patient or an animal.

More specifically, the DNA (also referred to as “nucleic acid”) for use in the present invention may be obtained by collecting a biological sample from a patient and subsequently directly applying the sample to further microarray analysis or extracting (i.e., isolating or substantially purifying) the DNA from the sample for further analysis. In this regard, the biological sample includes, but are not limited to, peripheral blood; exfoliated cells, including cells obtained from a buccal swab, stool, sputum, nasal wash, nasal aspirate, nasal swab, throat swab, bronchial lavage, vaginal swab, and rectal swab; blood, blood cells (e.g., white cells), tissue or fine needle biopsy samples, urine, peritoneal fluid, visceral fluid, and pleural fluid, or cells therefrom.

Further, the present invention is not limited to biological samples obtained from humans. The present invention may also be applied to biological samples obtained from any animal species including domestic and/or farm animals including, but not limited to: dogs, cats, horses, cows, pigs, goats, sheep, rabbits, mice, rats, etc. In addition, the present invention may also be applied to biological samples obtained from any animal species that may be found in the wild or traditionally thought of as zoological animals, for example: monkeys, giraffes, elephants, zebras, tigers, lions, lemurs, etc. Further, the present invention may also be applied to biological samples obtained from any avian species. In this embodiment it is understood that the gene targets embedded on the microarray chip to be detected would contain the genes for the respective species selected.

Further, the sample of the present invention is not limited to biological samples, the sample of the present invention may be environmental (air, water, soil, etc.), animal (see above), or plant (e.g., cells obtained from any portion of a plant where the species of plant is without limit). Again, in this embodiment it is understood that the gene targets embedded on the microarray chip to be detected would contain the genes for the respective species selected, when that species is known (i.e., animal or plant). Further, when the sample is environmental, the gene targets embedded on the microarray chip can be any predetermined collection that is used to detect and identify any pathogen or organism of interest, for example.

In some embodiments, the sample is a processed or unprocessed food product. In other aspects, the food sample comprises at least one of meat, turkey, chicken and other poultry, milk, eggs, eggs products, dairy products, fresh or dried fruits and vegetables and their juices, grains, fish, seafood, pet food, baby food and infant formula.

In another embodiment, the sample is a bacterial isolate, to confirm or identify the strain and its antibacterial resistance profile.

It is anticipated that the organism-specific and antibiotic resistance (AR) probes in an array system can be used as a clinical diagnostics tool in hospitals, to aid in epidemiological research and tracking as well as for infection control. In addition, current methodology can be applied to redesign a new microarray containing different antibiotic resistance genes and organisms geared towards other uses. These would include but not limited to biodefense, product and food safety and assessment of air, soil and water quality.

Some embodiments relate to methods of detecting an organism in a sample using a resequencing array system. These methods include contacting a sample with one organism or a plurality of organisms to the array system of the present embodiments and detecting the organism or organisms. In some embodiments, the organism or organisms to be detected are bacteria. In some embodiments, the antibiotic resistance elements detected represent new or emerging resistance in the organism or organisms detected in the sample.

Sample processing for Affymetrix platform microarrays has already been extensively validated, however as one of the primary goals is to produce a rapid diagnostic tool, optimization for time to result will require some re-focusing of existing protocols. Affymetrix microarrays are now being used in clinical laboratories, demonstrating their potential as a rapid diagnostic. In one embodiment, a processing method combining an automated robotic DNA/RNA extraction system with a streamlined resequencing microarray protocol to rapidly provide high quality material for analysis allowing for high sensitivity, accuracy and reproducibility in detecting and profiling antibiotic resistance phenotypes of select nosocomial pathogens. In another embodiment, a random DNA amplification approach may be used to provide the sensitivity necessary to be comparable with current clinical culture methods.

In one embodiment, the assay for clinical sample processing including a pre-treatment process comprising longer periods of bead beating to increase the amount of DNA extracted. Typically 30 s of bead beating are used to extract DNA, however periods of 45 s, 60 s, 90 s and 120 s of bead beating was found to be effective in increasing the amount of DNA extracted particularly in samples of Gram positive organisms, without sacrificing the integrity of DNA extracted from Gram negative species in the same sample. This approach has been compared to enzymatic lysis methods and found to be more rapid and efficacious in retrieval of nucleic acids from samples.

In another embodiment, a protein such as bovine serum albumin (BSA) is added to the sample (e.g., extracted nucleic acid sample) to decrease PCR inhibition in blood samples and further enhance the sensitivity of detection. In some embodiments, the protein is added to the sample prior to extended bead beating period. In one embodiment, the protein is added in amounts of 80 to 100 CFU per mL of sample.

In another embodiment, the pre-treatment method further comprising cell lysis methods using enzymatic lysis of bacterial cells, such as in the case of Gram positive organisms that are difficult to lyse due to a thick peptidoglycan cell wall. Enzymes such as lysostaphin may be added to the clinical sample and processed.

Thus, in some embodiments, an assay and methods for clinical sample processing is described. Such a method comprising steps of (1) collecting a sample from a patient; (2) adding sample to beads; (3) performing bead beating of said sample for 45 to 120 seconds to extract DNA from the sample and (4) adding 80 to 100 U mL⁻¹ of bovine serum albumin to the PCR reaction with nucleic acids from said sample as DNA template.

In some embodiments, identification of organisms will define antibiotic treatment regime. For example if a Gram-positive organism is determined to the primary pathogen, the patient would receive a gram-positive appropriate antibiotic such as vancomycin. In addition, if the assay determines that a specific organism is present that possesses resistance determinants for a number of antibiotics, these therapeutic options would be avoided for that particular patient.

Depending on the culture source, organism, and clinical microbiology laboratory practices, final identification and susceptibility results are usually not available for 48 to 72 hours; for some organisms (for example, fungi or Mycobacteria), susceptibility testing may take weeks or may never be available. This delay creates a window during which patients are exposed to the toxicity and selective pressure of unnecessary broad-spectrum antimicrobial therapy. Indeed, for less-severe infections there is the potential that final microbiology results will not be available before the patient is discharged, and the information may never be used to modify antimicrobial therapy. Thus the present invention further provides for a rapid detection and antibiotic susceptibility testing method to substantially reduce the lag time to appropriate antimicrobial therapy from days to hours, thus optimizing patient outcomes and preventing the development and dissemination of antibiotic-resistant organisms.

Standard methods for drug susceptibility testing can fail to identify the presence of important mechanisms of resistance that are associated with clinical failure, despite apparent phenotypic susceptibility. Examples of this phenomenon include extended-spectrum beta-lactamase production in Klebsiella species, ribosomal methylation in Staphylococci and Streptococci, and heterogenous vancomycin resistance in Staphylococci (See Lewis J S, 2nd, Jorgensen J H. Inducible clindamycin resistance in Staphylococci: should clinicians and microbiologists be concerned? Clin Infect Dis 2005; 40:280-5; Paterson D L, Ko W C, Von Gottberg A et al. Outcome of cephalosporin treatment for serious infections due to apparently susceptible organisms producing extended-spectrum beta-lactamases: implications for the clinical microbiology laboratory. J Clin Microbiol 2001; 39:2206-12; Liu C, Chambers H F. Staphylococcus aureus with heterogeneous resistance to vancomycin: epidemiology, clinical significance, and critical assessment of diagnostic methods. Antimicrob Agents Chemother 2003; 47:3040-5). In these cases, standard microbroth-based susceptibility techniques must be supplemented by additional phenotypic tests, further delaying the provision of important susceptibility data to the clinician. Rapid identification of organisms harboring these resistance characteristics could reduce the risk of application of inappropriate therapies.

Strain-specific virulence factors, such as those responsible for toxin production or biofilm formation, are increasingly recognized as potentially important contributors to clinical outcomes in infected patients. Recognition of strains possessing these factors has the potential to alter management through more aggressive surgical intervention, removal of prosthetic material (such as intravascular catheters), and use of agents targeting protein synthesis to prevent toxin production. There is growing interest in the development of pharmaceutical agents that block or modulate virulence factors (Marra A. Targeting virulence for antibacterial chemotherapy: identifying and characterising virulence factors for lead discovery. Drugs R D 2006; 7:1-16). However, current routine clinical microbiology laboratory techniques are insufficient to identify organisms possessing these factors. For example, a new strain of the intestinal pathogen Clostridium difficile producing a novel toxin and with an expanded antimicrobial resistance profile has emerged as a cause of severe colitis (McDonald L C, Killgore G E, Thompson A et al. An epidemic, toxin gene-variant strain of Clostridium difficile. N Engl J Med 2005; 353:2433-41; Loo V G, Poirier L, Miller M A et al. A predominantly clonal multi-institutional outbreak of Clostridium difficile-associated diarrhea with high morbidity and mortality. N Engl J Med 2005; 353:2442-9). Critically, this new strain co-exists with previous, less pathogenic strains making diagnosis more difficult. For example, using the presently described system and methods for identification of patients carrying this new strain could lead to a lower threshold for surgical intervention and more intensive and focused antimicrobial therapy.

Finally, routine clinical microbiologic laboratory techniques provided do not allow infection-control practitioners to identify relatedness of isolates from different patients. Isolate relatedness is useful in determining if an outbreak is occurring and in pinpointing its source. Current methods, using pulsed field gel electrophoresis are time consuming and do not typically provide meaningful results until after the outbreak has occurred. In some embodiments, the present invention provides for the ability to identify markers allowing strain differentiation of infecting pathogens in the course of routine microbiological workup and represent an extremely useful advance in technology from an infection-control standpoint.

Other applications of the presently described system and array is the monitoring of organisms and antibiotic resistant bacteria in food and beverage production, animal husbandry, water supplies, treated human waste that may be applied to agricultural land, etc.

Other applications of the presently described system and array is the monitoring of organisms modified for biological warfare.

### Example 1

### Genotypic and Phenotypic Analysis of a Collection of Over 600 Strains to be Used for Array Validation and Predictive Model Building

To date, we have collected at least 20 representative strains for 22 of 44 pathogenic bacterial species (Table 1, 3), generating a bank of 604 clinical isolates of which 567 strains are characterized with respect to their antibiotic resistance phenotype (determined by standard clinical laboratory techniques appropriate for that strain and antibiotic-susceptibility profile). On-going work includes characterization, in our CLIA-approved laboratory of the resistance profile of Haemophilus influenzae which is not routinely profiled for antibiotic resistance in the clinical lab. All strains have been successfully cultured under species-specific optimal culture conditions (aerobic and anaerobic). Stock cultures have been made, banked and cataloged for every strain and parallel DNA and RNA extractions have been performed for every strain, quantified, cataloged and stored at −80° C. We are currently a BSL II approved laboratory and as such are not permitted to work with viable cells of BSL III pathogens such as Neisseria meningitis and Mycobacterium tuberculosis. To ensure that we had representative strains of these species for array validation and model building, we obtained pathogen-free nucleic acid extracts from Drs. Joanna MacKichan (Environmental Science and Research, New Zealand) and Midori Kato-Maeda (San Francisco General Hospital). In addition, due to the limited species banked at the clinical laboratory at UCSF, we obtained additional strains of specific organisms from investigators who specifically study these species, including Drs. John LiPuma (Burkholderia cepacia), Tim Murphy (H. influenzae) and the California State Reference laboratory (Bordatella pertussis). A database, which we generated, has been populated with unique identifiers, antibiotic resistance phenotype and source of isolate (if available). This extensive collection of characterized strains (Table 3) will be made available to other researchers at the end of the study.

TABLE 3
Banked strains/samples to be used for array validation.

| | | Frozen | RNA & DNA |
|---|---|---|---|
| OrgCode | Species | stocks (n) | extracted |
| 1 | P. aeruginosa | 30 | 30 |
| 2 | E. coli | 30 | 30 |
| 3 | K. pneumonaie | 30 | 30 |
| 4 | K. oxytoca | 21 | 21 |
| 5 | E. cloacae | 30 | 30 |
| 6 | E. aerogenes | 20 | 20 |
| 7 | A. baumanii | 20 | 20 |
| 8 | S. marcescens | 31 | 31 |
| 9 | S. maltophilia | 30 | 30 |
| 10 | C. freundii | 20 | 20 |
| 11 | B. cepacia | 31 | 31 |
| 12 | P. mirabilis | 30 | 30 |
| 13 | H. influenzae | 37 | 37 |
| 14 | S. aureus | 30 | 30 |
| 15 | S. epidermidis | 30 | 30 |
| 16 | S. pneumonaie | 34 | 34 |
| 17 | S. viridans | 30 | 30 |
| 18 | E. faecium | 30 | 30 |
| 19 | E. faecalis | 30 | 30 |
| 20 | N. meningitides* | 0 | 30 |
| 21 | C. difficile** | 0 | 0 |
| 22 | M. tuberculosis* | 0 | 30 |
| | TOTAL | 544 | 604 |

*Pathogen-free DNA obtained from collaborators;
**Stool samples from clinical lab

The UCSF Clinical Microbiology Lab is in the process of collecting 80 clinical stool samples from patients with suspected Clostridium difficile-associated diarrhea. Nucleic acids will be extracted and used to determine C. difficile genotype at the loci chosen for the array-based assay, as well as validate the sensitivity and specificity of the oligonucleotide probes tiling this region on the array. With the addition of these stool samples, we will have a collection of DNA and RNA from 684 strains and samples, with parallel clinically determined, antibiotic resistance data. All data on these strains/extracts, nucleic acid concentration extracted, antibiotic phenotype has been inputted into a searchable database.

We have commenced genotyping of these strains using bi-directional Sanger sequencing of the 16S rRNA gene from each isolate. 16S rRNA gene sequencing has been performed for every strain in our collection. Contiguous sequences spanning the length of the entire 16S rRNA gene (˜1.5 Kb) have been generated for each strain and searched against existing publically available databases to determine the identity of each strain. We have performed agreement statistics on the data generated and demonstrated that of the 11 species and 380 strains genotyped to date, the kappa score is 0.87 with a statistically significance value of p<0.001. Comparative analysis of these sequences at the species level has demonstrated that the lack of agreement is primarily restricted to a small number of species, where clinical identification appears to be relatively inaccurate e.g. Enterobacter cloacae.

On-going work involves performing similar genotypic analysis on the target loci and whole genome sequencing of strains with poor representation in publically available databases prior to their use to validate the prototype resequencing array-based assay to complement the large phenotypic database of information we have generated for these strains.

We continue to enroll patients in our sepsis study and in our intravenous drug users with suspected endocarditis and to store blood collected from consented subjects in these studies for validation of the resequencing array-based assay. In addition we have collected and banked more than 300 cystic fibrosis sputum samples that will also be used to validate the assay. Through one of our other NIH-funded airway microbiome studies, we are also collecting HIV positive airway samples from San Francisco General Hospital, and Mulago Hospital in Kampala (high rate of Mycobacterium tuberculosis infection). A subset of these bronchoalveolar lavage samples that have been clinically examined by culture-based methods and by culture-independent approaches will also be used to validate the resequencing array-based assay.

### Example 2

### Optimization of Clinical Sample Processing

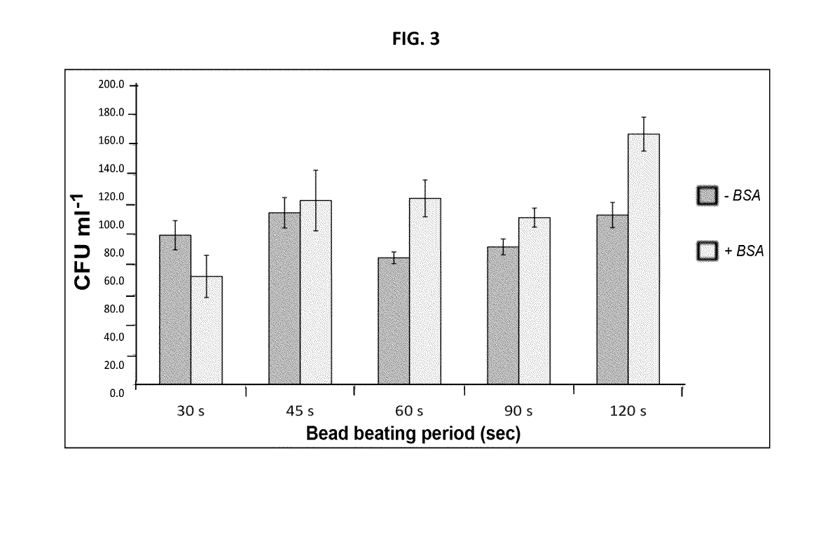

Building on our method for sample collection, handling and storage, we have continued to examine aspects of this protocol that may impact efficiency of the extraction protocol and, consequently the ability of the resequencing array-based assay to detect and profile strains. Further optimization of bacterial DNA extraction, yield, and PCR-based quantification from blood samples is being pursued. We have found that longer periods of bead beating increases the amount of DNA extracted by the Qiagen BioRobot method (an automated system for rapid extraction of nucleic acids from samples) particularly that of gram positive organisms, without sacrificing the integrity of DNA extracted from Gram negative species in the same sample (dark gray bars, FIG. 3). We have also found that the addition of bovine serum albumin [BSA] decreases PCR inhibition associated with blood samples and further enhances the sensitivity of detection. This improvement in detection upon the addition of BSA to the reaction is illustrated by Q-PCR amplification of the 16S rRNA gene of S. aureus across a range of bead-beating time periods (with the exception of 30 sec; light gray bars, compared with dark gray bars; FIG. 3). Further investigation of the use of bead beating plus enzymatic lysis of bacterial cells is being conducted to see if this affords higher extraction yields of bacterial DNA, especially in the case of gram positive organisms that are difficult to lyse due to a thick peptidoglycan cell wall.

### Example 3

### Design of Target Loci Oligonucleotides Probes

As described above, we compiled a comprehensive and solid set of antibiotic resistance elements and their associated annotations. Of the actual sequences tiled as detectors throughout the RPM-BAR 1.5 designed array, there are 3 classes: antibiotic resistance (AR) gene targets (SEQ ID NOS: 1-970), Organism/species identification (BUG-ID) gene targets (SEQ ID NOS: 971-1232), and SNP targets (SEQ ID NOS: 1233-1323) selected genes known to be associated with bacterial antibiotic resistance bearing mutations in those genes that are deemed determinants of resistance.

The AR-genes are consensus sequences of clusters of sequence-similar antibiotic resistance genes. The target loci in each of the sequence records was from a manually curated set and seeded from the University of Maryland ARDB (database of sequence records for AR genes) to identify in excess of 1 million sequence-similar sequence records from the entire GenBank. This large aggregate of sequence records was condensed first to about 16,000 consensus sequences of aligned records, using a constraint of approximately 98% sequence similarity, and another constraint that each cluster contain at least one record attributing its origin from one of the 22 target bacterial species.

The 16,000 clusters were further merged to about 1,600 by sequence alignments using less stringent constraint of 95% sequence similarity, and then purged of clusters representing a single outlier sequence, or of length less than about 400 bp. The length limit was to ensure that detector tiles of 200 to 224 bp length could be identified from each cluster's consensus sequence, leaving enough flanking sequences to the detector tile for selection of multiplex amplification primers.

The BUG-ID gene targets are selected genes for RPM array-based detection and identification of selected species. An initial list of targeted bacterial genera (genuses) was selected based on ongoing objectives and clinical requirements of the overall project.

The subset of GenBank sequence records attributed to the previously-selected target genera was surveyed. This extended to multiple species per genus, and extended to other genera from other TessArae RPM applications that were suggested to be candidates for inclusion in the RPM-BAR 1.5 assay.

We selected the final list of genera and species to be targeted in the assay and designed detector tiles of 224 bp lengths, following established assay design protocols. In general, alignments of sequence records for each target genus and for such genes represented in the database (GenBank) were aligned, and multiple gene targets were selected to represent multiple strains and variants of the target genus/species. In most cases the targeted genes represented housekeeping functions, not necessarily linked to antibacterial resistance mechanisms. When large numbers of different strains and variants contributed to a target organism gene cluster, multiple detector tiles were selected from sub-cohorts of sequence records based on sequence record similarity.

The SNP targets represent selected genes known to be associated with bacterial antibiotic resistance bearing mutations in those genes that are deemed determinants of resistance.

We then conducted: a thorough literature search which provided citations of particular mutations in particular genes of particular species, and these were aggregated to define regions of the target genes that could be targeted sequences of the application.

Following selection of target sequences, we then selected intervals spanning proximal mutations as re-sequencing detector tiles of variable lengths, for the purpose of evaluating sequences of targeted bacterial genes in selected specimens for determination of bacterial genotype (mutations known and previously unknown).

We have also created a prototype database of antibiotic resistance elements named BARChipDB. BARChipDB will be used to store three types of information: (1) annotated sequence clusters represented on the array, (2) related sequence clusters harvested from GenBank, and (3) clinical isolate metadata (antibiotic resistance phenotype, clinical source, geographic origin etc.). BARChipDB is implemented as a PostgreSQL relational database. The generic model organism database (GMOD) schema Chado was chosen to store sequence and annotations of the ˜16,000 sequence clusters. The main advantage of Chado is that it is driven by ontologies, which makes it highly flexible and generic. In addition, Chado SQL API has a library of SQL functions for efficiently performing useful operations on biological data. We are in the process of designing a second customized schema expanding Chado to organize the available experimental and in silico analyses data. We envision that BARChipDB will be instrumental in handling continuous updates of antibiotic resistance sequence diversity from GenBank, streamlining future assay updates, and facilitating array-based assay output interpretation.

### Example 3

### Prototype Array Design

We completed tiling probe sets for all candidate detector tiles, apportioned to Bacterial genera/species (262 tiles) and Resistance determinants (1030). The array platform we are using has the capacity to accommodate 1,345 tiles of up to 224 bp each, we therefore dramatically expanded the targets included on the array from 11 to 44 clinically relevant bacterial species and their associated antibiotic resistance determinants and are currently at 99.96% of the array capacity. These additional species were chosen based on their clear role in pathogenesis and their detection in a number of recent airway microbiome studies. The final list of all bacterial species to be detected by this tool are described in Table 4 or Table 7. These targets are represented by 262 tiles totaling 58,688 bp (of a possible 303,000 bp) on the array. The 262 tiled sequences are identified herein as SEQ ID NOs: 971-1232. The probes for each tile are the same as the sequence for each tile/target plus the complement of that sequence and 3 mismatch probes for each perfect match. Each base across that tile is covered by 8 probes on the resequencing array.

TABLE 4
Target species detected by RPM-BAR 1.5 Bact Antibiotic Res Panel.

Acinetobacter baumannii
Burkholderia ambifaria
Burkholderia cenocepacia
Burkholderia cepacia
Burkholderia dolosa
Burkholderia multivorans
Burkholderia pyrrocinia
Burkholderia stabilis
Burkholderia
vietnamiensis
Bordetella parapertussis
Bordetella pertussis
Clostridium difficile
Citrobacter freundii
Chlamydophila
pneumoniae
Chlamydia trachomatis
Enterobacter aerogenes
Enterobacter cloacae
Enterococcus faecalis
Enterococcus faecium
Escherichia coli
Haemophilus influenzae
Haemophilus
parainfluenzae
Klebsiella oxytoca
Klebsiella pneumoniae
Legionella pneumophila
Mycobacterium avium
hominissuis
Mycobacterium avium
paratuberculosis
Mycobacterium kansasii
Mycobacterium
tuberculosis
Moraxella catarrhalis
Mycoplasma pneumoniae
Neisseria meningitidis
Pseudomonas aeruginosa
Pseudomonas
pseudoalcaligenes
Proteus mirabilis
Proteus vulgaris
Staphylococcus aureus
Stenotrophomonas
maltophilia
Serratia marcescens
Streptococcus mitis
Streptococcus mutans
Streptococcus
pneumoniae
Streptococcus pyogenes
Streptococcus viridans

Resistance determinants are split into two categories—genes in which SNP accumulation leads to antibiotic resistance and genes for which their presence in the genome alone confers a resistance phenotype. Although the resequencing array will, by the nature of the data it generates (actual nucleotide sequence), will report novel SNPs detected in any target, those genes specifically linked to SNP-associated resistance targeted by the array-based assay are listed in Table 3. These targets are represented by 198 tiles, totaling 58,568 bp on the array. The regions of the targets are identified as SEQ ID NOS: 1233-1323.

TABLE 5
Antibiotic resistance SNP surveillance loci.

| | | | | | | |
|---|---|---|---|---|---|---|
| 16S rRNA | 23S rRNA | ahpC | alr | embA | embB | embC |
| ethA | fabG1 | folP | gyrA | gyrB | kasA | katG |
| ndh | parC | parE | pncA | rlmN | rpoB | rpsL |

Bacterial species known to house these antibiotic resistance genes and resistance-associated SNPs that are targeted by the resequencing array-based assay are listed in Table 6.

TABLE 6
Species housing known antibiotic resistance
gene SNPs targeted by the array

Acinetobacter
baumannii
Bordetella pertussis
Burkholderia cepacia
Citrobacter freundii
Clostridium difficile
Enterobacter aerogenes
Enterobacter cloacae
Enterococcus faecalis
Enterococcus faecium
Escherichia coli
Haemophilus influenzae
Klebsiella oxytoca
Klebsiella pneumoniae
Mycobacterium tuberculosis
Neisseria meningitidis
Proteus mirabilis
Pseudomonas aeruginosa
Serratia marcescens
Staphylococcus aureus
Stenotrophomonas maltophilia
Streptococcus pneumoniae
Streptococcus pyogenes

Finally, the remaining antibiotic resistance targets represent all other antibiotic resistance determinants and are covered on the array by 832 consensus sequencing tiles totaling 217,280 bp. In summary the novel resequencing array content includes detection of 44 clinically relevant bacterial species, 21 target genes with documented SNP-associated resistance and 16,000 clusters of antibiotic resistance determinants whose presence is associated with resistance to clinically relevant antimicrobial agents.

This system has major implications for early detection and antibiotic profiling of nosocomial pathogens. Several studies have demonstrated that early and appropriate antimicrobial administration significantly increases patient survival rates, and decreases length of hospital stay and associated health-care costs. This diagnostic, and particularly the expanded and more comprehensive array-based assay we are developing, will significantly decrease the time taken to diagnose patients.

Resequencing Array Fabrication.

We have fabricated a 49-format Affymetrix Custom Resequencing microarrays capable of sequencing a total of 350 kb. Each nucleotide base across the chosen loci (typically 224 bases) will be interrogated using four 25 mer probes for each strand differing only at the central 13^{th} nucleotide position where each of the four base possibilities (A, T, G, C) is represented. As validated for other Affymetrix microarrays, the probe that perfectly matches the base at that position in the target will have a significantly greater hybridization intensity thus producing a base call at the position. Not only will this enable detection of previously characterized strains and resistance genotypes but may also allow new variants to be detected. We believe this additional aspect will prove highly valuable in epidemiological studies where novel strains or novel antibiotic resistance variants may be detected.

### Example 4

### Sample Protocol for Resequencing Prototype Antibiotic Resistance Array

A sample assay protocol that may be used is similar to most other nucleic acid-based protocols, and contains several standard procedures. Beginning with a clinical or environmental specimen, the steps are: (a) Extraction of total nucleic acids (TNA) using one of several commercially available methods; (b) Reverse Transcription to convert any RNA to cDNA using random primers; (c) PCR endpoint amplification of pathogen-specific gene sequences using locus-specific, multiplexed primers; (d) Sample pooling and purification combines the multiplex PCR reactions, removes any residual primers, enzymes, salts, dNTPs, and concentrates the amplified DNA; (e) Fragmentation and labeling prepares the amplified targets to be hybridized to the microarray; (f) Hybridization, staining and washing allows for the detection of the target sequences on the microarray; (g) Internal Controls monitor the efficiency of amplification and hybridization steps.

An optimized assay protocol should combine the following:

Nucleic Acid Extraction Optimization.

Further optimization of bacterial DNA extraction, yield, and PCR-based quantification from blood samples is being pursued. We have found that longer periods of bead beating increases the amount of DNA extracted by the BioRobot method, particularly that of Gram positive organisms, without sacrificing the integrity of DNA extracted from Gram negative species in the same sample (orange bars, FIG. 3). We have also found that the addition of bovine serum albumin [BSA] decreases PCR inhibition associated with blood samples and further enhances the sensitivity of detection. This improvement in detection upon the addition of BSA to the reaction is illustrated by Q-PCR amplification of the 16S rRNA gene of S. aureus across a range of bead-beating time periods (with the exception of 30 sec; yellow bars, compared with orange bars; FIG. 3). Further investigation of the use of bead beating plus enzymatic lysis of bacterial cells is being conducted to see if this affords higher extraction yields of bacterial DNA, especially in the case of gram positive organisms that are difficult to lyse due to a thick peptidoglycan cell wall.

Target Amplification Optimization.

We will test random multiple displacement amplification (MDA) of bacterial DNA following subtraction of human DNA to determine the suitability of this target amplification method. This will be compared with a multi-locus PCR approach. Using the data obtained from our comparative genomics, we performed an initial demonstration of multiple pathogen detection capability. We developed the Multi-Pathogen Identification (MPID) microarray capable of detecting eighteen pathogenic viruses, prokaryotes, and eukaryotes, including several CDC Category A and B biothreat agents. Pathogens targeted are shown in Table 1 and Table 7.

Reduction and Optimization of Array Processing Time.

We will reduce the minimum times for template fragmentation, labeling, and hybridization and also the minimum times for array washing, staining and scanning so that the entire array process takes only 6 hours from receipt of template.

Analysis.

Both the resequencing and gene expression arrays are run on the GCS 3000 instrumentation system, Cartridge-based instrument system used in microarray applications. Standard cartridge-based array processing involves hybridization to an array, washing and staining, and scanning step. Analysis of array hybridization patterns will be used to generate a sequence readout of each locus according to an approach previously published by us (Metzgar D, Myers C A, Russell K L, Faix D, Blair P J, Brown J, Vo S, Swayne D E, Thomas C, Stenger D A, Lin B, Malanoski A P, Wang Z, Blaney K M, Long N C, Schnur J M, Saad M D, Borsuk L A, Lichanska A M, Lorence M C, Weslowski B, Schafer K O, Tibbetts C. Single assay for simultaneous detection and differential identification of human and avian influenza virus types, subtypes, and emergent variants. PLoS One. 2010 Feb. 3; 5 (2):e8995. PubMed PMID:20140251; PubMed Central PMCID: PMC2815781.). Each assay of a specimen generates sequence data as base calls (A, G, C, or T) across each detector tile of Affymetrix CustomSeq-formatted microarrays (see Affymetrix Technical Note, 2006, GeneChip CustomSeq Resequencing Array Base Calling Algorithm Version 2.0: Performance in Homozygous and Heterozygous SNP Detection). Image data acquired from GeneChip Instrument System scanning under GeneChip Operating Software control is translated by GSEQ sequence analysis software (Affymetrix Inc., Santa Clara, Calif.). TessArray Sequence Analysis (TSEQ) software evaluates a “C3 Score” for each array detector tile, as a metric of detected DNA sequence quantity and quality. The C3 Score is the total number of GSEQ-identified nucleotides that appear in runs of three or more consecutive (non-N) base calls, expressed as percentage of the length (nucleotides) of each array detector tile sequence. This approach to definition of a detected sequence reduces background noise from spurious false-positive hybridizations of 25-base oligonucleotide probes that could lead to isolated and relatively uninformative single base calls. Relaxed and stringent target organism detection and reporting thresholds for the assay can been statistically and empirically established for all of the resequencing detector tiles. All sequences from an assay that meet a detection and reporting threshold are automatically subjected to alignment-based search of the BARChipDB Reference Sequence Database, using a dedicated server-based implementation of the Basic Local Alignment Search Tool, BLAST.

Final confidence in identification of an organism species/strain or resistance element will comprise of a single ‘score’ reflecting a combination of probability scores from the BLAST scores of independent loci for each strain/resistant element.

### Example 5

### Validation Studies of Prototype Array

We are currently validating primer pairs which will be used in multiplex PCR reactions to amplify the target loci of interest. We confirm that each primer pair for identification of a specific species is capable of amplifying the target from DNA extracted from a known strain of that species (strain identification has been confirmed by sequence analysis). In the same manner, primer pairs to amplify specific target loci aimed at determining the presence or SNP content of specific antibiotic resistance determinants will be validated using strains known to encode those resistance determinants. We will perform the initial validation of the resequencing microarray made in Example 2 in two phases. First we will assay representative cultures of the pathogenic bacteria that have complete or near complete genome sequences available. Knowing the exact genetic composition of the strains applied will provide the most confidence in predicting hybridization and base-calling patterns during the optimization phase. Then we will apply all sequenced strains in a Latin Square type study to determine detection sensitivity and base-call accuracy under polymicrobial conditions in the absence of human DNA.

Following this we will assay spiked blood or stool samples containing either genome sequenced representatives or clinical isolates whose antibiotic resistance phenotype has previously been characterized. This will provide detection specificity and sensitivity data in the presence of any potential background interference from clinical samples. Target interference will be detected by comparing probe-set response to the organism from pure-culture (II-B: Pure genomes) versus mixed genomes (II-B: Latin Square assay) or spiked clinical material (II-B: Clinical spike-ins). All probes affected by interference will be removed from the final probe sets.

Pure Genomes:

DNA will be extracted from representative bacterial strains with fully sequenced genomes and hybridized to the microarray. Where available we will initially assess locus quality and sequence base-call accuracy using two sequenced representative strains of each target species. Only a single concentration of template DNA will be tested at this point. This will allow initial evaluation of base-call accuracy and locus response in a well-characterized genetic background. Each locus will pass QC if 99% of the base calls from PM probes match the expected gene sequences and are differentiated from the 3 mM probes with quality score thresholds of at least 12 (i.e. the maximum for homozygous data). For this test with sequenced genomes in a non-complex background, if the array-derived sequence diverges from the database sequence due to ambiguous base-calls at non-SNP positions the probe set will be retained but truncated to remove dependence upon ambiguous positions.

Latin Square Assay:

We will perform a latin square type hybridization study by spiking in genomic DNA from each of the bacterial species using DNA from genome sequenced representatives (where available) or well characterized clinical isolates in some cases. In this assay DNA from each species is added to the hybridization in rotating concentrations such that the total DNA quantity on each array is the same. Using for example 10 arrays each organism will be assayed at 10 different concentrations in a complex background together with the other bacteria. This represents a polymicrobial infection scenario and will allow us to determine the limits of detection and interference (e.g. probe cross-hybridization) in such cases. At this point we will also assay pure cultures of a defined number of selected clinical isolates with defined antibiotic resistance phenotypes from each species to determine accuracy in phenotype prediction. The concentration of DNA to be used for this assay will be based upon the results of the latin square assay described above.

Clinical Spike-Ins:

The final step in determining detection sensitivity, base-call accuracy and locus reliability is to spike known concentrations of bacteria into clinical samples (sterile blood for most, stool for C. difficile). In this assay we will use one sequenced bacterium (where available) and one clinical isolate for each species to be tested. These intact organisms will be spiked into sterile blood or C. difficile negative stool samples at four concentrations in duplicate.

### Example 6

### Optimization of Target Preparation and Hybridization Processes to Decrease Time to Result

Nucleic Extraction Optimization.

Extractions using the Qiagen BioRobot EZ1 will be as sensitive and accurate as current “research lab” methods and will show reduced intra-sample variability. Nucleic acid extraction efficiency and integrity will be optimized using the automated EZ1 robotic system (Qiagen, CA). A modified protocol of the Qiagen All Prep DNA/RNA extraction kit we have developed in our lab will be used in parallel to compare nucleic acid extraction efficiency of both methods.

Pure Bacterial Cultures.

Pure cultures of clinical isolates of each of the 10 test organisms will be grown overnight prior to serial dilution in sterile PBS. One ml volumes of varying culture dilutions (in the range 10⁻¹-10⁻⁹) will be extracted by both EZ1 (Qiagen, CA) automated and a manual modified All Prep (Qiagen, CA) method (see protocol below) to determine the extraction efficiencies of each method. One hundred μl volumes of each dilution will also be plated in triplicate on LB agar plates, or appropriate agar e.g. blood or chocolate agar for specific organisms and incubated aerobically or anaerobically (i.e. C. difficile) at 37° C. overnight to enumerate bacterial CFUs so that extraction efficiency can directly be related to bacterial cell counts

Spiked Blood Samples.

Following assessment of each method for extraction of nucleic acids from pure culture, spiked-in experiments will be carried out as described above with the exception that in each case 1 ml dilutions of cultures will centrifuged and resuspended in a small volume (50 μl of sterile PBS) prior to addition to 950 μl of blood and subsequent extraction and CFU count determinations. This will evaluate extraction efficiency in the presence of blood components. One hundred microliters volumes of each sample will be plated in triplicate on appropriate agar plates and incubated at 37° C. overnight to enumerate bacterial CFUs so that extraction efficiency in the presence of clinically relevant fluids (i.e. components of blood) can be assessed. PCR inhibition is a well-documented problem when extracting from blood collected in blood-culture bottles. This is generally attributed to sodium polyethanol sulfate (86) and/or to haemoglobin and lactoferrin (87). To eliminate this potential problem, blood samples will be collected using PreAnalytiX PAXgene Blood DNA and RNA tubes. Blood samples are drawn directly into evacuated PAXgene Blood Tubes via standard phlebotomy technique. Tubes are sterile and contain additives that stabilize nucleic acids for subsequent purification. For long-term storage, samples will be stored at −80° C. PCR poison assays will be carried out using low concentrations of spiked bacterial DNA into blood extracts to test for PCR inhibition.

Clinical Blood Samples.

Following optimization of the extraction method, 10 ml clinical blood samples taken from septic patients will be used for extraction. Bacterial quantitative cultures will be carried out on the same blood samples. This will permit a direct comparison between the results of clinical culture and our extraction and detection method to be made

We will reduce the minimum times for template fragmentation, labeling, and hybridization and also the minimum times for array washing, staining and scanning so that the entire array process takes only 6 hours from receipt of template. Currently, the standard resequencing protocol can take 3 days to complete. Template DNA extraction, amplification, quantification and concentration normalization can be accomplished on the first day. Fragmentation, labeling of the fragmented DNA and hybridization to the arrays is carried out on the second day, followed by washing and staining and then scanning of the arrays on the third day. The current standard protocol calls for an overnight hybridization of 16 hours for the array. Successful resequencing of microbial targets has been accomplished with this method (Lin B, Wang Z, Vora G J et al. Broad-spectrum respiratory tract pathogen identification using resequencing DNA microarrays. Genome Res 2006; 16:527-35, Davignon L, Walter E A, Mueller K M et al. Use of resequencing oligonucleotide microarrays for identification of Streptococcus pyogenes and associated antibiotic resistance determinants. J Clin Microbiol 2005; 43:5690-5, Wang Z, Daum L T, Vora G J et al. Identifying influenza viruses with resequencing microarrays. Emerg Infect Dis 2006; 12:638-46). However, other reports using targets found in high abundance in the microbial cell, e.g. 16S and 23S genes, have used shorter hybridization times of an hour or less at higher temperatures (Troesch A, Nguyen H, Miyada C G et al. Mycobacterium species identification and rifampin resistance testing with high-density DNA probe arrays. J Clin Microbiol 1999; 37:49-55, Couzinet S, Jay C, Barras C et al. High-density DNA probe arrays for identification of staphylococci to the species level. J Microbiol Methods 2005; 61:201-8, Gonzalez R, Masquelier B, Fleury H et al. Detection of human immunodeficiency virus type 1 antiretroviral resistance mutations by high-density DNA probe arrays. J Clin Microbiol 2004; 42:2907-12).

The acceptability of using a standard volume of amplified template without concentration normalization will be determined based on the reproducibility of the accuracy of the sequencing calls over a range of starting template concentrations. This will result in a saving of both time and the need for associated laboratory equipment.

The fragmentation and labeling steps of the protocol are enzymatic reactions that have been optimized for human genomic DNA templates. The concentration of the fragmentation reagent and terminal deoxynucleotidyl transferase will be optimized to enable shorter reaction times than the current 30 minutes and 2 hours respectively. The new conditions will be tested over a broad range of input concentrations to ensure that the signal to noise ratio on the arrays is not adversely affected.

The relationship between the hybridization time and temperature will be investigated with all targets present on the array and optimized for improved signal to noise ratios to produce high quality sequencing calls. The effect of known DNA hybridization enhancers, e.g. single stranded binding protein, on the hybridization time to produce acceptable signal intensity will also be investigated. These factors will be optimized using amplified material from negative samples spiked with either high or low genome copies of the target organisms. The final outcome of this optimization will be a hybridization time of less than three hours producing high quality sequence data.

### Example 7

### Clinical Diagnostic Array and Methods

The final diagnostic will comprise of an optimized protocol in which, for example, blood, will be collected in a Paxgene tube, which we have determined results in maintenance of sample integrity and is most amenable to downstream molecular assays. Collected samples will be subjected to our optimized nucleic acid extraction protocol which includes increased periods of bead-beating (45 s-120 s) and followed by automated extraction using the Qiagen Biorobot. Following this, multi-plex amplification reactions will be performed with the addition of BSA to improve amplification efficiency and diminish amplification inhibitory effects. Amplified products will be fragmented, labeled with biotin and applied to the microarray. Optimization of hybridization conditions to minimize time while maximizing sensitivity and specificity is on-going. Finally data is fed through the TessArae analysis pipeline (Potomac Falls, Va.) to provide an interpretable data report for each sample.

### Example 8

### Examples of Sample Extraction Methods—Airway and Stool

Nucleic extraction methods to be used represent one or more approaches based on our prior publications (Roediger, F. C., Slusher, N. A., Cox, M. J., Pletcher, S. D., Goldberg, A. N. and Lynch, S. V. Optimizing Bacterial DNA Extraction from Maxillary Sinus Samples. Am. J. Rhinol Allergy. 2010 July; 24(4):263-5. PMID: 20819463; Huang, Y. J., Nelson, C. E., Brodie, E. L., DeSantis, T. Z., Baek, M. S., Liu, J., Woyke, T., Allgaier, M., Bristow, J., Wiener-Kronish, J. P., Sutherland, R., King, T. S., Icitovic, N., Martin, R. J., Asthma Clinical Research Network Investigators, Boushey, H. A., and Lynch, S. V. Airway Microbiota and Bronchial Hyperresponsiveness in Patients with Sub-optimally Controlled Asthma. J. Allergy Clin. Immunol. J Allergy Clin Immunol. 2011 February; 127(2):372-381.e1-3. Epub 2010 Dec. 30. PMID: 21194740; Cox, M. J., Allgaier, M., Taylor, B., Baek, M. S., Huang, Y. J., Daly, R. A., Karaoz, U., Andersen, G. L., Brown, R., Fujimura, K. E., Wu, B., Tran, D., Kofff, J., Kleinhenz, M. E., Nielson, D., Brodie, E. L. and Lynch, S. V. Airway Microbiota and Pathogen Abundance in Age-Stratified Cystic Fibrosis Patients. (PLoS One PLoS One. 2010 Jun. 23; 5(6):e11044). PMID: 20585638; Cox, M., Huang, Y. J., Fujimura, K., Liu, J., McKean, M., Brodie, E. L., Boushey, H., Cabana, M. and Lynch, S. V. Lactobacillus casei Abundance is Associated with Profound Shifts in the Infant Gut Microbiome. PLoS One. 2010 Jan. 18; 5(1); Ivanov, LI., Atarashi, K., Manel, N., Brodie, E. L., Shima, T., Karaoz, U. Wei, D., Goldfarb, K. C., Santee, C. A., Lynch, S. V., Imaoka, A. Itoh, K., Takeda, K., Umesaki, Y., Honda, K., and Littman, D. R. Cell. 2009 Oct. 30; 139(3):485-98.

### Example 9

### Second Prototype Array Design

As described above, we compiled a comprehensive and solid set of antibiotic resistance elements and their associated annotations. Of the actual sequences tiled as detectors throughout the designed array, there are 3 classes: antibiotic resistance (AR) gene targets (SEQ ID NOS: 1-970), Organism/species identification (BUG-ID) gene targets (SEQ ID NOS: 971-1232), and SNP targets (SEQ ID NOS: 1233-1323) selected genes known to be associated with bacterial antibiotic resistance bearing mutations in those genes that are deemed determinants of resistance.

The same methods as described in Example 1 were used to curate the sequences and select the target sequences.

We completed tiling probe sets for all candidate detector tiles, apportioned to Bacterial genera/species (262 tiles) and Resistance determinants (1061). The array platform we are using has the capacity to accommodate 1,345 tiles of up to 224 bp each, we therefore dramatically expanded the targets included on the array from 11 to 44 clinically relevant bacterial species and their associated antibiotic resistance determinants and are currently at 99.96% of the array capacity. These additional species were chosen based on their clear role in pathogenesis and their detection in a number of recent airway microbiome studies. The final list of all bacterial species to be detected by this tool are described in Table 7. These targets are represented by 262 tiles totaling 58,688 bp (of a possible 303,000 bp) on the array. The probes for each tile are the same as the sequence for each tile/target plus the complement of that sequence and 3 mismatch probes for each perfect match. Each base across that tile is covered by 8 probes on the resequencing array.

TABLE 7
Targeted 44 common nosocomial pathogens

Acinetobacter baumannii
Burkholderia ambifaria
Burkholderia cenocepacia
Burkholderia cepacia
Burkholderia dolosa
Burkholderia multivorans
Bordetella parapertussis
Bordetella pertussis
Burkholderia pyrrocinia
Burkholderia stabilis
Burkholderia vietnamiensis
Clostridium difficile
Citrobacter freundii
Chlamydophila pneumoniae
Chlamydia trachomatis
Enterobacter aerogenes
Enterobacter cloacae
Escherichia coli
Enterococcus faecalis
Enterococcus faecium
Haemophilus influenzae
Haemophilus parainfluenzae
Klebsiella oxytoca
Klebsiella pneumoniae
Legionella pneumophilia
Mycobacterium avium
Moraxella catarrhalis
Mycobacterium kansaii
Mycoplasma pneumoniae
Mycobacterium tuberculosis
Neisseria meningitidis
Pseudomonas aeruginosa
Proteus mirabilis
Pseudomonas pseudoalcaligenes
Proteus vulgaris
Staphylococcus aureus
Staphylococcus epidermidis
Stenotrophomonas maltophilia
Serratia marcescens
Streptococcus mitis
Streptococcus mutans
Streptococcus pneumoniae
Streptococcus pyogenes
Streptococcus viridans

Genes specifically linked to SNP-associated resistance targeted by the array-based assay are listed in Table 7 above. These targets are represented by 91 tiles, totaling 58,568 bp on the array. The regions of the targets are identified as SEQ ID NOS: 1233-1323.

Finally, the remaining antibiotic resistance targets represent all other antibiotic resistance determinants and are covered on the array by 970 consensus sequencing tiles totaling 217,280 bp. In summary the novel resequencing array content includes detection of 44 clinically relevant bacterial species, 21 target genes with documented SNP-associated resistance and 16,000 clusters of antibiotic resistance determinants whose presence is associated with resistance to clinically relevant antimicrobial agents.

This resequencing array can be fabricated and used in applications as described above.

It will be appreciated, however, that the present embodiments may be practiced in many ways and should be construed in accordance with the appended claims and any equivalents thereof. Any patents, publications or references cited herein are hereby incorporated by reference in their entireties for all purposes.

TABLE 2
Numbers of antibiotic resistance element clusters

| | | | Number of |
|---|---|---|---|
| Mechanism | | | Clusters |
| drug destruction | beta lactamase | class A | 225 |
| drug destruction | beta lactamase | class B | 30 |
| drug destruction | beta lactamase | class C | 45 |
| drug destruction | beta lactamase | class D | 80 |
| drug modification | aminoglycoside | acetylation | 75 |
| | resistance | | |
| drug modification | aminoglycoside | adenylation | 60 |
| | resistance | | |
| drug modification | aminoglycoside | phosphorylation | 56 |
| | resistance | | |
| reduce drug | efflux | major facilitator | 25 |
| concentration | | superfamily (MFS) | |
| reduce drug | efflux | ATP-binding cassette | 34 |
| concentration | | superfamily (ABC) | |
| reduce drug | efflux | small multidrug | 10 |
| concentration | | resistance family (SMR) | |
| reduce drug | efflux | resistance-nodulation-cell | 103 |
| concentration | | division superfamily | |
| | | (RND) | |
| reduce drug | efflux | Multi antimicrobial | 5 |
| concentration | | extrusion protein family | |
| | | (MATE) | |
| reduce drug | cell wall | | 120 |
| concentration | alteration | | |
| reduce target | dhfr | | 89 |
| binding | | | |
| reduce target | ribosomal | | 34 |
| binding | protection | | |
| reduce target | gyrase | | 32 |
| binding | protection | | |
| unclassified | unclassifed | unclassified | 369 |
| TOTAL | | | 1392 |

1. (canceled)

2. (canceled)

3. A method for determining a disease condition of a subject, the method comprising the steps of:
(a) obtaining a sample from a patient;
(b) isolating nucleic acid material from said sample;
(c) amplifying a target locus from said nucleic acid material;
(d) contacting said target locus with a set of probes (resequencing probes), wherein said set comprising 8 probes (4 probes based on the sense strand and 4 probes based on the anti-sense strand) per nucleotide base interrogated in each target locus;
(e) determining hybridization signal strengths across the set of probes;
(f) determining hybridization signal strengths for a plurality of different interrogation probes, each of which is complementary to a section within said target locus;
(g) determining the sequence of the target locus by analysis of the hybridization signal strengths of the resequencing probes;
(h) comparing the target locus sequence with a set of known sequences to determine the presence and/or antibiotic resistance repertoire of one or more target organisms;
(j) defining therapeutic strategy for said patient based on the results of step (h);
(l) classifying, diagnosing, prognosing, and/or predicting an outcome of said condition based on the results of step (h).

4. A method for parallel detection and strain level-identification of a panel of more than 44 organisms, in parallel with antibiotic resistance profiling of said organisms comprising the steps of:
a) extraction of nucleic acids from a patient sample using a rapid optimized protocol;
b) amplifying target loci from that sample using multiplex polymerase chain reaction;
c) pooling target locus amplified products;
d) labeling pooled amplified products;
e) contacting the labeled amplified pool of products with a plurality of resequencing probes which target both the sense and anti-sense strands of the target loci represented in SEQ ID NOS:1-1323;
f) determining hybridization signal strength for each of said probes, wherein said determination identifies the specific sequence of the target locus, providing either strain level organism identification in parallel with single nucleotide polymorphism resolution antibiotic resistance determinant sequence information.

5. (canceled)

6. An array system comprising: a resequencing microarray configured to simultaneously detect a plurality of organisms and antibiotic resistance elements in a sample, wherein the microarray comprises resequencing probes for organism identification, antibiotic resistance element detection, and detection of polymorphisms related to said antibiotic resistance, whereby said resequencing probes for organism detection can provide strain-level detection and identification of a organism in a sample and whereby said resequencing probes for antibiotic resistance elements and said resequencing probes for antibiotic resistance related polymorphisms provide for detection of emerging antibiotic resistance of organisms in a sample.

7. The array system of claim 6, wherein the plurality of organisms comprise bacteria.

8. The array system of claim 6, wherein the fragments are about 25 nucleotides long.

9. The array system of claim 6, wherein the sample is an environmental sample.

10. The array system of claim 9, wherein the environmental sample comprises at least one of soil, water or atmosphere.

11. The array system of claim 6, wherein the sample is a clinical sample.

12. The array system of claim 11, wherein the clinical sample comprises at least one of tissue, skin, stool, bodily fluid or blood.

13. A method of simultaneously detecting an organism in a sample and its antibiotic resistance comprising the steps: applying a sample comprising a plurality of organisms to the array system of claim 6; and simultaneously identifying at least one organism in the sample and determining its antibiotic resistance.

14. The method of claim 13, wherein the plurality of organisms comprise bacteria.

15. The method of claim 13, wherein the fragments are about 25 nucleotides long.

16. The method of claim 13, wherein the antibiotic resistance elements detected represent new or emerging resistance in the organism or organisms detected in the sample.

17. A method of designing and fabricating an array system comprising: identifying organism-specific and antibiotic resistance element sequences corresponding to a plurality of organisms and resistance elements of interest; selecting target loci unique to each organism and resistance element; selecting resequencing probes; and fabricating said array system.

18. The method of claim 17, wherein the plurality of organisms comprise bacteria.

19. The method of claim 17, wherein each fragment has a corresponding set of 8 variant fragments per nucleotide base interrogated, 4 based on sense strand and 4 on anti-sense strand.

20. The method of claim 17, wherein the fragments are about 25 nucleotides long.

21. (canceled)

22. A computer-readable storage medium comprising a database of antibiotic resistance elements (BARChipDB) comprising 262 bacterial genera/species isolated polynucleotides (SEQ ID NOS:971-1232) and 1,061 isolated polynucleotides comprising resistance determinants sequences (SEQ ID NOS:1-970 and 1233-1323).


SEQUENCE LISTING (1323 sequences)

SEQ ID NO: 1 - DNA, 224 bp, Streptococcus mitis
agttttcttactggttggtagttttctatttttagctgctattttgactatttgctttatcaaggaagattttcaaccagtagccaaggaaaaggctattccaacaaaggaattatttacctcggttaaatatccctatcttttgctcaatctctttttaaccagttttgtcatccaattttcagctcaatcgatcggccctattttggctctttatgtacgcg

SEQ ID NO: 2 - DNA, 224 bp, Pseudomonas aeruginosa
gcagccgacctacgcgccgggcagccagcgcctctattccaacccgagcatcggcctgttcggctatctcgccgcgcgcagcctgggccagccgttcgaacggctcatggagcagcaagtgttcccggcactgggcctcgaacagacccacctcgacgtgcccgaggcggcgctggcgcagtacgcccagggctacggcaaggacgaccgcccgctacgggtcg

SEQ ID NO: 3 - DNA, 224 bp, Staphylococcus epidermidis
tagtcaaaatgagtcgatcagttactgctatagaaattgatggaggcttatgtcaagtgactaaagaagcggtaaacccctctgagaatataaaagtgattcaaacggatattctaaaattttccttcccaaaacatataaactataagatatatggtaatattccttataacatcagtacggatattgtcaaaagaattacctttgaaagtcaggctaaatat

SEQ ID NO: 4 - DNA, 224 bp, Streptococcus pneumoniae
tttgacgcgtaatccggaagaaaagatagatggggttgctacttttcaggatgtccagtctgtcttgactggtatcaggctcaagaaaagaatctctatattctcggtgggaagcaaatttttcaggcttttgaaccctaccttgatgaagtgattgtcactcacattcatgctcgggtggagggagatacctatttccctgaagagtttgacttgtctctttt

SEQ ID NO: 5 - DNA, 224 bp, Aeromonas veronii
cttactgagagaaattggcggacctgctgcaatgacgcagtattttcgtaaaattggcgactctgtgagtcggctagaccggaaagagccggagatgggcgacaacacacctggcgacctcagagatacaactacgcctattgctatggcacgtactgtggctaaagtcctctatggcggcgcactgacgtccacctcgacccacaccattgagaggtggctga

SEQ ID NO: 6 - DNA, 224 bp, Staphylococcus aureus
tcaattgtattgattcacgatccgaaaaagtctacgacaagtggtttccaaaagttagagccacaattgctaacgaaaattaactggaaagtgtttattacaccagttattttaacacttgtattatcgtttggtttatctgcatttgaaacattgtattcactatacacagctgacaaggtaaattattcacctaaagatatttcgattgctattacgggtgg

SEQ ID NO: 7 - DNA, 224 bp, Enterococcus gallinarum
cggctgcgcttggctatggcttgcttttatgggatggatatcggccaagaactgcggtggactgcttcctgcgttgggcagcgcaaccggaggacaatctcacaaaagaaaaattttaccccaatatagagcgagccgagttgattacaaagggttatgtggcctcacaatccagccatagccgtggaagcgcaattgatcttacgctctaccacctggataca

SEQ ID NO: 8 - DNA, 224 bp, Burkholderia mallei
ccggccaggcgtcgctgtcgctcacgttcaagcagggcgtgagcgccgatctcgcggccgtcgacgtgcagaaccgcctgaaaatcgtcgaggcgcggctgcccgagcccgtgcggcgcgacggcatctcgatcgagaaggcggccgacaacgcgcagatcatcgtgtcgctcacgtcggaggacggacggttatcgggcgtggagctcggcgaatacgcgtcg

SEQ ID NO: 9 - DNA, 224 bp, Leptothrix cholodnii
gcaacatctaccggcagttcgcgttcgcgctggcggtgtcgatcggcttctcggcgtttctcgcgctgtcgctcacgccggcgctctgcgcgacgctgctcaagcccgtcgccgacgaccatcacgagaaggacggcttcttcggctggttcaaccgtttcgtcgcgcgctcgacgcaccgctacacgcggcgcgtcgggcgggtgctcgagcgcccgctgcgc

SEQ ID NO: 10 - DNA, 224 bp, Ralstonia eutropha
tacgagagctggtcgattccgttcgcggtgatgctcgtcgtgccgctcggcgtgatcggcgcggtcgcgggcgtcacgctgcgcgggatgccgaacgacatctatttcaaggtggggctgatcgcgacgatcggtttgtccgcgaagaacgcgatcctgatcgtcgaggtcgcgaaggatctggtcgcgcagcgcatgtcgctcgccgacgcggcgctcgaggc

SEQ ID NO: 11 - DNA, 224 bp, Staphylococcus epidermidis
ggcatatttagttaataatgaatacatttttaaaacaaaatttagtactaataagaaaaaaggttatgcaaaagaaaaagcaatatataattttttaaatacaaatttagaaactaatgtaaaaattcctaatattgaatattcgtatattagtgatgaattatctatactaggttataaagaaattaaaggaacttttttaacaccagaaatttattctacta

SEQ ID NO: 12 - DNA, 224 bp, Staphylococcus epidermidis
aagactaaatgcaacaacagtttttgagggtaaaaagtgtttatgccataatgattttagttgtaatcatctattgttagatggcaataatagattaactggaataattgattttggagattctggaattatagatgaatattgtgattttatatacttacttgaagatagtgaagaagaaataggaacaaattttggagaagatatattaagaatgtatggaa

SEQ ID NO: 13 - DNA, 224 bp, Klebsiella pneumoniae
accccagcatagggctgttcggccacctggcggcgagcagcctgaagcagccgtttgcccccttgatggagcagaccctgctgcccgggctcggcatgcaccacacctatgtcaatgtgccgaagcaggccatggcgagttatgcctatggctattcgaaagaggacaagcccatccgtgtcaaccctggcatgctggcggacgaggcctatggcatcaagacc

SEQ ID NO: 14 - DNA, 224 bp, Neisseria meningitidis
gccgtatacgacaccatcgtccgtatggcgcaaaatttcgctatgcgttatgtgctgatagacggacagggcaacttcggatcggtggacgggcttgccgccgcagccatgcgctacaccgaaatccgcatggcgaaaatttcccacgaaatgctggcagacattgaggaagaaaccgtcaatttcggcccgaactacgacggtagcgaacacgagccgcttgt

SEQ ID NO: 15 - DNA, 224 bp, Neisseria gonorrhoeae
gttttggtcgacggacaaccgcgcctgttgaacctgaaacagattctctccgaattcctgcgccaccgccgcgaagtcgttacccgacgtacgcttttccggctgaagaaggcacgccatgaagggcatattgccgaaggcaaagccgtcgcactgtccaatatcgatgaaatcatcaagctcatcaaagaatcgcccaacgcagccgaggccaaagaaaaact

SEQ ID NO: 16 - DNA, 224 bp, Staphylococcus aureus
tattacatgtattagtgggtatggtgccagcaggtattttaggcttactatttgatgatttcatcgaagaacatttatttagtgtgccaactgttatgattggcttattcgtaggtgctatctatatgattattgctgataaatactcagctaaagttaaaaacccacaaacagtagatcaaatcaattatttccaagcatttgtaattggtatttctcaagca

SEQ ID NO: 17 - DNA, 224 bp, Enterobacter cloacae
atcttcgtgatgcaagtttccaacaatgccaacttgcgatggcaaacttcagtaatgccaattgctacggtatagagttccgtgcgtgtgatttaaaaggtgccaacttttcccgaacaaactttgcccatcaagtgagtaatcgtatgtacttttgctcagcatttatttctggatgtaatctttcctatgccaatatggagagggtttgtttagaaaaatgt

SEQ ID NO: 18 - DNA, 224 bp, Shigella flexneri
ataatctcatgtatatgtcatctcagagcagtggcaccggtcaggcatctgtcactttaagttttaaagcaggcaccgatccggacgaagccgtgcagcaagtacaaaaccagctgcaatcagccatgcgaaagttaccgcaggcggtgcaaaatcagggcgtgacggtgcgtaaaaccggcgataccaacattctgaccattgccttcgtctctaccgatggt

SEQ ID NO: 19 - DNA, 224 bp, Shigella sonnei
attgtgatttatgtcctgctgcttggcggcatggtgttcctgttcctgcgtttgccgacgtcgttcttaccgctggaagaccgtggcatgtttactacctcggtacaattgcccagcggttcaacccaacaacagaccctgaaagtcgttgagcaaatcgagaaatattacttcacccatgaaaaagacaacatcatgtcggtgtttgccaccgttggttctgg

SEQ ID NO: 20 - DNA, 224 bp, Neisseria flavescens
tcaaacgctggcttatgacgagttgaacaaggctgtggcttatcacaaagccaaagcgggtgcggttgtggtattggatgcgcaaaccggcgagattttggctttggtcaacagcccggcttacgatccgaatcaaccgggtcaagccaatagcgaacagcgccgcaaccgcgctgtaaccgacatgatcgaacctggttctgccatgaagccgtttaccattg

SEQ ID NO: 21 - DNA, 224 bp, Neisseria gonorrhoeae
atgcattcaggctttcctggcgagactgcaggtttgttgagaagctggcgcagatggcaaaatatcgaacaggcgacgatgtctttcggttacggcctgcaattgagcctgctgcaattggcgcgcgcctataccgcactgacgcacgacggcgttttgctgccggtcagctttgaaaaacaggcggttgcgccgcaaggcaaacgcatattcaaagaatcgac

SEQ ID NO: 22 - DNA, 224 bp, Acinetobacter sp.
gcgcgcggcgcagcgcgcaccccgatgcatcgatggtcgcggttggtccgctagctgaaacgctgacggagcctcacgaactcggtcacgccttgggggaagggtcgcccgtcgagcggttcgtccgccttggcgggaaggccctgctgttgggtgcgccgctaaactccgttaccgcattgcactacgccgaggcggttgcggatatccccaacaaacgatgg

SEQ ID NO: 23 - DNA, 224 bp, Unknown
  Description of Unknown Bacterium DT40 polynucleotide
tattccatgcacttagaaagatgaacatacctataatattttttattaacaaaattgatcaaaatggaataaatttgccagatgtttatcaagatattaaggacaaactttctgacgacatcataattaagcagactgtgaatctaaatttgaaaccttatgtaatagattatactgaaccagaacaatgggagacagtaattgtgggaaatgattatttatta

SEQ ID NO: 24 - DNA, 224 bp, Listeria monocytogenes
ccaactgtcatttatatggaaagaccattaaaaaaatctgaatttaccattgatatcgaagtacctccaaatcctttctgggcttctattggtttatctgtaacaccacttcctttgggtagtggcattcagtatgagagcctggtttctctaggttatttaaatcaatcatttcaaaatgcagttatggaaggtatacgctatgggtgtgaacaaggattgta

SEQ ID NO: 25 - DNA, 224 bp, Burkholderia cepacia
ccgcaaggtgccggtcgagctcggcctcgcgaacgaaaccggctactcgcgcagcggcgagatcgattcggtcgacaaccggctcgacacgtcgtccggcacgatccgcgtgcgcgcgcgcttcgacaacgcggacggcacgctcgtcccgggcctgtacgcacgcgtgaaggtgggcggcagcgcgccgcacgaggcgctgctcgtcgacgacgcggcgatca

SEQ ID NO: 26 - DNA, 224 bp, Shigella sonnei
ccgttaaaacccccgtccagtgcaggctggcgtagggtaaaatcagcgtaaacggcgcaacgccgaggatagagccccaaatcacatatttccgtccaattttatcccctacaggcccgccgatcaccgtacctgccgcaacggcaaacaggaaggcaaacagatgaagctgagcattctggatagataatccgaatttttgcatcagataaaaggtgtaatag

SEQ ID NO: 27 - DNA, 224 bp, Shigella sonnei
aagccatacgggctacgcgagaagattccggatgaaagaccgatgaaccggtaccgaccagcgccgccgccagcagaactgcgccaaaactgcccgccagcgcaagcagcaccagaccacttaaggtaaagcacatgccaattggcaacgaccacggcatcggatatttatcggtccagtagccgaccactggttgcagtagcgaagaggcgagctggaaggtg

SEQ ID NO: 28 - DNA, 224 bp, Salmonella enterica
gcatgccgaacttcatggtggaccctaaaactcttcaatgcacgggtctgatcgaccttgggcggctcggaacagcagatcgctatgccgatttggcactcatgattgctaacgccgaagagaactgggcagcgccagatgaagcagagcgcgccttcgctgtcctattcaatgtattggggatcgaagcccccgaccgcgaacgccttgccttctatctgcga

SEQ ID NO: 29 - DNA, 224 bp, Klebsiella sp.
gacgtaatcggtaaataccacccgcacggcgactccgcggtatacgacaccatcgtgcgtatggcgcagccgttctcgctgcgttacatgctggtggacggccagggtaactttggttccatcgacggcgactccgccgcggcgatgcgttataccgaaattcgtctggcgaaaatcgctcatgagctgatggccgatcttgaaaaagagacggtcgatttcgt

SEQ ID NO: 30 - DNA, 224 bp, Staphylococcus aureus
ccttttgggttattaataaagatgataggccggtggcagctacgtttacctatcctgtttttgttaagccggcgcgttcaggctcatccttcggtgtgaaaaaagtcaatagcgcggacgaattggactacgcaattgaatcggcaagacaatatgacagcaaaatcttaattgagcaggctgtttcgggctgtgaggtcggttgtgcggtattgggaaacagt

SEQ ID NO: 31 - DNA, 224 bp, Streptococcus sanguinis
atgtcctcatctctattgatacttggaagagtcaggtagcagaggctgctttggctgctggtgccaatctagtcaatgatatcactggtctcatgggtgatgaaaaaatggcccatgtggttgctaaggctggagcgaaagtggtcatcatgtttaatccagttatggctcgacctcagcatcctagctcgctcatcttccctcattttggttttggtcaagct

SEQ ID NO: 32 - DNA, 224 bp, Staphylococcus epidermidis
tgacgctcaacacactcaaaaagttatacaaaaattagaagatataggatacaaaatagaagttgattggatgccttcacgtatggaacttaagcatgaagaatatgggtatttagatattcatcctataaatctaaatgatgatggatcaattacccaagcaaacccagaaggtggtaattatgttttccaaaatgactggttttcagaaactaattacaaag

SEQ ID NO: 33 - DNA, 224 bp, Thermophilic bacillus
tagaagagctgtttgaatatgcaggcaaatggcgtaatattcgtgtgcaaggaccgacaacatttctaccatccttgactgtacaggtagcaatggcaggtgccatgttgattggtctgcatcatcgcatctgttatacgacgagcgcttcggtcttaactgaagcagttaagcaatcagatcttccttcaggttatgaccatctgtgccagttcgtaatgtct

SEQ ID NO: 34 - DNA, 224 bp, Burkholderia rhizoxinica
acgatgccgcccatcagcaggatcggcaggaacaccgcgacgagcgagatgctcatcgacagcaccgtgaagccgacttcgcgcgcgccgtcgaacgcggcctgcatccgcggcttgccgttctcgatgtgccgcgagatgttctcgagcacgacgatcgcatcgtcgacgacgaagccggtcgcgacgatcagcgccatcagcgacaggttgtcgatcgagaa

SEQ ID NO: 35 - DNA, 224 bp, Staphylococcus aureus
gtttattacgaactttaattggttatatatgattggtgccttaattgcgattatcgtttttgcgctatatattaaaaatgcgcaacgtccattagtaaataaatcatttttccaaaataaacgttatgcttcatttttatttatagtatttgtaatgtatgctatccaattgggttatatttttacgttcccattcataatggagcaaatttatcatctgcaac

SEQ ID NO: 36 - DNA, 224 bp, Gemella haemolysans
gcaaatggcgattattttttaccttacagaaaaaactggatctgcgatggtcttgtctatggcttcactattaggttttttaccctatgcggtctttggacctgcaattggtgtgttagtggatcgtcatgataggaagaagataatgattggtgctgatttaattatcgcagcagctggttcggtgcttactattgttgcattctatatggagctacctgtct

SEQ ID NO: 37 - DNA, 224 bp, Gemella haemolysans
tgattgcatctattacggtagcaattgtacgtattcctaagctgggtgatcgcgtgcaaagtttggacccaaatttcataagagaaatgaaagaaggaatggctgtactacggcaaaataaaggattatttgctttattactcgttggaacattatatatgtttgtttatatgccaattaatgcactattccctttaattagcatggattactttaatggaaca

SEQ ID NO: 38 - DNA, 224 bp, Streptococcus pneumoniae
tctttactttgaaggggaagacaatggttttcaagtggaagtagccctccagtacaatgacggattctcagataacattctatcctttgtcaataacgttcgcaccaaggacggtggaacgcacgagacaggactcaagtctgccattaccaaggtcatgaatgactatgcacgtaaaacaggtcttctcaaggaaaaagataaaaaccttgaaggttcagact

SEQ ID NO: 39 - DNA, 224 bp, Streptococcus pneumoniae
cgtggtaaggttatcaatacagccaaggccaagatggcggatatcctcaaaaatgaagagatcaataccatgatttataccattggtgcgggtgttggagcagacttctctattgaagatgccaactatgataagatcattatcatgaccgatgcggataccgacggtgcccatatccagaccttgctcttgacatttttctaccgttacatgcgtccgctagt

SEQ ID NO: 40 - DNA, 224 bp, Staphylococcus aureus
gctttaacaagtaattatttgaaagtaatgtttttaagtgcaccttttgtaattttgttcttcatattagaacaatttgcacgtgcaattggggcaccaatgatttctatgattggtatgttagctagtgtaggcttaaatattattttagatccaattttaatttttggttttgatttaaacgttgttggtgcagctttgggtactgcaatcagtaatgttgc

SEQ ID NO: 41 - DNA, 224 bp, Staphylococcus aureus
cgcgattgcaagttatggtatctcatttagacttgtgcaatttccagaacttattatcatgggattatgtgaaggtgttgtaccactaattgcatataactttatggcaaataaaggccgtatgaaagacgttatcaaagcagttatcatgtctatcggcgttatctttgttgtatgtatgattgctgtatttacaattggacatcatatggtcggactattta

SEQ ID NO: 42 - DNA, 224 bp, Staphylococcus epidermidis
ttacctgatattgcaaatcattttaatactactcctggaattacaaactgggtaaacactgcatatatgttaactttttcgataggaacagcagtatatggaaaattatctgattatataaatataaaaaaattgttaattattggtattagtttgagctgtcttggttcattgattgcttttattggtcacaatcacttttttattttgatttttggtaggtt

SEQ ID NO: 43 - DNA, 224 bp, Staphylococcus rostri
cttcctatgattacaatagtaactataccttttcttattaaagtaatggtacctggtaaatcaacaaaaaatacattagatatcgtaggtattgttttaatgtctataagtattatatgttttatgttatttacgacaaattataattggacttttttaatactcttcacaatcttttttgtgatttttattaaacatatttcaagagtttctaacccttttat

SEQ ID NO: 44 - DNA, 224 bp, Artificial Sequence
  Description of Artificial Sequence Synthetic shuttle vector polynucleotide
ccaaacaagaatttatttatggagaatggttacaagagctttatgaacaaggatacattcctcagaaggaattaaattcagatttaaccataatgctttaccaagcaaaacgaaaaaataaaagaatatacggaaattatgacttagaggaattactacctgatattccattttctgatgtgagaagagccattatggattcgtcagaggaattaatagataat

SEQ ID NO: 45 - DNA, 224 bp, Pseudomonas sp.
gagaaggctgctcaaaaacaagctaagcatattgaaaaaagaatggaacatttggaagaagttgaaaaaccacaaagttatcatgaattcaattttccacaaaataaaatttatgatatccataataattatccaatcattgcacaaaatctaacattggttaaaggaagtcaaaaactgctaacacaagtacgattccaaataccatatggcaaaaatatagc

SEQ ID NO: 46 - DNA, 224 bp, Human herpesvirus
aaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtc

SEQ ID NO: 47 - DNA, 224 bp, Serratia marcescens
cggcagcgccttcgacggcgtcagcctgctgaatctggcgacgcatacctccggcctgccgctgttcgtgccggacgacgtcaccaacaacgcccagctgatggcctactaccgggcctggcagccgaaacacccggcgggcagctaccgcgtctattccaacctcggcatcggcatgctgggcatgatcgccgccaagagcctcgaccagccgtttatccagg

SEQ ID NO: 48 - DNA, 224 bp, Enterobacter asburiae
gaccatacctggattaacgttccgaaagcggaagaggcgcattacgcctggggataccgtgacggtaaagcggtacacgtttcgccagggatgctggacgcggaagcctatggcgtaaaaaccaacgtgaaggatatggcaagctgggtgatggtcaacatgaagccggactcccttcaggataattcactcaggcaaggcattgccctggcgcagtctcgcta

SEQ ID NO: 49 - DNA, 224 bp, Proteus vulgaris
cgtggcctgtgatttgttatttgaactggttggtggaccagctgctttgcatgactatatccagtctatgggtataaaggagaccgctgtggtcgcaaatgaagcgcagatgcacgccgatgatcaggtgcagtatcaaaactggacctcgatgaaaggtgctgcagagatcctgaaaaagtttgagcaaaaaacacagctgtctgaaacctcgcaggctttgt

SEQ ID NO: 50 - DNA, 224 bp, Artificial Sequence
  Description of Artificial Sequence Synthetic plasmid polynucleotide
ggtgcgttactggcgcgtgaactgggtattcgtcatgtcgataccgtttgtatttccagctacgatcacgacaaccagcgcgagcttaaagtgctgaaacgcgcagaaggcgatggcgaaggcttcatcgttattgatgacctggtggataccggtggtactgcggttgcgattcgtgaaatgtatccaaaagcgcactttgtcaccatcttcgcaaaaccggc

SEQ ID NO: 51 - DNA, 224 bp, Shigella boydii
gatcagccattttgcacattccagatgcgccagttgcataatttcttcgttcgccgcattgcccaatactgcgcggaccccgcgctctcgcagctcatcaacacgggttcgtgacgtctcaatcaccaccagcggaatatcagaggcgagcaatttctcccccagcaggctgcctacacgaccgtaacccaccagtagcgcatggttgcaaatatccactggga

SEQ ID NO: 52 - DNA, 224 bp, Shigella sonnei
aatcaaccaaccgatggcgatttgcccacgctgactgtcaattaattgccgttcttcaagtgcgcgcagtaacaccacggtactggcggtggaaagacataaaccgaacacgataccggtcattaacgaccagcccagcacggcagagagcgccatacccagcagcgtcgccacggctatctgggcgatcgcaccgggaatggcgatggcctttaccgccatca

SEQ ID NO: 53 - DNA, 224 bp, Staphylococcus aureus
aaacattgatacaaatcaagattctgagtataattcatacgttaactttgtaaaatctgaattaatgaataataaagcattcaaagatgaaaatttaggtaatgtattacaaagtggtattaaaatttatacaaacatggataaagatgttcaaaaaacattacaaaatgatgttgataatggtagcttctacaagaataaagaccaacaagttggtgcaacga

SEQ ID NO: 54 - DNA, 224 bp, Staphylococcus aureus
tctgaagtacttggtggttctgcttcagaattctcaccaacacaattagcatcagcatttgctgcaatcgctaacggtggtacttataacaacgcgcattcaattcaaaaagtagttactcgtgatggtgaaacaatcgaatacgatcatactagccataaagcgatgagtgattacactgcatacatgttagctgagatgctaaaaggtacatttaaaccata

SEQ ID NO: 55 - DNA, 224 bp, Stenotrophomonas maltophilia
gctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccag

SEQ ID NO: 56 - DNA, 224 bp, Aeromonas sp.
cagatgcagatcctgatatgcccgcttttgatcctgccatcattgagtatcaccaacgggcggaatcgctggattggtctgatagagatgctgtcgtcgcgtatcaggtcggagcgtggcgaatcaactcaggtactgcgcatgcttttgacgctgagaagatacaaaatatcgctgagttaaattttgatcgcactccgaatatcctgacaacattcaaccac

SEQ ID NO: 57 - DNA, 224 bp, Staphylococcus haemolyticus
tgatatatactctcttgttttagctggctttataattccattgagtgcctttgctgataaatggggaagaaaaaaagcattattaactggatttgctttatttggcctcgtttcattagctatatttttcgcagaaagtgcagagttcgtaatagctattcgatttttacttggtattgcaggtgctttaataatgccaactactctttcaatgataagagtaa

SEQ ID NO: 58 - DNA, 224 bp, Staphylococcus epidermidis
tatttttattaccagagtctaagttatcaaaagaaaagtctcactcgtgggatattccttctacaattttatcaattgcaggcatgattggactggtatggagtatcaaagaattttcaaaagaaggactagcagatattattccatgggttgtaatagtattagcaattaccatgatagtgatatttgttaaacgtaatttatcaagttctgatccaatgtta

SEQ ID NO: 59 - DNA, 224 bp, Corynebacterium resistens
cggtgtcgctgtccccggaaatagtcctggaggaaaataccgacatagaagcatgggatgcggtcatcgaaaataacgataaattattggaaaagtatatcgcaggagaaccaatcagccgggaaaaacttgcgcgggaggaacagcggcgggttcaagacgcctccctgttcccggtctattatggcagcgccaaaaagggccttggcattcaaccgttgatg

SEQ ID NO: 60 - DNA, 224 bp, Artificial Sequence
  Description of Artificial Sequence Synthetic construct polynucleotide
caagcccggtcgcccgtgtccatgaactcgatgggcaggtacttctcctcggcgtgggacacgatgccaacacgacgctgcatcttgccgagttgatggcaaaggttccctatggggtgccgagacactgcaccattcttcaggatggcaagttggtacgcgtcgattatctcgagaatgaccactgctgtgagcgctttgccttggcggacaggtggctcaag

SEQ ID NO: 61 - DNA, 224 bp, Salmonella enterica
ttgtggaagaaatcaaaagactcaaacctgtcattaaggctttaaaagaaaaaggcatttctatttctgttgatacatttaaacctgaggttcagagtttttgcatagaacaaaaggttgattttattaatgatattcaaggttttccttatcctgagatttattcaggcttggcaaagtcagattgcaaacttgtgttgatgcactccgttcagcgaattggt

SEQ ID NO: 62 - DNA, 224 bp, Clostridium difficile
gtattatgttttctaaaaaaaatgagggtaacgaatttagtaatgaaggaaaaggaagctctaagaaaataattaaattctttaagagcacaaagggtattgctctactggcatttattttaggcgtgttttttggcaatatatcctcaccagcttgttctgaagaccatgaggaggtcatttctaatcaaacatcagttatagattctcaaaaaacagaaata

SEQ ID NO: 63 - DNA, 224 bp, Staphylococcus epidermidis
gtgtaatgaggttcggattcatctatgggaggcaagtgatgaaggctggcgctctcgtagtaatgattcaccggtttgtacaggtgcggagtcgtttattgctggtactgctagttgccgcattgaagtagagggaattgatgaattatatcaacatattaagcctttgggcattttgcaccccaatacatcattaaaagatcagtggtgggatgaacgagact

SEQ ID NO: 64 - DNA, 224 bp, Salmonella typhimurium
cgaaagcgaaaaacggcgtgattggttgcggtccagacataccctggtccgcgaaaggggagcagctactttttaaagcattgacctacaatcagtggcttctggtgggtcgcaagacgtttgaatctatgggcgcactccccaataggaaatacgcggtcgttacccgctcaggttggacatcaaatgatgacaatgtagttgtatttcagtcaatcgaagag

SEQ ID NO: 65 - DNA, 224 bp, Pseudomonas aeruginosa
caactttgacgattgaacaaatactaaattatacagtatcagagagcgacaatattggttgtgatattttgctaaaattaatcggaggaactgattctgttcaaaaattcttgaatgctaatcatttcactgatatttcaatcaaagcaaacgaagaacaaatgcacaaggattggaatacccaatatcaaaattgggcaaccccaacagcgatgaacaaactg

SEQ ID NO: 66 - DNA, 224 bp, Salmonella enterica
gagttgcgtggcagcagctttgaccacgttagcgtactcaatgtgggcacgcatacctcaggcctacagctatttatgccggaagatattaaaaataccacacagctgatggcttatctaaaagcatggaaacctgccgatgcggctggaacccatcgcgtttattccaatatcggtactggtttgctagggatgattgcggcgaaaagtctgggtgtgagcta

SEQ ID NO: 67 - DNA, 224 bp, Streptococcus pneumoniae
aatttgcttctagttttatcggtttagctcaactccatgaaaatgaagatggaagcaagagtttgctgggaacttctgggatggagagttccttgaacagtattcttgcagggaaagacggtattattacttatgaaaaggatcgtttgggtaatattgtccctggaacagaacaagtttcccaacaaacagtagatggcaaggatgtttatacaactatttcc

SEQ ID NO: 68 - DNA, 224 bp, Streptococcus pneumoniae
ttcagctgtatcgatgaaccctgctgaaaatcctgattttatcttgtatgtgacggttcaacagcctgagcatttttcaggtatccagttgggagaatttgccaccccaatcttggagcgggcttcagctatgaaagaatctctcaatcttcaatctcctgctaaaaatttagatcaagtgacgacagaatcttcttatgcaatgcctagcatcaaggatattt

SEQ ID NO: 69 - DNA, 224 bp, Neisseria gonorrhoeae
gacggcatcggcaacttcggttcgcgcgacggcgacggggcggcggcgatgcgttacaccgaagcgcggctgacgccgattgcggaattgctgttgtccgaaatcaatcaggggacggtggattttatgccgaactacgacggcgcgtttgacgagccgctgcaccttcccgcccgcttgcctatggtgttgctcaacggcgcgtcgggcatcgcggtgggtat

SEQ ID NO: 70 - DNA, 224 bp, Aeromonas salmonicida
atttacctgatctcccgggctgttaatcagtttccggagttccggatggcactgaaagacaatgaacttatttactgggaccagtcagacccggtctttactgtctttcataaagaaaccgaaacattctctgcactgtcctgccgttattttccggatctcagtgagtttatggcaggttataatgcggtaacggcagaatatcagcatgataccagattgtt

SEQ ID NO: 71 - DNA, 224 bp, Haemophilus parasuis
acccaaaaatacgttggcaaaggcatgacgattgcccaattatgtgaagcagccgtgcggtttagcgacaacagcgcgaccaatttgctgctcaaagaattgggtggcgtggaacaatatcaacgtattttgcgacaattaggcgataacgtaacccataccaatcggctagaacccgatttaaatcaagccaaacccaacgatattcgtgatacgagtacacc

SEQ ID NO: 72 - DNA, 224 bp, Xanthomonas maltophilia
cgaccacgccggcccggtcgccgagctcaagcgtcgcaccggcgcgcatgtggcggccaatgcagaaacggcggtgctgctggcgcgcggcggcagcaacgacctgcactttggcgacggcatcacctatccgccggccagcgccgaccgcatcatcatggatggcgaagtggtcacggtgggcggcatcgcattcaccgcgcacttcatgccggggcacaccc

SEQ ID NO: 73 - DNA, 224 bp, Vibrio anguillarum
tgggtgctctatatcggccgactcgtgtccggcgtcacgggcgcaaccggagctgtagcagcctcaaccattgccgattcgacgggggaaggttctcgcgcacgctggttcggctacatgggggcctgttatggggcgggcatgattgccgggccagcacttggtggcatgctcggtggtatctctgctcatgccccgtttatcgccgccgcccttctcaacgg

SEQ ID NO: 74 - DNA, 224 bp, Salmonella enterica
ggcccgctttcaagccggcttggagagcggcgcacgctgctgtttggcatggctgcggatgcgactggcttcgttcttctggcttttgccacgcagggatggatggtgttcccgattctgttgctgcttgccgccgggggtgttggcatgccggccttgcaggcaatgctctcaaacaatgtcagcagtaacaagcaaggggctttgcaaggaacgctaacgag

SEQ ID NO: 75 - DNA, 224 bp, Acinetobacter sp.
aaaggtaattcatacattacttgaagccatggttctggttttcattgtgatgtatctatttttacacaatgtccgctatacgcttattccagcaattgtggcgcctattgccttactcggtacttttaccgtgatgttgcttgccggcttttcaattaacgtactcaccatgttcggtatggtgcttgccatcgggattattgtcgacgatgccattgttgtcg

SEQ ID NO: 76 - DNA, 224 bp, Acinetobacter sp.
ggccgtattaccacccgctattgatgagttaggtactttttcaggtttcagtttacgtttacaagaccgcgctaacttaggtatgcctgctttactggctgctcaagatgaacttatggcaatggcagccaagaataaaaagttctatatggtttggaatgaagggttgccacaaggtgacaatatttctttaaaaattgaccgtgaaaagcttagtgcacttg

SEQ ID NO: 77 - DNA, 224 bp, Acinetobacter sp.
tctgtgatgctggttgtgccactcggtatttttggagcaatcattgccattatgtctagagggttaatgaatgatgtgttcttcaaaatcgggctaattaccattattggtctatcggcaaagaatgcaattttgattgttgaatttgcgaaaatgctaaaagaggaaggcatgagtttgattgaagccactgttgccgcagccaaacttcgcttacgaccaat

SEQ ID NO: 78 - DNA, 224 bp, Pseudomonas aeruginosa
acgaggtattccacttcaacgtccagcgtgaagaggacggcgtgggtgtggaagtcgccttgcagtggaacgacagcttcaacgagaacctgctctgcttcaccaacaacatcccgcagcgtgacggcggcacccacctggccggtttccgttcggcgctgacgcgtaacctgaacaactacatcgaggccgaaggcctggcgaagaagttcaagatcgccacc

SEQ ID NO: 79 - DNA, 224 bp, Pseudomonas aeruginosa
gacaagatgctctcctcccaggaggtcggtacgctgatcaccgccctgggctgtggcatcggccgcgaggaatacaacatcgacaagctgcgctaccacaacatcatcatcatgaccgatgctgacgtcgacggttcgcacatccgcaccctgctgttgaccttcttcttccgccagatgcccgagctgatcgagcgtggctacatctacatcgcccagccccc

SEQ ID NO: 80 - DNA, 224 bp, Streptococcus gallolyticus
ctttgagttttatattgaagataaagagggaagcgtactctatgccacaccaaatgccaatacatcaaatagttttagacccgactttctttatgtggtacatagagatgataatatttcgattgttgctcaaagcaaggcaggtgtgggattgctttatcaagggctgacaattcggggaattgttatgattgcgataatggttgtattcagccttttatgcg

SEQ ID NO: 81 - DNA, 224 bp, Eggerthella lenta
cattccatgtatgtcaggctgaaagaaaccatcgcaaggctggaggatgaaatcgcaagggaacatgagttggaggaaacacagcgatatttctttgcggcagcttctcatgagctaaaaacgcccatcgcggctacaagcgttctgttggagggaatgcttgaaaatatcggtgactacaaagatcattctaagtatctgcgcgaatgcatcaaaatgatgga

SEQ ID NO: 82 - DNA, 224 bp, Stenotrophomonas maltophilia
tcagcgagtgccgcgcgcagggcgcggatttcagcaacgccagcttcatgaaccagatcaccacgcgcagctggttctgcagcgccttcatcaagaagtcgaacctgcgctatgccaatttctcgcgggtgacgctggaaaagtgcgagctgtgggagaaccgctgggacggcgcgaatgtgagtggcgccagcttcgccggctcggacctgtccggcggccag

SEQ ID NO: 83 - DNA, 224 bp, Staphylococcus aureus
aatacatttttatagagatattttacttgggaaattgctattgactggtaaaaaaactgcttattttgagcttgcaggcctatggattgctttaaatgaagaaaaagatataccacgtaatgaaattcacttttcatatacacatatagctttcactatagatgacagcgaatttaaatattggcatcagaggttaaaagataataacgtgaatattttagaag

SEQ ID NO: 84 - DNA, 224 bp, Escherichia coli
atgtcactggctgagcttagcgcggccgcgctacagtacagcgataacgtggcgatgaataagctgattgctcacgttggcggcccggctagcgtcaccgcgttcgcccgacagctgggagacgaaacgttccgtctcgaccgtaccgagccgacgttaaacaccgccattccgggcgatccgcgtgataccacttcacctcgggcaatggcgcaaactctgcg

SEQ ID NO: 85 - DNA, 224 bp, Haemophilus influenzae
cggcattgcccgtgcattatttgtcgcagtgagtaatggcggtgcatcacaaggcgcaagtacgattactcaacaattagcgcgtaactttttcttaacctcagaaaaaaccattattcgtaaagctcgtgaagccgtgcttgcggtagaaatcgaaaatactctcaacaaacaagaaatcttagagctttatttaaacaaaatctttttaggctatcgttctt

SEQ ID NO: 86 - DNA, 224 bp, Haemophilus influenzae
gaagaattagatcaacaacctgaaactgtgcctgaactgccagaattacagtcaaatttcaccgcacttaaagaagatgcaattgatttaatggctgcagcaaaaaatgcttcgtcgaaaatagaatatgcaccacgtgtcattagtggcgaacttgcttttctcattcgtagtgccttaaatacggcaatttatggcgaacaaggtttagactggaaaggcac

SEQ ID NO: 87 - DNA, 224 bp, Shigella sonnei
tgtcaactgactatggtgtaacgattcgcgtcgtccccacagcgtcattgtcaacgacgtaccaactgatcctgacaaggtacgaaaaaaattactcatactcgaggcattggcaaatttattatctggcaagcctgaaaacgaaatcgttgttaagggtaaaaagaaacaggcaacggcgaatccctgaaaaaactgtggcaaaatgatgcctgtaaaatcaa

SEQ ID NO: 88 - DNA, 224 bp, Shigella flexneri
acatatcggagcgataatcacggtcattgaccataatgccagagcaaatgttcttttttctggtggataattccttaataataaactctgtgacagtggaattaacggccccgccattaacccctgaacgactctaaaaaatatcagcacatcaagattggtcgataaactacacattaaggaagacagcgaaaaaaaagtgactgaaagtaaaaataacctta

SEQ ID NO: 89 - DNA, 224 bp, Comamonas testosteroni
ggtgcagaccctgatcgaagcgatcgtgctggtgttcgtggtgatgtacctgttcctgcagaacttccgcgccaccctgatcccgaccatcgccgtgccggtggtgctgctgggtaccttcggcgtgctggcgatgctgggcttctcggtgaacatgctgaccatgttcgcgatggtgctggcgatcggcctgctggtggacgatgccatcgtggtggtggaga

SEQ ID NO: 90 - DNA, 224 bp, Herminiimonas arsenicoxydans
caagcactgggattacggttcgccgcgcctggaacgctacaacggtgtgtcggccatggaaatccagggcgaaccggccccgggtgtcgcctccggtgatgccatggccgagatcgaacagctggccaagcagctgccggcgggcttcggcatcgaatggacggcgatgtcctaccaggaacgccaggccggctcgcagacgccgctgctgtacacgctgtcgc

SEQ ID NO: 91 - DNA, 224 bp, Clostridium hathewayi
tgcgaaaaatgatactcccattctgatgatgacagccctgtcggatgacgaaaaccaaatccgggcgtttgatgcagaggcagacgactatgtaacaaagccattcaagatgcagattttactaaagcgggtggaagccctgctgcggcgcagcggtgcgctggcaaaggaaatccgtgtcggcaggctgacacttctgccggaggatttcacggtactttgtg

SEQ ID NO: 92 - DNA, 224 bp, Neisseria gonorrhoeae
gatgttgatgcattgcgtccgattgatgcgttgatgatcggcgttgcccaagtgtttgcactggttccgggtacgtcccgttcgggcagtacgattatgggcgggatgctttggggaatcgagcggaaaacggcaacggagttttcatttttcttggccgttccgatgatggttgcagcaacggcttatgatgtcctgaaacattaccggtttttcaccctgca

SEQ ID NO: 93 - DNA, 224 bp, Escherichia coli
acactatttgagctcggatctgtaagtaaaactttcacaggtgtgctgggtgcggtttctgtggcgaaaaaagagatggcgctgaatgatccggcggcaaaataccagccggagctggctctgccgcagtggaaggggatcacattgctggatctggctacctataccgcaggcggactgccgttacaggtgccggatgcggtaaaaagccgtgcggatctgct

SEQ ID NO: 94 - DNA, 224 bp, Aeromonas veronii
atggccagtcggcggttatttcatcggcaaaatggggggagttggaaacggctcagcaggagaaagctatctcaggggtaaccagattgaagctccgtttggcgtcgcttgcccctgtcctgaaaaatcacgtcaacagcgattttttccgaaaagcctctgatcgaatagagtcgatagagtatacgttggaaaccttgcgtgtaatgaaagctttcttcgat

SEQ ID NO: 95 - DNA, 224 bp, Shigella sonnei
aaccaatgcgcaaggggtgttccatgagttcttttggatcaagagaaacttcaaccggtacacgctgaacgattttgatccagttccctgtcgcattttgtgcaggtaataaggagaacgcattgccggttcccatattgatccctgtcacccgaccatgaaacacaacattttcaccataaagatcgctgataatattgaccgattgaccaatccgtacatcc

SEQ ID NO: 96 - DNA, 224 bp, Shigella boydii
gaagcgacttcggcactgtattgtttatcctgtaagtatagtttattcgtttgccgaacaatatttgccagattatttttagctttattgagtgcgatagtggcatcagttttatccagtgaaactaaaatgtcaccttgtcgaacgtagttcgtatctttatgattaacgacagtgacactacctgagacttgtgcagaaattggatctgcattccccgtgac

SEQ ID NO: 97 - DNA, 224 bp, Stenotrophomonas maltophilia
tgcgcgacctgtgccgtgccacgatcatcaccagcgacaacaccgccgccaacctgctgttcggcgtggtcggtggcccgcccgcagtgaccgcgttcctgcgcgccagcggcgacacggtgagccgcagtgaccgcctggagccggagctgaacagctttgccaagggcgacccgcgcgacaccaccacgccggcggcgatggccgcgaccctgcagcgcgtg

SEQ ID NO: 98 - DNA, 224 bp, Shigella flexneri
aatcctcgcgcggataacgcttcgtcagttcacgagagatatccagcccggtcatatctggcaactgaatatccagcaacaccaggtcgtattcgcccggtttaaacatctccagcgccgccttgccggtcatggcgacatcaacgctgttacctaatttttccagcacagaacgcgcgacaatcacgttcagttcaatgtcttccaccagcagcacattcagc

SEQ ID NO: 99 - DNA, 224 bp, Shigella sonnei
tttcaggtgaaccagttgtttttcgctttttccggtcagcagctccatcgcgcggttacagccggaaaactctttatcttcgttacgataaaaaaccaggtcgggtgaagcatcaaggaaggaacgtaagaatgaggattgctgctcgagctgaatttgtgtctcttcgcgctctttgatttcaattttcagttggccgaaggtttcctgcagttctgcttccg

SEQ ID NO: 100 - DNA, 224 bp, Pseudomonas aeruginosa
tgtcgtgcatgacgacatcatcccgtggcgttatccggctaagcgcgagctgcaatttggagaatggcagcgcaatgacattcttgcgggtatcttcgagccagccatgatcgacattgatctagctatcctgcttacaaaagcaagagaacatagcgttgccttggtaggtccggcagcggaggaattctttgacccggttcctgaacaggatctattcgagg

SEQ ID NO: 101 - DNA, 224 bp, Escherichia coli
ggtcagtaacgttcaggacccgctggcgcgtgttgaaggtgtggggagtctgcaagtctttggcgcggaatacgctatgcgcatctggcttgacccggccaaactggcgtcttactcgctgatgccttcagacgtgcaaagtgctattgaagcgcaaaacgtgcaggttactgccgggaaaataggggcattgccttcaccgaatactcagcaactgaccgcaa

SEQ ID NO: 102 - DNA, 224 bp, Shigella dysenteriae
gacagcactgaatttattaaaatctccgtagaggacgtaattcagacgctgtttgaagccatcgtattggtggtttgcgtgatgtatttattcctgcaaaacctgcgtgccacactgattccggcgttagctgtccccgtcgttttgctgggcacattcggcgttcttgcgttgtttggctattcgattaataccctgacgttgtttgcaatggtgctggcgat

SEQ ID NO: 103 - DNA, 224 bp, Mycobacterium tuberculosis
acgctgggtgggatgcacgccctgatctggcatcacggggcgatcatcgcgcatgccgcggtgatccagcggcgactgatctaccgcggcaacgcgctgcgctgcgggtacgtcgaaggcgttgcggtgcgggcggactggcggggccaacgcctggtgagcgcgctgttggacgccgtcgagcaggtgatgcgcggcgcttaccagctcggagcgctcagttc

SEQ ID NO: 104 - DNA, 224 bp, Vibrio sp.
ggacatatggatttcttagcagaagtatatcgttcattatcagttttagatggggcaattctactgatttctgcaaaagatggcgtacaagcacaaactcgtatattgtttcatgcacttaggaaaataggtattcccacaatcttttttatcaataagattgaccaaaatggaattgatttatcaacggtttatcaggatattaaagagaaactttctgcgga

SEQ ID NO: 105 - DNA, 224 bp, Vibrio sp.
aaaaacaatatagggattgataaccttatagaagtgattacgaataaattttattcatcaacacatcgaggtcagtctgaactttgcggaaaagttttcaaaattgagtattcggaaaaaagacagcgtcttgcatatatacgtctttatagtggcgtactgcatttgcgagattcggttagaatatcggaaaaggaaaaaataaaaattacagaaatgtatac

SEQ ID NO: 106 - DNA, 224 bp, Enterobacter sp.
ccgcgaaaggagagcagctactctttaaagccttgacgtacaaccagtggcttttggtgggccgcaagacgttcgaatctatgggagcactccctaataggaaatacgcggtcgttactcgctcagcctggacggccgataatgacaacgtaatagtattcccgtcgatcgaagaggccatgtacgggctggctgaactcaccgatcacgttatagtgtctggt

SEQ ID NO: 107 - DNA, 224 bp, Staphylococcus aureus
agggacatacgagttggctgaatcgcttttgaaggcaaaagaactggctgctacccaagggtacggattgcttctatgggacggttaccgtcctaagcgtgctgtaaactgttttatgcaatgggctgcacagccggaaaataacctgacaaaggaaagttattatcccaatattgaccgaactgagatgatttcaaaaggatacgtggcttcaaaatcaagcc

SEQ ID NO: 108 - DNA, 224 bp, Mycobacterium bovis
caacctgcgtgagctggccgacgcggtgttctgggcgctggagaatcacgacgccgacgaagaggagaccctggccgcggtcatggggcgggttaaaggcccggacttcccgaccgccggactgatcgtcggatcccagggcaccgctgatgcctacaaaactggccgcggctccattcgaatgcgcggagttgttgaggtagaagaggattcccgcggtcgta

SEQ ID NO: 109 - DNA, 224 bp, Mycobacterium bovis
ccaatcgctcgggcggaatcgtggcggtcaacctgcgcgacaacgacgagctggtcggtgcggtgctgtgttcggccgacgacgacctgctgctggtctcggccaacgggcagtccatcaggttctcggcgaccgacgaggcgctgcggccaatgggtcgtgccacctcgggtgtgcagggcatgcggttcaatatcgacgaccggctgctgtcgctgaacgtc

SEQ ID NO: 110 - DNA, 224 bp, Escherichia coli
actttgtacagcggcggctgagcgatgtagacgtgaccgcgttcaacgatttccggcatctgacgatagaagaaggtcaacagcagcgtacgaatgtgcgagccgtcgacgtccgcatcggtcatgatgatgatgctgtgataacgcagtttgtccgggttgtactcgtcacgaccgataccacagccaagcgcggtgataagcgtcgccacttcctgagaaga

SEQ ID NO: 111 - DNA, 224 bp, Leptothrix cholodnii
cgagatctcgatcaggaaggtggtcagcaccctgctcgaggcgatgctgctggtgttcgccgtgatgtacctgttcatgcagaacttccgcgccaccctgatcccgacactggtggtgccggtggccctgctgggcaccttcacggtgatgctcggcctgggcttctcgatcaacgtgctgaccatgttcggcatggtcctggcgatcggcatcctggtggacg

SEQ ID NO: 112 - DNA, 224 bp, Pseudomonas aeruginosa
gccgccgaggacccgcgcctggccaacgtgatgttcgccggccagggcgaggcgccgcagatccgcctggacatcgaccggcgcaaggcggagaccctcggcgtgagcatggacgagatcaacaccaccctggcggtgatgttcggctcggactacatcggcgacttcatgcacggcagccaggtgcgcaaggtggtggtccaggccgacggcgccaagcgcct

SEQ ID NO: 113 - DNA, 224 bp, Enterobacter aerogenes
gtggtttgtcattgcaggagaaactggccagcggtcaggcattaagtctgtatgcagtttctattctggtggttttcctctgccttgcagcactgtacgaaagctggtcagtcccgttctcggtcattctggtgatccctctggggcttcttggcgcggcgctggcggcctggatgcgtgatttaaacaacgacgtttacttccaggtagcgctattaactact

SEQ ID NO: 114 - DNA, 224 bp, Streptococcus thermophilus
aaagtgtttagaacaggaattaatagtgagaataaattaggttattgggataagttaaatcctttgtatacagtttttaataagcaaactgaaaaatttactaacatttggactgaatctgataacaacttcacttctttttataataattataaaaatgacttgcttgaatataaagataaagaagaaatgtttcctaaaaaaccgatacctgaaaacaccat

SEQ ID NO: 115 - DNA, 224 bp, Aeromonas hydrophila
ttgatgaatgcgcgcgatacttgcttccagatcgtgatgacgttgataaattgatcattggcagcttttgttctataggaagcggggcttccttcatcatggctggcaatcaggggcatcggcatgactgggcatcatccttccccttcttctatatgcaagaggagcctgctttctcaagagcactcgacgccttccaaagagcaggtgataccgtcattggc

SEQ ID NO: 116 - DNA, 224 bp, Shigella flexneri
cagatgctcgccaggcgtttgaaaaagccaaaactgcactggcttccagcgttcgccaaacccaccagctgatgattaacagcaaacagttgcaggcgaatattgaggtgcaaaaaatcgccctcgcgaaagcacaaagcgactacaaccgccgtgtgccgctgggcaatgccaacctgattggtcgcgaagagctgcaacacgcccgcgacgccgtcaccagt

SEQ ID NO: 117 - DNA, 224 bp, Shigella boydii
caacgacgccgctgatggcggtcgttccagccaccaatatgtgggtggatgccaactttaaagagacgcagattgccaatatgcgtatcggtcagccggtcactatcaccaccgatatttacggcgatgatgtgaaatacaccggtaaagtggttggtctggatatgggcacaggtagcgcgttctcactgcttccggcgcaaaatgcgaccggtaactggatc

SEQ ID NO: 118 - DNA, 224 bp, Escherichia fergusonii
gcgcgggtcgtggtcagcaacgttttgcaccgctgaactcctggccggataacgtaagcctcgataaagcgcgtcgcctgttgtggccaatcaaacagaaatatggtcagaaaatctcctgggccgacctgtttatcctcgcgggtaacgtggcgctggaaaactccggcttccgtaccttcggttttggtgccggtcgtgaagacgtctgggaaccggatctg

SEQ ID NO: 119 - DNA, 224 bp, Shigella dysenteriae
aacgaagcctttgcccgtgcctggttcaaactgacgcacagggatatggggccgaaatctcgctacatcgggccggaagtgccgaaagaagatctgatctggcaagatccgctgccgcagccgatctacaacccgaccgagcaggacattatcgatctgaaattcgcgattgcggattctggtctgtctgttagtgagctggtatcggtggcctgggcttctgc

SEQ ID NO: 120 - DNA, 224 bp, Shigella flexneri
aaacctgttttgccatccactctgcatcatcatgcagatctttacggtgcatatcggcgaagaacttgcgacctttatcgttcgcggctttcgcttccggcgtggcaaagcggccatcggcatcttcctggtcaacaaccagcttcagccaggcatcgtccatcgcggtttttgcacagaacaccacgacgtgcgaggcatcaagtattttacgttcgttgaac

SEQ ID NO: 121 - DNA, 224 bp, Pseudomonas mosselii
ttcaaccgtgacggcaagagcggcagcggggaccgcgtcgactggacccaaggcttcctcaccacctatgaatccggcttcacccaaggcactgtgggcttcggcgtcgatgccttcggctacctgggcctgaagctcgacggcacctccgacaagaccggcaccggcaacctgccggtgatgaacgacggcaagccgcgcgatgactacagccgcgccggcgg

SEQ ID NO: 122 - DNA, 224 bp, Pseudomonas cichorii
gacatcagcaacaccacttggtccctggcggcgcgtacactctggacgcgcacactttcaccttggcctaccagaaggtccatggcgacgagccgttcgattatatcggcttcggcgagaacggttccggcggcggcggtgactcgattttcctcgccaactccgtgcagtactccgacttcaacggccccggcgagaaatcctggcaggcccgctacgacctg

SEQ ID NO: 123 - DNA, 224 bp, Shigella boydii
caaaattacctgcgggcattggttatgactggacgggtatgtcgtatcaggaacgcttatcgggaaaccaggctcccgctctggtagcaatttcctttgtggttgttttcctgtgccttgctgcactctatgaaagctggtcaattcctgtctcggttatgttggtcgtgccgttagggattgtcggcgtgctactggcggcgacactctttaatcaaaaaaat

SEQ ID NO: 124 - DNA, 224 bp, Unknown
  Description of Unknown Uncultured bacterium polynucleotide
tctacccaagttcgagcaagcgcgaagcgagatctacatttatgacttggcagtcgcttccagccatcgaaggctaggagtcgcaactgccctgattagccacctgaagcgtgtggcggttgaacttggcgcgtatgtaatctatgtgcaagcagactacggtgacgatccggcagtcgctctctacacaaagcttggagttcgggaagacgtcatgcacttcg

SEQ ID NO: 125 - DNA, 224 bp, Stenotrophomonas maltophilia
atatcgtcgagtggtggggcggagaagaagcacgcccgacacttgctgacgtacaggaacagtacttgccaagcgttttagcgcaagagtccgtcactccatacattgcaatgctgaatggagagccgattgggtatgcccagtcgtacgttgctcttggaagcggggacggatggtgggaagaagaaaccgatccaggagtacgcggaatagaccagtcactg

SEQ ID NO: 126 - DNA, 224 bp, Salmonella typhimurium
actgggtcgtgccgcaggactcggaggtcttcgcggagagcttcgcgaccgcgctcgccgccctgcatgccgtccccatttccgccgccgtggatgcggggatgctcatccgtacaccgacgcaggcccgtcagaaggtggccgacgacgttgaccgcgtccgacgcgagttcgtggtgaacgacaagcgcctccaccggtggcagcgctggctcgacgacgat

SEQ ID NO: 127 - DNA, 224 bp, Artificial Sequence
  Description of Artificial Sequence Synthetic Plasmid pIP816 polynucleotide
ggttatagtgagcataattcaggtttatcactagatgtaggatcaagcttgacgaaaatggaacgagcccctgaaggaaagtggatagaagaaaatgcttggaaatacgggttcattttacgttatccagaggacaaaacagagttaacaggaattcaatatgaaccatggcatattcgctatgttggtttaccacatagtgcgattatgaaagaaaagaattt

SEQ ID NO: 128 - DNA, 224 bp, Shigella sonnei
ttcggagaggcgatcgatccccatatcgtaggccacctgatagaagaaggtatccgcagattcttccagcgatcttgtgacattcagacgcccgtggccccattttttccagtcacgataacgtttttccgaacctggcagttgccaccagcctgggtcaaacagcgtcgtattgcgcgtgatcaccccggcgctcaatgccgaaaccgccacatagggtttaa

SEQ ID NO: 129 - DNA, 224 bp, Artificial Sequence
  Description of Artificial Sequence Synthetic reporter vector polynucleotide
tttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgc

SEQ ID NO: 130 - DNA, 224 bp, Klebsiella pneumoniae
gaatgacttggttgagtactcaccagtcacagaaaagcatcttacggatggcatgacagtaagagaattatgcagtgctgccataaccatgagtgataacactgctgccaacttacttctgacaacgatcggaggaccgaaggagctaaccgcttttttgcacaacatgggggatcatgtaactcgccttgatcgttgggaaccggagctgaatgaagccatac

SEQ ID NO: 131 - DNA, 224 bp, Artificial Sequence
  Description of Artificial Sequence Synthetic reporter vector polynucleotide
cctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccg

SEQ ID NO: 132 - DNA, 224 bp, Klebsiella pneumoniae
ttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgat

SEQ ID NO: 133 - DNA, 224 bp, Klebsiella pneumoniae
tttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgc

SEQ ID NO: 134 - DNA, 224 bp, Klebsiella pneumoniae
ccttgagagttttcgccccgaagaacgttttccaatgatgagcacttttaaagttctgctatgtggtgcggtattatcccgtgttgacgccgggcaagagcaactcggtcgccgcatacactattctcagaatgacttggttaagtactcaccagtcacagaaaagcatcttacggatggcatgacagtaagagaattatgcagtgctgccataaccatgagtg

SEQ ID NO: 135 - DNA, 224 bp, Klebsiella pneumoniae
gatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatga

SEQ ID NO: 136 - DNA, 224 bp, Streptococcus pneumoniae
actaaagaaggaatcactgaggactttgtttggcgtgatattctttatcaaagtaactatgaaccaggatcagccatgaaggttatgacgttagcttcttctattgataataataccttcccaagtggagaatacttcaatagcagtgaattcaaaatagcggatgcgacgactcgagattgggatgttaatgatggtttgactactggtgggatgatgacttt

SEQ ID NO: 137 - DNA, 224 bp, Streptococcus pneumoniae
cttcgtgcctttacagctattgctaatgatggagttatgctggagccaaaatttataagtgctatttatgatactaacaatcagtctgtacgtaagtcacaaaaagaaatagtaggaaatcctgtttccaaagaggcagcaagcacaactcgaaatcacatgatcttagttgggacggaccctctatatggaactatgtataatcactacacaggaaagccaat

SEQ ID NO: 138 - DNA, 224 bp, Klebsiella variicola
ccgctggtggatggtcagggaaactggggggcgccggacgatcccaaatctttcgccgccatgcgttacaccgaatcccgcctgtcgaagtatgccgagctgctgctcagcgagctggggcaggggacggtcgactgggtgccaaactttgacggcacgctgcaggagccgaaaatgctgccagcgcgtctgccgaacatcctgctgaacggcaccaccggcat

SEQ ID NO: 139 - DNA, 224 bp, Mycobacterium bovis
ggtttggcagcccggaagatccgtttggctggtattataacctgctggcgctgatgacccatgtgagcgatgcgagcctgtggatgcgtctgccggatctggcggcgggcctggtgtgctggctgctgctgagccgtgaagtgctgccgcgtctgggcccggcggtggaagcgagcaaaccggcgtattgggcggcggcgatggtgctgctgaccgcgtggatg

SEQ ID NO: 140 - DNA, 224 bp, Shigella dysenteriae
ttgtccatgtaggcgttcagggtacgggtcatcgccgcacggaagcctgccaggtgagtaccgccgtcacgctgcggaatgttgttggtaaagcagtagatgttttcctggaagccatcgttccactgcaacgccacttcgacgccaataccgtctttttcggtggagaagtagaagatattcgggtggatcggcgttttgttcttgttcagatattcaacgaa

SEQ ID NO: 141 - DNA, 224 bp, Salmonella enterica
gccgtgcggcaagccctgctcgtcgatctcttggaggtttcagcttcccctggccaaaacaaggcactccgcgccttggaagtgaccatcgtcgtgcacagtgacatcgtaccttggcgttatccggccaggcgggaactgcagttcggagagtggcagcgcaaagacatccttgcgggcatcttcgagcccgccacaaccgattctgacttggcgattctgct

SEQ ID NO: 142 - DNA, 224 bp, Shigella sonnei
tgttgatgacactgcaaactctgcgcggacgtgaaacccgcaccgaacggcggcggattgatgccgtggggctggcactgctggttattggtatcggcagcctgcagattatgctcgaccgcggtaaagagctggactggttttcatcacaggaaattatcatccttaccgtggtggcggtggtggctatctgcttcctgattgtctgggagctgaccgacgat

SEQ ID NO: 143 - DNA, 224 bp, Mycobacterium marinum
gtgatattcggcttcctgctctggcatgtcatcggcgcgaattcgtcggacgacggctacatcctgggcatggcccgagtcgccgaccacgccggctacatgtccaactatttccgctggttcggcagcccggaggatcccttcggctggtattacaacctgctggcgctgatgacccatgtcagcgacgccagtctgtggatgcgcctgccagacctggccgc

SEQ ID NO: 144 - DNA, 224 bp, Mycobacterium tuberculosis
gcgggtttcatgaagccgctggacggcgattcgggttcttggggccccttgggcccgctgggtggagtcaacccggtcggcttcacgcccaacggcgtaccggaacacacggtggccgaggcgatcgtgatgaaacccaaccagcccggcaccgactacgactgggatgcgccgaccaagctgacgagtcctggcatcaatggttctacggtgccgctgcccta

SEQ ID NO: 145 - DNA, 224 bp, Anaeromyxobacter sp.
ggtagcccaggaccagaatgcgctggtcctgctgctgggctccgggatcccggcgaacctgccgcaaggcctgggcctggaccagaccctgctgaccgaagtgccggcgggtctgccgtcggacctgctgcaacggcgcccggacatcctcgaggccgagcaccagctcatggctgccaacgccagcatcggcgccgcgcgcgcggcgttcttcccgagcatca

SEQ ID NO: 146 - DNA, 224 bp, Shigella sonnei
tacgtttgcggcgatgtacctgtgcggattcagtctcaataatctttcgttaatggcgctcaccatcgctactggtttcgtggtggatgacgccatcgtggtgctggaaaacattgcccgtcatctggaagcggggatgaaaccgctacaggccgcgctgcaaggtactcgcgaagtcggctttacggtgctgtcgatgagtctgtcactggtggcggtgttcc

SEQ ID NO: 147 - DNA, 224 bp, Enterococcus faecium
aattaaaccctttatatacagtctttaataaagaaactgaaaaattttctaacatttggacagaatcaaatgctagttttaactctttttataatagttataagaatgatttacttaaatataaagataaaaatgaaatgtttcctaaaaagccgatacctgaaaacacagttcctatctcgatgattccttggattgattttagttcatttaatttaaatatt

SEQ ID NO: 148 - DNA, 224 bp, Pseudomonas aeruginosa
tgatcgagcagcagatgaacgggatcgacaatctgcgctacatctcctcggagagtaactccgacggcagcatgaccatcaccgtgaccttcgaacagggcaccgaccccgacatcgcccaggtccaggtgcagaacaagctgcaactggccaccccgctactgccgcaggaagtgcagcgccaggggatccgggtgaccaaggcggtgaagaacttcctcatg

SEQ ID NO: 149 - DNA, 224 bp, Pseudomonas aeruginosa
accagggcgtactgttcgcccaggtacagaccccgccgggctccagtgccgagcgtacccaggtggtggtggactcgatgcgcgaatacctgctggagaaggaaagctcttcggtcagctcggtgttcaccgtgaccggcttcaacttcgccggccgcggccagagttcgggcatggcgttcatcatgctcaagccctgggaagagcgtcccggtggcgagaac

SEQ ID NO: 150 - DNA, 224 bp, Ralstonia eutropha
gcaagtgggaatacggttcgccgaagctggagcgctacaatggcgtgccggcgatggagatcctcggcgagccggcgcccggcctgagttccggtgacgccatggcggcggtcgaggagatcgtcaagcaattgccgaaaggcgttggctactcctggaccggcctgtcctacgaggagcgcttgtccggctcgcaggcgccggcgctgtatgcgctgtcgctg

SEQ ID NO: 151 - DNA, 224 bp, Escherichia coli
tttctccgatggaggccggtatctggcgccagacgcagccattgcgcaggcgcgtaagctgatggccgagggggcagatgtgatcgacctcggtccggcatccagcaatcccgacgccgcgcctgtttcgtccgacacagaaatcgcgcgtatcgcgccggtgctggacgcgctcaaggcagatggcattcccgtctcgctcgacagttatcaacccgcgacgc

SEQ ID NO: 152 - DNA, 224 bp, Unknown
  Description of Unknown Uncultured bacterium polynucleotide
gcgcttcgtcgacgtggccatcgagaaggtgatccacaccctgatcgaagcgatggtcctggtgttcctggtgatgttcctgttcctgcagaacgtccgctacaccctgatcccgtccatcgtggtgccggtgtgcctgctgggtacgctgatggtgatgtacctgctggggttctcggtgaacatgatgaccatgttcggcatggtcctggcgatcggcatcc

SEQ ID NO: 153 - DNA, 224 bp, Pseudomonas aeruginosa
ggaaggactggccgaagcgccgcaactgcgcctgttgatcgaccgggagaaggcccgcgccctgggggtgagcttcgagaccatcagcggcacgctgtccgctgccttcggctcggaggtgatcaacgacttcaccaatgcggggcgccaacagcgggtggtgatccaggccgaacagggcaaccggatgaccccggaaagcgtgctcgagctatacgtgccta

SEQ ID NO: 154 - DNA, 224 bp, Azoarcus aromaticum
caacgccttcctggtcgccgaagtgcgcccgcaggtcaacggcatcgtcgccaagcgcctgttcaccgaaggcggcatggtcaaggccggcgagccgctgtaccagatcgaggacgccagctaccgcgcccaggccaacagcgcccgcgcccagctggcccgcgccgaagccaccgccaacgccgcgcgcctgagcgccaagcgcatcaccgagctggccaagg

SEQ ID NO: 155 - DNA, 224 bp, Klebsiella oxytoca
cacaagggtcagattttcgcggcgcaagttttatgaatatgatcaccacccgcacctggttttgtagcgcctatatcaccaataccaacttaagctacgccaacttttcaaaagtcgtactggaaaagtgcgagctgtgggaaaaccgctggatgggtactcaggtgctgggcgcaacgttcagtggatcagacctctctggcggcgagttttcatccttcgac

SEQ ID NO: 156 - DNA, 224 bp, Enterococcus faecium
tgagcaactatcaaatggtgagcggatagatattcggatttctccctatcttcaagatttgtttgatgccgcaagaactgatggagtttacccgattgtcgcatccggataccgaacaacagaaaaacagcaagaaattatggatgaaaaaattgccgaatataaggcgaaaggctacacctctgcacaggctaaagcggaagcagaaacttgggtggccgtgc

SEQ ID NO: 157 - DNA, 224 bp, Enterococcus sp.
tccgaaagcgatggggcttgtgtcagctgcaattagtggtggttttattatcggaccaggagttggtggttttattatcggaccaggagttggtggttttattgcttatttaggtattcgcgctccattttttgcggccgcatttttagcgtttattggttttattttgacattaactgttttgaaggagccagagaaacgaattttagccgctgttgaagcga

SEQ ID NO: 158 - DNA, 224 bp, Lactobacillus fermentum
tcggtaaattttgttctatcgccagtggcattgaatttatcatgaacggtgccaaccacgtaatgaaaggtatttcgacttatccatttaatattttaggtggcgattggcaacaatacactcctgaactgactgatttgccgttgaaaggtgatactgtagtcggaaatgacgtgtggtttgggcaaaatgtgaccgtcctaccaggcgtaaaaataggtgac

SEQ ID NO: 159 - DNA, 224 bp, Escherichia coli
cccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggaga

SEQ ID NO: 160 - DNA, 224 bp, Artificial Sequence
  Description of Artificial Sequence Synthetic reporter vector polynucleotide
aatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctg

SEQ ID NO: 161 - DNA, 224 bp, Acinetobacter baumannii
tcgggatcgtgaccctggaagcgcagacggtgaccctgaataccgagctgccgggccggaccaatgcgttccgcatcgccgaggtgcgtccccaggtgaacggcatcatcctcaagcgcctgttcaaggaaggcagcgacgtcaaggccgggcagcagctctaccagatcgaccccgccacctacgaggccgactaccagagcgcccaggccaacctggcttcg

SEQ ID NO: 162 - DNA, 224 bp, Pseudomonas aeruginosa
cccgctggaaggtcgcctcgaattctccgaggtttccgtcgacgaaggcaccggctcggtcaccatccgcgccgtgttccccaacccgaacaacgagctgctgcccggcatgttcgttcacgcgcagttgcaggaaggcgtcaagcagaaggccatcctcgctccgcagcaaggcgtgacccgcgacctcaagggccaggctaccgcgctggtggtgaacgcgc

SEQ ID NO: 163 - DNA, 224 bp, Acidovorax avenae
gaattcgtggaacaggcgcccgaccacgtcgcccatgaacagcagcgggatcagcaccgcgatcagcgacaccgtcagcgagatgatcgtgaagccgatctgcctcgagcccttcagcgcggcctcgagccccgattcgccttcctcgacgtagcgcgcgatgttctcgatcatcacgatcgcgtcgtcgacgacgaagccggtcgcgatggtgagcgccatca

SEQ ID NO: 164 - DNA, 224 bp, Burkholderia phytofirmans
acgatcgtgcggatggcgcaggacttctcgctgcgctacatgctgatcgacgggcagggcaacttcggctcgatcgacggcgacaatgccgcggcgatgcgttacaccgaaattcgcatggcgaagatcggtcacgagctgctcgccgacatcgacaaggaaacggtcgatttcgagccgaactacgacggcaacgaaatgcagccgtcggtcctgccgtcgcg

SEQ ID NO: 165 - DNA, 224 bp, Burkholderia cenocepacia
gatgtcgcatgcgggctacgtgaagtcgcagccgctgtccgagtaccgcgcgcagaagcgcggcggtcgcggcaagcaggcgacgcagatgaaggaagacgactggatcgagacgctgttcatcgcgaacacgcacgactacatcctgtgcttctcgaaccgcggccgcgtgtactgggtcaaggtctatgaagtgccgcagggctcgcgcaactcgcgcggcc

SEQ ID NO: 166 - DNA, 224 bp, Burkholderia stabilis
cctgctgatgaagctgatcggcggcccgtcggcggtcaccgcctacgcgcgctcgatcggcgacgacgcgttccggctcgatcgatgggagaccgaactgaataccgcgttgccgggcgacccgcgcgacacgacgacgcccgccgcgatggccgccagcatacgcgtgctgacgctcggcgacgcactgccggccgcgcagcgtgcgcagctcgtcgcgtggc

SEQ ID NO: 167 - DNA, 224 bp, Escherichia coli
atttgcggttctacagccagcgcgcgcgccagcgccacacgctgtttctggccgccggaaagctgcgccggataacgatccgccagatgcgcaagctgcaccatctccagcaattttgtcactttcgctttgatggctgcggcattcgggcgctcgcgacgcggcagcaccgtcaggccaaaagcgatattgtcgaacaccgtcatatggcggaacagcgcgta

SEQ ID NO: 168 - DNA, 224 bp, Salmonella enterica
tggaaaatcccaggtgagcaaaagatttttcgcaggctcaccgagggcaaagtggtcgttatgggccgcaagacgtttgagtccataggcaagcccttaccaaaccgccgcacagtggtgctctcgcgccaagctagttatagcgctgctggttgtgcagttgtttcaacgctgtcgcaggctattgccatcgcagccgaacacggcaaagaactctacgtggc

SEQ ID NO: 169 - DNA, 224 bp, Staphylococcus aureus
ataattgattctttgcttttttactaaaatggttattttgttccatcatatttttaaaaactattacttgttcaagattagatattttcaaactatcttccatccaatagcttttataacttcccaaattttcattaccataatttaattgcttgagttgagccgcagtatagttctttggtatttgattactaatacgttcgaagtaccaattaactgaattt

SEQ ID NO: 170 - DNA, 224 bp, Shigella sonnei
tgacgaaagacgagatcctcgagctttatctgaacaagatttacctcggttaccgcgcctatggtgtcggtgctgcggcacaagtctatttcggtaaaacggtcgaccaactgacgctgaacgaaatggcggtgatagccgggctgccgaaagcgccttccacctttaatccgctctactcaatggatcgtgccgtcgcgcggcgtaacgtcgtactgtcgcgg

SEQ ID NO: 171 - DNA, 224 bp, Mycobacterium avium
cgatcaaggagttcttcggcaccagccagctgtcccagttcatggaccagaacaacccgctgtcggggctcacccacaagcgccgcctgtcggcgctgggcccgggtggtctgtcccgggagcgggccgggctggaggtccgcgacgtgcacccgtcccactacggccggatgtgcccgatcgagaccccggagggtcccaacatcggtctgatcggctcgctg

SEQ ID NO: 172 - DNA, 224 bp, Mycobacterium chimaera
ctgcggcacgcacagccgaaccagatcgtgtcgacaccggtgttcgacggcgccaaggaagaggagctggccggcatgctgtcgtgcacgctgcccaaccgcgacggcgaggtcatggtggacggcgacggcaaggcggtgctgttcgacggccggtccggggagccgttcccgtacccggtgaccgtcggctacatgtacatcatgaagctgcaccacctggt

SEQ ID NO: 173 - DNA, 224 bp, Corynebacterium urealyticum
ccagcgaaccagaaggggcgtgcactgtcgatcctgctctccggcacgacgatcgcaaccgtcgtgggcgtccccgccggggcactgctcggcacagcgctgggctggcgaacgacgttctgggcgatcgccatcctctgtattcccgcggccgttggagtcattcgtggcgtcacgaacaatgttggtcggagcgagactagcgcgacctcaccaaggctccg

SEQ ID NO: 174 - DNA, 224 bp, Enterococcus faecalis
acgtaaaatcgattgtgcgctctgtggaaaaacatgatttcaggttggacagcgaccgtggcaaggtactcagcgacatgacagttggtgtggtgggaacgggccagataggcaaagcggttattgagcggctgcgaggatttggatgtaaagtgttggcttatagtcgcagccgaagtatagaggtaaactatgtaccgtttgatgagttgctgcaaaatagc

SEQ ID NO: 175 - DNA, 224 bp, Enterococcus faecalis
caacaagtggctgaatatttgaagaaaaagaaacacgggtttattttagtcagccacgatcgggcatttgttgatgaagtggttgatcatattttggcgattgaaaaaagtcaattgacgctgtatcaagggaatttttctatttatgaagagcaaaaaaaattaagagatgcttttgaactagcagaaaatgaaaaaatcaaaaaagaagtcaatcgcttgaa

SEQ ID NO: 176 - DNA, 224 bp, Enterococcus sp.
gccaatttctttttcaataaacgcgggagaaattgtcggaataacaggaaaaaatggctcaggaaaatcgagcttgattcagtatttgttggataatttttctggggattcagaaggcgaagccactttggctcaccaattaaccatttcttatgtgcgccaagattatgaagacaatcaaggaactttatccgaatttgcagagaaaaatcagttagattaca

SEQ ID NO: 177 - DNA, 224 bp, Azoarcus sp.
cgatcccgagcggctggcgcagctcggagcccgcgccggagccgacgatcagcggcaccgcgccgagcagcgcggcgagcgtcgtcatcaggatcggccggaagcgcagcaggcacgcctggtagatcgcctcgcgcggcggcttgccttcgacgcgttcggcctcgagcgcgaagtcgatcatcatgatcgcgttcttcttcacgatgccgatcagcagcacg

SEQ ID NO: 178 - DNA, 224 bp, Artificial Sequence
  Description of Artificial Sequence Synthetic reporter vector polynucleotide
tcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcg

SEQ ID NO: 179 - DNA, 224 bp, Enterococcus faecium
ccgggggaaagaactgggggatatgactgtgggagttattggaaccggccatatcgggcaagcggtcgtcaaaaggctgcggggatttggatgccatgtgctggcctatgataacagccgaaaaatggatgcagattatgtccagcttgatgagcttctaaaaaacagcgatattgttacgctccatgtgccgctttgtgcggatacccgccatctgatcggtc

SEQ ID NO: 180 - DNA, 224 bp, Acinetobacter sp.
ttaccaaaaatggctataaagcgttgaatcaaagcaatacgctctttcgtatctgaatttccacgtttattaagcaatgtccaaaggataggtatcgctattccacgataaacgattgcgagcatcaggatattaatatttcgttttccccatttccaattggttctatctaaagtcagttgcacttggtcgaatgaaaacatattgaaaatcaactgagaaat

SEQ ID NO: 181 - DNA, 224 bp, Artificial Sequence
  Description of Artificial Sequence Synthetic shuttle vector polynucleotide
gtttaggacggcaatcaatcaagatggtgaattggggatatatgatgagatgataccaagctatacaatatttcacaatgatactgaaacattttccagcctttggactgagtgtaagtctgactttaaatcatttttagcagattatgaaagtgatacgcaacggtatggaaacaatcatagaatggaaggaaagccaaatgctccggaaaacatttttaatg

SEQ ID NO: 182 - DNA, 224 bp, Stenotrophomonas maltophilia
atggaacttacatgcaccgttctttgctgctgcttttatgaatgggattaatttaataatgacagcagtcttattaaaagaatcaaaacacagcaataaaatgactgagaaggttcaggagcaatcaatattaaagaaattatcctatttgatcactcaacctaatatggctccattgcttggtatctttttaattatcacattggtttcacaagtccccgcaa

SEQ ID NO: 183 - DNA, 224 bp, Burkholderia cenocepacia
cgacgcgcgccaggccctggcgcagatcgcctcggccaaggccgaactggagcaggcccgcctgcgcctgggctacgccacggtcaccgcgccgatcgacggccgcgcgcggcgtgcgctggtcaccgaaggcgcgctggtcggcgaggactcgccgacaccgctgacccgcgtcgagcagatcgatccgatctacgtgaacttctcccagccggccggcgaag

SEQ ID NO: 184 - DNA, 224 bp, Culex pipiens
ccgccaggccggtcgcgacacgcgcagcgagagcccggtgtacctcggcctgagcagcgaggacggcaacccgcacctgggccggctggacttcctcgacaaccaggtcaacccgcgtaccggcaccatccgcggccgcgccgtgttcgacaacgccaagggcgagttcaccccgggcctctacgtgcgcctgaagctggtcggcagcaagacctacgccgcca

SEQ ID NO: 185 - DNA, 224 bp, Pseudomonas aeruginosa
agccgcttcgaaagcctgtggtggaaacagttcgacgatccgaccctgaaccagttggtcgaacagtcgctgagcggcaaccgcgacctgcgcgtggccttcgcccgcctgcgcgccgcccgcgccctgcgcgacgacgtggccaacgatcgcttcccggtggtcaccagccgcgccagcgccgacatcggcaagggccagcaaccgggagtgaccgaggaccg

SEQ ID NO: 186 - DNA, 224 bp, Pseudomonas entomophila
cggccaacggccggaagagttgacagtggacctttcgccgcgcgacctgccggcgatcaccaaggccctgccgatcggcgatcccggcgaactgctgcgccgccggccggacatccgcgccgccgaacggcgcctggccgccagcaccgccgacgtcggcgtggccaccgccgacctgttcccgcgggtcagcctcagcggcttcctcggcttcaccgccgggc

SEQ ID NO: 187 - DNA, 224 bp, Escherichia coli
catcatttttttagatgctatagggattggcataattatgcctgtcttgccggcgttattacgggagtttgttggaaaggctaatgttgcagagaactacggtgttttattggcgctgtatgcaatgatgcaagtgatttttgcccctcttctcggccgctggtcagatcgcataggtcgtcgccctgtattgttactttcacttttaggtgcaacactggact

SEQ ID NO: 188 - DNA, 224 bp, Aeromonas salmonicida
ccattaacgggctggcatttctggtctccctattcattttacatgagacccataatgctaatcaggttagtgacgagataaagaatgaaacaatcaatgaaaccacatcctccatacgcgagatgatctccccattatcgggattgctagttgtctttttcatcattcaattgattggccaaatccctgcaacattatgggttttattcggagaagagcgcttc

SEQ ID NO: 189 - DNA, 224 bp, Salmonella typhimurium
ggtgtaatggttttctcctcaattcaggatgccttaataaatttagaggaaatcacggatcatgttatcgtttctggtggtggtgaaatatacaaaagcttgatttccaaagtagatactttgcatatttcaacagtcgacatcgagcgagatggagacatagtttttcctgaaatcccagatacattcaagttggtatttgagcaagatttcgagtctaacat

SEQ ID NO: 190 - DNA, 224 bp, Pseudomonas putida
cgtttgatggcgcagtctacccgtccaatggtctcattgtccgtgatggtgatgagttgcttttgattgatacagcgtggggtgcgaaaaacacagcggcacttctcgcggagattgagaagcaaattggacttcctgtaacgcgtgcagtctccacgcactttcatgacgaccgcgtcggcggcgttgatgtccttcgggcggctggggtggcaacgtacgca

SEQ ID NO: 191 - DNA, 224 bp, Shigella sonnei
catgcacgtttcgcttacgacaaagaaaaaaccgatcgctataacgagcgaccgtggggtggcggttttggtcagtcgcgttgggatgaaaaaggaaactggcatggcctgtatgccatggcatttaaggactcgtggaacaaatgggaaccgattgccggatacggatgggaaagtacctggcgaccgctggcggatgaaaattttcatttaggtctgggatt

SEQ ID NO: 192 - DNA, 224 bp, Shigella sonnei
atggatagttcgtttacgcccattgaacaaatgctaaaatttcgcgccagccgccacgaagattttccttatcaggagatccttctgactcgtctttgcatgcacatgcaaagcaagctgctggagaaccgcaataaaatgctgaaggctcaggggattaacgagacgttgtttatggcgttgattacgctggagtctcaggaaaaccacagtattcagccttc

SEQ ID NO: 193 - DNA, 224 bp, Shigella sonnei
agcgccaatatagcggaattattattatcgtctccgctaaaaatgaccatttttacgggaaacattgtgctgatgctggcgctaatggtttcgtgagtaaaaaagaaggcatgaacaatatcattgcggctattgaagctgcaaaaaatggctactgctatttccccttctctctcaaccggtttgttggaagtttaacgtccgaccagcaaaaactcgactcc

SEQ ID NO: 194 - DNA, 224 bp, Eggerthella lenta
caaggtgttcaacttaaagctggcagcaaagaccttaaacggcttattgataagaccgggggaaaccttttctttctggtggctggtacgccatgcggacaaagataccccctataaagacggccttacggtgaccaatggtaaactcaccaccatgtcgggcggcggtatgtgccagatgagcaatttactattttggatgttcctgcatacgccattgacaa

SEQ ID NO: 195 - DNA, 224 bp, Enterobacter cloacae
aaggagtatcggatcccggggatggcggtcgccgtactgaaagatggcaaggcccactatttcaactatggggttgccaaccgcgagagtggccagcgcgtcagcgagcagaccctgttcgagattggctcggtcagcaagaccctgaccgcgaccctcggtgcctatgctgcggtcaaggggggctttgagctggatgacaaggtgagccagcacgccccctg

SEQ ID NO: 196 - DNA, 224 bp, Klebsiella oxytoca
tattccaaccccagcataggcctgtttggtcacctggccgcaaatagtctgggccagccatttgagcaactgatgagccagaccctgctgcccaagctgggtttgcaccacacctatatccaggtgccggagtcggccatggcgaactatgcctacggctattcgaaggaagataagcccgtccgggtcactccgggcgtgctggcggccgaggcttacgggat

SEQ ID NO: 197 - DNA, 224 bp, Streptococcus salivarius
tatttctgatttggacaaatgggatatgattatttccggaagcgatgaactattagaacgatatgttgcagaggattctttggatatacaggaattacaatatgaaaagtgcaaaagaaccagatgctgctctttgtttcctgtttatcatgggagtgcaaaagacaatttaggaacagaaaaactgattgaagcgattacagaaactttcattacagaaacag

SEQ ID NO: 198 - DNA, 224 bp, Streptococcus salivarius
tggagggtgtgcgttatggaatggagcagggcttatatggctggggagtgacagactgccaaatttgttttgattatggagtttattacagcccggtcagcacccccgctgattttcgttttcttgcgcctgtcgtgttggagcaggcattgaaaaaagcaggaacacaactgttggaaccatacctttcctttaccctttttgcaccgcaggaatatctttca

SEQ ID NO: 199 - DNA, 224 bp, Escherichia coli
tatccaaggatttcctgaccctgcgctctatcccgatattgctgaggcggactgcaggctggtggttatgcactcagcgcagcgggatggcatcgccacccgcaccggtcaccttcgacccgaagacgcgctcgacgagattgtgcggttcttcgaggcgcgggtttccgccttgcgacggagcggggtcgctgccgaccggctcatcctcgatccggggatgg

SEQ ID NO: 200 - DNA, 224 bp, Enterobacter aerogenes
ccgcagtgggcgccgggtagtaaacggctgtacgccaatgccagcattggcctgttcggcgcgctggcggtgaaaccttccggcatgagctttgaacaggcgatgagccagcgggttttgcagccgttaaaactgagccacacctggattaacgtaccgcccgcccagagcaaggactacgcctggggttatcgtgacggtaaagcggtacatgtttcgccggg

SEQ ID NO: 201 - DNA, 224 bp, Enterobacter aerogenes
gccaaaggcgacctggcaaaagcccaggcggcggcaaacatggatcaactgacggtcaagcgttatcagaaactgttgggcaccaaatatattagtcaacaagactacgataccgccgttgcgacggcgcagcagagcaatgcggccgtggtcgcggcgaaagctgccgttgaaaccgcgcgcatcaatctggcctacaccaaagtcacctcgccgatcagcgg

SEQ ID NO: 202 - DNA, 224 bp, Klebsiella variicola
ccgtctggaagaagggattaaccctgacgccctgctggtaccgcaacagggtgttacccgtacgccgcgcggcgacgccagcgtcatggtagtgggggaaggcgataaagtcgaagtccgccaggtcactgcttctcaggcgatcggcgataaatggctggtcactgacggtctgaaatccggcgatcgcgttatcgtcaccggcctgcaaaaaatcaaaccag

SEQ ID NO: 203 - DNA, 224 bp, Mycobacterium tuberculosis
gcggctctgaagggcagcccgcagcgtcgtggtgtatgcacccgcgtgtacaccaccactccgaagaagccgaactcggcgcttcggaaggttgcccgcgtgaagttgacgagtcaggtcgaggtcacggcgtacattcccggcgagggccacaacctgcaggagcactcgatggtgctggtgcgcggcggccgggtgaaggacctgcctggtgtgcgctacaa

SEQ ID NO: 204 - DNA, 224 bp, Staphylococcus rostri
atggttacagttatataatgtattttaaagatggcataaaaatggatattacattaattaatttaaaagatttaaatcgttattttagtgattctgatggtcttgtaaaaattttagttgataaagataatttagtaactcaagaaattgttccagatgactcaaattattggttaaaaaaaccaacagaacgagaattttatgattgctgtaatgagttttgg

SEQ ID NO: 205 - DNA, 224 bp, Klebsiella variicola
acagaatatgaacggtatcgatcacctgatgtacatgtcctcaaatggcgactccaccggtacggcaaccattacgctgaccttcgaatccggtaccgatccggatatcgcccaggttcaggttcagaacaagctggcgctggcgacgcctctgctgccgcaagaagtacagcagcaagggattagcgttgagaaagcgtccagcagcttcctgatggttgtcg

SEQ ID NO: 206 - DNA, 224 bp, Salmonella typhimurium
tgggccacgaaaaactgacccaggcgcgtaaccaactgtttggcatggtggcgcagcatcctgacgtgctgaccggcgtgcgccctaacggtctggaagatacaccgcagtttaaaatcgatatcgatcaggagaaagctcaggcgctgggcgtctccatcagcgacattaacaccacgctgggcgcagcctggggcggaagctatgtcaacgactttatcgac

SEQ ID NO: 207 - DNA, 224 bp, Enterobacter aerogenes
gaagatatcggcaagtggtatgttcgcggcagcgatggtcagatggtgccgttctctgctttctcgacctcgcgttgggaatacggttcgccgcgtctggaacgctacaacggtctgccgtcactggaaatcctcggtcaggccgcgccaggcaagagtaccggtgaggcgatggcgctgatggaagagctggcgggtaaactgccttccggtatcggctacga

SEQ ID NO: 208 - DNA, 224 bp, Burkholderia cenocepacia
aggatcagcgacaccttcgccgcgcccgggccgctcgtcttcagacggccgctctgcacgtcctgacgcagcttcagcccttcgaggctcgactgcgtgaggtcgacgtacaccggatcgagctgctgcacggtcgacatcagcgtcgcctggctcgcctgcacgtaagcgcccggcgtcacttgcgagatgccgacgcggccgctgatcggcgacacgacgtc

SEQ ID NO: 209 - DNA, 224 bp, Enterococcus faecium
aatcggatttttacctaagtttgcttttgtactggttttaagtcttacttttgaaataattcaatttatcttcgctattggagcgacagacataacagatgtaattacaaatactgttggaggctttcttggactgaaattatatggtttaagcaataagcatatgaatcaaaaaaaattagacagagttattatttttgtaggtatacttttgctcgtattat

SEQ ID NO: 210 - DNA, 224 bp, Shigella boydii
atttgacggtatttgacgccacctgtccgctggtgaccaaagtgcatatggaagtcgcccgcgccagtcgccgtggcgaagaatctattctcatcggtcacgccgggcacccggaagtggaagggacgatgggccagtacagcaacccggaagggggaatgtatctggtcgaatcgccagacgatgtgtggaaactgacggtcaaaaacgaagagaagctctcc

SEQ ID NO: 211 - DNA, 224 bp, Acinetobacter baumannii
aaaccgttcgtattaccaatagccacgggcaaccttataacgtcacagcaaaaatgttgtttgaagatattaatgttgacccggaaacgggcgatgtcacattccgtattgaagttaataacactgaacgaaaattacttccgggtatgtatgtgcgtgtcaatattgaccgtgcttctattcctcaagcgctattggttccggcgcaagcgatccaacgtaat

SEQ ID NO: 212 - DNA, 224 bp, Clostridium difficile
gaagcattacagctactagaggaagagattcaaaatcctcaatttgataatatgaaattttacaaaaaaaggatggaatttatatatgatagacaaaaaaggtttataaattattataaatctcaaatcaataaacctacagtacctacaatagatgatattataaagtctcatctagtatctgaatataatagagatgaaactttattagaatcatatagaac

SEQ ID NO: 213 - DNA, 224 bp, Clostridium difficile
atattgaatcttctattggtgattacatttattatgaaaaattagaacctgttaaaaatataattcacaattctatagatgatttaatagatgagttcaatctacttgaaaatgtatctgatgaattatatgaattaaaaaaattaaataatctagatgagaagtatttaatatcttttgaagatatctcaaaaaataattcaacttactctgtaagatttatt

SEQ ID NO: 214 - DNA, 224 bp, Streptococcus pneumoniae
gaagaaagagctgaaaaaattgcttcagatattgacagagctgaagaagcccgtcaaaaagcagaagtattggctcaaaaacgcgaagatgaattggctggtagccgtaaagaagctaagacaatcattgaaaatgcaaaggaaacagctgagcaaagtaaggctaatatcttagcagatgctaaactagaagcaggacgcttaaaagaaaaagcaaaccaaga

SEQ ID NO: 215 - DNA, 224 bp, Stenotrophomonas maltophilia
ggttgcccatgtgctgagccttgccgatgcaggccgcgtttcgctcgacacccgtgtgcccatcgccgggaaggatctgctgtcctacgccccggtggcgcgccgccatgtgggcaaggacctgactgtgcgcgacctgtgccggggcacgctgaccaccagcgacaacacggcggccaacctgctgctggaggtggtgggcgggccgtcggcgctgacggcat

SEQ ID NO: 216 - DNA, 224 bp, Helcococcus kunzii
gaggatattttgaagtttagctttcctaaaaatacagactataaaatatttggtaatattccctacaatattagtactgatattgtaaaaaagattgcttttgatagtcaagcgaaatatagctaccttattgtagagaggggatttgctaaaaggttgcaaaatacccaacgagctttaggtttgctgttaatggtggaaatggatataaaaattcttaaaaa

SEQ ID NO: 217 - DNA, 224 bp, Actinobacillus pleuropneumoniae
ttcagtcgacccacaattcaccgtattccatcaagaaacagagacattttcagcactgagttgcccatactcatccgatattgatcaatttatggtgaattatttatcggtaatggaacgttataaaagtgataccaagttatttcctcaaggggtaacaccagaaaatcatttaaatatttcagcattaccttgggttaattttgatagctttaatttaaatg

SEQ ID NO: 218 - DNA, 224 bp, Acinetobacter sp.
aaccagaaaacggatattaatgaaatatttaaatggaagggcgagaaaaggtcatttaccgcttgggaaaaagacatgacactaggagaagccatgaagctttctgcagtcccagtctatcaggaacttgcgcgacgtatcggtcttgatctcatgcaaaaagaagtaaaacgtattggtttcggtaatgctgaaattggacagcaggttgataatttctggtt

SEQ ID NO: 219 - DNA, 224 bp, Salmonella enteritidis
attatcggcagcttctgctccatcggatcaggcgcagcttttattatggctgggaatcaaggccaccgatatgattgggtctcttctttccctttcttctacatgaacgaggagcccgcgtttgcaaaatcagtcgatgcattccagcgggctggcgacacagttataggaagtgatgtgtggatcggttcggaggccatgatcatgcccgggatcaagatcgg

SEQ ID NO: 220 - DNA, 224 bp, Acinetobacter baumannii
tgtcggcgcgcttattattttctgggttgaatctaaacaatttgaacataaaacggatgatgcgaccaagatcacgtttaaacaggcgcttttagttggtctggctcaatgtgttgcgatgattcctggaacctctcgttctggtgcgacaattgtaggcgggatgtttgcaggcttgtctcgtaaagctgcaacagagttttcttttttccttgcaatgccga

SEQ ID NO: 221 - DNA, 224 bp, Staphylococcus epidermidis
tatataaaaaaattgtgcctgaagaaggtattgtaaaacaattttcacattgtgaacttattcctcaattgaagctcatagaatcaactaaaagtggtggtgaagtaacacgaaactatattcggcaagcgcttgataaaaatccagaactgctattagcagatgaaccaacaactaacttagataataactatatagaaaaattagaacaggatttaaaaaat

SEQ ID NO: 222 - DNA, 224 bp, Staphylococcus epidermidis
attttagccaaaaaatagatacattagaattagataagagtattttagaaaatgttcaatcttcttcacaacaaaatgaaactcttattcgaactattctagctagaatgcatttttttagagatgatgtttataaaccaataagtgtcttaagtggtggagagcgagttaaagtagcactaactaaagtattcttaagtgaagttaatacgttggtactagat

SEQ ID NO: 223 - DNA, 224 bp, Neisseria meningitidis
ctggctggcagaacttgcaccaccggcaatatacaggggaaccacgcccaaaataaaggcgaacgaggtcatgataatcggacggaaacgcaggcgggctgcttccaaagcggcttcgaccgcgcttttcccttgcgcttgaaggtctttggcaaattcgataatcaaaatcgcgtttttcgcactcaaacccatcacggtaacgaaaccgacttgaaagtaga

SEQ ID NO: 224 - DNA, 224 bp, Burkholderia vietnamiensis
ccgcctgccgcagcggcacgccgctcccggccagatcgcgcgcgtactgcacgatcaggatcgcattcttcgccgccagcccgaccaccgtgatcatcccgaccttgaaatacacgtcgttcggcatcccgcgcgcgagcgccgccgcgatcgcgccgatcatgccgagcggcacgatcgtcagcacggacagcgggaccgtccagctttcgtacagcgcggca

SEQ ID NO: 225 - DNA, 224 bp, Burkholderia vietnamiensis
gcggccgatttcgacacgcgcgacgtcggcgacgcgcaccaccgaaccgtcctgcttcgacttcagcacgatccggccgaactcctccggcgacgttagctgtcccttgacgacgatcgtcgcggtgagctgctggccgcgctcgaacggcgcatcgccgatcgcaccggccgtcaccgtcgcgttctgcacgccgatcgccgcgatcacgtcgtcggcgccga

SEQ ID NO: 226 - DNA, 224 bp, Providencia stuartii
ttcatgatgaaaaaatcgttatgctgcgctctgctgctgacagcctctttctccacatttgctgccgcaaaaacagaacaacagattgccgatatcgttaatcgcaccatcaccccgttgatgcaggagcaggctattccgggtatggccgttgccgttatctaccagggaaaaccctattatttcacctggggtaaagccgatatcgccaataaccacccagt

SEQ ID NO: 227 - DNA, 224 bp, Edwardsiella ictaluri
tgtttggcgcgctggcggtgaaaccctcaggaatgagttacgaagaggcaatgaccagacgcgtcctgcaaccattaaaactggcgcatacctggattacggttccgcagaacgaacaaaaagattatgcctggggctatcgcgaagggaagcccgtacacgtttctccgggacaacttgacgccgaagcctatggcgtgaaatccagcgttattgatatggcc

SEQ ID NO: 228 - DNA, 224 bp, Citrobacter freundii
ttcatgatgaaaaaatcgatatgctgcgcactgctgctgacagcctctttctccacgtttgctgccgcaaaaacagaacaacaaattgccgatatcgttaaccgcaccatcacaccactgatgcaggagcaggctattccgggtatggccgtggcgattatctacgaggggaaaccttattactttacctggggtaaagccgatatcgccaataaccacccagt

SEQ ID NO: 229 - DNA, 224 bp, Unknown
  Description of Unknown Mixed culture polynucleotide
tctgtttggtgcgctggcggtgaaaccttcaggtatgagctacgaagaggcaatgaccagacgcgtcctgcaaccattaaaactggcgcatacctggattacggttccgcaaagcgaacaaaaaaattatgcctggggctatcgcgaagggaagcctgtacacgtttctccgggacaacttgacgccgaagcctatggcgtgaaatccagcgttatcgatatgg

SEQ ID NO: 230 - DNA, 224 bp, Pseudomonas stutzeri
aggacgaactggcccgcctcgacggcgtcggcgacgtccagttgttcggcctcggcgactattcgctgcgcgtctggctggacccgaacaaggtcgcctcgcgcaacctcaccgccaccgacgtggtcaacgccatccgcgagcagaaccgccaggtcgccgccggcaccctgggcgcgccgccggcgccgagcgataccagcttccagttgtcgatcaacacc

SEQ ID NO: 231 - DNA, 224 bp, Pseudomonas entomophila
ggccgtacctatcaggtcaacgtccaggccgagcagcagttccgcctcgaacccgagcagatcggccagctgaaggtgcgcaacaacctcggcgagatggtcccgctggcgtccttcatcaaggtcagcgacacctccggtccggaccgtgtgatgcactacaacggcttcatcaccgccgaactcaacggcgccccggccgcgggctacagctccggccaggc

SEQ ID NO: 232 - DNA, 224 bp, Artificial Sequence
  Description of Artificial Sequence Synthetic construct polynucleotide
ctgcgcccgctacctgatgccggaccgcgacgacgtggacaagctggtcatcggcagtttctgctcgatcggcagtggcgccgccttcatcatggccggcaaccagggacaccgcgccgaatgggcgtcgaccttccccttccacttcatgcacgaagagcctgccttcgccggcgccgtgaacggctatcagccagcgggcgacacgctgatcggccatgacg

SEQ ID NO: 233 - DNA, 224 bp, Paracoccus denitrificans
catgcccagcaccatcgaggcgaccgtaccaccggccacgccggtacccagcgaacggcgcgcggccgaaccggcgccggtggcgaacaccagcggcagcacgcccatcaggaacgccagcgaggtcatcacgatcggccgcagccgcaggtagaccgcctgcaggatcgcctcgcgggtgctcttgccctgctgttcgaggatgcgcgcgaactcgacgatca

SEQ ID NO: 234 - DNA, 224 bp, Cupriavidus metallidurans
atcgcggcggcgatcgtcaccgagaactggcgatagatcaccccggttacaccgctgaagaacgccatcggcaggaacaccgcggtcagcaccagcacgatgccgaccagcgcgccggtgatctggcccatcgccttcagcgtggccggcttcggtgccagcccttcgaaggtcatcacccgctccacgttctccaccaccacgatggcatcgtccaccagcag

SEQ ID NO: 235 - DNA, 224 bp, Laribacter hongkongensis
ggccatggcggcctgcagctggttctgcacctgcacctgcgcggtatccggattggtgccggtggcgaacaccaggttgacctgcgccgagccgtcggaggcactggtcgaggtcatgtacatcaggtggtccaggctctgctgggactgctcgatgacctgggtgaccgcgttttccacggtctgcgacgaggcgccggtgtaggtggcgcggatggtgatgg

SEQ ID NO: 236 - DNA, 224 bp, Enterobacter cloacae
gctgctgtaactgttctgcaatggccggtgagcacagcggagcgtgtggtgcactgaaaatcagttccgcatctgactcatgccacgcgccgttaccgaaacggatcgtataatcatgcccttccgccgccgggtccacatgattgttatgggtggagatatgcagatcaatatgcggatggctgtcatagaatccggccagacgcggcagcagccagcctgcg

SEQ ID NO: 237 - DNA, 224 bp, Burkholderia cepacia
ttccagccgcagggcaccggcccgaccaacgcgacgctgtggcgcgcgcagggcacggtgtcgtacgaagccgacctgttcggccgcgtcggccgcaacgtcgaggcgtcgcgtgccgaccaggcgcagagcgaagcgctgttccgctcggtgcagctcgcgttgcaggccgacgtcgcgcagaactacttcgaactgcgtcagctcgattcggatcaggacct

SEQ ID NO: 238 - DNA, 224 bp, Clostridium difficile
gcaaatcaatatgaagttagaataaatagtgaaggaagaagagaattattagaccattctggtgaatggataaacaaagaagaaagtattataaaggatatttcatcaaaagaatatatatcatttaatcctaaagaaaataaaattatagtaaaatctaaaaatttacctgaattatctacattattacaagaaattagaaataattctaattcaagtgatat

SEQ ID NO: 239 - DNA, 224 bp, Clostridium difficile
agattattttaaacatgtttcattagttgaaactgaaggagtatttactttattagatgataaaataatgatgccacaagatgatttagtgatatcagaaatagattttaataataattcaatagttttaggtaaatgtgaaatctggagaatggaaggtggttcaggtcatactgtaactgatgatatagatcacttcttttcagcaccatcaataacatata

SEQ ID NO: 240 - DNA, 224 bp, Clostridium difficile
aaaatggatttattaattgttctacaaaagaaggtttatttgtatctgaattatctgatgtagttcttataagtaaagtttatatggacaatagtaaacctccatttggatattatagtaatgatttgaaagatgttaaagttataactaaagatgacgttattatattaacaggatattatttaaaagatgatataaaaatctctctttcttttactatacaa

SEQ ID NO: 241 - DNA, 224 bp, Neisseria lactamica
tctcgggcggacagcagcagcgcgtctccatcgcccgcgccctgatgaacggcggagaaatcatcttcgccgacgagccgaccggcgcgctcgataccgccagcggcaaaaacgtgatggaaatcatccataagctgcacgaagccgggcataccgtcattatggtcacgcacgaccccggcatcgccgccaatgccaaccgcgtcatcgaaatccgggacggc

SEQ ID NO: 242 - DNA, 224 bp, Stenotrophomonas maltophilia
aggtccagcccttcgccggccaggtcgatgcggttgttgtgggtctgcaggcgcagctcgatgtccgggtgcgccgcctcgaacgcggccagccgtggcagcagccagccggtggcgaaggtgccgaccacgcccagcgtcagcacttcccgggccggcccgccggtgtagcgcgcgatgctggcggagatgcgttcgaacgattcgttgagcaccggatacag

SEQ ID NO: 243 - DNA, 224 bp, Enterococcus raffinosus
ttgcggaacagcggaaaaacgaccttgtcatgtacctggctcatgacttgaagaccccgctttcatcggtcataggatatttgaacctgttaagggatgagaatcagatctccgaggaactccgggaaaaatatctgtccatttcactggataaggctgaacgtctggaagagctgatcaatgagttttttgaaattacgaggtttaatctttcaaacatcacg

SEQ ID NO: 244 - DNA, 224 bp, Acinetobacter sp.
taaaaagcagtggcaagccaaaaaaatcagcgcgtacagtgggtgatgtacttggtaaataccacccacatggtgactcggcatgttatgaagccatggtactcatggctcagccatttagttaccgctatcctttaatcgaaggtcaggggaactggggttcacctgatgatcctaaatcttttgctgcgatgcgttataccgaagccaaactctcggcttat

SEQ ID NO: 245 - DNA, 224 bp, Staphylococcus epidermidis
aagcgataaatagtgctattttgttagaacaagtaccttataataagttaaataaaaaagtacatattaacaaagatgatatagttgcttattctcctattttagaaaaatatgtaggaaaagatatcactttaaaagaacttattgaggcttcaatgacatatagtgataatacagcaaacaataaaattataaaagaaatcggtggaatcaaaaaagttaaa

SEQ ID NO: 246 - DNA, 224 bp, Staphylococcus intermedius
gaacaagtaccttataataagttaaataaaaaagtacatattaacaaagatgatatagttgcttattctcctattttagaaaaatatgtaggaaaagatatcactttaaaagaacttattgaggcttcaatgaagtatagtgataatacagcaaacaataaaattataaacgaaatcggtggaatcaaaaaaattaaaaaacgtttaaaaaaattgggagataa

SEQ ID NO: 247 - DNA, 224 bp, Shigella flexneri
gtatggcggtggcggtaatttatcagggtaaaccttattactttacctggggctatgcggacatcgccaaaaagcagcccgtcacacagcaaacgttgtttgagttaggttcggtcagcaaaacatttacgggcgtgcttggtggcgacgctattgctcgaggggaaatcaagttaagcgatcccgcaacaaaatactggcctgaacttaccgctaaacagtgg

SEQ ID NO: 248 - DNA, 224 bp, Escherichia coli
tcggcgcactggctgtgaagccgtctggtttgagttttgagcaggcgatgcaaactcgtgtcttccagccactcaaactcaaccatacgtggattaatgtaccgcccgcagaagaaaagaattacgcctggggatatcgcgaaggtaaggcagtgcatgtttcgccaggggcgttagatgctgaagcttatggtgtgaagtcgaccattgaagatatggcccgc

SEQ ID NO: 249 - DNA, 224 bp, Xanthomonas maltophilia
cgcaccgctgccgcagctgcaggcctacaccgtggacgcctcgtggctgcagccgatggcaccgctgcagattgccgaccacacctggcagatcggcaccgaggacctgaccgcgctgctggtgcagaccgccgacggtgcggtactgctcgacggcggcatgccgcagatggccggtcacctgctggacaacatgaaggcacgcggggtggcagcacaggatc

SEQ ID NO: 250 - DNA, 224 bp, Zea mays
accgcatcatcatggatggcgaagaggtcaccgtaggcggcatcgcgttcaccgcgcacttcatgccggggcacaccccaggcagcaccgcctggacctggaccgatacccgcgatggccagccggtgcgcatcgcctacgccgacagcctgagtgcgccgggctaccagctgcagggcaacgcccgttatccccgcctggtcgaggactaccggcgcagcttc

SEQ ID NO: 251 - DNA, 224 bp, Shigella boydii
cgttgtgaacattgtcgcggcgcaggcaagggtaaaaattagcgccccggtaatcagcaccggcctgcggccaatcctgtcggaaagcggccccagcagccactgtaacgccataccgccagcgagatagagactgacggcagccggggccagactgacatcggcattaaaatcacggaccacattaatgatccccggctggatcagatccgtcgacaggtacg

SEQ ID NO: 252 - DNA, 224 bp, Artificial Sequence
  Description of Artificial Sequence Synthetic construct polynucleotide
cggctcggcgaagaaagacgccggcggtttcgccggctacccgccggtaaaggtcgccctcgcctcggtggagcggcgggtggtgccgcgcgtcttcgatggcgtcggcgagctggaggccggtcgccaggtccaggtggccgccgaagcggcaggacggatcacccgcatcgccttcgaatcgggccagcaggtgcagcaagggcagttgctggtgcaactca

SEQ ID NO: 253 - DNA, 224 bp, Artificial Sequence
  Description of Artificial Sequence Synthetic construct polynucleotide
ggcgcagcttcccggcgcgcatcagcgccatcgacccgctgatcggcaagtcgcgcacggtgcaggtccaggccttgctggacaaccccgaaggcctgctcgccgccggcatgttcgccagcatccgggtctcgcgcaaagccgacgcgccgtcgctgagcgtgccggaaaccgcggtcacctataccgcctacggcgacaccgtgttcgtcgcccaccaggac

SEQ ID NO: 254 - DNA, 224 bp, Pseudomonas aeruginosa
ggtgcgcctggtcgaccaggcgctggcgcgcaaccacgacatccgcgaggcccgcgccaacctgcgcagcgcccgcgcgctgttcgacgaccgctggctcgaccagttgccgcaggtcaccagccaggccggctacagccgcagcatcgaacaacagctggactacgacggcgagccgcgccggcgcctggcggagagctaccgcgccggcttcgacgcgcagt

SEQ ID NO: 255 - DNA, 224 bp, Pseudomonas aeruginosa
tggagaacgcccaggccaacctgctgcgcagcgaagcggcgattccgccactgacgaccgcgctggagagcgcccgctatcgcctcgacgtgctgcgcggcgaggcacccggcagcggcgcgccgatcctcgacggcggcgccgccgcgccattggcgaagaacctgccgctgggcgacgtcgaccgcctgatcctccagcgccccgacgtagtcagcgccgag

SEQ ID NO: 256 - DNA, 224 bp, Pseudomonas aeruginosa
tcgccctggtcgccgaggtcagccaggcctacctgagctacgacggcgccctgcggcgcctggcgctgacccgtcagaccctggtcagccgcgagtattccttcgccctgatcgaccagcgccgcgcggccggcgccgccaccgcgctggactaccaggaagcccttggcctggtggagcaggcgcgcgccgagcaggagcgcaacctgcggcagaaacagcag

SEQ ID NO: 257 - DNA, 224 bp, Burkholderia vietnamiensis
ataccgaccggcagcttcttcgcgagcgcttccatcgcggccatcgcctgaccggtcgacttgcccggtgcggcctgaccctggatttccatcgccgacacgccgttgtagcgctcgagcttcggcgaaccgtacgtccagtgaccggtcgcgaacgcgctgaacggcaccatcccgcccgacccgttgcgcacgtaccagatgttcatgtcttccggcgtcat

SEQ ID NO: 258 - DNA, 224 bp, Thiobacillus denitrificans
actgacgatagatcgcgccgaccgagccgccggagaacgccaccggcacgaacaccgccgacagcacgagcgccacgccgacgagtgcgccggtgatctggcccatcgccttgcgggtcgcctccttcggtgacagcccctcttccgccatcacgcgctcgacgttctcgaccaccacgatcgcatcgtcgaccagcaggccgatcgcgagcacgaggccgaac

SEQ ID NO: 259 - DNA, 224 bp, Escherichia coli
atggagcgctggcgcctgctgcgcgacggcgagctgctcaccacccactcgagctggatacttcccgtccgccagggggacatgccggcgatgctgaaggtcgcgcgcattcccgatgaagaggccggttaccgcctgttgacctggtgggacgggcagggcgccgcccgagtcttcgcctcggcggcgggcgctctgctcatggagcgcgcgtccggggccgg

SEQ ID NO: 260 - DNA, 224 bp, Staphylococcus epidermidis
attatggaacagatggaaacaatggttagaaaacgacgaactatggcctcgacatgcgaccatgatacatggggacttacatccaggacatataatggtagataaccaagcaaacgtcacaggtctcatagactggactgaagcaacccactccgacccatcaatggactttataggacaccatcgtgtattcgacgacgaaggattagagcaacttataacag

SEQ ID NO: 261 - DNA, 224 bp, Pseudomonas aeruginosa
ccgagccagcgccaggcccagcgtttcaccgccaacctgctgatcgcgttcttcccggcggtagtcctcggcgtcctgttcgccgacctgatccacgagtggctgttcaatccgatcaccgtcgccctggcgctggtggtgggcggggtggtcatgctctgggccgagcgccggaagcatgtgatccgcgccgaacacgtcgacgacatgacctggaaggacgc

SEQ ID NO: 262 - DNA, 224 bp, Unknown
  Description of Unknown Uncultured bacterium polynucleotide
tgccgaaaaacacgtcaacggcacaatgacgctggcagaactgagcgcggccgcgttgcagtacagcgacaataccgccatgaacaaattgattgcccagctcggtggcccgggaggcgtgacggcttttgcccgcgcgatcggcgatgagacgtttcgtctggatcgcactgaacctacgctgaataccgccattcccggcgacccgagagacaccaccacgc

SEQ ID NO: 263 - DNA, 224 bp, Klebsiella pneumoniae
atgacattcggggagttgagcgcggcggcgctacagtacagcgataatactgccatgaataagctgattgcccatctcggggggccggataaagtgacggcatttgcccgtacgattggcgatgacacgttccggctcgatcgtaccgagccgacgctcaacaccgcgatccccggcgacccgcgcgataccaccacgccgttagcgatggcgcaggctctgcg

SEQ ID NO: 264 - DNA, 224 bp, Pseudomonas putida
cccgtcgtgtgtctccgtgctcgtcttttaaacttccattggcaatcatggggtttgatagtggaatcttgcagtcgccaaaatcacctacgtgggaattgaagccggaatacaacccgtctccgagagatcgcacatacaaacaagtctatccggcgctatggcaaagcgactctgttgtctggttctcgcagcaattaacaagccgtctgggagttgatcgg

SEQ ID NO: 265 - DNA, 224 bp, Acinetobacter baumannii
acagcaacaataccaaatcacaaaaaatagccagctcccaaccattggcgtaacgggaaatgcagtgcggcaggttagcccatcgattaaccccaataacccagtttctacatttcaggtgggtttaggaatgactgcatatgaactagacttctggggccgtgttcaaaatttaaaagatgctgcattaaataactaccttgcaactcaaagcgcgaaagagg

SEQ ID NO: 266 - DNA, 224 bp, Acinetobacter sp.
aagcctgacaggctcgacgggttatgcatcatctgaactgaaagatttatttaaaacaggcaattttgcatggtcgattggacctaatatcgatctaccaatttttgattggggaacaagaaaaactaatattaaaattgcggaaactgaccagaaaattgctttagctaaatatgaaaaagccattcaatcagcttttcgtgaagttaatgatgcgcttgcta

SEQ ID NO: 267 - DNA, 224 bp, Artificial Sequence
  Description of Artificial Sequence Synthetic plasmid R67 polynucleotide
atggaacgaagtagcaatgaagtcagtaatccagttgctggcaattttgtattcccatcgaacgccacgtttggtatgggagatcgcgtgcgcaagaaatccggcgccgcctggcaaggtcagattgtcgggtggtactgcacaaatttgacccccgaaggctacgccgtcgagtctgaggctcacccaggctcagtacagatttatcctgttgcggcgcttga

SEQ ID NO: 268 - DNA, 224 bp, Salmonella enterica
atttggcgattggtgacttgctaaccacagggttcatctctcatttcgaggacggtcgtattcttaagcacatctacttttcagccttgatggagccagcagtttggggagctgaacttgctatgtcactgtctggcctcgagggtcgcggctacatatacatagttgagccaacaggaccgttcgaagacgatccgaatcttacgaacaaaaaatttcccggt

SEQ ID NO: 269 - DNA, 224 bp, Xanthomonas maltophilia
gccgaccatgccggaccggtggcggagctgaagcgccgtacgggcgccaaagtggcggccaacgccgaatcggcggtgctgctggcgcgtggcggcagcgatgacctgcacttcggcgatggcatcacctacccgcctgccaatgcagaccgcatcgtcatggatggtgaagtgatcacggtgggcggcatcgtgttcaccgcgcacttcatggcggggcacac

SEQ ID NO: 270 - DNA, 224 bp, Klebsiella variicola
ctgtgcgccggggtgaccttcagcatggcgaagtggatgccggaaacgcgcccggaaggggcgccgcgcaccaaacttctggccagctacaaaacgctgttcggcaccggcgcctttaactgctatctgctgatgctgatcggcggcctggcggggattgcggtattcgaagcctgctccggggtgctgatgggcgcggtgctcggcctgagcagcatggcggt

SEQ ID NO: 271 - DNA, 224 bp, Ruminococcus gauvreauii
ggaaaagttttacttccctgttatcatgctgacagcaaaagtggaggacggggataaaatcatgggactgtccgtggcagatgattatattacgaagccgtttaacccgctggaagtggttgcgagggtaaaggcgcagctaaggcagtacatgcggtacaagcagcccagcttaaagcaggaggctgaatgcacagaatacgatatcagagggatgacaatca

SEQ ID NO: 272 - DNA, 224 bp, Klebsiella variicola
tccgttcctcggtccaaagctgaccatctatcagcaggatgcgatgaccatgaactttggcgaactggcggagaaaatgggccagccgctgcgcgtcttcggcaacctgccgtacaatatctcgaccccgctgatgttccatctgttcagctatactgatgccattgccgatatgcactttatgctgcagaaggaagtggtgaatcgtctggttgccggaccga

SEQ ID NO: 273 - DNA, 224 bp, Streptococcus pneumoniae
caacgggtaagattctttacgtagaaaaaacacaatttaacaaggttgcagaggtctttcataagtatctggacatggaagaatcctatgtaagagagcaactctcgcaacctaatctcaagcaagtttcctttggttcgaagggcaatgggattacctatgccaatatgatgtctatcaaaaaagaattggaaactgcagaggtcaaggggattgattttaca

SEQ ID NO: 274 - DNA, 224 bp, Streptococcus pneumoniae
tattgataataataccttcccaagtggagaatacttcaatagcagtgaattcaaaatagcggatgcgacgactcgagattgggatgttaatgatggtttgactactggtgggatgatgactttcttacaaggtttcgctcactccagtaatgttggaatgagtctacttgaacaaaaaatgggagatgctacttggttggattatctaaaacgctttaaatttg

SEQ ID NO: 275 - DNA, 224 bp, Streptococcus pneumoniae
gcaaggaatttcagtgacacaaacacaaatgcttcgtgcctttacagctattgctaatgatggagttatgctggagccaaaatttataagtgctatttatgatactaacaatcagtctgtacgtaagtcacaaaaagaaatagtaggaaatcctgtttccaaagaggcagcaagcacaactcgaaatcacatgatcttagttgggacggaccctctatatggaa

SEQ ID NO: 276 - DNA, 224 bp, Streptococcus pneumoniae
ggagtcccgattgctgaggacgcaacctcctataatgtctatgctgttattgataaaaaatacaaatcagcaacgggtaaaattctttatgtagaagatgcccagtttaataaggtagcagaggttttccataagtatctggatatggacgaggcttatgttaaagaacaattgtctcaaccgaatctgactcaagtttcctttggtgccaaaggaaatggaat

SEQ ID NO: 277 - DNA, 224 bp, Streptococcus pneumoniae
caaagtaactatgaaccaggatcagccatgaaggttatgacgttagcttcttctattgataataataccttcccaagtggagaatacttcaatagcagtgaattcaaaatagcggatgcgacgactcgagattgggatgttaatgatggtttgactactggtgggatgatgactttcttacaaggtttcgctcactccagtaatgttggaatgagtctacttga

SEQ ID NO: 278 - DNA, 224 bp, Streptococcus pneumoniae
ctggagccaaaatttataagtgctatttatgatactaacaatcagtctgtacgtaagtcacaaaaagaaatagtaggaaatcctgtttccaaagaggcagcaagcacaactcgaaatcacatgatcttagttgggacggaccctctatatggaactatgtataatcactacacaggaaagccaattataacagttcctggacaaaatgtagcagttaaatccgg

SEQ ID NO: 279 - DNA, 224 bp, Streptococcus pneumoniae
gtcagcaacgggtaagattctttacgtagaaaaaacacaatttaacaaggttgcagaggtctttcataagtatctggacatggaagaatcctatgtaagagagcaactctcgcaacctaatctcaagcaagtttcctttggagcaaagggcaatgggattacctatgccaatatgatgtctatcaaaaaagagttggaaactgcagaggtcaaggggattgatt

SEQ ID NO: 280 - DNA, 224 bp, Streptococcus pneumoniae
gataataataccttcccaagtggagaatacttcaatagcagtgaattcaaaatagcggatgcgacgactcgagattgggatgttaatgatggtttgactactggtgggatgatgactttcttacaaggtttcgctcactccagtaatgttggaatgagtctacttgaacaaaaaatgggagatgctacttggttggattatctaaaacgctttaaatttggggt

SEQ ID NO: 281 - DNA, 224 bp, Streptococcus pneumoniae
aatgatggagttatgctggagccaaaatttataagtgctatttatgatactaacaatcagtctgtacgtaagtcacaaaaagaaatagtaggaaatcctgtttccaaagaggcagcaagcacaactcgaaatcacatgatcttagttgggacggaccctctatatggaactatgtataatcactacacaggaaagccaattataacagttcctggacaaaatgt

SEQ ID NO: 282 - DNA, 224 bp, Burkholderia multivorans
acgtcaggacgacgcgtcagcagatccgacggcaggccagccggcacgtccgtcagcaggttctgcgcatcgagcggcatgcccgccggcaggtcgtccggcagcggctcgccgatcagcagcaccagcgcgttcagcgcctgcgcgcgggcacgggcctgcgcctgctggttcgcgagcgccgtctcgaccaccgtctgcgcctgacgcagctcgagctccga

SEQ ID NO: 283 - DNA, 224 bp, Artificial Sequence
  Description of Artificial Sequence Synthetic construct polynucleotide
tccagcatgtgcggacgcgcatccagagcgagctcgattgccagcgactcgacctgaccccgcccgacgtccatgtattgaagcttatcgacgaacaacgcgggctgaacctgcaggacctgggacgccagatgtgccgcgacaaggcactgatcacccggaagatccgcgagctggagggaagaaacctggtccgccgcgagcgcaaccccagcgaccagcgc

SEQ ID NO: 284 - DNA, 224 bp, Haemophilus influenzae
tcggtaaatatcacccgcatggtgacttagccgtgtacgataccatcgttcgtatggcacaaccattctcacttcgctatatgttggttgatgggcaaggtaactttggttcaattgatggtgatgcgccagctgcaatgcgttataccgaagtgcgtatgcaaaaaattacgcaagcattgctcacggatttggataaagaaaccgtcaatttctcgccaaac

SEQ ID NO: 285 - DNA, 224 bp, Enterococcus sp.
ttccacaaattgaatcctttctcaccaaagaaaaatggcgaagagaaaaaagatacttggattctttggtctaaagtcttagtcgcttgtttaccagcggcagttattggtctaaaatttgatgattatttagatgcacatttttataactttttaacagtatcgattatgttaattgtttatgggattgcttttattatcattgaaaaacgaaataaaaatgt

SEQ ID NO: 286 - DNA, 224 bp, Streptococcus salivarius
cgatatttctgatttggacaaatgggatatgattatttccggaagcgatgaactattagaacgatatgttgcagaggattctttggatatacaggaattacaatatgaaaagtgcaaaagaaccagatgctgctctttgtttcctgtttatcatgggagtgcaaaagacaatttaggaacagaaaaactgattgaagcgattacagaaactttcattacagaaa

SEQ ID NO: 287 - DNA, 224 bp, Unknown
  Description of Unknown Clostridium saccharolyticum-like polynucleotide
gagccgcaaaagccggagcaaagggaagccctgttaaatgccctcacagagattgctgatacagaccctcttttgcattttgacattgatactgttacacatgagattatgttatcttttttgggaaaagtacagttagaagttatttgttcgctattagaagaaaaatatcatgtgggcgtggctatgaaagagccttcggttatttatctggaaagaccgct

SEQ ID NO: 288 - DNA, 224 bp, Pseudomonas aeruginosa
gccgatccgccagtacccgctgctggaaagctcgaccatcaccgtcaccaccgagtaccccggcgcctccgccgatctcatgcaaggcttcgtcacccagccgatcgcccaggcggtgtcgtcggtggagggcatcgactacctttcctcgacctcggtgcaggggcgtagcgtggtgaccatccgcatgctgctcaaccgcgattcgacccaggcgatgaccg

SEQ ID NO: 289 - DNA, 224 bp, Bordetella parapertussis
ccatcggcctgatgggcggcctcaccggcgcgctgttccgcgagttcgccctgaccctggcgggcgcggtgatcgtgtccggggtggtggcgctgaccctgtcgccggtgatgagttcgctgctgctccaggcgcaccagaacgaggggcgcatgggccgcgccgccgagtggttcttcggcggcctgacgcggcgctacgggcaggtcctggagttctccctg

SEQ ID NO: 290 - DNA, 224 bp, Pseudomonas fluorescens
cgcgaccctccaggcgatccccgcgcccggcgtctccatgggccaggcggtggccttcctcgacgacgtggcgcgcggcctgccggccggcttcagccacgactggcaatccgactcgcggcaatacacccaggaaggcaacaccctggtgttcgccttcctcgccgccctggtggtgatctacctggtgctcgccgcgcagtacgagagcctggccgacccgc

SEQ ID NO: 291 - DNA, 224 bp, Burkholderia cenocepacia
gatcgggcggaagcgcagcaggcacgcctcgtggatcgcgtcgaacgacgacttcccgtggttgcgcgtctggtcgatcgcgaagtcgaccatcatgatcgcgttcttcttcacgatgccgatcagcaggatcacgccgatcagcgcgatgatgctgaactcggtcttgaacagcagcagcgcgagcagcgcgccgacgccggccgacggcagcgtcgacagga

SEQ ID NO: 292 - DNA, 224 bp, Stenotrophomonas maltophilia
tacttgccaagcgttttagcgcaagagtccgtcactccatacattgcaatgctgaatggagagccgattgggtatgcccagtcgtacgttgctcttggaagcggggacggatggtgggaagaagaaaccgatccaggagtacgcggaatagaccagtcactggcgaatgcatcacaactgggcaaaggcttgggaaccaagctggttcgagctctggttgagtt

SEQ ID NO: 293 - DNA, 224 bp, Escherichia coli
acttgctgacgtacaggaacagtacttgccaagcgttttagcgcaagagtccgtcactccatacattgcaatgctgaatggagagccgattgggtatgcccagtcgtacgttgctcttggaagcggggacggatggtgggaagaagaaaccgatccaggagtacgcggaatagaccagttactggcgaatgcatcacaactgggcaaaggcttgggaaccaagc

SEQ ID NO: 294 - DNA, 224 bp, Klebsiella pneumoniae
gtacttgccaagcgttttagcgcaagagtccgtcactccatacattgcaatgctgaatggagagccgattgggtatgcccagtcgtacgttgctcttggaagcggggacggatggtgggaagaagaaaccgatccaggagtacgcggaatagaccagttactggcgaatgcatcacaactgggcaaaggcttgggaaccaagctggttcgagctctggttgagt

SEQ ID NO: 295 - DNA, 224 bp, Artificial Sequence
  Description of Artificial Sequence Synthetic binary vector polynucleotide
gcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggc

SEQ ID NO: 296 - DNA, 224 bp, Staphylococcus epidermidis
aagagttagataattacctaaagggcgataaattaataaaggttctaattgataaagattgtagaattaaaagggacatagttccgactgatatagattatcatgtaagaaagccaagcgcaagggagtatgatgattgctgcaatgaattttggaatgtaacaccttatgttattaaaggattgtgccgtaaggaaattttatttgctattgatcattttaat

SEQ ID NO: 297 - DNA, 224 bp, Burkholderia mallei
atcacgtgaagaagtgcgacgtgatcctgccgctcgtcgcgatcgcgacgcccgccacctacgtcaagcagccgctgcgcgtgttcgagctcgacttcgaggcgaacctgccgatcgtgcgctcggccgtcaagtacggcaagcacctcgtgttcccgtcgacctccgaggtctacggcatgtgctcggacgagcagttcgacccggatgcgtcggccctcacc

SEQ ID NO: 298 - DNA, 224 bp, Ruminococcus gauvreauii
gcggaaaagaactgcgggatatgaccgtgggtgtgttaggaactgggcgaatcggacaggcagtcatggaacgcctggagggattcggctgtaaggtattggcgtatgaccgaaatcaaaaagcaggagcggactatgtttcgtttcatgaactgctgaaaaaaagtgacattgttacactgcatattccgttggcagaagatacccgccatatgattggatgt

SEQ ID NO: 299 - DNA, 224 bp, Ruminococcus gauvreauii
tccaatcagcaggccattcaaatattcatggataaggtaaatgagaaggccagggcaattggtgttacagactcgaattttgtggttccggatgggtatgatgccgaggggcagtataccacggcctatgatcttgccatcattgcaaaggcatgtttggaggatcccattatttcagaaattgtggcaagcaatacgtcgtatgaaaaatggccgaacggcag

SEQ ID NO: 300 - DNA, 224 bp, Burkholderia cenocepacia
gacgaggatcgcggcgaacgccgacgccagcacgacgccgatgatcacggtcagcacgctcgtccagaacaggttcgcgtacggcagcagcatcgtgaacggcgcgacgcccaggatcgacacccagatcacgtacttgcggccgatccggtcgccgacgggcccgccgatcagcgtgccggccgccaccgccgcgaggaacacgaacaggtggatctgcgcgg

SEQ ID NO: 301 - DNA, 224 bp, Burkholderia cenocepacia
acgattcgggatggaacaccgacgagccgcagccgacgagcgcggcggccaccagcagcatcgggaaattcggcgcgaccgacatcagcagcagccccgcgagcgtgaagcccatgccgaccggcagcgaatacggcttcggatgcttgtcggtatagaggcccacgagcggctgcagcagcgacgcggtgatctggtaagtgagcgtgatcaggccgatctgc

SEQ ID NO: 302 - DNA, 224 bp, Escherichia coli
accctggtggtgcatctgttctcccccggcgggaagtacgactcgctgtatatgcgcaactacgccacgctgaaagtgaaggatgagctggcgcgcctgcccggcgtcggccagatccagatttttggctccggtgaatatgcgatgcgcgtctggctggatcccaataaggtcgcggcccgcggtctgacggcctcggatgtggtgacggcgatgcaggagca

SEQ ID NO: 303 - DNA, 224 bp, Escherichia coli
tgcaatgggcggcgccgtacgacccgacggttttcgtccgcgactccatccgcgcggtggtgcagacgctgctggaggcggtagtgctggtggtgctggtagtgatcctgttcctgcagacctggcgcgcgtcgattatcccgttgatcgctgtgccggtatcggtggtgggtaccttcagcattctctatctgctgggcttctcgctgaataccctgagcctg

SEQ ID NO: 304 - DNA, 224 bp, Klebsiella pneumoniae
caacgccaaaatcgcgcaaatccagcagggctttggcttctccatcctgccgccgccgattttaggactgggtcagggttccggctactccctgtacatccaggatcgcggagggctgggctatggcgcgctgcaaagcgcggtgaatgcgatgtccggggcgattatgcagacgccggggatgcacttcccgatctcgacttaccaggctaacgtgccgcagc

SEQ ID NO: 305 - DNA, 224 bp, Stenotrophomonas maltophilia
gatcggcgcccgcgccggctatacccgtggcgcggtctactggcacttcaagaacaagagcgaggtgctggccgcgatcgtcgagcgcgtgcacctgcccttcatgcaggaactggagcgcacctccaccgaccagcgcgacaccccggtgcacgacctgcgcgcggtgatgatccactcgttcatcgagctgtccgaggacgagcgcctgcgcaagaccatgg

SEQ ID NO: 306 - DNA, 224 bp, Klebsiella pneumoniae
aaacactgattctgaataccgcgacggcctattttaaagtgctggccgccatcgacacgctctcctataccgaagcgcagaaacaggctatttaccgccagttggatcaaaccacgcagcgctttaacgtaggcctggtggcgatcaccgacgtacagaacgcccgttcacaatacgatgccgtgctggcgaacgaagtcaccgcgcgtaacgatctcgacaac

SEQ ID NO: 307 - DNA, 224 bp, Klebsiella pneumoniae
agaaccaaatcggcctgaacttctctctgccactgtatcagggcggcgcggtgacctcgcaggtcaaacaggcgcaatacaacttcgttggcgccagcgagcagctggaaagcgcccaccgcagcgtcgtgcagactgtgcgttcatcgtttaacaacgtgaacgcctccatcagcagcatcaacgcctacaaacaggcggtggtctctgcgcaaagctccctg

SEQ ID NO: 308 - DNA, 224 bp, Shigella dysenteriae
atgttccatctgtttagctatactgatgccattgccgacatgcactttatgttgcaaaaagaggtggtgaatcgtctggttgcaggaccgaacagcaaagcgtatggtcgattaagcgtcatggcgcaatactattgcaacgtgatcccggtactggaagttccgccgtcagcctttacaccaccacccaaagtggattccgccgtcgtgcgcctggttcctca

SEQ ID NO: 309 - DNA, 224 bp, Proteus mirabilis
atgttttcaaatatctggaccgagtatatcggtgattatacggagttctgcaaacagtatgaaaaagatatgcagcaatacggtgaaaacaagggcatgatggcaaagccaaatccgcctgtgaatactttcccagtctctatgattccatggacaacctttgaaggatttaatttaaatttgcaaaagggatatgggtatctgcttcccatttttacgtttgg

SEQ ID NO: 310 - DNA, 224 bp, Pseudomonas fluorescens
gcctgcgacctggagcatgccgagcctctggaggcgttgcagcgcctgatcaaggaacacctcacccaccgcgagctgctggtattcctggtattccagtaccgcccggacttcctcgacccgcacggcgaaggcgcacgctggcagtcctacctggaagcgctggacgccttcttcctgcgcggacagcagaaaggcgtgtttcgcatcgacatcacggcggc

SEQ ID NO: 311 - DNA, 224 bp, Acinetobacter calcoaceticus
atgtaggtttagcattgggttataacggcggtgacatttcttggacagatgatgtttcagtaaatggcactaagtatgatttagacatggataataacaatgtctatctaaatgctgaaattcgtccttggggtgccagcacaaatccatgggctcaaggcctatatatagctgcaggtgcggcttacttagataatgattatgaccttgctaaacgtattggt

SEQ ID NO: 312 - DNA, 224 bp, Klebsiella pneumoniae
caattcatggccagatatgacgagtgcaacaaaagaagtaaaagaatgtattgagagtccaaacctttgtttcggtctgctaataaataactccttagttggctggataggcttaaggccaatgtacaaggaaacctgggaattgcatccattggttgtcagaccagattatcaaaataaaggtattggcaagatcctgcttaaggaattagaaaacagagcta

SEQ ID NO: 313 - DNA, 224 bp, Haemophilus influenzae
gtaccgcactggcggaaacggattataaattttatccggtgatgatttatctgatctcccgggttgttaatcagtttccggagttccggatggcaatgaaagataatgcactgatttactgggatcagaccgatcctgtatttactgtttttcataaagagactgaaacattttctgcgctcttctgccgttattgtccggatatcagtgaatttatggcgggc

SEQ ID NO: 314 - DNA, 224 bp, Escherichia coli
gaatggcgtcggtgacgtacagcttttcggcgcacagtatgcgatgcgtatctggctggacgccgatctgctaaacaaatataaactgacaccggttgatgtgattaaccagttgaaggtacagaacgatcagatcgctgccggacagttgggcggaacgccagcgttaccagggcaacagttgaacgcctcgattattgctcagacgcggttaaaaaatccgg

SEQ ID NO: 315 - DNA, 224 bp, Escherichia coli
agccattcttcccgcagggaatgaaggttctttacccttatgacaccacgcctttcgtccagctttctatccaggaagtggtgaaaacgctgttcgaagccattatgctggtgttcctggtgatgtatctgttcttgcagaacatgcgggcaacattgatacccaccattgctgtacctgtcgtgttgttagggacatttgccatactcgccgcttttggttac

SEQ ID NO: 316 - DNA, 224 bp, Burkholderia multivorans
catcggcgggatgctgttcggcctcgagcgtcgcgtcgcgaccgagttttcgttcttcctcgcgattccgatcatcttcggcgcgacgctctatgaaaccgtgaaggactggcacgcgttcaccgtcgacgcgctcgggttgttcgtgctcgggctcgtgtcggccttcgtcagtgcgttcgtctgcgtgcgctggctgctgcgctacgtcgcgacgcacgact

SEQ ID NO: 317 - DNA, 224 bp, Xanthomonas maltophilia
gccgccgacgcaccgctgccgcagctccgggcctacaccgtcgacgcttcgtggctgcagccgatggcaccgctgcagattgccgaccacacctggcagatcggcaccgaggacctgacggcactgctggtgcggaccaccgagggcgcagtgctattggatggcggcatgccgcagatggccggtcacctgctggacaacatgaaggcacgcggggtggcacc

SEQ ID NO: 318 - DNA, 224 bp, Zea mays
cattcaccgcgcacttcatgccagggcacacccccggcagcaccgcctggacctggaccgatacccgcgatggcaagcccgtgcgcatcgcctacgccgacagcctgagtgcgccgggctaccagctgcagtacaacgcacgctatccccgcctggtcgaggactaccggcgcagcttcacaacggtgcgcggcctgccctgcgacctgctgctgaccccgcat

SEQ ID NO: 319 - DNA, 224 bp, Salmonella enterica
ctggtccggcaattgcgcgacctcggcgtgcgctcaggcgatatggtgatgccgcatgtgtcgttgcgcgccgtcgggccgctggcggacggaccgcagacacttgtcgatgcgctgatcgaggccgtcggccccaccgggaatattctcgccttcgtctcgtggcgcgattcgccctatgaacagacgctgggtcatgatgcgccgcccgccgccatcgccca

SEQ ID NO: 320 - DNA, 224 bp, Staphylococcus aureus
acggtgttggtgcttcagttgtaaatgcattgagtgaatggcttgaagttgaaatccatcgagatggtaatatatatcatcaaagttttaaaaacggtggttcgccatcttctggtttagtgaaaaaaggtaaaactaagaaaacaggtaccaaagtaacatttaaacctgatgacacaatttttaaagcatctacatcatttaattttgatgttttaagtgaa

SEQ ID NO: 321 - DNA, 224 bp, Staphylococcus aureus
attaccattacgtggtaaggtaattaatacagagaaagcacgtctagaagatatttttaaaaatgaagaaattaatacaattatccacacaatcggggcaggcgttggtactgactttaaaattgaagatagtaattataatcgtgtaattattatgactgatgctgatactgatggtgcgcatattcaagtgctattgttaacattcttcttcaaatatatga

SEQ ID NO: 322 - DNA, 224 bp, Staphylococcus epidermidis
attccaggaatgcagaaagaccaaagcatacatattgaaaatttaaaatcagaacgtggtaaaattttagaccgaaacaatgtggaattggccaatacaggaacagcatatgagataggcatcgttccaaagaatgtatctaaaaaagattataaagcaatcgctaaagaactaagtatttctgaagactatatcaaacaacaaatggatcaaaattgggtaca

SEQ ID NO: 323 - DNA, 224 bp, Staphylococcus epidermidis
agattacaacttcaccaggttcaactcaaaaaatattaacagcaatgattgggttaaataacaaaacattagacgataaaacaagttataaaatcgatggtaaaggttggcaaaaagataaatcttggggtggttacaacgttacaagatatgaagtggtaaatggtaatatcgacttaaaacaagcaatagaatcatcagataacattttctttgctagagta

SEQ ID NO: 324 - DNA, 224 bp, Escherichia coli
tgtgctgggcgcgctgtcggatcgtttcgggcggcggccggtcttgctcgtctcgctggccggcgctgctgtcgactacgccatcatggcgacggcgcctttcctttgggttctctatatcgggcggatcgtggccggcatcaccggggcgactggggcggtagccggcgcttatattgccgatatcactgatggcgatgagcgcgcgcggcacttcggcttca

SEQ ID NO: 325 - DNA, 224 bp, Escherichia coli
tcggcatttcgcttgccgcatttggcattctgcattcactcgcccaggcaatgatcaccggccctgtagccgcccggctcggcgaaaggcgggcactcatgctcggaatgattgccgacggcacaggctacatcctgcttgccttcgcgacacggggatggatggcgttcccgatcatggtcctgcttgcttcgggtggcatcggaatgccggcgctgcaagca

SEQ ID NO: 326 - DNA, 224 bp, Mycobacterium caprae
catgtcgcggatggagcgggtggtccgggagcggatgaccacccaggacgtggaggcgatcacaccgcagacgttgatcaacatccggccggtggtcgccgcgatcaaggagttcttcggcaccagccagctgagccaattcatggaccagaacaacccgctgtcggggttgacccacaagcgccgactgtcggcgctggggcccggcggtctgtcacgtgagc

SEQ ID NO: 327 - DNA, 224 bp, Mycobacterium bovis
ccagaacaacccgctgtcggggttgacccacaagcgccgactgtcggcgctggggcccggcggtctgtcacgtgagcgtgccgggctggaggtccgcgacgtgcacccgtcgcactacggccggatgtgcccgatcgaaacccctgaggggcccaacatcggtctgatcggctcgctgtcggtgtacgcgcgggtcaacccgttcgggttcatcgaaacgccgt

SEQ ID NO: 328 - DNA, 224 bp, Actinobacillus pleuropneumoniae
gaaattatagtgaagcaaaaggttgggcagcatccccatataaatgtaacggacaatgacgatatggaacagtgggatgcggtaattatgggaaacgatgaactattagagaaatatatgtcagggaaaccgtttaaaatgtcagaactggaacaggaagaaaacaggagattccaaaacggaacgttatttcccgtttatcacggaagcgctaaaaacaatct

SEQ ID NO: 329 - DNA, 224 bp, Artificial Sequence
  Description of Artificial Sequence Synthetic expression vector polynucleotide
cgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagc

SEQ ID NO: 330 - DNA, 224 bp, Unknown
  Description of Unknown Transposon Tn10 polynucleotide
ccaacgcgtgaagtggttcggttggttaggggcaagttttgggcttggtttaatagcggggcctattattggtggttttgcaggagagatttcaccgcatagtcccttttttatcgctgcgttgctaaatattgtcactttccttgtggttatgttttggttccgtgaaaccaaaaatacacgtgataatacagataccgaagtaggggttgagacgcaatcga

SEQ ID NO: 331 - DNA, 224 bp, Shigella sonnei
agtgaatcaggatggttcccgcgtactgctgcgtgatgtggcgaagattgagctgggtggtgagaactacgacatcatcgcagagtttaacggccaaccggcttccggtctggggatcaagctggcgaccggtgcaaacgcgctggataccgctgcggcaatccgtgctgaactggcgaagatggaaccgttcttcccgtcgggtctgaaaattgtttacccgt

SEQ ID NO: 332 - DNA, 224 bp, Shigella sonnei
cggtattgcgttcgtttccttgaaggactgggccgatcgtccgggcgaagaaaacaaagttgaagcgattaccatgcgtgcaacacgcgctttctcgcaaatcaaagatgcgatggttttcgcctttaacctgcccgcaatcgtggaactgggtactgcaaccggctttgactttgagctgattgaccaggctggccttggtcacgaaaaactgactcaggcgc

SEQ ID NO: 333 - DNA, 224 bp, Shigella boydii
gaccagcgtacgtccaaacggtctggaagataccccgcagtttaagattgatatcgaccaggaaaaagcgcaggcgctgggtgtttctatcaacgacattaacaccactctgggcgctgcatggggcggtagctatgtgaacgactttatcgaccgcggtcgtgtgaagaaagtttacgtcatgtcagaagcgaaataccgtatgctgccggatgatatcggcg

SEQ ID NO: 334 - DNA, 224 bp, Streptococcus pneumoniae
aaaagtctgagtttattatctgtctttgtttttgccatttttttagtcaattttgcggtcattattgggacaggcactcgctttggaacagatttagcgaaggaagctaagaaggttcatcaaaccacccgtacagttcctgccaaacgtgggactatttatgaccgaaatggagtcccgattgctgaggatgcaacctcctataatgtctatgcggtcattga

SEQ ID NO: 335 - DNA, 224 bp, Streptococcus pneumoniae
taatacctttccaggaggagaagtctttaatagtagtgagttaaaaattgcagatgccacgattcgagattgggacgttaatgaaggattgactggtggcagaatgatgactttttctcaaggttttgcacactcaagtaacgttgggatgaccctccttgagcaaaagatgggagatgctacctggcttgattatcttaatcgttttaaatttggagttccga

SEQ ID NO: 336 - DNA, 224 bp, Streptococcus pneumoniae
tcctgattttatcttgtatgtgacggtccaacaacctgaacattattcaggtattcagttgggagaatttgccaatcctatcttggagcgggcttcagctatgaaagactctctcaatcttcaaacaacagctaaagctttggagcaagtaagtcaacaaagtccttatcctatgcctagtgtcaaggatatttcacctggtgatttagcagaagaattgcgtc

SEQ ID NO: 337 - DNA, 224 bp, Streptococcus pneumoniae
atctgtctttgtttttgccgtttttttagtcaattttgcggtcattattgggacgggcactcgctttggaacagatttagcgaaggaagctaagaaggttcatcaaaccacccgtacagttcctgccaaacgtgggactatttatgaccgaaatggagtcccgattgctgaagatgcgacatcttataatgtctatgcggtcattgatgagaactataagtcag

SEQ ID NO: 338 - DNA, 224 bp, Streptococcus pneumoniae
gtacatgaccgcgaccttggtcagtgcaaagactggtgaaatccttgctaccacccaacgaccgacctttaatgcagatactaaagaaggaatcactaaggactttgtttggcgtgatatcctttatcaaagtaactatgaaccagggtcagccatgaaggttatgacgttagctgcttctattgataacaataccttcccaggtggagaatatttcaatagca

SEQ ID NO: 339 - DNA, 224 bp, Streptococcus pneumoniae
aaaaagaaattgtgggaaatcctgtttctaaagatgcagctagtctaactcggactaacatggttttggtagggacggatccggtttatggaaccatgtataaccacagcacaggcaagccaactgtaactgttcctgggcaaaatgtagccctcaagtctggtacggctcagattgctgacgagaaaaatggtggttatctagtcgggttaactaactatatt

SEQ ID NO: 340 - DNA, 224 bp, Streptococcus pneumoniae
gtttattatctgtctttgtttttgccatttttttagtcaattttgcggtcattattgggacaggcactcgctttggaacagatttagcgaaggaagctaagaaggttcatcaaaccacccgtacagttcctgccaaacgtgggactatttatgaccgaaatggagtcccgattgctgaggatgcaacctcctataatgtctatgcggtcattgatgagaactat

SEQ ID NO: 341 - DNA, 224 bp, Streptococcus pneumoniae
ggtcatgacgctcgctgctgctattgacaataacactttcccaggaggagaagttttcaatagtagtgagttaaaagtagcagatgttactatccgagattgggacgtcaatgaaggattgaccggtggcggaatgatgaccttttctcaagggttcgcgcactcaagtaacgttgggatgacgcttcttgagcaaaagatgggagatgctacctggcttgatt

SEQ ID NO: 342 - DNA, 224 bp, Streptococcus pneumoniae
ttattagtgccatttatgatccaaatgatcaaactgctcggaaatctcaaaaagaaattgtgggaaatcctgtttctaaagatgcagctagtctaactcggactaatatggttttggtagggacggatccggtttatggaaccatgtataaccacagcacaggcaagccaactgtaactgttcctgggcaaaatgtagccctcaagtctggtacggctcagatt

SEQ ID NO: 343 - DNA, 224 bp, Shigella sonnei
gacagtgcgaaaggtgatctggcgaaagcccaggctgcagccaatatcgcgcaattgacggtgaatcgttatcagaaactgctcggtactcagtacatcagtaagcaagagtacgatcaggctctggctgatgcgcaacaggcgaatgctgcggtaactgcggcgaaagctgccgttgaaactgcgcggatcaatctggcttacaccaaagtcacctctccgat

SEQ ID NO: 344 - DNA, 224 bp, Escherichia coli
cgtctggaagaagggcttaatccaaacgctattttagtcccgcaacagggcgtaacccgtacgccgcgtggcgatgccaccgtactggtggttggcgcggatgacaaagtggaaacccgtccgatcgttgcaagccaggctattggcgataagtggctggtgacagaaggtctgaaagcaggcgatcgcgtagtaataagtgggctgcagaaagtgcgtcctgg

SEQ ID NO: 345 - DNA, 224 bp, Acinetobacter sp.
tttacaattattggcttataccgacacccaacaagcaattgccatgttggaagcttcaattcggtatgaatatgtgaatggcacacaaacatcgcctgtggcttttttggaagggatttttgtattgcctgaatatcgtcgttcaggtatcgcaacggggttggttcaacaagtggaaatttgggcgaaacagtttgcatgtacagagtttgcttcggatgcag

SEQ ID NO: 346 - DNA, 224 bp, Artificial Sequence
  Description of Artificial Sequence Synthetic Escherichia coli plasmid polynucleotide
tcaatcgtcaccctttctcggtccttcaacgttcctgacaacgagcctccttttcgccaatccatcgacaatcaccgcgagtccctgctcgaacgctgcgtccggaccggcttcgtcgaaggcgtctatcgcggcccgcaacagcggcgagagcggagcctgttcaacggtgccgccgcgctcgccggactcgctgtcgcacggcctgctcactcaagcagctg

SEQ ID NO: 347 - DNA, 224 bp, Serratia proteamaculans
cgacgaggtgatccacatcatccgcagcgaggatgagccgaagccggtgctgatgcagcgtttcggcatctccgatacccaggcggaagcgatcctcgagctgaaactgcgtcacctggccaagctggaagaggtcaagatccgcggtgagcaggacgagctggcgaaagagcgcgatcaactgcaggcgctgctggcgtccgagcgcaagttgaatacgctga

SEQ ID NO: 348 - DNA, 224 bp, Shewanella woodyi
cgaacagtgggggatgtattaggtaaatatcatccccacggtgatagcgcctgttatgaagccatggtattgatggcgcaacccttctcttatcgttatccattaattgatgggcaaggtaactggggggctccggatgatccaaaatctttcgcagctatgcgttataccgagtctcgcttatctaaatattcacagatcctgctaagcgaattgggacatgg

SEQ ID NO: 349 - DNA, 224 bp, Lactobacillus reuteri
gttcaaaaatgtaattatgtaacagttatcgagatagattcaaatttatgtattcaaacacaaaataaagttacaaattacgataactttcgaattataaataaggatatattacagtttaagttcccaaacaacaaagcatataaaatctacggtaatataccttattatataagtacggatatagtacgaaaaattgtctttgaaagtgaagcaacagttag

SEQ ID NO: 350 - DNA, 224 bp, Unknown
  Description of Unknown Transposon Tn4351 polynucleotide
cagaggtgaactgtcctggattttttcagctatgcaatggaaaccggctaatggctgctcatcaaggtaatttattatttgcgaatcctaataataatggtgcattgcattttggaataagttttaaaacacctgatgaatggaaaagcaaaacgcaggtagattttcaagacagaaatagtgtcgttgattttctcctgaaaaaattttccgattgggacgaa

SEQ ID NO: 351 - DNA, 224 bp, Unknown
  Description of Unknown Uncultured bacterium polynucleotide
tgaacggtatcgataacctgatgtacatgtcctccaccagtgattcctctggtagcgtcaccattaccctgacgtttgaatccggcaccgatcctgacatcgcgcaggttcaggtccagaacaaactgtcgttggccaccccgttgctgccgcaagaagttcagcaacagggcctgaaagtagaaaaatccagcagcagcttcctgatggtggccggcttcgtt

SEQ ID NO: 352 - DNA, 224 bp, Serratia marcescens
ttgaccatggtgcaagctgcctgccggcgccaccgagtcccgcacaaacaaagtgctggaagaagtctccgactacttcctgaacaaggaaaaggacaacgtggtttcggtgtttaccgtcgcgggcttcggcttcaacggtaacggccagaacaacggcctggcattcgtcagcctgaaagactggggcgagcgtccagggagcgagaacaaggtcgaagcga

SEQ ID NO: 353 - DNA, 224 bp, Serratia marcescens
gccaggccgcaccgggcaagagtaccggtgaagcgatgaacctgatggaacagctggcgtccaaactgccaagcggcatcggttacgactggaccggcatgtcctatcaggaacgtctgtcgggcaaccaggcgccggcgctgtacgccatctcgattctggtggtgttcctgtgtctggcggcgctgtatgaaagctggtccgtgccgttctcggtcatgctg

SEQ ID NO: 354 - DNA, 224 bp, Stenotrophomonas maltophilia
cccagcattgccgccttcgccgccgccaccgatttcgccgcactggaaaaggcgtgcagcggccgactcggcgtgaccctgctcgacaccggcagcggtcgccgcctcggccatcgccaggacgaacgcttcccactctgcagcaccttcaagagcgtgctggccgctacggtgctcaagcaggccgagcgtgatccggcactgctggaccagcgcctgccggt

SEQ ID NO: 355 - DNA, 224 bp, Citrobacter freundii
acaaaaatgcttatctgattgatacgcccatttcagccacagatactgaaaaattagtgaagtggattgacgcgcagggctttacggccaaggcaagtatttctacccatttccacaccgacagtacaggcggtattgcatttctcaactccaagtccattccaacctatgcctccaagctaactaaccagctgcttaaaaataaaggcgaagagcaggctacg

SEQ ID NO: 356 - DNA, 224 bp, Artificial Sequence
  Description of Artificial Sequence Synthetic construct polynucleotide
tcgatgccctggggcgtacgtacgcgcacgtacatgcccggcagcaggaccccgtcggggttggcgaacttgccgcgcagggcgatctggccggtgccgcgatccaccgccacgtcggcgaactgcaacgcgccctggcgctcgtagggcgtcccttcgacgcgcagggtcagcgcctggctgtcgccggcggccaaggtgcctttcttcagggcgtcgcgcag

SEQ ID NO: 357 - DNA, 224 bp, Aeromonas veronii
tcactcggcatggacatgtacttgccagcggtgcctttcatgccacatgctcttggtacgacagcgggcacaattcagcttacgctgacaacgtatttggtcatgataggggccggtcagcttttgtttgggccactgtcggaccggctgggacgtcgtcccgtgctactggcgggcggtgccgcctacgttgcggcctcaatcggcctcgtcgtcacgtcatc

SEQ ID NO: 358 - DNA, 224 bp, Shigella sonnei
cccattgctgggagcggtcatagacacctggttcgggtggcgggcgatctttgcgttcttgggattgggaatgatcgctgcattgacagcagcgtggcggctctggcctgagacccgggtgcagcgaccagcagctttgcaatggtcacaacttctgcttcccatcaagcaccttaacttctggttgtacacagtgtgttatgccgcaggaatgggcagcttct

SEQ ID NO: 359 - DNA, 224 bp, Artificial Sequence
  Description of Artificial Sequence Synthetic plasmid pIP630 polynucleotide
cctttgaagatcaagtcttatatcattatgaagtgattggagataagttgattataggaagattttgttcaattggtcccggaacaacatttattatgaatggtgcaaaccatcggatggatggatcaacatatccttttcatctattcaggatgggttgggagaagtatatgccttccttaaaagatcttcccttgaaaggggacattgaaattggaaatgat

SEQ ID NO: 360 - DNA, 224 bp, Staphylococcus aureus
aacaacaaaaagtggtggtgaagtaacacgaaaatatattcagcaagcacttgataaaaacccagaactgttactagcagatgaaccgacaactaaccttgatacaaattatatagaaaacttagagcgaaaattaaaggactggcatggagcatttatcatcgtttcacatgatcgtgctttcttagatactctttgtacgagtatatgggaaatcaaagacg

SEQ ID NO: 361 - DNA, 224 bp, Staphylococcus haemolyticus
aaaatataagttcatcggaagcaaggctaaaaggagcgaagccatacttcgcaaataaacagaagaagttacggaaaacggcaaaagctctagaaactagactggataagcttgaaaaggtagaaaaaacaaaagaactacccccacttaaaatggatttattgaattcagagacttttaaaaatcggattatactccgtgttgaagatgtttcgggtgtaatt

SEQ ID NO: 362 - DNA, 224 bp, Streptococcus pneumoniae
ggaaagcaaaatgtacttgagtatgtctatgactttgttattggatttacagaacctaacattggttcgcgctacatgaaagattactcactctttttcctttgtttattccttttcatggtgattgccaataaccttggcttaatgacaaagcttcaaacgatcgatgggactaactggtggagttcgccaaccgctaatttacagtatgacttaaccttatc

SEQ ID NO: 363 - DNA, 224 bp, Salmonella enterica
gacggcaagcccagagccatgaagcaatgggaaaaagacttaaagctaaggggcgctatacaggtttctgctgttccggtatttcaacaaattgccagagaagttggcgaaataagaatgcaaaaataccttaacctgttttcatacggcaacgccaatatagggggaggcattgacaaattctggctagaaggtcagcttagaatctcagcattcaatcaagt

SEQ ID NO: 364 - DNA, 224 bp, Enterococcus faecium
acctattttagaaaaattagaaaatgttgaggttggagaatactcatattatgattctaagaatggagaaacttttgataagcaaattttatatcattatccaatcttaaacgataagttaaaaataggtaaattttgctcaataggaccaggtgtaactattattatgaatggagcaaatcatagaatggatggctcaacatatccatttaatttatttggta

SEQ ID NO: 365 - DNA, 224 bp, Streptococcus pneumoniae
aatggagtcccgattgctgaggacgcaacttcctataatgtctatgctgttattgataaaaaatacaaatcagcaacgggtaaaattctttatgtagaagatgcccagtttaataaggtagcagaggttttccataagtatttggatatggatgaggcttatgtgaaagagcaattagctcaaccaaatctgacccaggtttcctttggagcaaaaggaaatgg

SEQ ID NO: 366 - DNA, 224 bp, Streptococcus oralis
tcagcttcctgcggataatattgtcaacattgcgcaaagctcatttgggcaagggatttcagtgacacaaacacaaatgcttcgtgcctttacagctattgctaatgatggagttatgctggagccaaaatttataagtgctatttatgatactaacaatcagtctgtacgaaagtctcaaaaagagattgtaggaaaacctgtatctgaagatgcagcaagct

SEQ ID NO: 367 - DNA, 224 bp, Streptococcus pneumoniae
ccatgcctagcatcaaggacatttcacctggcgatttggcggaagccttacgtcgcaatattgtgcaaccaatcgttgtaggaacaggaacaaagattaaagaatcatctgtagaagaaggaaccaatcttgctcctaaccagcaagttctcctcttatctgataaagcagaggaagttccagatatgtatggttggacaaaagcaaccgcagaagctttttct

SEQ ID NO: 368 - DNA, 224 bp, Klebsiella oxytoca
aagatgtggtgaataaaaggctggagattaaagcatcggatctggtggtctggagcccggtgactgaaaaacatctgcagagcggaatgacgttggcggaattaagcgccgccgcgctgcaatatagcgacaataccgcgatgaataagatgattggttatcttggcggaccggaaaaagtgaccgccttcgcccgcagtatcggcgatgtcacttttcgtctc

SEQ ID NO: 369 - DNA, 224 bp, Aeromonas media
cggcagcttttgctccatcggaagcggggcttctttcatcatggcgggcaatcagggtcaccggcatgactgggtaacatctttccctttcttctacatgcaagaagagccagctttttcaagttcaacggacgcctttcaaaaggccggtgacaccatcgtcggcaatgatgtctggataggatcagaggcaatgattatgcccggcatcaagattggagatg

SEQ ID NO: 370 - DNA, 224 bp, Xanthomonas maltophilia
gctggtccagacggctgatggcctcgtcctgatcgatggtggcatgccgcaaatggcctcttacctgctgaccaacatgaaggccaggggaaccaataccggtcccctgcgcatggtcctgctcagccacgcgcacaccgatcacgccggccctgtggccgagatcaagcgccgcacgggggcgcaggtggtcgtgaacgccgaaaccgcagtgctgctggccc

SEQ ID NO: 371 - DNA, 224 bp, Klebsiella variicola
agctatgaaagcgctaaaggcgacttagcgaaagcccaggccgccgcaaacatcgctcagttgacggtcaaacgttatcagaaactggttggcaccaaatacatcagccaacaagaatatgactcagccgttgccgacgctcagcaaagcaacgccgccgtcgtcgcggcgaaagccgcagttgaaaccgcgcgtatcaacctggcttataccaaagtgacgtc

SEQ ID NO: 372 - DNA, 224 bp, Enterobacter cloacae
gacgtggcgaaagttgaactcggcggcgaaaactacgacatcatcgcgaagttcaacggccaaccggcttctggtctggggattaaactggccaccggcgctaacgccctggataccgccaacgccatccgcgccgagctggcgaagatggaaccgtacttcccgtcaggcctgaaaatcgtctacccgtacgatactaccccgttcgtgaaaatctctattca

SEQ ID NO: 373 - DNA, 224 bp, Citrobacter koseri
gtgctgtatctgattatcgtcgtcggtatggcttacctgttcgttcgtctgccaagctccttcctgccggacgaagaccagggcgtgttcctgagtatggcgcagcttccggcgggcgcgagtcaggaacgtacgcagaaagtcctcgacgagatgactgactactacctgaccaaagagaagaacaacgttgaatcggtgttcgcggttaacggttttggttt

SEQ ID NO: 374 - DNA, 224 bp, Klebsiella pneumoniae
tcccattgggaatatggttcaccgcgtctggaacgttacaacggtctgccgtcgatggaaatcctcggtcaggcagcgccgggccgcagtacgggtgaagcgatggcgatgatggaacagctggcaagcaaactgccttcaggcgtcggttacgactggaccggtatgtcctatcaggaacgcctgtcaggcaaccaggccccggcgctgtatgctatctcgct

SEQ ID NO: 375 - DNA, 224 bp, Enterobacter aerogenes
ttcactgcaattaacgcaaaccctgttcgatatgtcgaaatggcgcgcgctaaccctgcaggaaaaaacggcgggcattcaggatgtcacctatcagaccgaccagcaaaagctgattctgaataccgcgacggcttactttaaagtcctggccgctatcgatacgctctcctataccgaagcacagaaacaggctatctatcgtcagttggaccaaaccaccc

SEQ ID NO: 376 - DNA, 224 bp, Enterobacter aerogenes
gcttccaccagtgtctctaataaccgttacagcggttccaaaaacatttcccaggacgccgatgtcgggcaaaacaaagtcggcctgagcttctccctgccgctgtatcagggcgggatggttaactcacaggtgaaacaggcgcaatacaactttgtcggcgccagcgagcagctggaaagcgcgcaccgcagcgtcgtgcagaccgtgcgttcgtcgtttaa

SEQ ID NO: 377 - DNA, 224 bp, Campylobacter jejuni
attacccaaccgaaagtatgcggtcgtaacacgttcaagctttacatctgacaatgagaacgtattgatctttccatcaattaaagatgctttacccaaccgaaagtatgcggtcgtaacacgttcaagctttacatctgacaatgagaacgtattgatctttccatcaattaaagatgctttaaccaacctaaagaaaataacggatcatgtcattgtttcag

SEQ ID NO: 378 - DNA, 224 bp, Pseudomonas aeruginosa
aggggataccaggttatggctagcatctcaactcattctcatgaagatcgcactgctggtatcaagttgctaaattcaaagtcaattcctacatacacatcagagttaactaaaaagcttcttgcccgtgaaggaaagccggttcctacccactactttaaagacgacgaattcacactgggaaatgggcttatagagctctactatccaggtgctgggcatac

SEQ ID NO: 379 - DNA, 224 bp, Pseudoxanthomonas suwonensis
ttcctgcgcggccgttctggcccaggccgaccgccacaagctgaacctggagcaggcgataccgatcgagcgcacagcgctggtcacatactcacccgtgacggaaagggtgccacctggcggcacgctgaccctgcgtgagctgtgcagggccgccgtcagtatcagtgacaacacagcggccaatttggcgttggatgcaatcggcggggcacggacattca

SEQ ID NO: 380 - DNA, 224 bp, Pseudomonas aeruginosa
ctgccgccgcgccatcgaaagagcgaaaaaaccggatgcctccccggtactggccgagaatgcgccgctcgttgccaacgtgcagcacgatcaccagcccgcgctctgtgttctcgatgacagcgcccttgatctcatgcggtgcgttcttcgcaaacgccttgaaatgagagggcggcagtacctccagactcaacggttccatttcaaccatcactttatac

SEQ ID NO: 381 - DNA, 224 bp, Streptococcus pneumoniae
tttgatcaaaacggtaagttaggatactgggatagtatgaacccaagttatactatctttcataaggataacgaaactttttcaagtatttggacagagtatgacgagaacttcccacgtttttactataattaccttgaggatattagaaactatagcgacgttttgaatttcatgcctaagacaggtgaacctgctaatacaattaatgtgtccagcattcc

SEQ ID NO: 382 - DNA, 224 bp, Unknown
  Description of Unknown Polynucleotide from a pathogenic organism
agtcatcatgggtgcaggcacgtataagagcctcccaagtcctctgaaagaccgcatcaatatcgtcatcaccaagaagtcagagatttcttggacggcttgctatgacgtgcgtgtggtcaacagtccagaagacgctttgcgcatggttggtcgcattatcgacgagaaagaagagcaaggtcgtgatcgacctcgtgtattcgttatcggcggggcttcga

SEQ ID NO: 383 - DNA, 224 bp, Clostridium difficile
ttctgtgaaagactcatattgtagcaatttaatagtaaaacaaatcgagaatagatataaaatattgaataatagtttaaatccagctattagcgaggataatgattttaatactacaacgaatacctttattgatagtataatggctgaagctaatgcagataatggtagatttatgatggaactaggaaagtatttaagagttggtttcttcccagatgtta

SEQ ID NO: 384 - DNA, 224 bp, Cronobacter turicensis
ttatacctacactaacggttttcgcgataacaatggcatcgattcgaatgccaccagcgcctctttacagcttacacaaaccctgttcgacatgtccaaatggcgtgaactgagtttgcaggaaaagagcgcaggcattcaggatgtcacctatcagaccgatcagcagacgctgatcctgaataccgcgaccgcctatttccaggtactgagcgcgattgacg

SEQ ID NO: 385 - DNA, 224 bp, Enterobacter cloacae
agcactgcgtcaggtcaccggtaactactatcctgaactggcctctctgaacgtggacagtttcaaaacggacaaaccgcaggcggttaatgctctgctgaaagaagcggaaaaccgcaacctgaccttattgcaggcgcgcctgagccaggacctggcacgcgagcagatccgccttgcacaggatggtcacctgccgacgctgagcctgagcgcctcgaccg

SEQ ID NO: 386 - DNA, 224 bp, Enterobacter sp.
ggacgcgatggaagcgggctactccgtcggtacgcgtaccatcgttgacgtgttagacgcaacgaccacgctgtataacgcgaaacagcagctctccagtgcgcgttaccagtacctgatcaaccagctgaacatcaagcaggcgctgggtacgctgaacgagcaggatctgcaaatgctgaacagcacgctgggcaaaccggtttccacgtcgcctgacagcg

SEQ ID NO: 387 - DNA, 224 bp, Bordetella pertussis
ggggcgcaaaacctgggaatcactcaatggtgccttgccggggcgcaccaacatcgtggtaacgcgtcaacaaggttatgaagccgaaggtgctcgcgtggtcgatagcatcgaagaagccattagcttggcacagtctatcgccttaatcgaagccgttgatgaaatcatggtgctgggcggcggcgaaatctatacccaagccttaccgcaagccgacattc

SEQ ID NO: 388 - DNA, 224 bp, Variovorax paradoxus
tgctggccaccggacagctggcccggcttgtggtgcatgcggtcgcccaggcccagccgctgcagcagctgctcggcgcgcgcgttgcgcactgcgcccgggctgccggcatacacggccggtacctccacgttgccgcgcgcatccaggtcgcccagcaggtggtagcgctggaagatgaagccgaaatgctcgcggcgcagttcggccagttcatccggcgc

SEQ ID NO: 389 - DNA, 224 bp, Burkholderia multivorans
cacgcgcagcagcacgtcgccggtgcccggatcgacgttgacgcccgagaacagcatgcggccgcgcacgtcgtaacgcgtgtcgtcgtcgcgcagcacgtcgaccggcaggccgttgctgtccgacgcttgcggttgagcggcaagcgcgccgcgcagcgcgtcgagcgacgcggccggttgacgcacgtccacgtagacctggtcgatctgctggacgcgcg

SEQ ID NO: 390 - DNA, 224 bp, Unknown
  Description of Unknown Uncultured bacterium polynucleotide
gtcgcggccgctgacgagctgcacgcgcgcatagaggcccggcgtgaacgtatggtccgcattcggcagccgcacgcgggcgcggatcgtgccggtttgcggatcgagccggttgtcgaggaaatcgacggtgcccgcatgcgggaagcccgtctcgttcgcgaggccgacgcgcaccggatcggcgccgatcgcgcgatgcgtcggatcggcgcgccgcgcgt

SEQ ID NO: 391 - DNA, 224 bp, Burkholderia sp.
atccagacgctcgtgcccgccgacgtcgccgcgctcgccgagcagccgtacgtcgacagcgcgacgcccgaaacgtcgcgcacgctgctgctgcgctaccgcaacgtcgacgtgaacgcgctcgtgagcggcgtcggcgaccgctacttccaggcgcgcggaatgcgcttcgcgctcggcgtcgcgttcgacgacgatgcggtgcgccgccaggcgcaggtggc

SEQ ID NO: 392 - DNA, 224 bp, Escherichia coli
gacacaaacccttgatattaaagaagcgaaaacttggttactggagacgcaaatttctgactaccgcctcggacctacctttgcgggcgatgtgcgcagctggctggcggacatgccgcaccgtgaactggcgcgaagattaagcggcggattaacttacggtgaaattccggctgccattaacaatatggtggtggatacccacacgtctaatgactttatta

SEQ ID NO: 393 - DNA, 224 bp, Enterobacter sp.
agggcgtggagttcctcgccaacagcctgttcaaacatcgtcaggccgagcgagtgatcgccgttgagctgccaaaacaccgctcctgtatgcaccttgacaccgtcatgacccacatcgacgttgacactttctccgtttacccggaagtggtgcgcaaagacgcccagtgctggacgctcacttcgaacggacgcgatggcctacaacggacccaggaaacc

SEQ ID NO: 394 - DNA, 224 bp, Artificial Sequence
  Description of Artificial Sequence Synthetic plasmid pB3 polynucleotide
agcgccgacggtgaagtagctgattgccatcaacgcataggtcgcgtcacctgccgaaaagccagcatcgcaaaggaagcgaagctgcgcgtcggctttttccatctgcggcgcggctggccgcgtcccggcatgaatacgcgcgccatcgcgataagcgagcaacgcccgtcgaaaactgcatgcattgcccttcaggaacgaacgccagtcgtcgtcatccc

SEQ ID NO: 395 - DNA, 224 bp, Streptococcus oralis
ggattacctatgccaatatgatggctattaaaaaagatttaaaagatgctagtgtggaagggattgactttacaactagccctaacagaagctacccaaatggacaatttgcttctagctttattggcttagcccaacttcatgaaaatgaggatggcagtaagagtttgttagggacttctggtttggagagttctttaaataccattcttgctgggacagac

SEQ ID NO: 396 - DNA, 224 bp, Unknown
  Description of Unknown Polynucleotide from a pathogenic organism
aagtaactatgaatccatgatcaaccatgaacggttatgacggtatcttctctactgatgatattacattcccacatgtataataccttcataagcgatgtactaaaaatcagcgatgagtacgcatcgagtattgactcttcgatgcaaggcttggacttactggacgtaggatgtactcgaggatgggagttttaggatgttgagcctctcgtagagatgat

SEQ ID NO: 397 - DNA, 224 bp, Streptococcus oralis
aaatggaggatacttggttggttctaccaattatattttctcagttgtgactatgaatcctgctgaaaatcctgattttatcttgtatgtaacggttcaacagcctgagcattattcagctgcccagttgggagagttttccaatccaatattggagcgggcttcagcgatgaaagaatctctgaaccttcaatctccagctaagaatttagatcaagtgacga

SEQ ID NO: 398 - DNA, 224 bp, Acinetobacter sp.
aacaggtccaacgacaaggggtaaatatttggaaaaccacaggtgatatgctgttaattgtggggctatacgatgaaacaggaaaagccagcaatatcgaattatcggactatatggtcaatcactttgaacaaccactctcacaattgcaaggaattggtgaagtggatgtgtttggttcaagttatgcaatgcgtatttggttaaatccaaatcaacttaga

SEQ ID NO: 399 - DNA, 224 bp, Vibrio parahaemolyticus
aacagtcgatgattttaaatcggttgtcgttaaagctaatgtagatggcagtctggtttatttaaaagatgtggcgagagttgaattaggcgccgaaaactatgaaagtatcaatacgctaaacggctacccttccgctggtttaggtatttcactcagtcctgatgcgaacgcgattgaaacctctggtctaattaaagcaaaaattgaacagctcaagcagc

SEQ ID NO: 400 - DNA, 224 bp, Methylotenera mobilis
aatatgcttgaaagtagtgaccaaacaagtatttcacagttattaattaacttaggtatcactattggtaaagtagttgcctttattttaattatgatgattgtcggtcgtaagcttattccgtggatcttagctaaaacggctgccactggctctcgtgagctttttaccctatgtgtgttagcactagctttaggtatcgcttacgcagcagtgacgctatt

SEQ ID NO: 401 - DNA, 224 bp, Photorhabdus luminescens
gaaccgcattaaccatttcagccagtcttgcgcaaattggtgaatttgcctttattttagctggaatgggggttgcactaaatgttttagagccagatgcacgtaacttggttcttgccggcgccttagtctctattatgcttaatcctgttctcttctctttgttagaccgttatctagcaaaaacagaaaccaaagaagagcaagagcaacttcaacaagaa

SEQ ID NO: 402 - DNA, 224 bp, Acinetobacter baumannii
actaattgcaatcataaatggaataataaacatcgccgcctgtaatggagaaaacccgtgtacaaactgcaactcttgagacagtaataattcaaagccaaccaaagcaatcatggaaacaacagccataacaatacttgtagaaattactggatgtttaaataattccagatcaatcattggtgtcatagctcttttttggcttcgaatgaagtgaattaatg

SEQ ID NO: 403 - DNA, 224 bp, Acinetobacter baumannii
gatttaattgaatagatgaggcttaaaattgctacaaccaaaactaaagcttgccctaagttaattggctgatcagttttctcttgttgtttaggaatgatcatcacgatcataactagaacaactaaaataatcggaatattgatgaggaatacagccccccaatgaaaatgttctaatacaaaaccaccgactaatgggccaaaagcagctccgcctccccc

SEQ ID NO: 404 - DNA, 224 bp, Photorhabdus asymbiotica
taaaaataccgagtctaactctcttggtgcgaatttaacgctgactcagactatttttgatatgtctttatggcgtcaattgaccatgcaagaaaaaacggcgggtatgagtgatgttacctatcaaacaagccaacaacagcttattttagataccgcaacagcttattttaatgttctgcgtgccattgactccttatcatttattgaagcacaaaaagagc

SEQ ID NO: 405 - DNA, 224 bp, Unknown
  Description of Unknown Polynucleotide from a pathogenic organism
gtcataatcatggtagtgctttaccaccagaaacacctgctaacagccgtaatagctataatggtcaaagcagcatcggactctctttaagcattccattatatacaggtggcagaacgagctcacaagtagaacaagctcaatatggctttaccagtgccagtgagcagttagaatcggtataccgctctatcgtacaaattgcacgttcttcttataacaat

SEQ ID NO: 406 - DNA, 224 bp, Photorhabdus asymbiotica
gcttgcagaaaaagccattggatttattcatgccattatgtggggggctcctggttgtctctattatcaagtgctaagaagccaatgtgagggattatcaaaaacaaaaccaggcatgatcatcggctttattggattattaattaacattcctgttaactacgcctttatttacggtaaatttggtgccccacaattaggtgggattggttgtggtgtggcaa

SEQ ID NO: 407 - DNA, 224 bp, Xenorhabdus nematophila
ttatgttccctctttccttgggaatagctgcaacaattcgtgtgggttataaccttgggcaacgttctacagagcaagctcgtacttcagccattacagcccttgctgtcggattaatgctggcaagttgtaccgctattttctctattatttttagagaaaaaattgctttaatgtataacgataatattgaagtggtgacattagcctctcatcttatgtta

SEQ ID NO: 408 - DNA, 224 bp, Burkholderia glumae
gcgagatgcgcgggaagaatgccgcgcgcgccgcgccgatgttcgcgttcgcggcgagcagcgtctgctcggcctgcatgatgtcgggacggcgcgtcagcagatccgacggcagcccggccggcacgtcggcgagcagattttgcgcatcgagcggcagccccggcggcagatcgtccggcagcggctcgccgatcagcagcaccagcgcgttgagcgcctgc

SEQ ID NO: 409 - DNA, 224 bp, Pantoea sp.
tggctcgcctgccgggcgtcggccaggtgcagatcttcggcgccggtgaatacgcgatgcgcatctggctcgatccgaacaaggtcgccgcgcgcgggctgaccgcctccgacgtggtgagcgccatgcaagagcaaaacgtgcaggtctccgccggccagctcggcgcggagccgatgccgacgcgcagcgactatctgctgtcgatcaacgcccagggccgc

SEQ ID NO: 410 - DNA, 224 bp, Pantoea vagans
gacggttttcgtgcgcgattcgatccgcgcggtggtggataccctgctggaagcggtgatcctggtggtgctggtggttattctgttcctgcagacctggcgcgcctcgatcattccgctgttggcggtgccgatctccgtggtcggcaccttcgccgcgctgtatctgctgggcttctcgctcaacaccctcagtctgttcgggctggtgctggcgatcggca

SEQ ID NO: 411 - DNA, 224 bp, Serratia proteamaculans
attatgcaaacgccaggcatggggttcccgatctcctcttatcaggccaacgtgggccaacgtgacgccaaaatcgatcgcgacaaggccaaggcccagggggtgccgctcaatgcgctgttcagcacgctgcagacctacctcggttcgtcttatatcaacgacttcaaccgctatggccgcacctggaaggtgatggcgcaggccgacgcccagttccgcga

SEQ ID NO: 412 - DNA, 224 bp, Pseudomonas aeruginosa
aaacgccggaggtcggggtcatcaccgtcaggcgcagacggtgaccctgaataccgagctgcccggccgcaccgtgccgttccgcatcgccgaggtgcgtccccaggtgaacggcatcatcctcaagcggctgttcacggaaggcagcgacgtcaaggcagggcagcagctctaccagatcgaccccgccacctacgaggccgactaccagagcgcccaggcca

SEQ ID NO: 413 - DNA, 224 bp, Ralstonia eutropha
ggagcgcgccggcgacaacgcggcgcaggtctccctgaagctggaggacggcagcacctacccgctggaaggccgcctggagttctccgaggtttccgtcgacgagggcaccggctcggtcaccatccgcgccgtgttccccaacccggacggccagctgctgccgggcatgttcgttcacgcgctgttgcaggaaggcgtcaagcagaaggccatggtggtgc

SEQ ID NO: 414 - DNA, 224 bp, Leuconostoc gasicomitatum
taaacctatggactggtgtcgcttttagtattacttttctcttttctgccatagcggcacctttttggggaaaattgtcagatagaaaaggacgcaaattaatgctattacgttctgccttaggtatggcgatcgtcatggtactcattggttttgctcaaaatatttggcaactattaattttacgcgctttattaggggtattagggggttttgtgcctaat

SEQ ID NO: 415 - DNA, 224 bp, Streptococcus gallolyticus
cgcggttttatttatctgttttatagtgaccctattttttgttagcgaaaattttacccctgtttctaaaaaagatgcattatccacccagcaagttttttcatcactcaaaaataaaagattagtcatttgcctgtttttcaccaccatgattatccaagtggcaacaggctctgttacgcctattttaacactttatatccgtgatctagccggctctatca

SEQ ID NO: 416 - DNA, 224 bp, Pseudomonas aeruginosa
gatcgcccggctgtcggtgccctgcggcgcccgcacgcgcacgtacatgcccggcagcagcacgccgtcgggattggcgaacttgccgcgcaaggtgatctggccggtgccgcgatccaccgccacgtcggcgaactgcaatgcgccccggcgctcgtaggaggtgccttcgacacggatgctcaacgcctcgctgtcgccggcggccaggctgccatccttca

SEQ ID NO: 417 - DNA, 224 bp, Photobacterium damselae
tagcgcaatactgccgccggcgagtaagattaatgtagggtaaatcatccaaccctttgtcaataatgacaaaataatgaatgctgctccatcaacaacaaagcccacaataatcgtcactttttcattgaatttcttggcgattgcgcccgctacaaatgcttggaacaaggcatgcatgatgcctaaccccgctaaagataggccgatctccatactgcccc

SEQ ID NO: 418 - DNA, 224 bp, Photobacterium damselae
taaataacaatagtatgactggcttgatcacttgcaggaaaggtcgcgaattttctgctgtttctgtggtgtttttttcagtatttttgattttattatctttaaatatcaacataataactaaaaaagagagcgcatttaaaatggcagcaataataaaaggaagatgagctgagaattgccctgtaaagccgccaattgcagggccagagattaacccgaca

SEQ ID NO: 419 - DNA, 224 bp, Xenorhabdus bovienii
ttgttctaggcgaaaatgtttttcactccaacgggcaataggataaagcagtgttaatgataataaagcttcaattgcatacatccattttactgcgctaggggtgccggcaatttcattaacaacgatcggaaacatcagcataacttgtaccgatagtacaaaatatcccgtaagagtaagaacatactgaacaaaacgtttatcaagaataacgcgtttta

SEQ ID NO: 420 - DNA, 224 bp, Arsenophonus nasoniae
aggcaggtagtaaccaagcattaaaaatagccgcaagaacgaatatgccagcgccaacccaacaaactaaatggaaatcataaaggagtaaccaactgccaattaaagccccaataacggcacccgcactatcttgcattaataataaagaataaaaccgaccccgttcataagggcgagttaattttatcacgagggcagttcttggcggatcaaataaagtg

SEQ ID NO: 421 - DNA, 224 bp, Shigella sonnei
cgcgacgcgaacggcatcaactctaacgcgaccagtgcgtccctgcagttaactcaatccatttttgatatgtcgaaatggcgtgcgttaacgctgcaggaaaaagcagcagggattcaggacgtcacgtatcagaccgatcagcaaaccttgatcctcaacaccgcgaccgcttatttcaacgtgttgaatgctattgacgttctttcctatacacaggcgca

SEQ ID NO: 422 - DNA, 224 bp, Shigella sonnei
caggtgaaacaggcacagtacaactttgttggtgccagcgagcaactggaaagcgcgcatcgtagcgtcgtgcaaaccgtacgttcctccttcaacaacattaatgcttctatcagtagcattaacgcctacaaacaagccgtagtttccgctcaaagctcattagacgcgatggaagcgggctactcggtcggtacgcgtaccattgttgatgtgttggatgc

SEQ ID NO: 423 - DNA, 224 bp, Stenotrophomonas maltophilia
ctaccgtgcgcttctggccgtccaccgtcgccttccacgacaacgtggagttccactacgacatcctggcccgccgcctgcgcgaactgtcgttcctgaactccggcgtcaagatcgtccttgccgacgagcgtggcgacggccgtcgcgatgacttccactacgagggtggcatccgcagcttcgtggagcatctggcccagctgaagacgccgctgcacccg

SEQ ID NO: 424 - DNA, 224 bp, Burkholderia sp.
gaacccgatcgcgcacgacgcttcggccagctcggcgcgatgatgggcatcggcttcatcgcgggcccgctgatcggcggcctgctcggcgcgctgcacctgcgtgcaccgttcgtcgcggccgccgtgctcaacgcgctcaatctcgtgctgggctggcgtgcgttgccggaatcacgggcgccttcggcccgcgaaggccatgcagttggcgcgctcaatcc

SEQ ID NO: 425 - DNA, 224 bp, Streptococcus pneumoniae
actaaggatggcattacaaaagactttgtctggcgcgatatcctttatcaaagtaactacgagccagggtcaaccatgaaggtgatgatgttggcagcagctattgataataagactttccctggtggagaatactttaacagtagcgagttgaaactggcagatgccaccattcgagactgggacgttaacgaaggtttgacgagtggtggaaccatgacctt

SEQ ID NO: 426 - DNA, 224 bp, Escherichia fergusonii
tactgcgttacgctatgccaatagcttattaaatatgggggtgttagtcagcccactcttagcgccaatgatcggtggggtggtggcgcacttctttggctggcatgcctgttatatcttcctattattattaggaggaagtgtactctttagtatgtatcactggatgccagaaacccgacctgtacaggctgaaagacgcagtatgttgtcatctttttatt

SEQ ID NO: 427 - DNA, 224 bp, Burkholderia cenocepacia
ggctggctcaccgcgaaggcgatgcaccaggatttcgcgacgctgatggagcagcggctctttcccgcgatcgggatgacgcatacgtacatcaacgtgcccgccgcgcgcatggccgactatgcgcagggctacacgaaggacggcaagccggtgcggatgacggaaggcatgctgtggcaaccggcttacggcgtgcggaccacggcggccgacctgctgcg

SEQ ID NO: 428 - DNA, 224 bp, Arsenophonus nasoniae
tgacatttagtcaatttattagcttatttagagcacgtcgagtactttgctatattctggcatcgggattttcttttgcagggatgttttcatttttaagtgcagggccttttgtttatatcgaactacatggtattccattcgaccaatttggcttgtactttggttttaatattattttcttgattgtcatgaccagtatcaatgggcgctatgtgcgccga

SEQ ID NO: 429 - DNA, 224 bp, Burkholderia multivorans
gcacgctcgggtggatcaccgcgaaggcgatgaagcgcgacttcgccgagctgatggaacgacggttgtttccggcgctcggcatgacgcacacatatatcgacgtgccggacgcgcgccgcgccgattacgcgcagggctatacgaccgacggcacgccgatccggatgaagccgggcgggctgtggcagccggcgtacggcgtgcgcacgacggccaccgat

SEQ ID NO: 430 - DNA, 224 bp, Unknown
  Description of Unknown Polynucleotide from a pathogenic organism
tacgcacttctgttgcagcttgtaatacagaaggttgttgtgcaataggcgtggttaagataatggcttgattagcattatattgctcttttgctacatctaatgccgcttttgccgtggcaacagcttctcttgcatgttggagttcttcttttccaatgacatttcgctcgcctaatacttctctacgacgcagatcattttgcaattttgccaattcagtt

SEQ ID NO: 431 - DNA, 224 bp, Photorhabdus asymbiotica
ggcaacttgatagaagaaggtatctgctgactcaataatggatttagtaacattgagttttccatgaccccaacgtttccagtcacgatagcgtttttcagagccgggcagttgccaccagcccggatcatggatagttgtgttaggagtaataaccccttcacttagggctgcaacagacataaagggtttaaccgttgatgcaggaggatagagcccttggg

SEQ ID NO: 432 - DNA, 224 bp, Paenibacillus apiarius
attggttacaatcatatagatatgaaggcagcggaaaaaatgggtattgctgtcgagaacgtcacttattcaccagatagtgttgccgattatacattgatgctgatacttatggcaatacgccatacgaaatctactttgtgctctatggaaaaacatgatttcagactgaacagcgtccgtggtaaagtactgcgtgacctgacagtaggtgtactgggaac

SEQ ID NO: 433 - DNA, 224 bp, Staphylococcus epidermidis
gtctgttaagatcggttactttagtcagaatctaatgacgctagatgtaaacaaatctattttagagaacgttcaaaattcttcgaaacaagaagaatcactgattcgaacggtactagctagaatgcatttctttgatgaagatgtatacaaacctgtgcatgtgctaagcggtggtgaaagagtaaaagttgcactgacaaaactattagtgagcgatatca

SEQ ID NO: 434 - DNA, 224 bp, Stenotrophomonas maltophilia
gaagtggccgaaacgcagtcgcgcaatcgcctggcggttctgctgggccagcgtccggaaacgctgaccgccatgcttgcgccgcacgacgtgccggcatttgccaaggcgctgccgctgggcgatacccgcgagctgctgcgccaacgcgccgacgtgcgcgtggccgagcgtcgcctggccgctgccaccgcgcgggtgggcgtggccactgcggacctgtt

SEQ ID NO: 435 - DNA, 224 bp, Xanthomonas axonopodis
gcgtcgcgcggccgccgaccagtccggtgccgccgtgcaggccgcgcaggctgcgctggacgcggcccggctcaacctggagttcaccaaggtgcgtgcaccgatcgacggccgtgccggtcgcgcgatggtcaccgccggcaacctggtcaccgccggcgacagtgccagcgtgctgaccacgctggtctcgctggataccgtctttgtctacttcgatgccg

SEQ ID NO: 436 - DNA, 224 bp, Yersinia enterocolitica
tgattattgtcactcacgatccattaattgcccaacaagccgatcgtattattgaaattaaagatggtcaaattattagcgataataataatcaccattctgcaccagtgaaaaaggcaccacctgcgatccaaaccgcctcttattttcatcaagtcattggacgttttacgcaggcattaaacatggcttggcgagcaatggtggtgaataaaattcgtacc

SEQ ID NO: 437 - DNA, 224 bp, Enterobacter cloacae
aacacctggataccgggatgacccttgcagagctcagtgccgccgccatccagtacagtgacaacacggcgatgaacaagatccttgagcatcttggcggcccggcaaaagtgacagagtatgcgcgtactattggtgataaaacctttcgtctcgatcgtaccgagcctactttaaatactgccattcccagcgataagcgtgacactacctcgccgctggcg

SEQ ID NO: 438 - DNA, 224 bp, Paenibacillus apiarius
gaagagagtataaaaacagacgttgtaaatttatttaaacatgatgaattgacaaaaggatatgaattgctcaatagggaaatttatttatcggagaaagttgcccgtgaattttcagagatggtagatgcggctgaaaaagaaggagttcgccatttttcaatcaatagtgggtttcgaaactttgatgagcaaaatgccctttatcaagaaatggggtctga

SEQ ID NO: 439 - DNA, 224 bp, Photorhabdus luminescens
ataaagtgggtttagtgtcgaaggggctttaggtaaacctgcaataacagccatctcacttaagctaagatcatgcacacttttaccaaagtaaacatatgcagcagcacccacaccataagcgcgatttcctagataaattttattgagataaagtgccaagatctcatctttagtaagctctttctcaatacggatagccaaaaaggcttctttgattttac

SEQ ID NO: 440 - DNA, 224 bp, Xenorhabdus bovienii
tgctatttttattgaagaaatctccgaagtgccggataacgctattttgatcttctcagcacacggtgtttctcaagctattcgccaagaggcacgctctcgtaatttaacgatgctttttgatgccacttgtcctttagtgaccaaagtacatatggaagtcgcaagggcgagccgtaaaggtaaagaggccattttaattggccatgccggacacccagaag

SEQ ID NO: 441 - DNA, 224 bp, Acinetobacter baylyi
gctacaacaactgagattttcaaatgggacggtaaaaagagatcttatcccatgtgggaaaaagatatgactttaggtgatgccatggcactttcagcagttcctgtatatcaagaacttgcaagacggactggcttagacctaatgcaaaaagaagttaaacgggttggttttggtaatatgaacattggaacacaagttgataacttctggttggttggccc

SEQ ID NO: 442 - DNA, 224 bp, Enterobacter asburiae
gcgtcgatcccgcggcggaagggctggattatacgattcgctacggtggcggggcgtggcacggcactgaagcgcagttcctgtgttctgcaccgctgtccccgctttgctcacccgatattgctctcgggctccagtctccggccgacattctgaagtttaccctgctgcgctcctaccgacgcgacgaatggtcggcgtggatgcaggcggcaggtgaacat

SEQ ID NO: 443 - DNA, 224 bp, Ruminococcus bromii
aaccaaatgaaacattttctttttggcaactcgtcagatgggcagacagacaagaaaaatacaaggacggattaaactttgttaacggaaaaattgtcggttcttatggtggcggactatgtcagcttagcaatatgctgttttggctgtttttacacactccattgacagtgattgaacgacacggacacgctgtagaatcgtttccctctacaacggaagaa

SEQ ID NO: 444 - DNA, 224 bp, Acinetobacter calcoaceticus
ggcgtgctggtgattcagcagggccagacccagcagagctatggcaacgatctggcgcgcgcgagcaccgaatatgtgccggcgagcacctttaaaatgctgaacgcgctgattggcctggaacatcataaagcgaccaccaccgaagtgtttaaatgggatggcaaaaaacgcctgtttccggaatgggaaaaaaacatgaccctgggcgatgcgatgaaagc

SEQ ID NO: 445 - DNA, 224 bp, Serratia proteamaculans
aagccaccttcctgcgttatcagcagcaaggccggcacgatgcccgcctgccggtgaaagtcggcctggtgggcgaagacggcactccgcatcaagggctggtggacttcactgataaccaactgaacgccgggaccggcaccatccgcatgcgcgccctgctcgacaaccgcgatcgccgcttcacgccggggctgttcgctcgggtacagatgcccggcagc

SEQ ID NO: 446 - DNA, 224 bp, Photorhabdus asymbiotica
caacctctgcgggtttttggtaacctaccttataatatttcaacgccattaatgttccatctatttagttttgctgatgccatttctgacatgacttttatgttgcaaaaagaggtggttaatcgcctagtggcaagtcatggtagtaaaacttatggccgcttaagtgtcatggcacaataccactgccaagtcattcctattattgaagtgccaccaagctc

SEQ ID NO: 447 - DNA, 224 bp, Mycobacterium abscessus
acccgctggtggacggacagggcaacttcggctcaccgggtaacgacccggcggccgccatgcgttaccaccgaggctcggttcacgcccgctggccatggagatgttgcgtgaaatcgttgaaggaaacagtcgatttcattccgaactacgacggccgcgtcatggaaccgacggtgttgccgaaccggtttcccaaccttgctggccaccgttcgggcggt

SEQ ID NO: 448 - DNA, 224 bp, Stenotrophomonas maltophilia
ggcgctggtgaccgaaggtgcactggtcggcgtggccgatgccgagccgatggcggtggtgcagcagatcgaccaggtctacgtcgatgtgcgccagccagcgacgcagctcgagtcgctgcaacgcagtgccggtggcggcgagctgccggtgacgatcatcggtgcggccggcaagccgctgcccgaacgtggccagatgctgttctccggtatcaatgtcg

SEQ ID NO: 449 - DNA, 224 bp, Acinetobacter baumannii
attatctctattttatttatttttttatgtttagccgctttatatgaaagctggaaagtacctcttattattttactaggacttccactaggaattactggaactcttgtatttgctcaaatctttagcttaccaaacgatgtgtatttccaaatcgcgttactaacgggtattggtttgtcttgtaaaaatgcgattttaattgtggagttcgcaactaaagc

SEQ ID NO: 450 - DNA, 224 bp, Acinetobacter calcoaceticus
ataacaacgtttatttaaacgctgagattcgtccatggggcgcaagtactaaccgttgggcgcaaggtttatatattgccgctggtgctgcatacttagacaatgattatgatttggctaaacgtattggtaatggcggaacattaacaattgatggtaacaattatcagcaagcagctgccggtcaggaaggcggtgttcgcggtaaaatgaagtatgagaat

SEQ ID NO: 451 - DNA, 224 bp, Serratia marcescens
cggcattatcggttggcgatgtgcaaaaggcactgtcgctgcacgcctctaccaaggagcggttggccgaattggactgcctctacgattttatcttttctggcggggtgccccatcgtgtgttggatatcgcttgcggcctaaacccgctggccctctttatacgtgacataacatctgtatgggcgtgcgacatccatcaggggttgggcgatgtgatcacc

SEQ ID NO: 452 - DNA, 224 bp, Photorhabdus luminescens
aaacagacgttgaagtcaaaaaagcccgagtggcgacttatgaagcacaaattagaaaaaatcaggcaacactggataccgcaagaactaatttgcaatatacccgtattactgcgccgatggatggtgtggtcacttttatcaaaacccttgaagggcagacagtcattgcagctcaagaagcaccaaccatattaacattagctgatttagataccatgtta

SEQ ID NO: 453 - DNA, 224 bp, Enterococcus flavescens
gggtaaaaacacaactaagacgagtccaaaagtataaccggaaagtggaaaatgaatcagtcatagagtttaacaaagacggactaacgctaaaaaaagacagtcatcaagtatttttatttgataaagaaataactgtaacacctattgaattcaatttgcttttatatttatttgaacaccaaggagtggttgttagttcagaagaactatttgaagctgtt

SEQ ID NO: 454 - DNA, 224 bp, Eubacterium siraeum
atgcttgaacaaattacatacgagttcaaacctatgcttgtaaacaaacacttaaattgtgagcttaacgcaccgcaagacataatgataagctgtgatataaataaaatgcaaagagtttttgataatttgctaagaaacgccgtaaattatagttacgaaggaacaaatatcaaagtattcgctgaactatttaacaataatgtgtgtgtaaaagtaatcaa

SEQ ID NO: 455 - DNA, 224 bp, Clostridium saccharolyticum
acggcaaaggaagaagatgctgacaagaacacggggctgacgattggagcggatgattatattacaaagccatttatcccagtagaggttgctgcaggggtcaagacacagcttcgccgccatgtaggttacaataatgccgcagccatagacaaagaacatgtattggttacggaatatgatattaacggactggtccttcatgagaatactcataaatgcac

SEQ ID NO: 456 - DNA, 224 bp, Unknown
  Description of Unknown Mixed culture polynucleotide
cccctcggcggcggggtcgacgcgattattatgggtggatatctgcaaatcgatatgcggacactcgcgccagaaggctggcagcagcgggaacagcacacccgtggcgaaagtgccgactacgccgatcttgagcttctcctgcgcctggtgaccggtgaaacggtcgagcatcccggcaatcctttcaaacgactcattgagtaccggtaacaggccttctc

SEQ ID NO: 457 - DNA, 224 bp, Burkholderia cepacia
gatgcgcttgtcgccgaccttgttaccgcgcagccactcgatcagctgcgcgcgctgcgcgggcggcaacgcgtcgccgagcacgagcacgcgcaggttcgcggccatcgcagcgggtgtcgtcgtgtcgcgaagatcgcccggcagcgcggtgttcagttcggtctcccagcgatcgaggcggaacgtgtcgtcgccgatcgagcgcgcataggccgtgaccg

SEQ ID NO: 458 - DNA, 224 bp, Photorhabdus luminescens
catcgcccgcatagcgcgcaccataactacgtttttagattgacctaaaccttgacgtaaacgaataggccctgcataagtaggtggtgagtttttagggcgccaatcagttccagctcccgcatcccaacgactaataggtaagtcatttagtaatgtcgataatgttaaccctttatctaatgctgccgcatataagaaaggtttgatatttgaaccaactt

SEQ ID NO: 459 - DNA, 224 bp, Escherichia coli
cagcaaaaccttagactcaatgggagttctaccaaatcgcaaatatgcagtagtgtcaaagaacaatatttcaagctcaaataacgacgttctagtttttccttcaatagaaaatgctttgaaagagctatcaaaagttaccgatcatgtttatgtctctggccgcgggcaaatgtataagagccttatggaaaaagctgataaaattattttgtctaccgttt

SEQ ID NO: 460 - DNA, 224 bp, Burkholderia sp.
cgctcgaggaactgctcgcacgcatccgcgccgtgaaccgccgccatgccggccgcgcgcagacgacgctcgcgatcggcccgctgcgcctcgatcccgtcaagcatcaggtctggctgcacgacgacgaagtgccgctgtcgccgaaggagttcgtgctgctgcacgagctgatgcgcgagcccggcgccgtgatttcgcgcgagcagttcgaggaacgcctg

SEQ ID NO: 461 - DNA, 224 bp, Burkholderia multivorans
cagcgggaagaacgcgcgcgtcatgatcgcgttcgcgtcgatcggcttgacgggcgtatcgccgccgcccggcacgtccggttcgttcagcatctgccagcccgacaccgcgaccgcgaagccgccgccgatgcgcagcgcctccatcgagatcccgaagaaatgcagcaccggcgtgccgacgaagaacgcgaccatcagcacgatgaacgcgttgaacgcca

SEQ ID NO: 462 - DNA, 224 bp, Ralstonia pickettii
gccgctcgtggatggcatggatgtcctgcagcgcattcgtcgcgacggcatggcgttgccgattcttatcctgactgctcgcgatgccgtgcccgaccgcatcagggcgctcgacgtgggcgccgacgactacgtgatcaaacccgtcgacctgtacgagctcggcgcgcgcctgcgcgcgctggtccggcgcgcccacggccggccgaccgagcggcttcagg

SEQ ID NO: 463 - DNA, 224 bp, Photorhabdus luminescens
aacgcaccaaaaggctcatcgagtaataaaatttgcggctctacagccaatgctctcgctaaagctacgcgctgtttttgcccccctgaaagttgtgcaggataacgctgtgctaaatgaggcagttggatcatctctaataattgcgtgacttttttatcaatcgcggctttgttaggacgttcgcgacgtggtaatacgcttaagccaaaggcaatattttc

SEQ ID NO: 464 - DNA, 224 bp, Sphingomonas wittichii
ggaacggatcatggccgaggaaggcctgccgccgcgcgaggccaccatcaaggcgatgcgcgaactgaccggtgcggtgatcggcatcaccctggtgctgaccgcggtgttcatcccgatggcgctggccagcggctcggtgggtgcgatctaccgacagttcaccgtggcgatggcggtgtcgatcctgttctcggcgctgctggcattgagcctgacgccgg

SEQ ID NO: 465 - DNA, 224 bp, Enterobacter sp.
atgcttcccacgtggtggtgttctgcgcgaaaaccgcgatggatgacgcctggctggagcgcgtcgtggatcaggaagaggccgatggccgtttcaacacgccggaagccaaagccgcaaaccataagggccgcacctacttcgccgacatgcaccgcgtggatctgaaagatgacgaccagtggatggcgaagcaggtttacctgaacgtcggcaacttcctg

SEQ ID NO: 466 - DNA, 224 bp, Paenibacillus thiaminolyticus
ctattgaaggttcaagaattagcttttaaccaaggttatggattgcttttatgggacggttaccgtccccaacaagctgtaaattgttttttgcaatgggcggcacagccggaagataatcgaacaaaggcaaaatattatcccaatattgaccgaactgagatggtttcaaaaggatacgtggcttcaaaatcaagtcatagccgcggaagtgcaattgatct

SEQ ID NO: 467 - DNA, 224 bp, Burkholderia vietnamiensis
ctgccgatcttcgacggcggacgcaaccgcgccaatctcgacctcgccgaagtgcgcaaggacatcgccgtcaaccgctacgaggaaagcatccaggtcgccttccgcgaggtcgccgacgcgctcagcgccggcgaccagctggagctccagctgcgcgcgcagcgcgctgtccgcgacgccgaccgtgaacgcctgcaactggtacgcaagcgctatgccaa

SEQ ID NO: 468 - DNA, 224 bp, Cucumis sativus
tcggcctgcgcgtgcggcaccgttcgcagcgcccctcggaggcgcggatccgcgccaaggtgctcgacctgctggggctggtgcagctggactggctggccgaccgctatccggcccagctctccggcgggcagcgccagcggattgccctggcgcgcgccctggccgtcgagccgcgcgtgctgctgctggacgagccgttcggcgcgctggacgccaaggtg

SEQ ID NO: 469 - DNA, 224 bp, Enterobacter cancerogenus
ttccgctgccggatcgacgcgattgttgtgcgtggaaagctgaagatcgatatgcggataactgcgacggaaatcatccagctgtgaaaataacaccccagtggcaaaggtccccaccacgccaattttcagcttctcctgcgcccgatggttagcaaagcaatccagcatgcctgcgatacgatcgaaggagtcattcagcaccggcagcagattctcgcctt

SEQ ID NO: 470 - DNA, 224 bp, Paracoccus denitrificans
accgagaccagcatgatgttcatcacgccgatgccgccgaccagcagcgcgatggcggcgatggcgccgatcagcagggtcatcgtgcgcgtggtctgctcgatggtctggcggatctcggcgctgttgctgaggaagaagtcctcggtgccgtggcgcagggtcagcaggcgggtgatcgcttcctgcgccgcatccatcggcgtgtcgtcgtccacgcgcac

SEQ ID NO: 471 - DNA, 224 bp, Burkholderia multivorans
ctcggctgcgcattgttcacgcgtttgccgcgcgggctcgatctgacggacgaaggccgcgcgctgctgcccgtgctcagcgatgcgttcagccgcatcgagacggtgctccagcagttcgacggcgggcggctgcggcaggtgctcacgctcggtgtcgtcggcacctttgccctaggctggctaatgccgagactgaagcgcttccgcgatacgcacccgtt

SEQ ID NO: 472 - DNA, 224 bp, Bacillus cereus
taatcgtattgatttgacattaattccacttttagaaaaggatgattatttttctaaaagtgatggtttagtaaaagttctgttagataaagatgaagtaaccaatccaaatattactccaagtgataggaagtattgggttaaaaaaccgtctgaaagagaatttgatgattgttgtaatgagttttggtgggtttcaacttatgtggttaagggattatgtc

SEQ ID NO: 473 - DNA, 224 bp, Cucumis sativus
gccgtccgaagccgcgattcgcgacaaggtgcacgagctgctgaagctcgtgcagctggactggctcgcgcagcgctatccgtccgagctgtcgggcggccagcgccagcggatcgcgctcgcgcgcgccctcgcggtggagccgaaggtgctgctgctcgacgagccgttcggcgcgctcgacgcgaaggtgcgcaaggagctgcgcggctggctgcggcgcc

SEQ ID NO: 474 - DNA, 224 bp, Pseudomonas entomophila
gaggaccatggcgaggcggtgctgaaccaggtcatcgagacctgcgacctggaacgcagcgagcccctggcggcgctgcagcggctggtcaaggagcacctcgcccatcgcgagatgctggtcttcctggtgttccagtaccgcccggactacctcgaccccgacggcgaaggcgcccgctggcagtcctacatcgccgcgctggataccttcttcctgcgcgg

SEQ ID NO: 475 - DNA, 224 bp, Bacillus megaterium
tgatcatcgggaaattttgttcaatcggtcctggagtcacttttattatgaatggagctaatcatcgcatggatggctccacttatccatttaatatctttgggcatgggtgggaaaagcatacacctacactagatatgctgcctttaaagggggatactattgttggtaatgacgtatggattggactagatgctacaattatgccaggcgtaaaaatagga

SEQ ID NO: 476 - DNA, 224 bp, Enterococcus sp.
aaaagagaaaaaacgatttgattgtttatttagctcatgatttacgtacgccattaactagcgtaattgggtatttaacgctgttaaaagaagaaccggaaatatcggttcaaactagagctaagtatacgaacatcgctttgagtaaagcttttcgccttgaagaattattgagtgaattttttgatgtgacgagatttaatttgactaacttaacaataaat

SEQ ID NO: 477 - DNA, 224 bp, Clostridium difficile
agagataaaaaacatacacagaaaacgattcgtgagcattatacagagcaatgggcggatgagatttatcgagtcatcattgaatatgatacaattcctatcggttacgcacaaatatatagaattcagggggaacttttcgacgaatataattaccatgagacggaagaaaagatttatgcgatggaccaatttatcggtgagccggaatattggaatatggg

SEQ ID NO: 478 - DNA, 224 bp, Salmonella enterica
catatgtcttggaatacatgaaaatgaaaaattgcttggatggattggccttaggcccatgtacaaattaacatgggaattacatcccttggtaataagtactcaatatcagaataaaggtattggaagacttttaataaatgaattagaaaaaaaagcaaagcaaattggaataattggaatagtattgggaactgacgatgaatactttaaaacttcattat

SEQ ID NO: 479 - DNA, 224 bp, Klebsiella pneumoniae
gatagaaccccaaatcacatatttacgcccaattttatcgccaacaggcccgccaattaccgtcccggcagccactgcgaacaggaaggcaaacagatgaagctgggcgttttgcaccgttaagccgaatttatgcatcagataaaaggtgtaatagctgctaatgctcgccatatagaaatatttcgagaaaatcagcatcagcagaatgcctaccgccagaa

SEQ ID NO: 480 - DNA, 224 bp, Sphingobium japonicum
gtggtgccacgcttggaggccaccagcgggggctcgcccaggtcgatgcggcgctgcacgttttccaccaccacgatcgcatcgtccaccaccaggccgatgcacagcaccagggccagcagggtcagcaggttgatcgagaaatcgaacgcatacagcgcgatgaacgccgcgaccaggcagaccggcacggtcaccgccgggatcaacgcggcgcggaaact

SEQ ID NO: 481 - DNA, 224 bp, Thauera sp.
ccaccggccaccaccagcggaatcgcgccgaccacggtggcgatcgaggtcatcaggatcgggcgcaggcgcaccatcgccgattcgatgatcgcctcgcgcacggttcttccgtcgtcgcgcagctggttggcgaattcgacgatgaggatgccgttcttcgctgccaggccgaccagcatgacgatgccgatctggctgaacaggttcaccgtgccgccgct

SEQ ID NO: 482 - DNA, 224 bp, Pseudomonas entomophila
agcgcttgccgacgctggaggccagcggtcgctacgagcgcgagcgcatgcgcggcgaaacccgcgaggccggccaggtcgaacagcgctatcgggtcgcagccggcatcagcgccttcgagctggacttcttcggccgggtgaaaaacctcggcgacgccgccctggccgactacctggccagcgaggaggcccagcgcagcgcgcggatcgccctgatcgcc

SEQ ID NO: 483 - DNA, 224 bp, Citrobacter braakii
gttgcgcttaaaatcgctcagcagcgggaaaagacagccgaccgcaaaagtccccactacgccaattttaagtttttcctgagtctgttttgtggcaaaacgatccagcatcccggccatacgatcgaaagagtcattaagtacaggtaacagactctctccttcagtcgtcagcattaaccctcgagagccacgcacaaaaagctgacaattcagctgttgct

SEQ ID NO: 484 - DNA, 224 bp, Yersinia pseudotuberculosis
tcgctggtgggcaccttcgccgccatgtacttccttcgcttctccatcaacaacctgacgctgatggcgctgaccatcgccaccggcttcgtggtggatgacgccatcgtggtgatcgagaacatctcgcgctacatcgagaaaggggaaaaaccgctcgacgccgcgctgaaaggggccggcgagatcggcttcaccaccatctcgctgaccttctcgctggt

SEQ ID NO: 485 - DNA, 224 bp, Proteus mirabilis
gaaattagtgagtgtttagctcaaggagctgattttcgaggggctaattttatgaatatgataactacaaggtcatggttttgtagtgcttatataaccaagacaaatcttagttacgctaatttttctagagtcatattagaaaagtgcgaactgtgggaaaatcgctggaatggcactgtgataactggcgccgtgtttcgtggctccgatctttcttgtgg

SEQ ID NO: 486 - DNA, 224 bp, Streptococcus parasanguinis
gaaagtgatgtcctcatttccattgatacttggaaaagtcaggtggcagaggctgctttggctgctggtgccaatctagtcaatgatatcactggtctcatgggtgatgagaaaatggctcatgtggttgctaatgctggtgcgaaagtagtcatcatgtttaatccagtcatggctcgacctcagcaccctagctcgctcatatttcctcattttggttttgg

SEQ ID NO: 487 - DNA, 224 bp, Pseudomonas aeruginosa
tctcactttgagcaaggtcgcaagctcaaacacatctactttgccgccctgatggaaccagccatctggggagcggagcttgcgatgtcgctgtcaagcctagaggggcgcggccacatctacatcgttgaaccgctcggcccatttgaggacgacccgaaccttacaaacaagaaattcccgggaaatccaaccaagtcctatcgcaccactgagccgctgcg

SEQ ID NO: 488 - DNA, 224 bp, Yersinia pseudotuberculosis
ttgtttttctaattcatctgcaatacgagtggcatttgttcttgatgagcctaacgccgcactcaattctgatggttgaatactacgattttccgttgtatctaaaatcataagtgccataaataatgtctcgttaatgccctgagactttaacatattattacgactttcaagaagtttgctctggacgtgcatagacaggcgagttaacaaaatttcttggt

SEQ ID NO: 489 - DNA, 224 bp, Photorhabdus asymbiotica
ggtcgtgcggtgttagcgaactttaccgcaggtagagcggtacaagggggaagtactttaacgcagcaattagtaaagaacttattcctaaccaatgaaagaacgctaaaaagaaagcttaatgaagcctatatggcgattattatggatgcgagctatagcaaagatcgtattttagagctgtatcttaatgaggtgttcttagggcaaagtggtgatgagca

SEQ ID NO: 490 - DNA, 224 bp, Pseudoxanthomonas suwonensis
tgtactcaccggtgctcgatgccgcggtagcgatcggcaggaagccggccgcggtgatcagcgtaccggtcagcatcgggaacgcggtcgaatcccaggcgaaactggccgcgcgcaggcggtcgtaaccctgctccatcttggtggccatcatctccaccgcgatgatcgcatcgtccaccagcaggcccagcgccagcaccagtgcacccagcgaaatcttg

SEQ ID NO: 491 - DNA, 224 bp, Salmonella enterica
tttggaaaaatactaattaatgaaatagaaaaaaaagcaagagaaagaaatttagaaggtattgtacttggaacagatgatgaaacatatagaacttcattatcaatgattgaattaaataatgaaaatattttgcaagaaataaagaatattagaaatttagaaaatcatccttatgaattttataaaaaatgtggatattgtattattggtgtaattccaaa

SEQ ID NO: 492 - DNA, 224 bp, Yersinia pseudotuberculosis
ctacgtacgccgcttaatgggattgtggggttaagtcgtattttattggataccgaattatcaccagaacagctcaactacttaaaaactatccatgtcagtgccataacgttaggtaatatttttaatgatgtgattgaaatggataaaattgagcgcagaaagatccagcttgataatcaacctatcgactttactgaatttatctctgatttagaaaacct

SEQ ID NO: 493 - DNA, 224 bp, Photorhabdus asymbiotica
agtcgaacaatggacaggcattattgccgtcagtcgtggtggattagttcctgccgcattacttgctcgtgaattgggtatccgccatgtggatactgtctgtatttctagctatgatcatgaccatcaacgtgacctcaaaatcttaaaaaaagcagaaggtgatggtgaaggttttatcgttgttgacgatttagttgatacaggtggcaccgcaaaagtta

SEQ ID NO: 494 - DNA, 224 bp, Staphylococcus saprophyticus
ttggtggcttatgggttgcgttaaacgaagaaaaagacattcctcgaaatgaagttcaatattcgtacacacatgtagcgtttactatagatgaaagtgaatttaatgattggtatcaatggttcaaggaaaatgacgtgaatatattagaagggcgtactagagatgtaagagataagcaatcaatttattttactgatcctgacggacacaagttagagtta

SEQ ID NO: 495 - DNA, 224 bp, Enterococcus faecalis
tatcttagaatatataggaattgacttttttgcagttgctactatagatgaagctattgctttaagaaaaaatggcattacaagtaatattttaattttaggatacactacaccaaccaaggtagatgatcttatccattacgaacttacccaaacaatagtaagcaaagaacacgcgtattttcttaataaaacaggaaagaagataatgtgtcatttaaaag

SEQ ID NO: 496 - DNA, 224 bp, Clostridium tetani
accttacagcagaggaaaatttgttagtacatacaaaaatattgggaatacctaaagataaaatttatgaagtgttggaaatagtaaatttgaaaaatacaggtaaaaagagagcttcacaattttctatgggtatgaaacaaaggttaggaatagctattgcacttttgagtgacccagagttgttaatactagatgaaccaaccaatggtcttgaccccttt

SEQ ID NO: 497 - DNA, 224 bp, Stenotrophomonas maltophilia
gatcggtcaccggcgcatgttcctcatcggcctcgtgctgttcggtgtcgcgtcgattgtggcggcctattcgcctacagcggaaatcctgatcggggcgcgggcctttctggcggtcggcgcggcggcgatgatgcccgcgacactcgccctcatccgcgtcaccttcgaggatgaccgggaacgcaacatcgccattgcgatctgggggtcgttgtcggtcg

SEQ ID NO: 498 - DNA, 224 bp, Staphylococcus epidermidis
accgtgaagaccgccagatactttgtatccgccacctccgaatttacctccagcgtgaagtacagttaagataacttcgacagcagggcgtcccatcttttcttgaatatcaacaggaataccacggccattgtcagtaactttaatccaattgtctttctcaattacaacttcaatatgactagcataacctgctaatgcctcgtcaatactattatcaacaa

SEQ ID NO: 499 - DNA, 224 bp, Pseudomonas aeruginosa
ctttcgcctggacacccgtctcactttgagcaaggccgtaggctcaaacacatctattttgccgcactgatggagccagccatctggggtgctgagcttgcaatgtcattgtcacgccaagaggggcgcggttacatttacattgttgaaccgctcgggccgtttgaggacgacccaaaccttacaaacaaaaaatttccgggcaatccaaccaagtcctaccg

SEQ ID NO: 500 - DNA, 224 bp, Enterococcus faecalis
tacaccacttagtatgggaaatcgttgacaactctatcgacgaagccttagcaggatttgctaaatcaattcaagtaattatcgaaccagacgatagtattactgttatcgatgatggtcgtggaattccagtcgggattcaagccaaaacaggtcgcccagctgtggaaacagtgtttacagtgctacatgccggcgggaaatttggcggtggcggctataaa

SEQ ID NO: 501 - DNA, 224 bp, Salmonella enterica
agaaaaaacatcaaaataagggatttggtaaaatactaatttttgaaacagaaaagaaagcgaaagaaagaaatttagaaggaattgtacttggaacagacgatgaaacatttagaactacattatcaatgtcagaattaaataatgaaaatatattccatgaaattaaaaatataaaaaatctaaaaaatcatccatttgaattttatgaaaaatgtggttac

SEQ ID NO: 502 - DNA, 224 bp, Enterococcus casseliflavus
gaattttctttaaaagatagagggaaacgatatactcatgattatgttgcctatcctgggtgtagtgagcatcatactggacttgcattagatattggtcttaaaaaaacagcacatgatatcatagcaccaaaatttaatggagaagaggcaaaaaaatttttagagcatatgaaagattacggatttattttaaggtaccctccaaacaaaaaaaaggtaac

SEQ ID NO: 503 - DNA, 224 bp, Yersinia pseudotuberculosis
gtaaaccattacggttagtttgacggatattattgtaggtaaactctttttctttataaatggcatattcttctggaacatcacgaattaatgcggcgccaatcaatgccatatctcgtgcactactgtattgcccaggtgcatctaaaccatgtacagtttgaaattgtgtgttttgtaaacctagacgcttaacatagtcattcatcatagtgacaaaggta

SEQ ID NO: 504 - DNA, 224 bp, Ruminococcus bromii
agatagttattatggaattactgaaaagatttcctgcttaaatagcttatatacttatatttttcaagtgactgactatacaagaaacggcattttcttcgcccccccattttttatacttggtgggtttatagctgataatcgcccacagattacatttggaaaaagtttcttgggatttgctattagcttagctctgatgcttggagaagctatgatattgc

SEQ ID NO: 505 - DNA, 224 bp, Providencia stuartii
agtgccgagccaggagatgtgcgcgataccaccacgccgctggccatggcccacaccctgcaggccatgactctgggcaaggggttaccacaggcccagcaggcgcaactggtgagctggcttaaaggcaataccacgggggcacagcgtattcaagctggcgtaccggcaggctggcaggtaggcgacaagactggcacaggtggttacggcaccaccaacga

SEQ ID NO: 506 - DNA, 224 bp, Kluyvera cryocrescens
gaaaaacatgtcggtaggtcgatgtcgttgtctgagctcagcgccgcggccttgcagtacagcgataacgtggcgatgaataagcttatcgctcaactgggtggcccgcagggggttaccgcgtttgcccgtaagattggggatgagacgtttcgtctcgatcgcacggaaccgacgctgaacactgcaattcccggcgatccacgcgataccacatcaccacg

SEQ ID NO: 507 - DNA, 224 bp, Ruminococcus gauvreauii
gcgttactacccaaatatcaaaaggaacgagatgatcacgaaagggtatgtggcttctcagtccagccacagtcgcgggagcgcgattgatctcacgatttttcgtttggacacgggtatgcttgtgccaatgggcggagatttcgactttatggatgtacggtcgcatcatgccgccagtggtctgagcgaagaggaggccggaaaccgtgagcgcctgcgtg

SEQ ID NO: 508 - DNA, 224 bp, Granulibacter bethesdensis
tgccggccagcggcagcaggccaggcttgccgtgcagccggcgcaatgacgagaagggattaaggcgaattttctcggcggcgcgggggcgggatttacgcgattcgggcagcaggaaaaacgccatcaccaggttgagggcattcatcatcgccgccgccaggaagggcgcatgcagatgccattcgcccagcgatccgccgagcagcgggccgacgataaag

SEQ ID NO: 509 - DNA, 224 bp, Citrobacter freundii
caaacagtggcagggtattcgcatgctcgatctggcgacctataccgccggtggtctgcccctgcaggtaccggacgaggttacggatagcgcctctctgctgcgcttctatcaaaactggcagccacagtggaagccgagcaccacgcgcctttatgcgaatgccagcatcggtctttttggtgcactggcggttaaaccgtccggcatgagctatgcgcagg

SEQ ID NO: 510 - DNA, 224 bp, Stenotrophomonas maltophilia
ggcgcgctggtcgctgccggccaggacgcggcgctggcgaccattcaacggttggatccggtgtacctggatgtgacccagtccagcacgcagatgctggcactgcgcaagcagctcgatgccggcctggtgaaggccatcgatggcaaggcacaggtgaaggtgctgctggaggacggcagcacgtacgcgcatgaagggaccctggagttcgtcggcagtgc

SEQ ID NO: 511 - DNA, 224 bp, Enterococcus faecalis
gttggcacagcaagaaagaatgtaaattctgccgccgctacccgtgatactcctatcaataatgctccgattattgtagctcccgagcgtgatgtgccgggaatgagggatagcacttgaaaaacaccaatcatcaacgctgttttgtagctgatgtctgccagtgtacttgtagtggcggtacgctttttgttccgattttcaatcacaataaacaatacacc

SEQ ID NO: 512 - DNA, 224 bp, Enterococcus faecalis
ctttagtgaaattaaatataccaacacttatatttgtgaataaaatagatcgaaagggagtatgtttggatgagatatacactcaaatacaggagaaattaacttctaatcttgcaataatgcaatcagttaaaataaaagataaaggtgattttgaattgacaaatgtaagggatgataaagtaattcaaagtcaaataatagagaagttactggatataaat

SEQ ID NO: 513 - DNA, 224 bp, Streptococcus pyogenes
acaaaatagatgctagattttgtgaattgaaaacaatatataaagaacgacctaagagaaactctaaagcagtaatccatatagaggttccaccaaatccttattgggcatctattggactgtcaatagaaccactaccaatagggtcaggattattatataagacagaggtgtcctatggatatttaaataattcatttcaaaatgcagtaaaagatgctgta

SEQ ID NO: 514 - DNA, 224 bp, Enterococcus faecalis
gagtattattggatatatctcactacttcaaaatgaacaaacctacttggaattagattctacaaaaaggaaaaattatatagacatcatttcagacaaagctaacagattagagcatctgctcaatgatttttttgaaattgcaaaaacaggcaaaagtagagaagaggtgtataaagaagaagtagatttaagcttgatgttaagccaaattagttctgaat

SEQ ID NO: 515 - DNA, 224 bp, Citrobacter rodentium
ggaaggcgctctggtgcaaaacggtcaggcgaccgcactggcgaccgttcagcagttggatcccatctatgtagacgtgacgcagtcaagcaacgacttcctgcgcctgaaacaagagctggccaacggcacgctgaagcaagagaacggtaaagcgaaagtcgaactggtgaccaatgacggcattaaatacccgcagtccggtacgctggaattttttgatg

SEQ ID NO: 516 - DNA, 224 bp, Bordetella bronchiseptica
acggcaaaccagagcgcggcggtcgcggcggtccagcagatcgaccccatctacgtcgacgtgacgcagtccagcgtgcagctgctgcgcctgaaaagcgccctggacagcggtttgctgcagcgcgaggctggcgagcagtcggcgcgcgtgacgctgacgctggaagacggcacgcagtacgaccaggacggcaagctgcaattctcggaggtcaccgtcga

SEQ ID NO: 517 - DNA, 224 bp, Shigella dysenteriae
ggtcggtggctttgatttcaatcagagcaagtttaaccgcgccacccaggcactgcgtcaggtgggttccaacatcaaaccgttcctctacaccgcggcgatggataaaggtctgacgctggcaagtatgttgaacgatgtgccaatttctcgctgggacgcaggtgccggttctgactggcagccgaaaaactcgccgccacaatatgctggtccaattcgct

SEQ ID NO: 518 - DNA, 224 bp, Klebsiella variicola
aggccgagccaggccaccaatcccatggcggcgataatgccgaacagcgctttccagtgaatgaaatgcatcagcgccgcgccggagagcgggccgacgatcggcgccaccagcacgacggaggtgatcactgccatcagcctgatcgatcttttttcctcaaaggcctcctgtacggtgacgtagccgacggtggcgataaagcagatgctggtgccctgcac

SEQ ID NO: 519 - DNA, 224 bp, Shigella sonnei
cgttgaagtacccgccgtctatgctggtcttgagcggaaacagcggctgcttcgcgcccaggagttgctgcaacgactggggctggaagaccgtacagagtattatccggcacagctttcgggtggtcagcaacagcgcgtcagcatcgcgcgggcattgatgaacggcggtcaggtaattcttgccgatgaaccgaccggcgcactggacagccattctggcg

SEQ ID NO: 520 - DNA, 224 bp, Shigella flexneri
cgtttactttcactgcgccacggtaagaaggatttctttacctggaacatggacggcgtcttgaaaaccgttgaaaagaccacacgtactttacaactgtttctgacgctggtggcggtgatttcgctggtggtgggcggtattggtgtaatgaatattatgctggtgtcagtgaccgagcggacgcgggaaattggcattcgcatggctgtaggtgcgcgagc

SEQ ID NO: 521 - DNA, 224 bp, Pseudomonas entomophila
cgacgctgacgctgccgatcttcgacggcgggcgcaaccgcgccaacctgagcctggccgaggcgcgcaaggattccgcgctggcggcctacgaaggcgccatccagggcgccttccgcgaagtcgccgacgccctggcggccagcgataccctgcgccgcgaggagaaggccctgcgcgccctggccgacaccagcaacgaagcgctgaagctggccagggca

SEQ ID NO: 522 - DNA, 224 bp, Pseudomonas aeruginosa
aggacaagatgcgcctcgacgaccgcgccagccggcactggcccgccctgcagggcagccgcttcgacggtatcagcctgctcgacctcggcacctacagcgctggcggcctgccgctacagttccccgatgcggtgcagaaggatccggcgcagatccgcgactactaccgccagtggcaaccgacctacgccccgggcagccaccgccagtactccaacccg

SEQ ID NO: 523 - DNA, 224 bp, Escherichia coli
cgttgatgcgcgagatgacccatgcgggtgaactggaacacctgacgcctgaacgggtatggaaagagacggaaagcgccctcaccacccgcaatccacaggtgttcttccaggtactgcgcgattgcggcgcactgcgcgttttattcccggaaattgacgcactgtttggcgttccggcccctgccaagtggcatccggaaatcgatacgggtattcatacc

SEQ ID NO: 524 - DNA, 224 bp, Shigella sonnei
atcgaccagcgcgggtgcttgtttataaagatcataacccgccatattcattaaccagttttgaacaattccgctgaaggcaccatcaataataatcatcacaacatctaaatcgaggttatttgctacacaaccttgttgctgacacgcctgcaatacttcgcggagagtctgcggattaaagcccatcttttcgcgtatcactccctcggccagcatctcat

SEQ ID NO: 525 - DNA, 224 bp, Shigella boydii
tccgcctgaagcaaaactgcgtctatgcgcgcttcggcgctgcctgtcagacgcagtattagcgttatcccgacggccagcagcagaggcgcaacgaacatcagcaaccagaaacgcagaaaagcggtccggcgttgctgtcgccagggcaaaaaattaattggcgggttcatcagtacgccttcccaagcgccagccccagcgcgatggcaaagtctccacct

SEQ ID NO: 526 - DNA, 224 bp, Escherichia coli
caaaaaaccccgccggagcgaggtttcgtcagtcgcctgcggctggtaaccgcaaagcacactgtattatgtcaacactgaaagtatacgtgttccgcgcagaacgcgcaatttcggcacgaattttgacgtatttagtgcatagttgagtatcgatcacagtttgcgttttgtccaaatattactgtttatttatacagtaaacttctataatatcactgtac

SEQ ID NO: 527 - DNA, 224 bp, Shigella flexneri
cattatcagcggtattaccgttgccttcgttctctatatcctcacttatgcctatatctctgctaatggtgcgatcattagtgaaacgatatcaatgaatttgggctatcacgctaatccacgtattgtcgggatctgcacagccattttcgttgccagcgtattgtggataagttcgttagccgccagtcgcattacctcattgttcctcgggctgaagatta

SEQ ID NO: 528 - DNA, 224 bp, Shigella sonnei
gtccgctaacccgatgcgcctggcgagtctggcgttaatcgtattggggattattggtctgaaactcagcactcaataactaccgggctgctgtacccaaataaacttactgacatcaaacccttcccgggtcgcgactgccagcatctcctgtttcacttcgtcagaaatggttggcgtgcgggagagtatccacaggtagtcgcggtccgggccgcaaacca

SEQ ID NO: 529 - DNA, 224 bp, Vibrio anguillarum
gcgctttactggcatttccagaacaaacgagcgctgcttgatgcgctgcccgaggcgatgctgcgggaacgccatacccgctcgctacccgaagagaatgaggactggcgggtgttcctgaaagagaatgccctgagcttcagaacggcgttgctctcttatcgggacggcgcgcgtatccatgccggcactcgaccgacagaaccgaattttggcaccgccga

SEQ ID NO: 530 - DNA, 224 bp, Enterococcus sp.
tgctgataagattatggggctgacgattggagcggatgattatattacaaagccatttaatccactagaggttgctgcaagggtcaagacacagcttcgccgttatgtatgttacaataatgccgcagatatagaaaaagaaaatgtattggttacggaatatgatattaacggacttgtcattaataagaatactcataaatgcacactgtatggaaaggcag

SEQ ID NO: 531 - DNA, 224 bp, Xanthomonas maltophilia
ctggtgcagaccgccgagggcgcagtgctgctggatggcggcatgccacagatggccggtcacctgctggacaacatgaaggcacgcggggtggcaccgcaggatctgcggctgatcctgctcagtcatgcgcatgccgatcacgccgggccggtggcggaactgaagcgccgtaccggcgcgaaagtggtggccaacgccgaatcgcccgtgctgctggcgcg

SEQ ID NO: 532 - DNA, 224 bp, Klebsiella pneumoniae
aactattggcgggccggagggattaactcattttctgcgtagcactggtgatagttatacaaggcttgatcgacacgaacccagccttaatgaggcgaagcctggcgatgagcgtgataccaccactccggcagcgatggctcaaacgctacaaaaattgttaaacgaaagtgtacttacagaaaaatctcgaaaaaaattaataagctggatgcaggaagata

SEQ ID NO: 533 - DNA, 224 bp, Enterobacter asburiae
tctgggtggcgatgcaattgctcgcggtgaaatttcgctgggcgatccggtgaccaaatactggcctgagctgaccggcaaacagtggcagggcgttcgcatgctggacctggcaacctatactgccggtggcctgccgttacaggtgcccgatgaggttaccgataatgcctcgctgctgcgtttttaccagtcctggcaaccacagtgggcgccaggcacca

SEQ ID NO: 534 - DNA, 224 bp, Enterobacter dissolvens
ccagcatcggcctgtttggctcactggccgttaaaccgtccggcatgcgcttcgagcaggccatggcggagcgggtctttaagcccctgaaactcaaccatacttggataaacgttccacacgctgaagagcccaactacgcatggggttatcgtgagggaaaagcggtccacgtttcgcctggtatgctggatgcagaagcctatggtgtgaaatctaacgtc

SEQ ID NO: 535 - DNA, 224 bp, Shigella flexneri
ggaaggggctctggtgcaaaacggtcagaccactgcgctggcgaccgtgcagcagctcgatccgatctatgttgacgtcacgcagtccagcaatgatttcctgcgcctgaaacaggagttggctaacggcaccctgaaacaggaaaacggcaaagccaaagtggagctgattaccaacgacggtatcaagttcccgcaggaagggacgctggaattctctgacg

SEQ ID NO: 536 - DNA, 224 bp, Pseudomonas aeruginosa
tactgtgtggctctggcttcatcgcaatcgctgcgttacaggggcaagaggtcatcggtgggctcgccgcgtatgtgctcccaaagtttgaacaacagcgcaaagaaatctatatctacgacttaggcgtgcagggagcctatcgccgacgaggcatcgccacagccttgatcaatgaactccagcgtatcgcacatgatattggcgcttatgtaatttttgtc

SEQ ID NO: 537 - DNA, 224 bp, Stenotrophomonas maltophilia
agggagttaaattgaaaatatcattgatttctgcagtgtcagaaaatggcgtaatcggtagtggtcctgatatcccgtggtcagtaaaaggtgagcaactactctttaaagcgctcacatataatcaatggctccttgtcggaaaaaaaacatttgaatctatgggtgttcttccaaatcgcaaatatgtagtaatgtcaaaaaacggaatttcaagctcaaat

SEQ ID NO: 538 - DNA, 224 bp, Unknown
  Description of Unknown Mixed culture polynucleotide
tcagcgactgacctgctacgtttctatcaaaactggcagcctgaatgggctccaggaacacaacgtttatacgcgaactccagtattggtttgtttggcgcactggctgtgaagccgtctggtttgagctttgaacaggcgatgcaaactcgtgttttccagccactcaaactcacccatacgtggattaacgtgccgcccgcagaagaaaagaattacgcctg

SEQ ID NO: 539 - DNA, 224 bp, Acinetobacter berezinae
tccatccccacatatgcatctaaattgacaaatgaattgctaaataaaaatggcaaaactcaagccaagcactcttttgataaagagagcttttggttggtcaaaaataaaattgaaattttttatccaggcccaggacacactcaagataacgaagttgtctggatacctaataaaaaaatcctattcgggggctgttttataaaaccgaatggccttggcaa

SEQ ID NO: 540 - DNA, 224 bp, Streptococcus pneumoniae
agtttcctttggttcgaagggtaatgggattacctatgccaatatgatgtctatcaaaaaagagttggaaactgcagaggtcaaggggattgattttacaaccagtcccaatcgtagttacccaaatggacaatttgcttctagttttatcggactagctcagctacatgaaaatgaggatggtagcaagagtttgctgggaacttctgggatggagagttcct

SEQ ID NO: 541 - DNA, 224 bp, Citrobacter rodentium
aagctgctcggcacgcagtacatcagccaacaggattatgattctgccttggccgatgcacagcaagccaacgcagccgttgtggcggcgaaagcggcggtagaaaccgcgcgtattaacctggcctacaccaaagtgacctcccctatcagcggtcgcattggtaaatctgccgtcaccgaaggtgcactggtgcagaacggtcaaaccaccgcgctggccac

SEQ ID NO: 542 - DNA, 224 bp, Serratia rubidaea
gtggcctgaagccatacagtgacattgatttgctggttactgtgaccgcacggctcgatgagagtgtgcggcaagctctgttcgtcgatctcttgggggtttccgttttccctggtcaaagcagagttctccgcgccttggaagttaccattgtcgtgcacagtgacatcgttccttggcgctatccggccagacgggaactgcaattcggggagtggcagcgc

SEQ ID NO: 543 - DNA, 224 bp, Paenibacillus apiarius
ggactccgcaattgacttggcaagacaatatgacagcaaaatcctaattgagcagggtgttttaggttatgaggtcggttgtgccgtattgggaaacagtttcgacttgattgttggtgaagtggatcaaatcagactgcaacacggtatttttcgtattcatcaggaagccgagccggaaaaaggttctgaaaacgcaactataaccgttcccgcagaactat

SEQ ID NO: 544 - DNA, 224 bp, Coprococcus catus
gctataatcagcccataaacaaacaggctcttgaaaacattgaatacgatattagaggtctgtcaataaacaaaaacaaccataaatgcagtttgtttgggaaagaattgcaactgactccaatagagttttctattctgtggtacctttgtgaccatcaaggaaaagtagtatcttcagaagaattgtttgaggcagtttggaaagaaaaatacctcgataac

SEQ ID NO: 545 - DNA, 224 bp, Enterococcus faecalis
ggaatacctgttgttgggtgtgatacactctcgtctgctctttgtatggacaaagataaggcacataaacttgttagccttgcgggtatctctgttccaaaatcagtaacttttaaattctctggcaaaaaggcagccttgaaaaaaattgaaaaggagttaagctatccattatttgtgaagcctgtccgtgcaggctcgtcatttggaataacaaaagtaac

SEQ ID NO: 546 - DNA, 224 bp, Unknown
  Description of Unknown Polynucleotide from a pathogenic organism
gtcgcattttctacatgtctatatctagttggacttggtggagacagctggtacgggataaccaatcaagttccattgctaaataaactgtatacatttatttttagttggtcggactatacgcgttcaggtgttttctttacgcctgtgtttctttgcctaggaatatttgcctatagagtatctaaaaagttaactgcatcaaagatattaaacttgctttt

SEQ ID NO: 547 - DNA, 224 bp, Enterococcus casseliflavus
gctatggtattttaaattacccagaattaagctttgattttgtgagaataggaattctctgttatggatttttaagtgactataatagtccgactaaaatccctatagatttacaacctatagtaaaagtaaaagcctctttgattacagaaagaattgtggaggcaggtgaatatgttggctatggattaggcgctaaagttgaaaaaagaacaagaataggt

SEQ ID NO: 548 - DNA, 224 bp, Unknown
  Description of Unknown Polynucleotide from a pathogenic organism
cttttgaagtgttggctagagtgaaaacacagctacgtagatatcaaacgtataattctcaaagtatagatgaaacaaatgaatatgcaaaaaatggattaaatatatctgtcaacagtcgaaaagtattcttatatgatgaagaaattaaattaacgcctattgaatttgatatcttgtggtatttgtgtagaaatgaaggtcgcgtaatatcgtcagaagag

SEQ ID NO: 549 - DNA, 224 bp, Artificial Sequence
  Description of Artificial Sequence Synthetic promoter reporter polynucleotide
cgtctattgaattagacagtcatctattcaacttatcgtcagaaaaattaaaactgaatactcgtgtcactttaattcaccaagatattctacagtttcaattccctaacaaacagaggtgtaccttggatattcaccgaacactagggttgctcttgcacactcaagtctcgattcagcaattgcttaagctgccagcggaatgctttcatcctaaaccaaaa

SEQ ID NO: 550 - DNA, 224 bp, Clostridium botulinum
aggggtgataaacttgtaattggcaagttttgtgcaatagctgaaggtatagaatttattatgaatggagcaaaccatagaatgaattcaataacaacttatccttttaatataatgggaaatggttgggaaaaagcaactccatctcttgaagatttaccatttaagggagatactgttgttggaaatgatgtgtggattggtcagaatgttactgttatgcc

SEQ ID NO: 551 - DNA, 224 bp, Streptococcus pneumoniae
aaaggagaaatcaggctctgccttaggtactttgtctacaggcgtagttgcaggtactctaactggtccctttattggtggctttatcaaggaagattttcaaccagtagccaaggaaaaggctattccaacaaaggaattatttacctcggttaaatatccctatcttttgctcaatctctttttaaccagttttgtcatccaattttcagctcaatcgattg

SEQ ID NO: 552 - DNA, 224 bp, Pseudomonas aeruginosa
ttgggaggcacgatacgaggacctggtcgacgacgcgggccgcgtgcctccggaatggcgcgaacatgtcccacccttcgacccgcagcgctcgcgtgcgatccgcgacaatggtgtgctgccggaattcctgcggaccacgcccggcacgctccgcagcggcaaccccggcgcctcgctcgtcgcgctcggggcgaaggcggagtggttcactgccgaccacc

SEQ ID NO: 553 - DNA, 224 bp, Pseudomonas aeruginosa
cctcgatggtcgggctcggcgccagagcggaatggttcaccgcagaccatcccctcgactacggctatggcgagggttcgccgctggccaggctggtcgaagccggcggcaaggtgctgatgctcggggcgccgctcgacacgctgaccctgctgcaccatgccgagcatctggccgacatccccggcaagcgcatccggcggatcgaggtgccgctggcgacg

SEQ ID NO: 554 - DNA, 224 bp, Unknown
  Description of Unknown Polynucleotide from a pathogenic organism
gccgccgaaccagaggtggaagacacggtagaagcgctcgggaagatccggaatgcgatggttcggggtgacgaccttgcggtctatcaagctgcgcaggtcgtggggaaattgattccgacactcctggttcccatcaatccgcccacgtacgcacggttcgcacgcgaggcgatcgacaggattctcgccttcccgaatgtacccgaagggttcgcggccga

SEQ ID NO: 555 - DNA, 224 bp, Paracoccus denitrificans
aggcgcccgacgcacggcgcagcgcgcatcccgacgcctccatggtcgcggtcggcccccttgccgccacgctgacagagccgcaccggcttgggcaggcgctgggcgaaggctcgccgctggagcgcttcgtcgggcatggcggaaaggtcctgcttctgggagcgccgctcgactccgtcaccgtgctgcattacgccgaggccatcgcccccatcccgaac

SEQ ID NO: 556 - DNA, 224 bp, Staphylococcus aureus
gaaagcaattggagcaagctataaagctaaaagagaataaggcgcaaggaatgattaagcccccttcaaaaacaatgggaacatctgaatctagaatatggaaaatgcaacatgctactaaacaaaaaaagatgcatagaaatacgaaatcgttggaaacacgaatagataaattaaatcatgtagaaaaaataaaagagcttccttctattaaaatggattta

SEQ ID NO: 557 - DNA, 224 bp, Salmonella typhimurium
tctgccaagttcgttcctgccagatgaagatcagggggtattcctgtctatggctcagctgccagcgggtgcaacgcaagagcgtacccagaaggtgctggacgaaatgacggattactatctgaccaaagaaaaagcgaacgttgaatcggtgtttgccgttaacggcttcggttttgcaggtcgtggtcagaacaccggtatcgcattcgtctctttgaaag

SEQ ID NO: 558 - DNA, 224 bp, Streptomyces fradiae
tcacgcgctggattatggctacggagtcgagtcgccgctggctaaactggtggcaatagaaggatacgtgctgatgcttggcgcgccgctggataccatcacactgctgcaccacgcggaatatctggccaagatgcgccacaagaacgtggtccgctacccgtgcccgattctgcgggacgggcgcaaagtgtgggtgaccgttgaggactatgacaccggtg

SEQ ID NO: 559 - DNA, 224 bp, Vibrio cholerae
cgccacccgcagccgggacagccgcgtcttctaccggcgcaaccttctcgccatcctgcgggagcgcgaggtagccggcgtcggatcggatatggctttgagtaagggcctgccgttccgcgccgccacggacggcgagagcgtcagcggcaagtttaccggaaccgtgcatctatcgagcggcaagttcgccgtggtcgagaaatcccatgagttcacccttg

SEQ ID NO: 560 - DNA, 224 bp, Streptococcus pneumoniae
aatatgacattgaaacccaaaggtaaacaaaatgtactagagtatctctatgattttgtaaatggatttacagaacctaaccttggttcccactacataaaagattactcactcttttacttatgtttatttctttttatggtcatcgctaataatcttggtttgatggctaaacttcaaacaacagatgggacaaacctttggacatcgccaactgcaaatct

SEQ ID NO: 561 - DNA, 224 bp, Enterococcus faecalis
acaccattttctctacgacaaatttttcttacgaaattttagcagaacgtttgagagaatcagctttcttattaaaaggcgtcaaaattactttaacagatgaacgtggggaagaacccaaagaagaagtcttccactacgaagaaggtattaaagaatttgttgcctatctaaatgaagaaaaagatacattaacacccgttgtttatttttcaggagccaaa

SEQ ID NO: 562 - DNA, 224 bp, Enterococcus faecalis
aagacatcttgaaaaacgaagaaatcaatactatgatctatacgattggcgcaggcgttggtcctgaattttcaattgaagattgtaattatgataaaatcatcatcatgaccgatgcggataccgatggtgcccatattcaagtgttgctattaacgttcttctatcgttatatgaaaccattaattgaagccggcaaagtctacattgccttaccaccactt

SEQ ID NO: 563 - DNA, 224 bp, Burkholderia glumae
gcgctggataccgccgaagcggtgcgtgccaccgtggcgaggctcgaaggcaacttcccggccggcctgagcgcgcgcatcgcctacgacccgacggtgttcgtccgcgcttcgttgcagaccgtggccaccaccctgctggaggcgatcctgctggtcgtggtggtggtggtgctgttcctgcgtagctggcgtgcttcgctgattccgctgatggccgtgcc

SEQ ID NO: 564 - DNA, 224 bp, Caulobacter sp.
cggcctttctcagtggcctgcagggcgagttctatcgtcagttcgccctgaccatcgccatctccaccatcctgtcggcgctcaactcgctgaccctcagcccggccctggcgggcctgctgttgcgtccgcggccggccggaggtgcggtggcggggcgcttccagcgactgctccaggtcctcggccgaccgttgcgaaatgcccccgaggcctacggcaac

SEQ ID NO: 565 - DNA, 224 bp, Clostridium difficile
ctttggaagttgttgcaagagtaaagacacaattaagaagatatatgcgatataataattcttatgaacaacagtcgataattgttaatgagtatgatattaaaggattgattatcaataaagaaacacataaatgtagtttatttgggaaagaagtagcattaacaccgattgaattttctattctttggtatttatgtgaacatcaaggaaaagtggttcct

SEQ ID NO: 566 - DNA, 224 bp, Clostridium difficile
atgatgtgaggtcaattttaaaggcatttaaattaaaatacttagatatatgtggcatatttacacacttgtgtgtttcagatagtctgaaagatgaagatgtagactttacaaataaacaaattgaatgtttttataagttaattgattttctgttaaaaaaaggtataaaaattcctaaagtacatattcaaagtagttatggtttttttaattatcccaat

SEQ ID NO: 567 - DNA, 224 bp, Serratia marcescens
agctggcctcgttgaataccgatcgcttcagtacccagcgcccggaagcggtgaacaacctgctgaaagaagcggaagcgcgcaacctgagcctgctgtccgcccgtctgagccaggatctggcgcgtgagcaaatccgcgcagcccaaactggctatatgccgacgattgacgtcagcgcgtcgaccggcatcagcaataccaaatacaacggctccaacacc

SEQ ID NO: 568 - DNA, 224 bp, Klebsiella pneumoniae
gaataaaggcaaactcgccaatctgcgccaggctggcggcgatggtcagcgcggtgcgcggcgagtgaccaaacatccgcacaaggaaaaaggcggctgcggatttaccaaagataataatcgccagcgtcgccagcaccgccagcggctgttggaccagcaccatcgggtcaaacagcatgccgacggagacgaagaacagcaccgcgaaggcgtcgcgcagc

SEQ ID NO: 569 - DNA, 224 bp, Klebsiella pneumoniae
aggtcttccacaatcagccagccgatggcgatttgcccgcgctggctgtcaatcagttgtcgttcctccagcgcgcgcagcagcactacggtactggcagtggagaggcacaggccaaagacgatgccggtcatcagcgaccagccgagggcggcggagagcgccatacccagcagcgtcgccacggctatctgggcgatagccccggggatggcgatcgattt

SEQ ID NO: 570 - DNA, 224 bp, Marinomonas sp.
gacgttgataaattgataattggaagcttttgctccatagggacgggtgcatcttttatcatggctggaaatcaaggtcaccgatatgattgggttacatcattcccttttttctatatgaatgaggaaccggcattttcggaatcagttgatgctttccaggcggcaggtaataccgtcataggaagcgacgtgaggattggctctgaagcaatgattatgcc

SEQ ID NO: 571 - DNA, 224 bp, Bordetella parapertussis
ggcgctgacgcgccagacgctggcggcccagcaggagtcgtacaagctgacccgccagagctacgacctgggcgtggcgaccgaactggacctgagccaggccgagatttcgctgcgcaccgccgagcgcaatctgtcgcagtacacgcgcatggcggcgcaggaccgcaacgcgctggtgctgctggtgggccagccgctgccggccgggatcggcgcgcagc

SEQ ID NO: 572 - DNA, 224 bp, Bordetella parapertussis
tccaggtcgacggcgatgatgccggccttgcgcgggttcgagaagtcggacagcggggtcttcttgaccgtgccgcgcgacgtcgccatgaagacgtagtggtcttcgctgaattccttgaccggcagcaccacggtcaccttctcgccgtcggccagcgggaacatgttgacgatgggcttgccgcgcgagttgcgcgtgccctgcggcacctcccacacctt

SEQ ID NO: 573 - DNA, 224 bp, Mycobacterium bovis
cggcggttgtgcaggccgatccgcgagcaggcgaacgcggtcagcgcgcccagcgccccggccagcccggcgaaggcgccgaactggaccgcccacttggtcggcgtgaacgtcaacagcagcaggccgacggcggtggtgccgatcagccgccaggccgggccgctggccaccccggggacatggccgcggcgcagcaggatgaccagcatgccgaacaggca

SEQ ID NO: 574 - DNA, 224 bp, Klebsiella variicola
cggcgtgtcgcaggcggtacgcaacgaagcgaagagccgcgacctgacggtattcgatgccacctgtccgctggtgaccaaagtccatatggaagtcgcccgcgccagccgccgcggcgaagagtcgattctgattggccatgccggccatccggaagtcgaaggcaccatgggacagtacaacaacccgcaaggggggatgtacctggtcgagtcgccggaag

SEQ ID NO: 575 - DNA, 224 bp, Pseudomonas entomophila
tcgacatggccgccttcggcgtgccggggctgagcttcatgacccgctatggcaagggctgggacgccgattattcgaatgccaacagcgtctacatgcgcaccgacgccaacggcaacccgctgaccaaccagggccgctgggaacgcgacgtggaggtcaagtacgtggtgcagggtggcgcggccaaggacctggcgttccgcgtgcgccaggccaccgtg

SEQ ID NO: 576 - DNA, 224 bp, Artificial Sequence
  Description of Artificial Sequence Synthetic construct polynucleotide
gccttccacctcggggtgcccttcatgcccgatcagcacgcattcgtggccgtcgcggctgtagcgcaccacttccatgtgcactttggtcaccagcgggcaggtcgcgtcgaaaaccttcaggccgcgcccctcggcttccttgcggaccgcctgggaaacgccgtgggcgctgaagatgacgatgacgttgtccggcacctgatcgagttcctcgacgaaga

SEQ ID NO: 577 - DNA, 224 bp, Salmonella enteritidis
ttccggctattaccatggccattcgtttgacgagtgcgctcgctttctcttgccagatcgcaatgacatcgaccaactgatcgttggtagcttctgttccatcggcacgggcgcctccttcatcatggccggaaatcaggggcaccgttatgactgggcgtcttcttttcccttcttctacatgaaagaggagccagcattctcgggcgcacttgatgcattcc

SEQ ID NO: 578 - DNA, 224 bp, Clostridium difficile
gacactacttcgagatgaaagtcagatttctgaagaactgcgaaaaaaatatttatctatttcgttggataaggcagagcgtcttgaagatttaattaatgaattctttgaaattacaaggtttaatttatcaaatatatcacttgaatacagtacagtaagcttaactcgtatgcttgaacaactaacatatgagtttaaacctactttgatgagtaagaatt

SEQ ID NO: 579 - DNA, 224 bp, Klebsiella variicola
taggggtaataacgacgcttgtagcctttgacttccacgccgggaaagcggtactggttgacggcaaagcgggccacctgcacttcgctgaggttgacctttaccggaatcgaggtaaaacgatgggagcgggcgcgctcctttttgaaaccggcgatgtcatcgtcggttaaatcgacgacgtcgcgtagcgcatccagcgtctgctgtacgttatcgacttt

SEQ ID NO: 580 - DNA, 224 bp, Staphylococcus aureus
gtaagatattaaatgttgaaaaagcacgattagatagaattttgaataacaatgaaattcgtcaaatgatcacagcatttggtacaggaatcggtggcgactttgatctagcgaaagcaagatatcacaaaatcgtcattatgactgatgccgatgtggatggagcgcatattagaacattgttattaacattcttctatcgatttatgagaccgttaattgaa

SEQ ID NO: 581 - DNA, 224 bp, Escherichia coli
gtcaagaaaaaatacggctccagtatttcctggggagacctgatggtcctgactggtaatgttgcccttgaatccatgggatttaaaacgctgggatttgctggcggaagagaagatgactgggagtcggacctggtatactgggggcctgacaacaagcctcttgcagataaccgggataaaaacgggaaacttcagaaacctcttgccgccacgcagatggg

SEQ ID NO: 582 - DNA, 224 bp, Bordetella pertussis
ccgcgaggccgaggagccgcgcgccgcgctgatggcgcgtttcgggctgaccgagcgccaggccgaggacatcctggaaatgcgcctgcgccagctggcgcgcctggaaggcatcaagatcgaacaggaactggccggcaagcgcgaagaacagctgcgcctgcaggacctgctggacaacccgggctcgctcaagcgcctgctcatcaaggaaatcgagggcg

SEQ ID NO: 583 - DNA, 224 bp, Bordetella parapertussis
gctggcggtggcccagccgctgctgccgctggaagtgcagcagcagggcatccgcgtcaccaaggccaccaagaacttcctgatcgtcgccggcttcgtgtccaccgacggcaccatggacaagtccgacctggccgactacgtggcctcgtacatccaggacccgatcagccgcacgcccggcgtgggcgacttccagctgttcggcgcgccatacgccatgc

SEQ ID NO: 584 - DNA, 224 bp, Bordetella parapertussis
gtatccgtacgacaccacgccgttcgtcagcctgtcgatcgagggcgtggtcaagacgctgttcgaagcggtgctgctggtcttcctggtgatgtacctgttcctgcagaacgtgcgcgccacgctcatccccacgctggccgtgccggtggtgctgctgggcacgttcggggtgctggcggcgttcggctattcgatcaacacgctgaccatgttcggcatgg

SEQ ID NO: 585 - DNA, 224 bp, Pseudomonas putida
cgatggtccgcggttcgacgcccttgccgccgtcgagcaccatcagcgccgagtccaccgcggtcagggtgcggtaggtgtcttcggagaagtcttcgtggccgggggtgtcgagcaggttgatcatgtgctcgcggtaggggaactgcatcaccgaggtggtgatggagatgccgcgctgcttttccatctccatccagtcggacgtggcgtggcggtcggac

SEQ ID NO: 586 - DNA, 224 bp, Pseudomonas aeruginosa
gcttggacaccgagtagatccagcgctgcttgttgatcggcccgaccaccaggttcgagcggtcttcgtcgaagtccgggtcctggcacatgccgtagacctcggaggtggaagggaacaccacgcgtttgccgtatttcacgcagtagcggacgatccgcaggttttcctcgaagtccagttcgaacacccgcagcgggttgcgcgtgtactcgatgggcgtg

SEQ ID NO: 587 - DNA, 224 bp, Mycobacterium vanbaalenii
tgaccgtgattccgccgatcttcggcatggtgctgttgaacggcaccccgtagctggacacataccaccagccgttggtggtggcgaagcacagcgccatcatgaacagcagcgcggccaagaacgccatccggttgcgcgaccagcgcagcaccgccggcgacaccagcacggtggtcagcgcggccatcgccgcccccaccgcggcgaacaggccgaaatgg

SEQ ID NO: 588 - DNA, 224 bp, Mycobacterium avium
ggtcaggtcggtgaacacgccgacgatctggggccgcaggttggggtcggggaatccgccgccgagtggtttgcccgccgggtcggtgagcccgacgaaggtggcgaaggtgccggcgcgggtcgaggcgatctcgatgcgggaacacccggctccggccacctgatcccgggatgcgctggcgatcaccacgttgcggtcggtgacgtcgacgcgcttgccgt

SEQ ID NO: 589 - DNA, 224 bp, Escherichia coli
ctagagtgcgccgggaacacattggctttatattccagcgctaccatttgatacctgatcttagcgcacttggtaacgtggagatcccggccatttacgccaacagcgaacgcgacagccgtcggcaacgtgcaacggcgttgctgggcagactgggactggaaggacgtgagcatcataaaccttgcgaactttcaggcggtcagcagcaacgcgtcagtatt

SEQ ID NO: 590 - DNA, 224 bp, Enterobacter cloacae
ggaggcatcaccacgcggcgtgcgggtcaccccttgctgcggtaccaggatagcctgcggatcgacgccttcgtctatgcgggcgcggacgaacatccctggcagcaggacatgctgcgggttagggaagatagcgcgtagcgtgatagagcccgtgctttcatcgacagtgacgtcagagaactgcagcgaccctttaagcggataggcctggccgttctcca

SEQ ID NO: 591 - DNA, 224 bp, Acinetobacter sp.
taaacacgtctacttttcagctttgatggagcctgccatttggggggcggaacttgcaatgtcgttgtcacgcctagatggccgtggctacatatacatcgtcgaaccaactggaccgtttgaggacgacccgaatcttacgaacaaaagatttcctggaaatccaacaaagtcctatcgcacgtgcgatccgctacgaattgtcgggtcagtcgaagactggc

SEQ ID NO: 592 - DNA, 224 bp, Pseudomonas aeruginosa
actggtgggacggccagggcgcggccagggtgctggcctacgaccaccaggcgctgctgatggagcgtgccaccggcgggcgctcgctgctggagatggtcaggcgcggcgacgacgacgaggctacgcgtatcctctgccaggccatcgaacgcatccatgccccgcgcccgggtccgttgccggagctgaccccgctggagcgctggttcgactcgctgtat

SEQ ID NO: 593 - DNA, 224 bp, Clostridium difficile
tgatattaacaatgaatcttttactgtttctatatttttatcttcaaatttaaacttatttgtataaaaactttcaagtaattctacctgttcaataggtgatatttttaatgaagattctagccaaaagtttgagataccacctgaaacatcataattgccatatccaatttgcttaagataagtttgtatgttttttagccctgtttttttatctagagtct

SEQ ID NO: 594 - DNA, 224 bp, Clostridium difficile
acctaaagaatattgattaacttttttatttttttgattttctaaccttacaatttttaatgcttcatttacattttttttaggtaaatctaagagtctttgcattatggtcatgttttcaagccctgtaagatgaccatagtatgatggagattctatcaaagaacctatatcttttaaaattgatattctatttttttcatttaactctttacctaaaatat

SEQ ID NO: 595 - DNA, 224 bp, Klebsiella variicola
cgataaggcccgccgtctgctgtggccggtgaaacagaaatatggccagaaaatctcctgggccgacctttacatgctggcgggtaacgtcgctctggaaaacgccggcttccgcacctttggctttggcgccggtcgcgaagacgtctgggaaccggatctcgacgtcgactggggcgatgagaaagagtggctggcccaccgtcatccggaaagcctggcga

SEQ ID NO: 596 - DNA, 224 bp, Klebsiella pneumoniae
gataaagcgcagcagctgacgctaaccgccccggaaatgaccgtgctggtcggtggcttgcgcgtactgggcgcgaactacgacggcagcaaacacggggtgtttaccgaccgcgtcggcgtgctcagcaatgacttcttcgtcaatctgcttgatatggctaccgtctggaaagcggccgatgataacgcggagctgtttaccggcagcgaccgtaaaaccgg

SEQ ID NO: 597 - DNA, 224 bp, Shigella sonnei
tgatccagttaggatcaattctggcggtagtagtgatgttctggcggcgtctgtttggcctgattggcatccactttggccgcccgttgcagcacgaaggagaaagcaaaggtcgtttaacgctgatccacattttgctggggatgattccggcggtggtactggggctgttgttccacgacacgattaagtcattgtttaacccgataaatgtgatgtatgcg

SEQ ID NO: 598 - DNA, 224 bp, Klebsiella variicola
caccgccttcgccattgcctcctgggcctgcggcgtctccaacagcctgacgatgctgattttcttccgcgtcattcaggggatcgtcgccggaccgctgatcccgctgtcgcaaagtctgctgctcagcaactatcccccggcgaagcgctctatcgctctggcgctgtggtcaatgacggtgattgtcgcgcccatctgcggtccgatcctcggcggctata

SEQ ID NO: 599 - DNA, 224 bp, Enterobacter sp.
cgctggtcagttcggtatccagcaaaattcggctcagcccgacgatcccgtttagcggggtacgcagctcatgactgatggtggaaataaaggtggttttatcccggctggcgcgctcaagagcgtcctgatagcgtttacgttcggtgatatcgcggccgaagcccatcaggccgcgacgcttaccgacgcgatcgtagtaggggaccttgcggatttcaaag

SEQ ID NO: 600 - DNA, 224 bp, Klebsiella pneumoniae
ctcgcttttgccggtcagcagctccatcgcccggttacagcctgaaaactccttatcctcattgcgatagaagaccaggtccggggaagcatcaaggaaagagcgcagaaaggaggattgctgttcgagctggatctgcgtctcttcacgctctttcatctcaatcttcaactgctcgagggtggtctcgcgctcggcttccgctttttcacgttcgccaatct

SEQ ID NO: 601 - DNA, 224 bp, Klebsiella variicola
gcacaatctgaagagcgccaatgccgatatcggcgcggcgcgggcgaacttcttcccgtcgatttccctgaccgccagcgccggggtcggcagcgactcgctgtcgtcgctgttcagccacgggatgcaggtgtggtcgttcgccccctccatcagcctgccgctgttcaccggcggcagcaacctggcgcagctgcgctatgcggaagcggagaaaaaggggc

SEQ ID NO: 602 - DNA, 224 bp, Pseudomonas aeruginosa
gcgattaatgacgagactcggcggcgcatggatcgtccttacaaaacgcatccggccgccgaacccgaggtggagaataccgttgaggcgctaggcaaaattcgcaatgccattgctcgcggcgatgacctgggcgtgtatcaatccgcacagaccgtggcaaagttagtgccgacattgttgatccccatcaatccaccggtgaccgtgtcgcacgcacggca

SEQ ID NO: 603 - DNA, 224 bp, Serratia marcescens
gcagcgagacgttcatcgcgctagccgcatttgaccaaggaacagcaataggtgggctcgctgcctacgttttgcccaagttcgagcaagcgcgtagtgagatctacatttatgacttagcagtcgcatccagctatcgacggcaaggaatcgcaacttccctgattagccacctgaagcgtgaggcaatcaagattggggcatatgtgatctatgtgcaagca

SEQ ID NO: 604 - DNA, 224 bp, Haemophilus influenzae
atggtgcgatcgtgattttttctgcacacggtgtgtctcaagcggttcgacaagaagcgaaagatcgtaatctcaaagtatttgatgcaacctgtcctttggtgactaaagtgcatatgcaagttgctcgtgcaagtcgaaagggaacaaaagcgattttgattggacataaaggacaccctgaagttgaaggcacaatggggcagtatagtaatgaagatggc

SEQ ID NO: 605 - DNA, 224 bp, Haemophilus influenzae
gatgctttttgggcaaggtttattgttggcattgccgcctacaatttcttatttgaatggttcaggccaacgccatcgcattgcccatcaagttcgccaaggcatttggcttgtgttaggcgtgagtattcctttaggtttgctgatttatttctgtgaaattccgctgcaatatatgcaaatggaaagcaaaatgtcagatttagcacgggattatttacatg

SEQ ID NO: 606 - DNA, 224 bp, Brevundimonas diminuta
cggtgactcaggtgattgaacagaatatgaacgggcttgatggcctgatgtatatgtcatccaccagcgatgcggccggtaatgcgtcgattatcctgacctttaaaacggggacctcacctgatatagcgcaggttcaggtacagaataagctacagctggcgatgccttccttgccacaagaagtccagcagcaagggatcagcgttgataaatccagcagc

SEQ ID NO: 607 - DNA, 224 bp, Acinetobacter baumannii
ccgcagagttgcgccattgccgaggtgaagtggtatcacgcggatcgccggaatggcggtgttaacgtcggctcggtacggtcgagacggaacgttcgtctccagctgtcggcgaacgcggtgacgctagccggccgccaacgtgagcaatcagcttattcatcgccacgttatcgctgtactgtagcgcggccgcgctaagctcagccagtgacatcgtccat

SEQ ID NO: 608 - DNA, 224 bp, Pseudomonas aeruginosa
gcaccgcgaaagcgtcgcgcagcggcaacgagttctccgccgcctcgtggctgagttcggactcgttgaggatcatcccggcgaagaaggcgcctagcgcgaaggacaccccgaacagcaccgccgagccataggcgatgcccagggcaatggccagcacggccagggtgaagagctcccgcgaaccgctggcggcgatcctctccagcacccagggcaccacc

SEQ ID NO: 609 - DNA, 224 bp, Mycobacterium avium
acgggtcgggaatggtgcccgcgccggcgccgtccttggcggtgaactgccaggccccggcggggctcttggtcagctcccactcgtagccgtacagggtctccaggaaggtgttgtcccacttggtcggggtgggcgtccagaccacctccaggccgctggtgatggcgtccttgcccacgccggtcccgtacgagctcttccagcccagaccctgttgttcg

SEQ ID NO: 610 - DNA, 224 bp, Mycobacterium avium
ccggccagctgttgagcggggcgaaccgctgcatgccctgcccgccgccgccgcggccgtcgtggatgcggtaggtgcccgcggcatgccagctcatccggatgaacaggccaccatagtggccgtagtcggcgggccaccagtcctgcgaggtggtcatcaccgagatcacgtcggccttgagcgcctcgacgtcgagcttggcgaattcctcggcgtagtcg

SEQ ID NO: 611 - DNA, 224 bp, Staphylococcus epidermidis
attggtcatacttggattgcactgaatcaagaaaaaaatataccaaggaatgaaataagtcattcctatacgcacgttgctttctccatagatgaagaagattttcaacagtggattcaatggcttaaagagaatcaagtaaatattttaaaagggcgaccaagagacattaaagacaaaaaatcgatatattttacagatctggatgggcataaaattgaatt

SEQ ID NO: 612 - DNA, 224 bp, Clostridium difficile
atgataaattaataattggtaagttttgctcaatagcatgtaatgcaaaatttctaatgactagtggaaatcactcaatgaaatcaaaatccacatatacttttcccatcttttatgaagaatggaaactcgatatcaaccatataacggacgcttgggacaataagggtaacattataattggaaatgatgtgtggataggttatgattctataattatgtca

SEQ ID NO: 613 - DNA, 224 bp, Klebsiella variicola
ttaccgtcgcggcagagctcgacagcggcgctcatgccgtgcagaccgcggaaagcatgcagcctgacctgctcatcgtcgacgtcgatattccggagctcagcggtatcgaggtgctggagcagctgcgcaaacgccgctaccagggaacaatcatcatcatttccgctaagaatgaactgttctacggcaaacgcagcgccgactgcggagcaaatggtttt

SEQ ID NO: 614 - DNA, 224 bp, Klebsiella pneumoniae
tccttctcaacgccgttcgtcctgtcgctgtttaacccccgcaccttaatgctgacctcacaggtgctgtttctggcggcaggcgtgacgctgagcctcgccactcgccaggccgtcaccctgatcgggctgggcatgatttgcgccggcttctccgttgggttcggcgtcgcgatgagccaggcgctggggccgttcacgctgcgcgcgggcgtcgccagctc

SEQ ID NO: 615 - DNA, 224 bp, Pseudomonas syringae
ggaggagaacggcaccatggttccggcgctgttgcgtacgtaccacttcttcaggtcttcggggctcatgcgggcaccggcctgaccctggatgtacaccttcttcacccgaccacggtcgatgaagtcgttgacgtagctactgcccagggcaatcgacagggtgttgttgatatcggtgagggtcacgcccagggcactggcgcgctcgtcgtcgatggtca

SEQ ID NO: 616 - DNA, 224 bp, Pseudomonas syringae
gatgtagttcgacaggtcgtccttggacatgctgccgtcctcggaaaccacgccgatcaccagcaggaagttcttcactgccttggtcacgcggataccctgctgctgcacttcttgaggcagcagcggagtggccaggttgagcttgttctgtacctgaacctgcgcggtgtccgagttggtgccctgttcgaaggtcgcggtgatggtcatgctgccgtcgg

SEQ ID NO: 617 - DNA, 224 bp, Clostridium difficile
ctttaatactaacactgctatagcttcaactggttatacaattaataatggaaaacatttttattttaatactgatggtattataggagtgtttaaaggacctaatggatttgaatattttgcacctgctaatacggatgctaacaacatagaaggtcaagctatagtttatcaaagtaaattcttaactttgaatggtaaaaaatattattttgataataact

SEQ ID NO: 618 - DNA, 224 bp, Mycobacterium avium
cccaggtcgcaattgccaagggggactggatgattccggccagccgcgccccccagcgggttttcggcgggccgtcgtcgggcgccacgaaatggaaccaggccgccagcagcagcaccagcacggtcagtccgagcagcgcggtggcgaacgcgtaatgccacgccgggaacgcattcgaccacggcacaccgaaattcgagacgtaccaccagccgttgacg

SEQ ID NO: 619 - DNA, 224 bp, Mycobacterium leprae
ccatggtcaggatgtagccgtcgtcggaggtgttggcgccgacgaaatgccaccacgtcagcacggcgatgaccagcgcgtccagcccgccgatcgaccaccagcgggcaggcaggaagcggcggtgccgggtgccgtcggcggtgtccaggatgtgcagcgcgaccagcgccgcgcccgtcagcaccaggccgaggatcatcgcggccatcttcagcggggtg

SEQ ID NO: 620 - DNA, 224 bp, Klebsiella pneumoniae
cccgccgtccggacctgcaggcgctgcgcggctacgtcaccgcctccctgagccaggtcgatgcggcgaaagcggccttctatccccattttgatatcaaagcgttctggggctataacgccctgagcgtcggggatctgtttaagtcctctttccagcagatcaacctgctgcccgggctgtatctgccgatcttcgacggcggccgtctgaacgctaatctg

SEQ ID NO: 621 - DNA, 224 bp, Klebsiella pneumoniae
ctgagtgagatggcggtgattgccggtctgccgaaagcgccttccacttttaacccgctctattcgatggatcgcgcgaccgcccgtcgcaacgtggtgctgtcgcgtatgctgagcgaaggatacatcacccaggcgcaatacgatgaggcgcgcagcgaacccatcgacgccagctatcacgcgccgaaaatcgccttctccgccccttacctgagtgaaat

SEQ ID NO: 622 - DNA, 224 bp, Providencia stuartii
cgaaaccaccctgaacaccgcgattccgggcgatccgcgcgataccaccaccccgctggcgatggcgcagaccctgaaaaacctgaccctgggcaaagcgctggcggaaacccagcgcgcgcagctggtgacctggctgaaaggcaacaccaccggcagcgcgagcattcgcgcgggcctgccgaaaagctgggtggtgggcgataaaaccggcagcggcgatt

SEQ ID NO: 623 - DNA, 224 bp, Mycobacterium avium
catcgtcatcgagctcaagcgcgacgccgtcgccaaggtggtgctgaacaacctctacaagcacacccagctgcagaccagcttcggcgccaacatgctggccatcgtcgacggggtgccgcgcaccctgcggctcgaccagctgatccgccactacgtcgaccaccaactcgacgtcatcgtccggcgcaccacctaccggttgcgcaaggccaacgagcggg

SEQ ID NO: 624 - DNA, 224 bp, Mycobacterium avium
tggcgatcaacctgcgcgacaacgacgaactcgtgggcgcggtgttgtgctcggccgaggacgatctgctgctggtgtcggccaacggccagtccatccggttctcggcgaccgacgaggcgctgcgcccgatgggccgcgccacctccggtgtgcagggcatgcgcttcaacgccgacgactacctgctgtcgctcaacgtggtccgcgagggcacctacctg

SEQ ID NO: 625 - DNA, 224 bp, Proteus mirabilis
tcgacaatcaatatggcgttcttcgccgataacccaatggtggtaagtagcccaaccttaaagtaaacgtcgttttcaagccctctcaatgtagtaaatgctaatgcaccaatgacccctaaaggtacaactaacataactgagaaaggcactgaccaactctcatagagtgccgccaaacaaaggaatactacaattaatgaaatagtatacagtgctggcgc

SEQ ID NO: 626 - DNA, 224 bp, Proteus mirabilis
acatctttttctttagtattaaaatagtcattgacttgtgttaatacgtcttgagtttgctcttgggttgatccagcgggcaactgaaccattgtcagtaataccccttggtcttcttctggtaagaaagacgccggtaaacgcacaaacataattgccatagcagcaacaagtaagacgtagatcagtaagtaacgacctgtaccacgcagtgtacgagaaac

SEQ ID NO: 627 - DNA, 224 bp, Klebsiella pneumoniae
gtgcatatcggcaaagaaggtacgacccttgtggttagcggcttttgcgtccggggtggcgaaacggccatcggcctcttcctgatccaccacgcgctgcagccaggcgtcgtccatcgcggttttcgcgcagaacaccaccacgtgcgaggcgtcgagaattttacgttcgttgaacacgtaggtgccgctggcggctttcgccacgcgagctttcccttcat

SEQ ID NO: 628 - DNA, 224 bp, Enterobacter cancerogenus
acagagggcgagaatctgctgccggtgctgaacgattcgtttgatcgtatcgccggcatgctggatcgctttgccagtcatcgcgcgctggagaagcttaaaattggcgtggtggggacttttgcgaccggggtgttattcgcgcagctggatgatttccgtcgccgctatccgcatatcgatctgcatctttcgacccataataaccgcgtcgatccggccgc

SEQ ID NO: 629 - DNA, 224 bp, Haemophilus influenzae
gataattatttaataataaactttgagaaagagggatcactgggcctgcgacagcaccttgaatgactcggtaaataattaacgcctcaagactatctgcaatcccacagagccaagaagagacaacaaataaaaaagttgagactaaaaataaccgcacttcgccaaaacgttttgctaaccaacccgttattggaatagaaatcgcatttgctaccccaaaa

SEQ ID NO: 630 - DNA, 224 bp, Burkholderia cenocepacia
ggcctggtcggtcttcaccgcgccttcgtactgcttgacgagcgatgcctgcgtatcgacggtttgcgacgcgatcgagtcctgcgacagcagcgtctggtagcgcttcaggtcgaggcgggccgtcgcgagcagcgccgcgtcgcgcgcatgcgtgccttcggcgttctcgagcgcgacctggtacgggcgcgggtcgatctgcgcgagcacgtcgcctttct

SEQ ID NO: 631 - DNA, 224 bp, Vibrio cholerae
cgaagatgcttcatttaaaaactgtcagctttcaatgtcccattttaagggggcaaattgctttggtattgaactgagagattgtgatcttaaaggagcaaattttagtcaagttagttttgtaaatcaggtttcgaataaaatgtacttttgttctgcatacataacaggttgtaacttatcctatgccaattttgagcagcagcttattgaaaaatgtgacc

SEQ ID NO: 632 - DNA, 224 bp, Burkholderia cepacia
gtggccgcgaggacacgtgggagccggatcgcgacgtctactggggcagcgagacgacctggcttggcggcgacctgcgctacgacaagggcggcgcctgcgaatcgcaacacggtggcaacgccgggcgcaacctcgaaaatccgcttgccgcggtgcagatggacctcatctacgtgaatcccgaggaaggtccggacggcaatcccgatcccgtcgcggca

SEQ ID NO: 633 - DNA, 224 bp, Burkholderia cepacia
cgtactggccggcggcgccggcatcgagcaggcggcgaagcgcgctggccacgacgtggttgtgccgttcgcgcccggccggatggacgcctctcaggaacagaccgacgcgcactccttcgcggtgctcgaaccggtcgcagacggtttccgtaatttcgtcaagggcaaattcgcggtgccggccgaagcgctgctgatcgacaaggcgcaactgttgacgc

SEQ ID NO: 634 - DNA, 224 bp, Klebsiella pneumoniae
ttcatttcgacagtcggttatgtctccatccaggaggcttttgatgaaaaagagtctatccgcattatggcggcgctgacatccatcgttttgctggcgccggtaatcgggcccctggcgggcgctggcctgatgaatttcctgcactggaagttattgtttgcgatcatcggcgccatgagcttgctggcctgggcgttattgatttttaacatgccagaaac

SEQ ID NO: 635 - DNA, 224 bp, Klebsiella variicola
gcgacccttccaccaggttgaggatcagctgggtgatggcgcgggagctaccgatacgcgcggcgttgaggttatccagacgcggccccatccagttgaacggacggaacagggtgaacttcaggccgttcttatcgccataggcccagatcacgcgatccagcagctgcttggagaccgagtagatccagcgctgtttgttgatcggccccaccaccaggttg

SEQ ID NO: 636 - DNA, 224 bp, Bordetella bronchiseptica
cgctgatgcgcgaactgctggacgtgtcggcgttccccggcgcctcggcgtcgctgcgcgcgctggaagtcacggtgctggcgctggccgatgtcgtgccgtggcgctacccggcgcggcggctgatgcagtttggcgagtggctgcgcgacgatatccgcgccggcgtgttcgaagcgcctgtgttcgaccacgacctggccatcctgctgaccaaggcgcgc

SEQ ID NO: 637 - DNA, 224 bp, Pseudomonas cichorii
ggcaaggccaaggccggcgacatcagcaacaccacttggtccctggcgatcagccgtttgattatatcggcttcggcgagaacggttccggcggcggcggtgactcgattttcctcgccaactccgtgcagtactccgacttcaacggccccggcgagaaatcctggcaggcccgctacgacctgaacctcgcctcctatggcgttcccggcctgactttcatg

SEQ ID NO: 638 - DNA, 224 bp, Enterobacter cloacae
ggtgctccaggcggccactgaagtgcgtaacgcctggctggccctgcagcgtacgcagatcgtcagcccgattagcggctacgtttcccgtcgctcggtacagcctggcgcgcagatcggcaccaccacgccgctgatggcggtggtcccggcgaccaacctgtggatcgatgctaactttaaagagactcagctggcgcatatgcgtatcggccagccggcca

SEQ ID NO: 639 - DNA, 224 bp, Bordetella parapertussis
cggccagaaaccggtgcagcgccgcatcctgtacgcgatgcaggccatggggctggcggcaggcgccaagccggtgaagtcggcgcgcgtggtcggcgacgtgctgggcaagtaccacccgcacggcgaccaggccgcctatgacgccatggtccggatggcgcaggacttctcgctgcgctatcccctgatcgacggccatggcaacttcggctcgcgcgacg

SEQ ID NO: 640 - DNA, 224 bp, Haemophilus influenzae
tttatattggttgaaagtatatcaacttccgcaagcgagccgtggcgcacgcggtcgtccaattgtgaatattcttccgttacaagaaaacgaacgtattactgcaatcttgccagtttctgcttacgaagaagataaattcgtagtcatggcaactgctggcggtattgtgaagaaaatcgccttaaccgaatttagccgtccacgttcaaacggtatcatcg

SEQ ID NO: 641 - DNA, 224 bp, Acinetobacter baumannii
ttaaggcacgtgcaatcgacacacgttgctgttgaccacctgaaagctggcttggtcggttttgcgttttagtacctaaacctaactccgtgagcagggcagtagctcgctgtttacggtctgctggagtaacaccagcataaacagctggaacctcaacgttaccttcagctgataagtcacccagtaaatgatagcgctggaaaataaaaccaaaatattca

SEQ ID NO: 642 - DNA, 224 bp, Stenotrophomonas acidaminiphila
ggcgagaggcgcggcgagctcgaggcaatagttgaaatgctcggcgggcgcgtcatggaggagttggactatgggattcttagcggagatcggggatgagttacttgactgcgaacctgcttggtgggcagacgaagcgtatgaaatcgcggaggctccgcagggctcgtgcccagaggcggctgagggcgtcatcgccgggcggccagtccgttgtaacagct

SEQ ID NO: 643 - DNA, 224 bp, Pseudomonas aeruginosa
gcgatgtgcagcccgtcggagtcggcatccgcgaggatacagatcttgccgtagcgcagctgggccaggtcgctggcacccggatcgacgccgatggccaccgcgatgtcgtggacctcctggctggcgagcacctcgccgccgtccacttcccaggtgttcaggatctttccgcgcagcggcatgatcgcctggaattccttgtcccgcgcctgcttcgccga

SEQ ID NO: 644 - DNA, 224 bp, Filifactor alocis
caataagattgaccaaaatgggattgatttatcaacggtttatcaagatattaaagagaaactttctatggaaattataatcaaacagaaagtagagctgcaccctaatatgtgtgtgatgagctgtacggaacctgagcaatgggatgtggtaatagaaggaaatgattatcttttggagaaatatacacttgggaaatcattggagatattagaactcgaac

SEQ ID NO: 645 - DNA, 224 bp, Proteus mirabilis
tgaacaacaggtttaccttgctcttgacgaataatgccactttcaatttcattttttaatttcagataatcttcactcgattgtgtcacatcgacataaatcggatcgatttgctgaacagttgttaaagccacttgctgaccaggtgcaactaatgcgccttctgttaccgttgatttacctgaacggccagaaattggtgcggtgactttggtatattcaag

SEQ ID NO: 646 - DNA, 224 bp, Klebsiella pneumoniae
tgtcgctggatccgcagcggcgcgttactccgccggaagagagcgactacacccattatgcgtttagtattagcgaagccgattttgctagcttcgccgcccgccttgaggctgccggcgtggcgatctggaagctgaaccgtagcgaaggcgcttcgcactatttcctcgatcccgatggccataagctggagctgcacgtcggcagtctcgcccagcgtctg

SEQ ID NO: 647 - DNA, 224 bp, Klebsiella pneumoniae
ggactggtaccgggagccttactggcgcgtgaactgggcattcgtcatgttgataccgtatgcatctccagctacgaccatgataaccagcgcgagctgaaagtgctgaaacgtgcggaaggcgatggcgaaggctttatcgtcatcgacgatctggtggataccggcggtaccgccgtggcgatccgcgaaatgtatccaaaagcacactttgtcaccatttt

SEQ ID NO: 648 - DNA, 224 bp, Haemophilus influenzae
gtacagggcggaagtacgctcactcaacaattagtcaaaaatttatttttatcgcgcgaacgcaccattacacgtaaagccaacgaagccttgatgtcattggttttagattggcgttacgataaaaaccgaattctcgaaacctatcttaatgaaatttatctcgggcaaaatggcgatacgcaaattcacggttttgaattagccagccaattttattttgg

SEQ ID NO: 649 - DNA, 224 bp, Proteus mirabilis
cgtaatccattacgtgtatttgaattagattttgaagagaacttaaaaattgttcgttattgtgttaaatacaataaacgtattattttcccatccacatcagaagtatacggtatgtgtgatgataaagagtttgatgaagataattcacgcttaatcgtcggtcctattaataaacaacgttggatttactctgtttctaaacagttattagaccgagttat

SEQ ID NO: 650 - DNA, 224 bp, Serratia plymuthica
caccgagcagatcccggcggtcatgccgaccaagatccccaacctgctggtcaacggttccagcggtatcgccgtgggcatggccaccaacatcccgccgcacaacctgggcgaagtgatcgatggttgcctggcgctgatcgataactccgacatcacggtcgatgaactgatgcagttcatcccggggccggacttcccgacagcaggcctgatcaacggtc

SEQ ID NO: 651 - DNA, 224 bp, Pseudomonas putida
gacgctcctgctgttctccagcaagggcaaggtttactggctcaaaacttatgacattcccgaggcctcccgcgccgcccgtggtcgtccgctggtcaacctgctgccgctggaagaaggtgaacgcatcaccgccatgctgcagatcgacctggaagccctgcagcagcacgccgatccggaagaagagctggaagacggtgacgacggtgtgatcgagggtg

SEQ ID NO: 652 - DNA, 224 bp, Xanthomonas axonopodis
ccgcatgctggcctctgaccaggtcggtacgctgatcaccgcgctgggcaccggcatcggccgagacgagtacaacccggacaagctgcgttaccacaagatcatcatcatgaccgacgccgacgtcgacggcgcccacatccgcaccctgctgctgacgttcttctaccgtcagatgccggagctgatcgagcgcggttacgtctacatcggcctgccgccgc

SEQ ID NO: 653 - DNA, 224 bp, Shigella sonnei
ttcgtcaatacatcgccggtattgctgatggaaatcatgggttttgagcgcggagaatacgccaccattatggcgttgaccgctggcgtcagcatgaccgtttcattctccacgccatttgcgctgggaatttttaagccacgtacgttgatgatcacctcacaggtgttattcctggcggcggggatcacccttgccgtttcaccttcccatgcggtttctct

SEQ ID NO: 654 - DNA, 224 bp, Klebsiella pneumoniae
tgcatatgcaaggtaagctgctggaaaatcgcaataaaatgctcaaagcgcagggaattaacgagacattatttatggcgctgattacgctggagtcacaggaaaaccacagcatccagccgtccgagctgagctgtgcgctgggctcgtcacgtaccaacgccacccgtatcgctgatgaactggaaaaacgcggctggattgaacgtcgcgagagcgataac

SEQ ID NO: 655 - DNA, 224 bp, Delftia acidovorans
atcgacgtggcccaggtgcaggtgcaaaacaagctgcagtccgccgtgaaccgcctgcccgacgccgtcaagagccggggcgtgttcgtgaacaagggcggccaggacttcctggtcacctacagcttcttctccaaggacccggccatgaccgcggtcgatatcggcgactacctgaccagcaacctggtcgatgtgatcggccggctggacggcgtgggcga

SEQ ID NO: 656 - DNA, 224 bp, Anaeromyxobacter dehalogenans
gttcttccccaacgagatcgactacttcgtcagctcggactccacgcccttcgtgcgcgcctccatcaaggaagtggtcagcgcgctgggcgaggccatgatcctcgtggtcatcgtgatgttcatcttcctgcagaacttccgcgccacgatgatccccgccatcgccgtgcccgtggtgctgctgggcaccttcggcgtgctgtcgctggccgggtactcga

SEQ ID NO: 657 - DNA, 224 bp, Burkholderia sp.
gtcccggcatgcaggcgcgcgtcgtcgcgataggccagcagcgcacgacggaaactgcgcgcgttgtctaaaagccacgcatcccacgcttcgcccggccgcggcagcgacgcatcgtgacgctcgagcatgatcgcctcggccatcgcatcgagcagttcgcccttgttcctgaaatgccagtaaagcgtcggcgactgcacgccgagccgctcggcgagctt

SEQ ID NO: 658 - DNA, 224 bp, Klebsiella pneumoniae
tgacccagcgcaccatcaaagcgcaggagtataacgcccagtcggtggcggccgccgtcgagcgcgccagagcgctggttaagcagaccacctccacgcgtatccgccttgagccgctggtgccgcaggggtttgcctcccaggaagatctcgatcaggcgcgcaccgctgagaaagcggcgcgggcggagctggaggccacgctgctgcaggccagacaggcc

SEQ ID NO: 659 - DNA, 224 bp, Enterobacter sp.
cgattgttatgggtggaaagctgaagatcaatatgcggatagcagcggcgaaagtccgcgagcagcgagaataaaacacccgtggcaaaagtccctaccacacctattttgagcttctcctgagcgcgctggctggcaaaacgatccagcattccggcgatgcgatcgaacgagtcattcagcaccggcagcaaattctcgccttcggtggtcaacatcagccc

SEQ ID NO: 660 - DNA, 224 bp, Pseudomonas sp.
gcgcgatgctcacgcgctgctgctggccgccggacagttggccgggccggtgatgcgcgcgatccgcgagcccgaggcgcgcgagcagttcgcgggcgcgtgcgtgccgctcggcgcgcggggtgcccgcgtagatcgccggcatttcgaggttcgcgaccgcgtcgacgtgcggcagcaggtgatagcgctggaacacgaagccgaagtgctcgcggcgcagc

SEQ ID NO: 661 - DNA, 224 bp, Klebsiella pneumoniae
atactaaaaacagtgcctcaggggctgatatcaacaactatgccggacagataaaatcggcgattgaaagtaagttttatgacgcatcgtcctatgcaggcaaaacatgtaccttgcgtatcaaacttgctcctgacggcctgttgttaaatatacagtccgaaggtggtgatcctgctctgtgccaggccgctcttgccgcagcccggcaggctaagttcccg

SEQ ID NO: 662 - DNA, 224 bp, Xanthomonas axonopodis
tgatgttgaccggcaagcccatttcggtggtggtgttcatcggcctgatcctgctggtcggcctggtcaccaagaacgcgatcatcctgatcgacaaggtcaaccagctgcgcgaggctggcgtggccaagcacgaggcgctggtggaaggtgcacgctcgcgcctgcgcccgatcatcatgaccaccctgtgcacgctgttcggcttcctgccgctggcggtg

SEQ ID NO: 663 - DNA, 224 bp, Proteus mirabilis
tgaaagcaatagtatttgcctatcatgatattggctgtgttggtttaaaagcacttgaaaaagcgggtttcgatattcaagctgtatttacccatactgacgatcctaacgaaaatcatttcttctcatccgtagcgcgtgttatcgcaaaaatgggattaaccgtgtttgctccagaaaacgttaatcatccattatggattgagcgtattcatgagatgaag

SEQ ID NO: 664 - DNA, 224 bp, Burkholderia ambifaria
atgtcgtcgacctcgccgccgagggcctcgacttcgcgatccgtttcggcgaaggcaactggccggcgacgcgcaacgagcggctgctcgatgcgccgctcaccgcgctgtgcacgccggacatcgcacggcgcctcgcgcagccggccgatctcgcgaacgaaacgttgctgcgctcgtatcgcaccgacgaatggctcggctggttcgacgccgcacggctc

SEQ ID NO: 665 - DNA, 224 bp, Stenotrophomonas maltophilia
caccggtgcggtgtcgttgcgcgcgttgctgccgaacccgcagcagatcctgctgccgggcgccttcgtcagcttccaggccaacctgggcgaacgcaacaacgcctacctggtgccgcagcaggcgctgctgcgcgacaccaccggcggctacgtgatggtggtcggcaccgacggcaaggtcgtgcgcaagaacgtcaagaccgatggtgcgcagaacggca

SEQ ID NO: 666 - DNA, 224 bp, Serratia marcescens
gcgaaggcctctggcccgccacctgcaatattaagctgttcgacgcgccgctgacggtgctctgcccgccgacggtcgccgcgcgcctgagcacgccgcaggatctgcagcacgagcagctgatgcgcacctaccgcaaggacgagtgggagcactggttcgcggcggcgcaggtgacgccgtggcggatcaacggcccggtgttcgactcgtcaaggctgatg

SEQ ID NO: 667 - DNA, 224 bp, Stenotrophomonas maltophilia
cttgctgccggggccggcggccatgcggtcgaccacttccttctgcagcatgaaatgcatgtcgcggatcaccgcggcatgttccagtgcatggaacaggatcggcgaggagatgttgtagggcaggttgccgaccaggcggatcggctggccgtcggccagttcggtgaagtccacgcgcagcacatcgcggtggacgatggtcagctcgcccaacggttcgg

SEQ ID NO: 668 - DNA, 224 bp, Stenotrophomonas maltophilia
tcttcagtgcgcacggcgtgtcccaggccgtgcgccaggaagccgagcgacgtggtctgaaggtgttcgacgccacctgcccgctggtgaccaaggtgcacttcgaagttgcccgccactgccgtgccggccgtgacgtagtgctgatcggccacgccggtcacccggaagtggaaggcaccatgggtcagtggaaccgcgaagccggtaccggccagatctat

SEQ ID NO: 669 - DNA, 224 bp, Unknown
  Description of Unknown Uncultured bacterium polynucleotide
ttgctgggtaccagtaacatcagcaagcaggactacgacaacgccgtctccaccctgcagcaggcggatgccgccgtggtggccgccaaggccgccgtggaaaccgcgcgcatcaacctggcgtacaccaaggtgacctccccgatctccggccgcatcggcaaatccgccgtgaccgaaggcgcgctggtcagcaacggccaggcgaccgccctgtctaccgt

SEQ ID NO: 670 - DNA, 224 bp, Enterococcus sp.
atgatgatttctatttttgcgattgcccaattaatcacatcgccaatcgcaggtgtcctttcggataaaattggtcggaaaaaaatgattgcaacgggcatgttggtgttttcaatttctgagttattatttggtttagcccaagcgaaaagcggtttttatatttctcgtggtttaggtgggattgccgccgctttattaatgccgtcagtgacagcctttgt

SEQ ID NO: 671 - DNA, 224 bp, Klebsiella pneumoniae
gataagtataacgaacgtccgtggggggcaggtttcggcgtctcgcgctgggatgagaaagggaactggcatggcctgtacctgatggcgtttaaagattctttcaataaatgggagcctatcggcggctacggctgggagaaaacctggcgcccgctgaccgaccagaacttccaccttggactcggctatacccttggcgtgacggcgcgcgataactggga

SEQ ID NO: 672 - DNA, 224 bp, Artificial Sequence
  Description of Artificial Sequence Synthetic Escherichia coli plasmid polynucleotide
caagcacggccccaacagtgaagtagctgattgtcatcagcgcattgacggcgtccccggccgaaaaacccgcttcgcagaggaagcgaagctgcgcgtcggccgtttccatctgcggtgcgcccggtcgcgtgccggcatggatgcgcgcgccatcgcggtaggcgagcagcgcctgcctgaagctgcgggcattcccagtcagaaatgagcgccagtcgtcg

SEQ ID NO: 673 - DNA, 224 bp, Enterobacter cloacae
cgtacaaagcaaatgaacgatttccattatgtagttctttcaaaggttttttagctgctgctgtattaaaaggctctcaagataatcaactaaatcttaatcagatcgtgaattataatacaagaagtttagagttccattcacccatcacaactaaatataaagataatggaatgtcattaggtgatatggctgctgccgctttacaatatagcgacaatggt

SEQ ID NO: 674 - DNA, 224 bp, Serratia plymuthica
gagcgtattgggtaacgactggaataaaccatacaagaaatcggcccgtgtcgtcggggacgtgatcggtaaatatcacccgcacggtgacagcgcggtttacgacactatcgtgcgtatggctcagccgttttcactgcgctacatgctggtggacggtcagggtaacttcggttccgtcgacggcgactccgcggcggcgatgcgttataccgaagtgcgca

SEQ ID NO: 675 - DNA, 224 bp, Serratia proteamaculans
catcgagctgattcgccgtgcgccgaccccggcggaagcgaaagtcgcgctggtggcgcagccttgggatctgggcaacgtcagcgccatgctggaacgcgccggtgacgacgccgctcgccctgagtggctggagccggagttcggcatccgcgacggcaaatactacctgaccgagcagcaggctcaggcgattctggatctgcgcctgcagaaactgaccg

SEQ ID NO: 676 - DNA, 224 bp, Acinetobacter calcoaceticus
cagtgataattcagctgccaattttattctacaagcgattggtggacctaaggctctaacgaaatttttgcgttccattggcgacgatactacgcgccttgatcgctgggaaacagaacttaacgaagcggtgcctggagataagcgagacacgacaacaccaattgcaatggtaacgacacttgaaaagttactaattgacgaaacactatctatcaaatctc

SEQ ID NO: 677 - DNA, 224 bp, Acinetobacter baumannii
tatcaactattcgcaaaagaaataggcgaaaacaaagcacgaagctacctagaaaaattaaattacggcaatgcagacccctcgaccaagagcggtgactactggatagatggaaatcttgcaatttcagcaaatgaacaaatttccatcctaaagaagctttatcgaaatgagcttccttttagggtagagcaccaacgcttggttaaagacttgatgattgt

SEQ ID NO: 678 - DNA, 224 bp, Streptococcus mitis
gcaacgggtaaaattctttatgtagaggattcgcagtttaataaggtagctgaagtcttccataagtatttggatatggatgaagcttatgtgaaagagcaattagctcaaccaaatctgacccaagtttcctttggtgcaaaaggaaatgggattacctatgccaatatgatggctattaaaaaagatttgaaagatgctagtgtggaagggattgactttac

SEQ ID NO: 679 - DNA, 224 bp, Streptococcus pneumoniae
gtacagaactggtttcgcaacaaactgtagacggaaaggatgtttacacaaccctttctagtcctttgcaatcctttatggaaacgcagatggatgccttccaggaaaaggtaaaaggcaagtatatgaccgcgaccttggtcagtgcaaagaccggtgaaatcctcgctaccacccaacgacctacctttaatgcagatactaaagaaggaatcactgaggac

SEQ ID NO: 680 - DNA, 224 bp, Clostridium difficile
gtgaaaaattcagatggacaccataatacttctaattttatgaatttatttttggataatataagtttctggaaattgtttgggtttgaaaatataaattttgtaatcgataaatactttacccttgttggtaaaactaatcttggatatgtagaatttatttgtgacaataataaaaatatagatatatattttggtgaatggaaaacatcgtcatctaaaag

SEQ ID NO: 681 - DNA, 224 bp, Enterobacter hormaechei
ggtcacggataacgcctccctgctgcgtttctatcaacactggcaaccgcagtggaaaccaggcacaacgcgtctttatgcgaacgccagcatcgggctttttggcgccctcgcggttaaaccctccggtatgagctttgaacaggccatgacgaagcgggtcttcaagccactcaaactggaccatacatggattaacgttccgaaagaagaagaggcgcatt

SEQ ID NO: 682 - DNA, 224 bp, Zea mays
tcacggtgggtggcatcgcgttcaccgcacacttcatgccggggcacaccccgggcagcaccgcctggacctggactgatacccgcgatggcaagccggtgcgcatcgcctacgccgacagcctgagtgcgccgggctaccagctgcagggcaacgcccgctatccctacctggtcgaggactaccggcgcagcttcgctacggtgcgaggcctgccctgcgac

SEQ ID NO: 683 - DNA, 224 bp, Zea mays
cgccgggcagtaccgcctggacctggaccgacacccgcgatggcacgcctgtgcgcatcgcctacgctgacagcctgagcgcaccgggctatcaactgcaggacaacccgcgctacccgcacctgatcgacgactaccgccgcagctttgccacggtgcgcgccctgccctgcgatatcctgctgaccccgcatcctggtgccagcaactggaactacagcgct

SEQ ID NO: 684 - DNA, 224 bp, Citrobacter amalonaticus
tgagcgccgcggcgttgcagtacagcgataatacggccatgaacaagctgattgcccatcttggggggccggataaagtgacggcgtttgcccgtgcgattggggataacaccttccggctcgatcgtactgagccgacgctcaacaccgcgatccccggcgacccgcgcgataccaccacgccattagcgatggcgcagacgcttcgcaatctgacgttgggc

SEQ ID NO: 685 - DNA, 224 bp, Escherichia coli
atggaggttcatacttaccaaatatggagatagtgcatgacttttttagaacagcggataaagaagcactacacattatcgatgatgcatttaatattgcaaaaaagattgattacttctccacatcacaggcagccttaaatttacatgagctaacagattctgagaaatgccgtttaactagccaattagctcgagtaaaagttcgccttgaagctatggct

SEQ ID NO: 686 - DNA, 224 bp, Ruminococcus gauvreauii
ttcaaccatattgatatacaggctgcaggtcagttgggtatggctgttggcacagtggcatactcaccgggaagcgtggccgattataccgtcatgctgatgctcatgctgctgcgcggcacaaagtcggttctacgaggaacccagaagcagaattattgtctgaatgactgccgtggaaaagaactgcaggatttgacggttggcgtcctgggaaccggacg

SEQ ID NO: 687 - DNA, 224 bp, Enterococcus raffinosus
ctcttttggcgtgaataaggtatgccgggcagaggaactgcaggcagcggtcacagaggcgggtaagtatgacagcaaaatattggttgaggaggccgtttccgggagtgaggtaggatgtgccatactgggaaacggaaacgatctcatcaccggcgaggtcgatcagattgaattgaaacacgggttttttaagatccatcaggaagcacagccggaaaagg

SEQ ID NO: 688 - DNA, 224 bp, Arsenophonus nasoniae
ttaatggaatatattaccgggcctgattttccgactgctgcgattattaatggtcgcagaggaatattagatgcttatcgtacagggcgtggaaagatttatatccgtgctcaggctgatattgaaactgatgagaaaacgggtcgcgaaaccattatcgtgacagaaattccttatcaggtgaataaagcccgtttaattgaaaaaattgcggagcttgtaaa

SEQ ID NO: 689 - DNA, 224 bp, Citrobacter freundii
ggtagggcgcaaaacatttgagtctatggggccgctgcccaataggaaatacgcggttgttacccgctcaaactggacagcggctaatgaaaacgtagtggttttcccgtcgattgacgaagcgatgggtagattaggcgagatcactgaccatgtcatcgtcgccggtggtggagaaatctaccatgaaacgatacccatggcctctactctgcatgtgtcga

SEQ ID NO: 690 - DNA, 224 bp, Streptococcus salivarius
aacttgcctatattccccagttggacgaagtaactctgcaggaggaaaaagattttgcacttgtaggcaagctaggtgttgagcaattaaatatacagactatgagcggtggtgaagaaacaaggcttaaaatagcacaggccttatcggcacaggttcatggtattttagcggatgaacctacgagccatttagaccgtgaaggaattgattttctaatagga

SEQ ID NO: 691 - DNA, 224 bp, Citrobacter freundii
ccatggttataaaaactgaatatcaaggcaagggttttgggaaagtactactaagagaactagagacgagagcgaagagtaggggaattatcggaatagctcttggaactgatgatgaatatcagaaaactagtttgtctatgattgatataaacgaacgaaatatcttcgatgaaatcgggaatataaagaacgttaataatcatccatatgagttttataag

SEQ ID NO: 692 - DNA, 224 bp, Acinetobacter sp.
aagcttaatgcaaagtgaattgcaacgtattggttatggcaatatgcaaataggcacggaagttgatcaattttggttgaaagggcctttgacaattacacctatacaagaagtaaagtttgtgtatgatttagcccaagggcaattgccttttaaacctgaagttcagcaacaagtgaaagagatgttgtatgtagagcgcagaggggagaatcgtctatatg

SEQ ID NO: 693 - DNA, 224 bp, Mycobacterium bovis
catcgccaccccactgctgccggtcaaccagaccaccgcgcaattgaactggccccaaaacggcacgttcgccagtgtcgaggcaccgctgattggctacgtggccaccgacttgaacatcaccgtcccctgccaggccgccgccggactggccggatcgcagaacaccggcaagacggtgttgttgtcaacggtgcccaagcaggcgcctaaggccgtcgatc

SEQ ID NO: 694 - DNA, 224 bp, Mycobacterium bovis
gcgaacccggcaccgaagggggcaccacggccgcaccgggaatcaacggctcccgcgcccggctgccctacaacctggacccggcccgtacaccggtgctgggcagctggcgagccggcgtgcaggtgcccgccatgctgcggtcgggctggtaccggctgcccaccaacgagcagcgggacagggcgccgctgctggtggtgacggcggccgggcgattcgac

SEQ ID NO: 695 - DNA, 224 bp, Mycobacterium ulcerans
cggcgtgctggcggtcctggtagccatggtggggctggccgcgctggaccggctcagcaggggccgcaccctgcgcgactggctgacccgatatcgcccgcgggtgcgggtcggattcgccagccggctcgctgacgcagcggtgatcgcgaccttgttgctctggcatgtcatcggcgccacctcgtccgatgacggctaccttctgaccgtcgcccgggtcg

SEQ ID NO: 696 - DNA, 224 bp, Mycobacterium tuberculosis
tccagctgtgcgatggccgacgacgtgctggccgagcccgaccccaatgccggcatgctgcaaccggttccgggccaggcgttcggaccggacggaccgctgggcggtatcagtcccgtcggcttcaaacccgagggcgtgggcgaggacctcaagtccgacccggtggtctccaaacccgggctggtcaactccgatgcgtcgcccaacaaacccaacgccgc

SEQ ID NO: 697 - DNA, 224 bp, Azotobacter vinelandii
ggtcctcggcgcgtaggccgccggcggcgatcagcgcctgggcggcgtagtcgggcacggtggcgatgatgtcggtgccggccaggagggtgccgaggccgttgaactgcggcaccgccaggaccaccttgcgcttgcggccgaatttttccagctcctcgtcgacgaagccgctgaggtcgccggcgaaggacaccagcgcgtgcggtcgcgcgcaatagtcg

SEQ ID NO: 698 - DNA, 224 bp, Azotobacter vinelandii
tacgcgatgagcgtgatcgtcgggcgggccctgccggatgcacgtgacggcctgaagccggtgcaccgccgtgtgctttatgccatgagcgagctgggcaacgactggaacaagccctacaagaaatccgcccgtgtggtcggcgacgtgatcggtaagtaccacccgcacggcgacatcgcggtctacgacaccatcgtgcgcatggcgcagccgttctcgct

SEQ ID NO: 699 - DNA, 224 bp, Pseudomonas aeruginosa
ctgaccgtcgatgagctgatgcagtacatccccggtccggacttccccaccgccggcatcatcaacggccgcgccgggatcatcgaggcctaccgcaccggtcgcgggcgcatctacatccgtgcccgcgccgtcgtcgaggagatggagaagggcggcggtcgcgagcagatcatcatcaccgagctgccgtaccagttgaacaaggcgcggttgatcgagaa

SEQ ID NO: 700 - DNA, 224 bp, Pseudomonas aeruginosa
tgcagcacaccctgctgcgctcctaccgcgccgacgagtggccgctgtggttccaggcggccggactgccggcgcacgcgccactgacccggagcatcgtcttcgacacctcgctggccatgctcgaggcggcccgccagggtgtcggcgtggccctggcgccggcggcgatgtttgcccggcaactggccagcgagagcatccggcgtccgttcgccaccgaa

SEQ ID NO: 701 - DNA, 224 bp, Pseudomonas aeruginosa
cggccgcctgtcccttcgaccatcgcctggaacggcgtctggacaccgtgcgccagcgggtcgaggccgggctggtggacgaggcggacttcgacgacgaccatcgcggtcgcagcgccacggagctgtaccgcctgctgctcgaccggcgtccggcggtcgaagacctggtggtcgcccatggcgacgcctgcctgccaaatctgctggcggagggccggcgc

SEQ ID NO: 702 - DNA, 224 bp, Pseudomonas aeruginosa
cgatctcgacgaactgcaggcacgggtgcgcgccctgacccgccgcaccaccggtcgcgccctgccgcaactggtgcacggcgagctgcgcctggacccggcgacccaccaggtgaccctgtccgggcaggcggtggaactggcgccgcgcgaatacgcactgctgcgcctgctgctggagaacagcggcaaggtgctctcgcgcaaccaactggagcagagcc

SEQ ID NO: 703 - DNA, 224 bp, Staphylococcus haemolyticus
gttgtttcagaaaaattaccatattacttagaagaaaagggccaattatctaaatcattagttaaaaaagcaattaaagctcaacaagcacgcgaggctgctcgtaaagctagagaagatgcacgctccggaaagaaaaataaacgtaaagatacattgttatcaggtaagttaactcctgcgcaaagtaaaaatactgataaaaacgagttatatctagttga

SEQ ID NO: 704 - DNA, 224 bp, Staphylococcus capitis
atgtcatgggtaaatatcaccctcatggtgattcttcaatttatgaagcaatggtaagaatggcccaagactttagttatcgttatccacttgtagatggtcaaggtaactttggctctatggatggtgacggtgcagccgcaatgcgttataccgaagcacgtatgactaaaataacattagaacttttacgtgatatcaacaaagacacaattgattttatt

SEQ ID NO: 705 - DNA, 224 bp, Proteus mirabilis
cactctgccactggttttattaaaaagagtctctgtttttggcttattgcctattttattattcaactaatagggcaaattccggccactatttgggtgctattcacacaagtgcgtttcgcttggcacactactgaagtaggtttatctcttgcatttcttggtgtattacatattttttttcaagcggttctcgcaggaaaactggcgcaaaaatggggaga

SEQ ID NO: 706 - DNA, 224 bp, Staphylococcus aureus
ttggacgtattacggacgacggtaaaattcgtgaatacgagctgcctaataaaggatcttacccttcttttatcactttgggttctgataatgccctgtggttcacagaaaatcaaaataatgctattggtagaattacagaaagtggggatattacagagtttaaaattcctacacctgcatcaggaccagttggtattacaaaggggaacgacgatgcttta

SEQ ID NO: 707 - DNA, 224 bp, Ruminococcus gauvreauii
caaccgcatggcttatgaagggtgatacactgactgtcagacagctcctgattgcccttatgcttccatccggtaatgatgcagcctatacccttgcagtcaatactggaaagaagattgcaggagataaaagcctgacccatcagcaagcgattgaagtattcatggataaggtaaatgaaaaagccgtggaccttggcgccacaaactcgaaatttgtagct

SEQ ID NO: 708 - DNA, 224 bp, Ruminococcus gauvreauii
tcctttggcgtaaacaaggtatgcaaggcagaagaactgcaggcagcaatcgaagaagcaagaaaatatgacagcaagattttgattgaagaggccgttaccgggagtgaggtaggctgcgccatactgggaaacggaaatgatctcatggctggcgaggtggatcagattgagctgagacacggcttttttaagattcatcaggaagcacagccggagaaggg

SEQ ID NO: 709 - DNA, 224 bp, Enterococcus raffinosus
cgaaaaagcgttactatccaaatatcaaaaggaatgagatggttgcgaaggggtatgtggcctcacaatccagccacagccgtggaagtacggttgaccttacaatttttcatttgaatagcggtatgcttgttcctatgggtggagattttgactttatggatgaacggtcacaccatgccgcaagcggtctgagcgaagaagaatcaaaaaaccggcagtgc

SEQ ID NO: 710 - DNA, 224 bp, Bordetella parapertussis
cggcatttcgctggaggcgctgcgcgtggcgggcggcctgtcggtggccgtgtccggctgggccatgctcaacgccccggagccgcacccgcaggcccaggcgctgcagcagatcacgcccagcatggcgcgcgagatggtgttcttcccgctcaccatgcccctgaccaccggcccgggcacggtcgcggcgaccatcgccctgagcgccaaccgcaccaacg

SEQ ID NO: 711 - DNA, 224 bp, Klebsiella oxytoca
tggctgaaccgcccccacatagtcgagtggtggggcggcgaggatgaacgcccaactcttgacgaagtcttagaacactattcgcccgaagttctggcaaagcaagctgtagtgccttacatcgcaatgctagatgacgaacccatcggctacgcccaatcctacatcgcacttggaagtggcgatggatggtgggaagacgaaactgatccaggggtccgcgg

SEQ ID NO: 712 - DNA, 224 bp, Xanthomonas albilineans
gctggcgctgcgcaagcagctcgatgcgggcctggtgaaggccatcgatggcaaggcacaggtaaaggtgctgctggaggacggcagcacctacgcgcatgaaggcacgttggagttcgtcggcagcgcagtggatccgagcaccggcaacgtgaagctgcgcgcggtcattccgaacccggacggcctgctgttgccgggcatgtacctgaaggcggtgctgc

SEQ ID NO: 713 - DNA, 224 bp, Bordetella petrii
gtcgacgctgttctcggccggcacccgtggctggtcgttcgcgcccagcatcaccgcgccgattttccatgccggtgcactgaaggcctcgctggatgcctcgaagatcggcaaggacatcggcatcgcgcagtacgagaaggcgatccagcaggccttcagtgaggtggccgatgcgctggccacgcgcgatcacctgaccgcgcagctggacgcgcaacgcg

SEQ ID NO: 714 - DNA, 224 bp, Enterococcus faecalis
gagataatgcgattccagacaattacgaagtaaatctggcgaagtagagggcggggagcgtgtagatgagcgtatctatgaacctcttatggaaatgcttaatgcggcaagggaggaaaactggggcgaattgccgatggtagtatctggctatcggacgcaggaaaaacagcagagcctttatgatgaaaagattgcaaagttcaaaaaagaggggtattcag

SEQ ID NO: 715 - DNA, 224 bp, Enterococcus faecalis
gcagaccgtcacgagaaatataaggacggattaaatcttgttaatggaaagattgtaggctcttatggcggaggtttgtgtcaattgagtaatatgctattttggctttttttacacacgccgcttgttattgtcgagcgacacggacacgcagttgagtctttcccatcaacaaccgaagatttgccctgcggtactgatgctacgattaacgaaggttggtt

SEQ ID NO: 716 - DNA, 224 bp, Enterococcus faecalis
tacctgttgttggctgcgatacactctcatcagctctttgtatggataaggacagggcacataaactcgttagccttgcgggtatatctgttcctaaatcggtaacattcaaacgctttaacgaagaagcagcgatgaaagagattgaagcgaatttaacttatccgctgtttattaaacctgttcgtgcaggctcttcctttggaataacaaaagtaattgaa

SEQ ID NO: 717 - DNA, 224 bp, Enterococcus faecalis
cgttccgttcttttttatgacatctggattttttctgatttccagatatacctgtaatgccgaaaagctgggagcttttataaaaaagacaaccttaatttacggggttgcaatactcttatacatacctatcaatgtttataacggttatttcaaaatggacaaccttttgccaaatatcataaaagatattgtatttgatggtactttatatcacttgtggt

SEQ ID NO: 718 - DNA, 224 bp, Enterococcus faecalis
gaaaaaggcaaaacataccgccgatacggatagagcgtatctggaaatcaacctaaataatttagagcataatgtaaacactttgcaaaaagcaatgtcacctaaatgtgaattgatggcggttgtaaaagcggaagcctatggtcacggtatgtatgaagtgacgacatatcttgagcagataggagtttcttcatttgcggtagctaccattgatgaaggta

SEQ ID NO: 719 - DNA, 224 bp, Enterococcus faecalis
ctaattgtattacgattgtatctgaaaggcttcacaaagtattttaatgaaataaacagaggtattaatgccttgaaagaggaaagttcagaagatgttgtattatcttctgagcttgcggcgactgaaaaaacaatcaatacaattaagcatacccttgaacagcagaaaactgcggcgctggttgcagagcaaaggaagaacgaccttgtagtgtatcttgc

SEQ ID NO: 720 - DNA, 224 bp, Staphylococcus epidermidis
ttgctggaacattaggtgttgttgcattcattatgtcagttttattaattcataatcctcaaaaagcaactacagatggattccaccaatatcaacctgaattattcactaaaattaattggaaagtatttattactccagtcatattaacacttgtattagcatttggtttatctgcttttgaaacattattttctttatatacagctgacaaagtaaattat

SEQ ID NO: 721 - DNA, 224 bp, Staphylococcus epidermidis
gaatttaatacatgtattagtaggtatggtcccagcagggattttaggatttttatttgatgatttaattgaaaaatacttatttagtgtaccaacagtcttaattggtttatttataggtgccatttatatgattatagctgataagtattctaaaactgttcagcatcctcaaacagtagatcaaattaattatttccaagcatttgtcattggtatctctc

SEQ ID NO: 722 - DNA, 224 bp, Serratia marcescens
ttggcggtcctgaggggatgactaaatttatgcgttctattggagataatgagtttaggttagatcgctgggaactggaacttaacactgcaatcccaggagataaacgtgacacttcaacgccaaaagctgttgcaaatagtttgaataaactagctttggggaatgttctcaatgctaaagtgaaagcgatttatcaaaattggttaaaaggtaatacaact

SEQ ID NO: 723 - DNA, 224 bp, Burkholderia cepacia
ccgaaggtgcaatcgttatttcagacgaacgtcaagcggagcatgctttattggtttttggtcaagagcgagcagcaaagcgttactcgcctgcttcaaccttcaagcttccacacacactttttgcactcgatgcagacgccgttcgtgatgagttccaggtttttcgatgggacggcgttaaacggagctttgcgggccataatcaagaccaagacttgcga

SEQ ID NO: 724 - DNA, 224 bp, Enterococcus faecium
atttacgatgaaacagggcaattagaaaattatttatcagagataagtggtgcaagaccaaatagacttactgaagaaaatgctaattttttgttgtgtaatttctctaatctatggttgatgggaatcaacgttctaaaaagaggagaatatgctcgttcattagaactcttatcacaacttcaaaaaaatacactacaacttatacgtatggcagaaaaaaa

SEQ ID NO: 725 - DNA, 224 bp, Clostridium difficile
gaatcatttagagaaaatttaaatgatcctgaatttaaccacactgcatttttcagaaaacgtatgcaaataatctatgataaacagcaaaatttcataaattactataaagctcaaaaagaagaaaatcctgaccttataattgatgatattgtaaagacatatctttcaaacgagtattcaaaggatatagatgaacttaatgcttatattgaagagtcatt

SEQ ID NO: 726 - DNA, 224 bp, Clostridium difficile
atgaaatataaagaatatataccagaatatacttcaaaacattttgatacattggatgaagaagttcaaagtagctttgaatctgttttagcttctaagtctgataagtcagaaatatttttaccactaggagatatagaggtatcacctttagaagtaaaaattgcatttgccaaaggttctattataaatcaagctctaatttctgcaaaagattcatattg

SEQ ID NO: 727 - DNA, 224 bp, Serratia marcescens
gtagctgattgtcatcagcgcattgacggcgtccccggccgaaaaacccgcctcgcagaggaagcgaagctgcgcgtcggccgtttccatctgcggtgcgcccggtcgcgtgccggcatggatgcgcgcgccatcgcggtaggcgagcagcgcctgcctgaagctgcgggcattcccgatcagaaatgagcgccagtcgtcgtcggctctcggcaccgaatgcg

SEQ ID NO: 728 - DNA, 224 bp, Unknown
  Description of Unknown Uncultured bacterium polynucleotide
gattgcgaattggaaccaggaccagactctagacagtgcgtttaaagtgagttgtgtctggtgctaccagcagattgcccttcgagtgggggcactcaagtacccagcctatattcaacagacaaactatggtcatttactggaacccttcaatggaacggagttttggctggatggctctttgacgatcagcgcggaagaacaggttgcctttctccgacggg

SEQ ID NO: 729 - DNA, 224 bp, Pseudomonas aeruginosa
aatttcagcgctttttttaacagaccaagaattgcttgctatctataaggaggcactcaatctgttaaattcaattgctattattgattgtccatttatttcaaacattgatcatcggttaaaagagtcaaaattttttattgataaccaactccttgacgatatagatcaagatgattttgacactgaattatggggagaccataaaacttacctaagtctat

SEQ ID NO: 730 - DNA, 224 bp, Escherichia coli
ccgatgcaaagtgtgtttaaactgcatttagctatgttagttctgcatcaggtcgatcaggggaaactggatttaaatcagtctgttactgttaatcgtgctgcagtattacaaaatacctggtcgccaatgatgaaagatcatcagggcgatgaatttactgttgcagtacagcagttactgcagtattcggtgtcacacagcgacaatgtggcctgcgattt

SEQ ID NO: 731 - DNA, 224 bp, Staphylococcus lentus
gatagttagtggtattgcacttgttcttgcgattggagttccgatttatggtcgaatctcagacttttttgagttacgaaagctatatatctttgccattatgattctggcaagtggtagtcttttatgtgcaattgccccgaacctcccattgttggttttgggaagaatggttcagggtgctgggatgtccgcaattccagttctatcaatcattgcaattt

SEQ ID NO: 732 - DNA, 224 bp, Acinetobacter baumannii
tcccgaaaagtgactttggagaacaaacaattatctacattaaacccttttaaaatatttgctggtattagctctattcgtggtgtacttccacttattacgaccttttttatctttagtgccataggggaggtatatggagtctgctgggcattatggggacatgacacatttcagtggagcggtttctgggtaggtctttctctaggcgcatttggtctatg

SEQ ID NO: 733 - DNA, 224 bp, Clostridium difficile
ttataccatctggagttattggaatactgtttgaagatgatatagatagattattctttaattcaacagtagttgctattgctcttattgtatatggtataataatgatagggcttgaaaagagaaataaaagacctaagtataaagatttttctcaagttacatataaattagctctttgtataggattattccagtgtctagctcttataccaggtacatct

SEQ ID NO: 734 - DNA, 224 bp, Clostridium difficile
tgaatcatgtgaaaatgatattaaatacactaaacttgattttgtatttcctgtattgcatggaaaaaatggagaagatggaacagtgcaggggttatttgaattggcaggtatacaggttattggatgtggtactctttcttcagcattatgtatggacaaagacaaatcacataaacttgttcattcagaaggtattaaagtaccaaaatcaatattactaa

SEQ ID NO: 735 - DNA, 224 bp, Clostridium difficile
gtttaatttacaatctgtaattataggatttatagtgggtgggattttattattagtagtagaaactttgtttagaaaaaagaatcattccacagataatatagataaaattactccaatacaagctttaaaggttgggacattacaagttttgtctgcatggccaggtatgtcaaggagtgcatctactattatgggtggatggatagcaggacttaattctc

SEQ ID NO: 736 - DNA, 224 bp, Unknown
  Description of Unknown Uncultured bacterium polynucleotide
ctgaagtgctggagcaatacctaccaagcgccctggcgaaagagtccgtcactccctacatcgcaatgctggatgaagaaccgattgggtacgctcagtcgtacattgcactcggaagcggtgacggatggtgggaagacgaaaccgatccaggagtacgcggaatagaccagtctctggcgaatccatcgcagctgggcaagggcttgggaaccaagctcgtt

SEQ ID NO: 737 - DNA, 224 bp, Staphylococcus aureus
tatcaaacaaagaaattgaagcaatacttaataaatttttagaatatgtagtaccttttgaactacctagtccgcaaaaacttcagaaagtatttaagaaagttaaaaaaataaaaatacctcaatttgaagaatatgatttgaaggtaagttcatttgtaggatggaatgaacttgcatcaaatcggaaatatataatatattacgatgaaaaaaaacaatta

SEQ ID NO: 738 - DNA, 224 bp, Psychrobacter sp.
gttgctggccgtacatttgtacggctccgtagcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattggtccagcggcggaggaactctttgatccgg

SEQ ID NO: 739 - DNA, 224 bp, Pseudomonas stutzeri
gcaggaaccgacctggatgccagcgcggctgccgcacctgctgctgaacggcaccaccggcatcgccgtgggcatggccaccgacgtgccgccgcacaatctcaacgagatcgtcagcgcactgctgcacctgctggacaaccccgatgcgaccgtgcgcgacctgtgcgagcacgtgcagggcccggactacccgtccagcgccgagatcatcaccgccgcca

SEQ ID NO: 740 - DNA, 224 bp, Stenotrophomonas maltophilia
agatcgccggcctgcaggagggcttcgcgttcgcgatgatgccgccgccgatcctcggcctgggcaacggcaacggctaccagatgttcatcgaggaccgtggcaacctgggttacggcgcgctgcagaacgctgtgcaggcgatgcagggtaccgtcgcgcagaccccgggcatggccttcccgatcagcagttaccaggccaacgtgccgcagctggatgct

SEQ ID NO: 741 - DNA, 224 bp, Stenotrophomonas maltophilia
agcgcatgctggcccgcgtgtgggagccgggcctggtcggtgcgatgctgggtgccgccggcgccgaagcctcgcgcccggaagacctgcccaagggcgtgggcctgatcgagggcggctaccagctgaccgagatccaggccacccagatcctggaaatgcgcctgcaccgcctgaccgggctggagcaggaccgcctgaccgatgagtacaagcagctgctg

SEQ ID NO: 742 - DNA, 224 bp, Xylella fastidiosa
tcgatccaggaagtggtcaagacgctgtttgaagcagtgttcctggtgttcctggtgatgctgatcttcctgcagaacttccgcgccacgctgatcccgaccctggtcatcccggtggcgctgctcggtaccttcctgggcatgtggatgatcggcttcacgatcaaccagctgaccctgttcgcgatggtgctggcgatcggcatcgtggtcgatgacgcgat

SEQ ID NO: 743 - DNA, 224 bp, Burkholderia cenocepacia
gtcgacgcattgcgtcgcgcggtgaagagcgggcgcgcgacgggcatcgcgcagcacgacgtcgcggtgacgctgctgcgcgccgacggcaccgcgtatccgctgaagggcaagctgctgttcagcgatctcgcggtcgatccgaccaccgacaccgtcgcgatgcgcgcgctgttcccgaatccggagcgcgagctgctgccgggtgcgtatgtgcggatcgc

SEQ ID NO: 744 - DNA, 224 bp, Burkholderia cenocepacia
cgcgctttcgttcgcgagcagttcctcgagcttcgccttctcgtcgcgcagcgcttcgagttccttctcgatcttgatcttctcgagccgcgcgagctggcgcaggcggatttcgaggatgtcctcggcctgccgctcgctgaggccgaacgcgctcatcagcgcggccttcggctcgtccgactcgcggatgatgcggatcacctcgtcgatgttcaggaaga

SEQ ID NO: 745 - DNA, 224 bp, Caulobacter crescentus
ttcgtgcgcctgtcgatcgaggaagtggtcaagacgctgatcgagggcatcgtgctcgtgttcctcgtgatgtacctgttcctgcagaacctgcgggcgacgatcatcccgacgatcgcggtgccggtcgtgctgctcggtacgttcgcgatcatgggcctcgccgggttctcgatcaacacgctgtcgatgttcggcctcgtgctcgcgatcggcctgctggt

SEQ ID NO: 746 - DNA, 224 bp, Pseudomonas aeruginosa
cttatcgtccgcgatgctgatgagttgcttcttattgatacagcgtggggggcgaagaacacggtagcccttctcgcggagattgaaaagcaaattggacttccagtaacgcgctcaatttctacgcacttccatgacgatcgagtcggtggagttgatgtcctccgggcggctggagtggcaacgtacacctcacccttgacacgccagctggccgaagcggc

SEQ ID NO: 747 - DNA, 224 bp, Proteus mirabilis
tatgggatagtgttaaccctgggtacactatctttcatgaacagaccgaaacattttcatctttatggagttactaccacaaagatatcaatcattttttgaaaacttattcagaagatatagcacaatacggtgacgatctagcctattttccaaaagaatttatcgaaaatatgttttttgtgtcagcaaatccttgggtaagtttcaccagttttaacttg

SEQ ID NO: 748 - DNA, 224 bp, Cronobacter turicensis
atctggcaggcggcatgtttttacagtggctgctggggccgctgtcggatcgtattggccgccgtccggtgatgttgaccggcgtagtatggttcatcgtcacctgcctggcgacgctgctggcgcagaccattgagcagtttaccctgctgcgcttcctgcaggggatcagcctgtgctttatcggcgcggtaggctatgcggcgatacaggagtcctttgag

SEQ ID NO: 749 - DNA, 224 bp, Klebsiella pneumoniae
ctgaaagacattctgccgacgccggaaaaagtgaatgacgccatcttctattatgcccgcttcgaggtaccgaacccgcaggggatcctgcgcctcgacatgaccgcgcaggtgcacattcagctggctgaggtgaagaacgtgattactatcccgctgagcgccctgggcgacgcggtaggcgataaccgctaccacgttcgactgttgcgcaccggtgaggt

SEQ ID NO: 750 - DNA, 224 bp, Klebsiella pneumoniae
aatacccgtcggcagctgtttcccaataaagcgaaagtggtgggagaggtgattctggtgggcaatatgccggccacggtgattggcgttgcggatgaaaaacagtcgatgttcggcagcagcaaaattctccgcgtctggttgccctacaccaccatggcgggaagagtaatgggccagtcgtggctgaactccatcactgtgcgcgttcatgaaggctacga

SEQ ID NO: 751 - DNA, 224 bp, Klebsiella pneumoniae
aatccgcgaaaattttgtgccgatcgccaggaaagagatgctcagtgccagagaggtgttctcctcgctgcagaatcccaaactggtgctcagcctgtttgtgacctcgctgataatccaggtggccaccggttccattgcgccgatcctgacgctttatgtacgcgatctcgcgggcaacgtcagtaacatcgcctttatcagcggaatgatcgcctcggtgc

SEQ ID NO: 752 - DNA, 224 bp, Klebsiella pneumoniae
cagatgggctgggcagcgctgatggtcggcattgcgctcggcctgcgccagctggtacagcagggattaggcatctttggcggcgccatcgccgaccgcttcggcgcgaaaccgatgatcgtcaccggtatgctgatgcgtgccggcggtttcgcggcgatggccgtcgcccatgagccgtgggtgctgtggttctcgtgcatactctccggactcggcggtac

SEQ ID NO: 753 - DNA, 224 bp, Klebsiella pneumoniae
gcgctgctggtctccccgctcgggatcattgacgtcgctggtcaccagatcgcgcttaatttcagctcgctgatgttcgttctgccgctgtcgctggcggcggcggtcaccattcgcgtcggcttccgccttggccagggctcaacgatcgatgcccaggtttcggcccgcaccggcgtgggcgtgggggtctgcctggcggtatttaccgctatctttaccgt

SEQ ID NO: 754 - DNA, 224 bp, Cronobacter sakazakii
acctgacgctgatggccctgaccatcgccaccggcttcgtggtggatgatgccatcgtggtgatcgagaacatctcgcgctatatcgaaaaaggcgagaagccgctggccgccgcgctgaagggcgcgggggagatcggcttcaccatcatctccctcaccttctcgctgatcgcggtgctgatcccgctgctgtttatgggcgatatcgtcggccgcctgttc

SEQ ID NO: 755 - DNA, 224 bp, Citrobacter rodentium
agcctcaataacctgtcgctgatggccctgaccatcgccaccggcttcgtggtggatgacgctatcgtggtgctggagaatatctcccgccatctggaggcggggatgaaaccgctgcaggcggcgctgcaggggagccgcgaggtcggctttaccgtgctgtcgatgagcctgtcgctggtggcggtgttcctgccgctgctgctgatgggcggcctgcccgg

SEQ ID NO: 756 - DNA, 224 bp, Klebsiella pneumoniae
ccctggtggccctgctggcctcgctgatgatcggcctgtttgtccgcgaaacgctgccggctgaaaggcgtcagccgttccatctgcgcaccacgctgggtaactttgccacgctgttccgccacaagcgggtactgagctatatgctggcgagcggctttagcttcgcgggcatgttctctttccttagcgccggtccctttgtctacatcaatataaaccac

SEQ ID NO: 757 - DNA, 224 bp, Klebsiella pneumoniae
atcttccgccttctgcaccaccttcagcgtctgctgctgggtgctgccgctcggcaactggaccgaggtggtgaacatcccgcgatcctccagcggcaggaacgaggtcggcaaatgcagaaacaggaacaccatcccgccgagcaggacgacgtagatcagcatccagcgcagcgagcggtgcagtattttgccgacgaaggtttcgtagcggctggcgctgc

SEQ ID NO: 758 - DNA, 224 bp, Klebsiella pneumoniae
tgtcggcggcgtattgctgattgcagcggaagtgctgaagccgaaacagccgcgcgcggtcgggattgacgatatgacctaccgtcaggcgttcgtcatcggctgctttcagtgcctggcgctgtggccaggtttctcccgctcgggggcgactatctccggcggcatgctgatgggcgtcagccgctatgcggcctcggagttctctttcctgctggcggtac

SEQ ID NO: 759 - DNA, 224 bp, Citrobacter koseri
gtatgctgccgagcgacattaacaacatgtatgttcgtagcgccaacggcgaaatggtgccgttctccgcctttgtcacttcacgctggatatatggctcgccgcgcctggagcgctacaacgggttgccatctatggagatcctcggtgaagcttcgccaggcaaaagtaccggggaagccatggcgctgatggaaacgctggccagtaaactgccgagcggc

SEQ ID NO: 760 - DNA, 224 bp, Salmonella enterica
taccaaatacagtaataataatggctcgcagtccccgtggatggaagaacaaattcgggatgcctggggaagcatggtccttaaaaatgttgtgcgcgaaacggatgaagtcggtaaaggtcagatccgtatgagaaccgtttttgaacaggctattgatcaacgttcttcaacaggtgcctggcgtaatgccctctctatctgggagcctgtttgcaatgaga

SEQ ID NO: 761 - DNA, 224 bp, Acinetobacter sp.
catgaagtggtcaaacactacctgccgagggttttggcagaagaagccgtcacaccatacatcgcgatgttgggcgacgaacccatcggctacgctcagtcatacgtcgcactcggaagcggtgatggatggtgggaggatgaaaccgacccaggcgtacgagggatagaccaattcctgtcgaaccatacacagttgaaccagggcctaggtacaaagctcgt

SEQ ID NO: 762 - DNA, 224 bp, Pseudomonas aeruginosa
gtcgaggccgatgatgttcaggttgacccggtagctggtctccaggtcggtggtggcgaacagatgggtcatcagctcttcgacatcgacccggttcgaacgcgggatgatgacgatgcgggtcgggttctcgtggtccgactcgtcgcgcaggtcggccaccatcggcagcttcttggcctgcatctgcccggcgatctgttccagcaccttggaaccggaca

SEQ ID NO: 763 - DNA, 224 bp, Corynebacterium asperum
gtcgaggcaatggttgaaatgctcggcgggcgcgtcacggaggagttggactatggattcttagcggagatcggggatgagttacttgactgcgaacctgcttggtgggcagacgaagcgtatgaaatcgcggaggctccgcagggctcgtgcccagaggcggctgagggcgtcatcgccgggcggccagtccgttgtaacagctgggaggcgatcatctggga

SEQ ID NO: 764 - DNA, 224 bp, Stenotrophomonas maltophilia
ggtttcggccaggcccagtcgcggggccagctgctgccacagctgttcggtgatgaacggcgtgagcgggtgcagcaggcgcagcagcgcttccagcacgtacagcagggtgtggcgggtgctttcggcgtcggcggcatcggcaccgttcagcgccggcttgctcagttccaggaaccagtcgcagaactcgttccaggcaaactcgtacaggcactgcgcca

SEQ ID NO: 765 - DNA, 224 bp, Acinetobacter baylyi
caacaaatgagattttcaaatgggatggtaaaaaaagaacttatcctatgtgggagaaagatatgactttaggtgaggcaatggcattgtcagcagttccagtatatcaagagcttgcaagacggactggcctagagctaatgcagaaagaagtaaagcgggttaattttggaaatacaaatattggaacacaggtcgataatttttggttagttggccccctt

SEQ ID NO: 766 - DNA, 224 bp, Acinetobacter sp.
tgtaggtttagcattgggttataacggcggtgacatttcttggtctgatgatgtaaaagtcaatggatcaacttatgaccttgatatggataataacaacgtttatttaaatgctgagattcgtccatggggtgcaagcactaaccgttgggctcaaggcttatatgtagctgctggggcggcttaccttgataacgattatgatttaactcgtaacgttgatg

SEQ ID NO: 767 - DNA, 224 bp, Pseudomonas aeruginosa
catcctaaatgccctaggaggtcctgaaagcgtgacggattttctaagacaaatcggagataaagaaacccgtctagaccgtattgaacctgaattaaatgaaggcaagcttggtgatttgagggatacgacaactcctaatgcaatagtgaatactttaaatgaattattatttggttccacattgtctcaagatggccagaaaaaattagagtattggatgg

SEQ ID NO: 768 - DNA, 224 bp, Bordetella bronchiseptica
gactggatcaacttcgcgtccagttcgggcaaggtgttcgaggtggtcatccagttcggctccatcctggccgtgatgtggatattccgggcccgcctgtggcaattgatccgcggcaccctgaccggggtgcgccaggaggtcaatttcacgcgcaacctgctgctggcgttcctgccggcggcggtcatcggcgccatcttcatcaagtccatcaagcaggt

SEQ ID NO: 769 - DNA, 224 bp, Acinetobacter baumannii
cttctttatgaatgaaaaagataccgtggaatctattttcactgtttctggtttctcattcacaggtgttggtcaaaacgcgggtattggcttcgttaagttgaaagactggagcaaacgtacgacaccagaaactcaaattggttcattgattcagcgtggtatggcattaaatatgatcattaaagatgcatcatacgttatgccgttacagcttccagcaa

SEQ ID NO: 770 - DNA, 224 bp, Burkholderia phymatum
gccgtcgccgtcgcgcgagccgaagttgccctgcccgtcgatcagcgggtagcgcagcgagaaatcctgcgcgagacgcacgagcgcgtcgtacgccgactggtcgccgtgcgggtggtatttaccgagcacgtcgccgaccacgcgcgccgacttcaccggcttcgcgtcggggccgaggcccatttcgttcatcgcgaacaggatccggcgctgcaccggct

SEQ ID NO: 771 - DNA, 224 bp, Acinetobacter sp.
gagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcccgcgcagatcagttggaagaatttgttcactacgtgaaaggcgagat

SEQ ID NO: 772 - DNA, 224 bp, Stenotrophomonas maltophilia
gggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtg

SEQ ID NO: 773 - DNA, 224 bp, Shigella boydii
gccttttatggttcggtggctcgtctggcggcggaaagaggcattccggtttatgcgccggataacgttaatcatccgctgtgggtggaacgcattgcccaactgtcaccagaggtgattttctctttttattatcgccatcttatttgcgacgagattttgcagctcgctcccgcaggtgcatttaatctgcatggttcgctgttaccaaaatatcgtggtcg

SEQ ID NO: 774 - DNA, 224 bp, Acinetobacter baumannii
ctttatttatgtggtggagtggtgatttaagccatattaaaaatatatatcaacgtattttagcaaaaccttctttacttctattactcattacaacatccgttttgacttcggtgcagttatggctgtttttatggggacctatgcatggacgtggtttgcaagtttctttgggatatttcctgttaccgcttgttttggttttagcgggtagtgtcttatat

SEQ ID NO: 775 - DNA, 224 bp, Acinetobacter baumannii
aatgatgacaagctacatcattgaacaaaaattattaaaaggtgagcttaccgaaaatgaacaggttcgtatgaacgaatctgcttggtgtaaaggcagtagttctgaatcatgtatgtatgttccattaaatggcacagcaactgttttagaaatgcttcgcggtattattattcagtcaggtaatgatgcatcaaaagcaatggctgaacatattgctggga

SEQ ID NO: 776 - DNA, 224 bp, Acinetobacter baumannii
cttttatcatcaccacggtatttcaattcgtggtacagcacgtgctttggtaagtaacgtaactggaggacgtcgccaaggtggttcgaccctcacccaacagttagtcaaaaacttctatctaactcctgaacgcacacttaaacgtaaagttaatgaagcattaatggctttacttattgagttgcattacagcaaagatgaaattttagaagcatatttaa

SEQ ID NO: 777 - DNA, 224 bp, Acinetobacter baumannii
gtctcaaggaatttccgcgaaacaagattatcaacgagcttataacgcttatcagcaagcacaaattcaggttaaagcagctcgctcgcgcttaagtgcattcggggcaggttctggttcagcagggcgttataccttaacagcgccaattgcgggtatcgtgagtaataaagatattgtagtgggtgaaaacgtacagttagcagatcagctttttattatta

SEQ ID NO: 778 - DNA, 224 bp, Acinetobacter baumannii
cgctttgaaattctggccgctttatttaatgcacttatgctttttgtggtggcaatttatattttatatgaagcctatatccgcttttcgcagccacctgaaattcaaagtgtaggtatgctcattgtggcgaccattggtttggtaataaacctcatctcaatgaaaattcttatgtcgagtgccaataacagcttaaatgtgaaaggtgcttatctagaagt

SEQ ID NO: 779 - DNA, 224 bp, Acinetobacter baumannii
ggctgttttcgttgatgagctggaccaagtgccagatgattctattgtaattttcagtgcgcacggtgtttcaaaagcagttcagcaagaagcagagcgtcgaggcttaaaagtttttgatgcaacttgtccattggtgactaaagtacatattgaagtaactaaatatgctcgcgaagggacagaagctattttaattggtcatgaagggcacccagaagttg

SEQ ID NO: 780 - DNA, 224 bp, Acinetobacter baumannii
gtaacattgttttaattaaaattattgaccgtttagctttaggtaaaactgtcttaattggcttaccgattatgctcactggtacgctaattttaatattaggtgttgtctggcaagcctatttaattccttgcttgttaataggtatgacactcatttgttttggtgaaggaattagcttctcagtgttgtaccgttttgcgctcatgtcatctgaagtgtca

SEQ ID NO: 781 - DNA, 224 bp, Acinetobacter baumannii
tgccgacaacgcgtaatggacgcccatcttttacaagctgactaaaatcatattttaatgcatcagcttctacgatagttaaacgttctggatgtggcacgcgttcaggtaagccggcagctaaatcacggtctaattcaactacagttaatgcatcacattctccaattaaaggagaagttaaagcagctaaacctggaccaatttcgacgatattatcaccc

SEQ ID NO: 782 - DNA, 224 bp, Acinetobacter baumannii
ggataataccgcttgcatgttcaccttcaaaacgatttgcttgacccaagaaatcaaatacaaatggtgttgcaggtttttcgtaaacttcacgcggagaaccaatttgttcgacgttacctttattcatcacaataatttggtctgctacttcaagtgcctcttcttggtcatgggttacgaaaattgaagtgatatgtagctcatcatgcaagttacgtaac

SEQ ID NO: 783 - DNA, 224 bp, Acinetobacter calcoaceticus
atggttaaattattcgtttgcaacagcaaacttaagactggcagagcaaacgcttaaagcacagttagattcttacaatctcaacaaaaaacgttttgatgtaggtatcgacagtgaagttccattacgtcaagcacagatttctgtagaaactgcgcgtaatgatgtagcgaactacaaaactcaaattgctcaagcacaaaacttgttgaacttgcttgtag

SEQ ID NO: 784 - DNA, 224 bp, Escherichia coli
cagaggaactaatagcttataaaaagttagatggtgtgccagcaggtacgatagatcataacataggtaactatatttgggagatagacataaataatgttccagaattatttcacaagtcgctaggcagggtgttagcagagcttcatagcatacctagtaataaagccgcagcgcttgatcttgtagtacacacaccagaagaagcaagaatgtcaatgaag

SEQ ID NO: 785 - DNA, 224 bp, Salmonella enterica
gcagttcttccagaagactacagaaaattctgtagtcgttatgggattaaatacatggagatctctacctaagatgaagaagcttggtagagacttcattgtcatatcttcaactatcacagagcacgaagtgctcaacaataatatccaaatattcaaatcatttgagagcttcttagaagcattcagagacacaaccaaaccaatcaatgtcattggtggtg

SEQ ID NO: 786 - DNA, 224 bp, Acinetobacter baumannii
tgctggcgttcgtgcctgcgctcggccctatagccggtgcgctgatcggcgagttttggggatggcaggcgatcttcatcacactggctgcactggcttcgctcgcactcttaaacgccagtttcaggtggcatgaaacccgaccgttggatcaggccagaacgcaacgatctgttttgccgatcttcgcgagtccggccttttgggtttacacggtcggattt

SEQ ID NO: 787 - DNA, 224 bp, Shigella dysenteriae
acgacgaacaaagcggctgttaaagatggtcatcacaaacaggaaaacaatgttcagcgcaaagtaataaccaaaattttccggcgcgacgtggttaatttcaatgtagacaaacggtccggcgcttaagaatgagaacatcccggcaaagctgaaaccactggccagcatataactcaggacacgcttatggcggaacagtgccgcaaagttaccaatagtgg

SEQ ID NO: 788 - DNA, 224 bp, Enterococcus faecalis
aagcggttatcgctcattagaagaacagacagccatatatgacggctctctcaaagataatggagaggattttacaagaaaatatgttgctctgcccaatcatagtgaacatcaaacaggtcttgccattgatttaggactgaataaaaaggatatagactttatccgtcccgattttccctatgacggtatttgcgatgaatttaggagagctgccccagact

SEQ ID NO: 789 - DNA, 224 bp, Enterobacter cloacae
agctcgttcctgcccgatgaagaccagggcgtgttcctgagtatggcgcagctcccggccggtgcgacgcaagagcgtacccagaaagtgctggatgagatgaccgactacttcctgaccaaagagaaagacaacgtcgaatccgtgtttgcggttaacggctttggcttcgcgggtcgtggtcagaacactggtatcgccttcgtttctctgaaagactggtc

SEQ ID NO: 790 - DNA, 224 bp, Salmonella enterica
tcgttcgtgatctcctcgacgttaaaacaagaagatgtcataggcgctactgtcgttcctgatcttcgtgctgtgatcaacctgtatgagaataccgatcaacgcattgctgtcattggtggggagaagttgtacattcaagctctttcatcagcaacgaaactgcacatgaccataattccaagagagttcgactgtgatcgatttattcctgttgatccgat

SEQ ID NO: 791 - DNA, 224 bp, Serratia fonticola
tttaaaagctgtgatttatccatggcggattttcgcaacgtcagtgcgttgggcatagaaattcgccactgccgcgcacagggtgcagattttcgcggcgcaagtttcatgaatatgatcaccacgcgcacctggttttgcagcgcatatatcactaataccaatctaagctacgccaacttttcgaaggccgtgcttgaaaagtgcgaattgtgggaaaatcg

SEQ ID NO: 792 - DNA, 224 bp, Pseudomonas aeruginosa
ggccggcgacacgggattcgccgaccaggcgcaggtcgtaccaggaagccagctgggagggatcgaacttgcgcaccggccacagctgggcatcggccagttggtcggcaaggccgccgctgatgcactgggtgcgcccgtcgacccggaccacttcctggcgcgcgccgtcgagctggagcaggcgctcgcgaaccgcaccatcgctctccacgcgatgccag

SEQ ID NO: 793 - DNA, 224 bp, Artificial Sequence
  Description of Artificial Sequence Synthetic construct polynucleotide
ttcgacaaggcccgcccgggtcgccagcaatgctgccacgaaaaacccgagcagagggaaaagatagaagaggacggaggcgcgcaacaacaggtcttcatgaattcctaggacgaccgcatcgccgacccgcaggctcaggtcgctcaacgcgcgcacccgggcacgccccgcgccgacgcccaggcgctgcatcagcccctggccgcaaccggcattggccg

SEQ ID NO: 794 - DNA, 224 bp, Klebsiella variicola
taccgcgctggcgacgatccgcgggctcgacaccatgtatgtcgatctgacgcgatccagcgtcgatctcctgcgtttacgcaagcaaagcctggccagcaacagcgacaccctcagcgtgacgttaaccctggaagatggcagtacctatcccgaaaaagggcgtctggcgctcaccgaagtcgccgtcgatgaatctaccggctcggtgaccctgcgcgcta

SEQ ID NO: 795 - DNA, 224 bp, Enterobacter aerogenes
agtggaagaactccagcaacacgccgccgaccagcggaccgatcgcgctgcccagcgaaaacgcggcgatccacacgccgatggcgaactggcgctggcgcgggtcgtggaacatgttgcggaccagcgccatggtggacggcgcgatggtggcgccggccaggccgagcaaggcgcgcgccgcgatcaacagcgcggcggtatcggccagcgccgcgagcacc

SEQ ID NO: 796 - DNA, 224 bp, Serratia marcescens
cattgcatccgtgtggggctgtgatatccaccagggattgggggatgtcatcaccccctttgctagggaaaaagattgggattttacctttgccctgcaggatgtgctgtgtgcgccgcccgccgaagccggcgacctggcgctgatttttaagcttttgcccctgctggagcgggagcaggccggttctgccatggcacttttacaatccctcaataccccgc

SEQ ID NO: 797 - DNA, 224 bp, Unknown
  Description of Unknown Transposon Tn2921 polynucleotide
gacgctgcacgcccgctggaataccggggcctatctcacctgcggcgatctgtgggtctgcctgtcgtacgacgaggcgcgccagtacgtgccgccgcaggagagtgactacacccactacgcgtttaccgtggcggaagaggattttgaaccgctctcgcaaaggcttgagcaggcgggcgtcaccatctggaagcagaacaaaagcgagggggcgtcgttct

SEQ ID NO: 798 - DNA, 224 bp, Kluyvera ascorbata
atttgaggcaatgggggctctcccaaatagaaagtatgcagttgtcagccgctcaggatcggtagctactaacgatgatgtggttgtgtttccatctatagaagcagcaatgagggagctaaagactcttacgaaccatgttgttgtttctggtggtggagagatctacaagagtctgatcgcccatgccgacacgctacatatctcgacaatagattccgagc

SEQ ID NO: 799 - DNA, 224 bp, Escherichia coli
accaatggaatgaaaatgaaaaaataatatatcatgcatacgatattgatagagctgagatagcttttttgagtagcattattgggaagttaaagacgacgataaagtataggtttttgaataaagagagtgatgtctacaaaggtacttatgatgtagtattccttttaaagatgcttcctgtgctaaaacagcaagatgtaaatatcttggatttcctacag

SEQ ID NO: 800 - DNA, 224 bp, Artificial Sequence
  Description of Artificial Sequence Synthetic construct polynucleotide
cgaagggggtgagacggacgatggccgagtcgaccttgggcggcgggttgaacgcgccggggccgacgttgaacaggtgctccacccgacagtgatactggaccatgatcgagagccggccccagtctccgccgcccggcgtggccgccaggcgctccaccacttccttctgcagcatgaagtgcatgtcctcgatcaccggcgcgtgctccagcagatggaag

SEQ ID NO: 801 - DNA, 224 bp, Shigella boydii
ccaatgtgaaggtctggcaaaaaccaagccgggtatggtaatgggctttatcggcctgctggtgaacattccggtgaactatatctttatttatggtcatttcggtatgcctgagctcggtggcgttggttgtggcgtggctactgcggcggtgtattgggtcatgttccttgccatggtttcttacattaaacgcgcccgctccatgcgcgatattcgtaacg

SEQ ID NO: 802 - DNA, 224 bp, Shigella dysenteriae
cggtaactatccgcgtaggttatcgtctgggtcagggctcaacgctggatgcgcaaaccgctgcgcggaccgggcttatggtgggtgtctgtatggcaaccctgacggccatattcacggtttcactgcgggagcaaatcgccctgttgtacaacgacaatcccgaggttgtaacgctggctgcgcatttgatgttgctggcggcggtatatcagatttctgac

SEQ ID NO: 803 - DNA, 224 bp, Clostridium sticklandii
aggtattatggggattttatttgatgattatttggatgcttacctccaaacccccattgtgatttcaatcatgttaatcttttatggtttgttattcattctcatagaaaattggaataaaaagcgtactcctactactatggcactttctgacatcagctataaaacagcaatcttgataggggcatttcaagtgttatcacttatacccggcacatcacggt

SEQ ID NO: 804 - DNA, 224 bp, Haemophilus influenzae
caatttgattttggtgcgctttacacaaaagaaaatttagcagaaaaagggcaaaaattacgtgtgtttggtaacttaccttacaacatttctacgccattgatgttccacttgtttaaatatcatgatgtgattcaagatatgcactttatgctgcaaaaagaagtggtaaaacgcctatgtgccgcaccaaatagcaaagcttatggtcgtttaacgattat

SEQ ID NO: 805 - DNA, 224 bp, Neisseria meningitidis
gaaggcggagaatgcgattgatgacgccatcgtcagggcaaactgtttgtaaatattgcccgtcgccccgctgaacatcgccaacggtacgaacacggaaatcagaacggcggtaataccgatgaccgcgcccgaaatctgacccatcgcttttttggtcgcttctttgggcggcaagccttcacccgccataatgcgctcgacgttttcaaccaccacaatcg

SEQ ID NO: 806 - DNA, 224 bp, Delftia tsuruhatensis
caactcttgcaccgttcggatgtctgtgcctgcttgcagcaagtgggtggcgaatgagtggcgcagggtgtggacagatacgtgtttggcaatgccagcctgaactaccgctttttttagttgccggttcagtctttcctcaaacaagtggtggcggcgctcaacgccggtttgtgggtccacagacagcttggccgatggaaacacccagaaccaggcccagc

SEQ ID NO: 807 - DNA, 224 bp, Acinetobacter calcoaceticus
gtaggtttagcattgggttataacggcggtgacatttcttggtctgatgatttatcaatcaatggtaccaagtatgatatggatatggacaataaattagcatatctgaatgctgaaattcgcccttggggtgctagcacgaatccatgggctcaaggtttatatgtagctgcaggtgcagcttatgttgataaccaatatgatttaacaaaaaatgtaggtac

SEQ ID NO: 808 - DNA, 224 bp, Haemophilus parainfluenzae
gatgatgtacttgaaattgttcaaggcccagattttccaacagaagcggaaattatttcgccaaaatcagaaattcgtaaaatttatgagcaaggtcgtggctcaataaaaatgcgtgcaacttggaaaaaagaagacggtgaaattattatttctgcgcttccacatcaatcttcgccatccaaagttattgcacaaatagccgaacaaatgacggcaaaaaa

SEQ ID NO: 809 - DNA, 224 bp, Escherichia fergusonii
gccccccgaccaatcaccagcacatcaccaccttctaacgtggcgtggtcataattaatattctcgtcgccgaaatatttaataaattcgccgccagcaaattgtggatgccagcgataaatagcgcgtagattattggtttcacgttggcgagcaggttttgccattggattaatcgatacaccgttataaatccagcatgaggtatcacgcgtaaataaatg

SEQ ID NO: 810 - DNA, 224 bp, Streptococcus pneumoniae
gtttcaggctatccttccaattcgcggtaagattttgaacgttgaaaaagcaagtatggataagattctagctaacgaagaaattcgtagtcttttcacagccatgggaacaggatttggcgcagaatttgatgtttcgaaagcccgttaccaaaaactcgttttgatgaccgatgccgatgtcgatggagcccacattcgtacccttcttttaaccttgattt

SEQ ID NO: 811 - DNA, 224 bp, Corynebacterium urealyticum
tcagcacgtatcgttggtgacgtaatgggtaaatatcaccctcatggtgacttatctatttatgaagcaatggtacgtatggctcaagatttcagttatcgttatccgcttgttgatggccaaggtaactttggttcaatggatggagatggcgcagcagcaatgcgttatactgaagcgcgtatgactaaaatcacacttgaactgttacgtgatattaataa

SEQ ID NO: 812 - DNA, 224 bp, Burkholderia vietnamiensis
cgtgtgtacacgacgacgccgaagaagccgaactcggcactccgtaaggttgccaaggttcgtctgacgaacggcttcgaagtgatttcgtacatcggcggtgaaggccacaacctgcaggaacactcggttgtgctgatccgcggcggccgtgtgaaggacttgccgggtgtgcgttaccacatggttcgcggctcgctggatacccagggcgtcaaggaccg

SEQ ID NO: 813 - DNA, 224 bp, Burkholderia cenocepacia
caagcagaagtacggccgcaacatctcgtgggccgacctgctgatcctgaccggcaacgtcgcgctcgaatcgatgggcttcaagacgttcggctatgccggcggccgcgccgacacgtgggaaccggacgacgtctactggggctcggaaaagatctggctggaactgagcggcggcccgaacagccgctacacgggcaagcgcgagctcgaaagcccgctcg

SEQ ID NO: 814 - DNA, 224 bp, Klebsiella variicola
accctggtctcgcagaagacggtgtacgtctactttgacgtcgacgagtcaacctacctccactatcaaaacctcgcccgccgcgggcaaggcgcgtccagcgataatcaggcgctcccggtggagattggcctggttggcgaggagggttacccccaccagggcaaagtggattttctcgataatcagttaacgccgagtaccggcaccatccgcatgcgtgc

SEQ ID NO: 815 - DNA, 224 bp, Serratia marcescens
acgaaggcggcatcaaggcgtttgtcgagtacctgaacaagaacaaaaccccgatccacccgaacgtgttctacttctccaccgtgaaagacgacatcggcgtggaagtggcgctgcagtggaacgacggtttccaggaaaacatctactgcttcaccaacaacattccgcagcgcgacggcggcacgcacctggtcggtttccgtaccgcgatgacccgtacg

SEQ ID NO: 816 - DNA, 224 bp, Streptococcus pneumoniae
tgtttaatgtcgtgattcagcttggtgctattttagcagttatggtgatttattttaacaagctcaatccttttaaaccgactaaggacaaacaggaagttcgtaagacttggagactatggttgaaggtcttgattgctactttacctttacttggtgtctttaaatttgatgattggtttgatacccacttccataacatggtttcagttgctctcatgttg

SEQ ID NO: 817 - DNA, 224 bp, Enterobacter sp.
gcgatatcgggcgtacacagcggcgcgagcggcgcatgacaaagaaattcagcctcggtgccgtgccacgccccgccaccgtagcggatcgtatagtcaagcccttcggcagccggatcaacgcggttgttatgggtggaaagctgaagatcgatgtgcggatagccacggcgaaaatcctccagctgcgagaataaaaccccggtggcaaacgtacccaccac

SEQ ID NO: 818 - DNA, 224 bp, Enterobacter aerogenes
aggccgacggctcggtggtgcgcctgagcgatgtcgcgcgggtggagatgggcagcgaggattacaccgccaccgctaatctgaacggccacccggccgccgggatcgcggtgatgatggcccccggcgccaacgcgctggacaccgcgacgctggtgaaaagcaaaatcgccgaattccagcgtcagatgccgcagggttacgatattgcctatccgaaggac

SEQ ID NO: 819 - DNA, 224 bp, Pseudomonas aeruginosa
attgcccatctgggtggtcccgataaagtgacggcgtttgctcgctcgttgggtgatgagaccttccgtctggacagaaccgagcccacgctcaataccgccattccaggcgacccgcgtgataccaccacgccgctcgcgatggcgcagaccctgaaaaatctgacgctgggtaaagcgctggcggaaactcagcgggcacagttggtgacgtggcttaaggg

SEQ ID NO: 820 - DNA, 224 bp, Haemophilus influenzae
aggttttgcaaatacagtgacaaattttgcattagggtacatttgacgaatcgcacgcgcagtattacccgtatcaactaaatcatctaccacgataaatccttcgccgccatttggcacttgggctgcatgaagaacttgtagttcaccttgattgttatgatcgtgataacttgcgatacaaacggtttcaatatgacgaagacccaattctctagccaata

SEQ ID NO: 821 - DNA, 224 bp, Burkholderia ambifaria
gcagcatgaaatgctggtcgatgaccgcatcggcgaacgtcatcaggtgaaacagcagcgggctggaaatgttgtacggcaggttgccgacgatccgcagcgacggcttgtcgccgggcgccgcgagcgagcggaagtcgaacgcgagcgcgtcgcccgcgtgcagctccagcagcgcgccgaagcgttgctgcaggcgcccgatcaggtcgcggtcgagctcg

SEQ ID NO: 822 - DNA, 224 bp, Klebsiella oxytoca
ccgcaccgtcggtgacgtgctgggtaaataccatccgcacggcgacagcgcctgctatgaagccatggtgctgatggcgcagcccttctcttaccgctatccgctggttgacggtcagggaaactggggggcgccggacgatcctaaatccttcgccgcaatgcgttataccgaatcccgtttgtcgaagtatgctgaactgctgctgagcgaactggggcaag

SEQ ID NO: 823 - DNA, 224 bp, Neisseria gonorrhoeae
gagctgcacgagcttgagcaattcttcgactttggcgcggatttgtcctttggacgggcgttcggacttgggcaatacggtcaaaccgaaagcgacgttgtcaaacacgttcatatggcggaaaagggcgtagtgttggaacacgaagccgactttgcgctcgcgcacatgtttggcggttacgtcttgcccgtcaaacagaatattgccgccgtcggcgtttt

SEQ ID NO: 824 - DNA, 224 bp, Bordetella parapertussis
gaaggtgcccatcaatgccactggcacgaccagggtcgggatcagcgtgtagcgcaggttctgcaggaacaggaacatgactgcgaacaccagcagcatggcttcgaacagggtgttgatgacctgctcgatcgacaccttgacgaacggcgaggtgtcgtaggggatgtcgtacttgacgccttccgggaagtagcgcgccaggtcctgcatcttcgcccgta

SEQ ID NO: 825 - DNA, 224 bp, Shigella dysenteriae
aacgacgtggctggcgcgccctctgccgttaaatggatgtatgccattgaagcgtgtctgtcgttaacgttgctctaccctatcgcccgctggagtgaaaagcattttcgtctggaacaccggttgatggctgggctgttgataatgtcattaagcatgatgccggtgggcatggtcagcggcctgcaacaacttttcaccctgatttgtctgttttatatcgg

SEQ ID NO: 826 - DNA, 224 bp, Shigella sonnei
ggtggaacaatatcaggcgggcattgattgggttcctacttcgatgaccgcgtatctggcgggcgggatgtttttacaatggctcctggggccgctgtcggatcgtattggtcgccgtccggtgatgctggcgggagtggtgtggtttatcgtcacctgtctggcaatattactggcgcaaaacattgaacaattcaccctgttgcgcttcttgcagggcataa

SEQ ID NO: 827 - DNA, 224 bp, Shigella dysenteriae
gcgagcagttgagcagctatgaatatggcttgctgcaagtgcctattttcggggcgttaattgcgggtaacttgctgttagcgcgtctgacctcgcgccgcaccgtacgttcgctgattattatgggcggctggccgattatgattggtctgttggtcgctgctgcggcaacggttatctcatcgcacgcgtatttatggatgaccgccgggttaagtatttat

SEQ ID NO: 828 - DNA, 224 bp, Shigella sonnei
gcaccgctaatcggcggtctgctggatacgatgtggaactggcgcgcctgttatctctttttgttggttctttgtgccggtgtgaccttcagtatggcccgctggatgccggaaacgcgtccggtcgacgcaccgcgcacgcgcctgcttaccagttataaaacgcttttcggtaacagcggttttaactgttatttgctgatgctgattggcggtctggccgg

SEQ ID NO: 829 - DNA, 224 bp, Serratia fonticola
gctgaaagatgccatttttaaaagctgtgatttatccatggcggattttcgcaatgccagtgcgctgggcattgaaattcgccactgccgcgcacaaggcgcagatttccgcggcgcaagctttatgaatatgatcaccacgcgcacctggttttgtagcgcatatatcactaataccaatctaagctacgccaatttttcgaaagtcgtgttggaaaagtgtg

SEQ ID NO: 830 - DNA, 224 bp, Unknown
  Description of Unknown Centisome 49 polynucleotide
tttattatggcagcgtggatggtcatcagtgcgctgctggggctgggattctggtcgctggtggttggcgttgcggcgtttgtgggctgcgtgtcgatggtgtcatccaatgcgatggcggtcattcttgatgagtttccccatatggcgggaacggcatcttcgctggcaggaaccttccgttttggcataggggcaattgttggcgcattgctttctcttgc

SEQ ID NO: 831 - DNA, 224 bp, Shigella sonnei
gccggggcaaaaagcctggtttacggtacttggcgatccactgacgcgctacgaggggcaaatcaaggatgtactaccgacgccggaaaaggttaacgacgctattttctattacgcccgttttgaagtccccaaccccaatggtttgctgcggctggatatgactgcgcaagtgcatattcagctcaccgatgtgaaaaatgtgctgacgatccctctgtcgg

SEQ ID NO: 832 - DNA, 224 bp, Shigella sonnei
ctggaaatggcgaccgttcgcgcgccgtttgatggccgggtcatttccctcaaaacctccgtcgggcaatttgcttctgccatgcgccctatttttaccctaatcgacactcgtcactggtacgtgatcgccaacttccgcgaaaccgatctgaaaaatattcgctcaggtacacccgcaacgattcgcctgatgagtgacagcggcaaaaccttcgagggtaa

SEQ ID NO: 833 - DNA, 224 bp, Streptococcus salivarius
tattgggtgctgtgattgcatctattacggtagcaattgtacgtatacctaagctgggtaatcaagtgcaaagtttagaaccaaatttcataagggagatgaaagaaggagttgtggttctgagacaaaacaaaggattgtttgccttattactcttaggaacactatatacttttgtttatatgccaatcaatgcactatttcctttaataagcatggaacac

SEQ ID NO: 834 - DNA, 224 bp, Escherichia coli
ctctccagtatgaacaggaaagaatatgctatgaacccttatatttatcttggtggtgcaatacttgcagaggtcattggtacaaccttaatgaagttttcagaaggttttacacggttatggccatctgttggtacaattatttgttattgtgcatcattctggttattagctcagacgctggcttatattcctacagggattgcttatgctatctggtcagg

SEQ ID NO: 835 - DNA, 224 bp, Stenotrophomonas maltophilia
gcgttttagcgcaagagtccgtcactccatacattgcaatgctgaatggagagccgattgggtatgcccagtcgtacgttgctcttggaagcggggacggatggtgggaagaagaaaccgatccaggagtacgcggaatagaccagtcactggcgaatgcatcacaactgggcaaaggcttgggaaccaagctggttcgagctctggttgagttgctgttcaat

SEQ ID NO: 836 - DNA, 224 bp, Streptococcus gallolyticus
tgttttagatggggccatcttggtgatctccgctaaagatggcgtgcaggcccagacccgtattctgttccatgccctgcggaaaatgaacattcccaccgttatctttatcaacaagatcgaccaggctggcgttgatttgcagagcgtggttcagtctgttcgggataagctctccgccgatattatcatcaagcagacggtgtcgctgtccccggaaatag

SEQ ID NO: 837 - DNA, 224 bp, Escherichia coli
agtagtgctctgcggcgcagtgctggcgcgggtggatgccggtgacgaacagctggagcgaaagatccactatcgccagcaggatctggtggactactcgccggtcagcgaaaaacaccttgccgacggcatgacggtcggcgaactctgcgccgccgccattacgccaactctggccgcgcattaccatgagcgataacagcgccgccaatctgctacatgag

SEQ ID NO: 838 - DNA, 224 bp, Proteus mirabilis
aactgaatgaggcgcttcccggcgacgcccgcgacaccactaccccggccagcatggccgcgaccctgcgcaagctgctgaccagccagcgtctgagcgcccgttcgcaacggcagctgctgcagtggatggtggacgatcgggtcgccggaccgttgatccgctccgtgctgccggcgggctggtttatcgccgataagaccggagctggcgagcggggtgcg

SEQ ID NO: 839 - DNA, 224 bp, Enterobacter cloacae
accgctgggaaacggaactgaatgaggcgcttcccggcgacgcccgcgacaccactaccccggccagcatggccgcgaccctgcgcaagctgctgaccagccagcgtctgagcgcccgttcgcaacggcagctgctgcagtggatggtggacgatcgggtcgccggaccgttgatccgctccgtgctgccggcgggctggtttatcgccgataagaccggagct

SEQ ID NO: 840 - DNA, 224 bp, Klebsiella pneumonia
tgaatgaggcgcttcccggcgacgcgcgcgacaccaccaccccggccagcatggccgccacgctgcgcaaactactgaccgcgcagcatctgagcgcccgttcgcaacagcaactcctgcagtggatggtggacgatcgggttgccggcccgctgatccgcgccgtgctgccgccgggctggtttatcgccgacaaaaccggggctggcgaacggggtgcgcgc

SEQ ID NO: 841 - DNA, 224 bp, Streptococcus pneumoniae
atattatttcatgcacttaggaaaatggggattcccacaatcttttttatcaataagattgaccaaaatggaattgatttatcaacggtttatcaggatattaaagagaaactttctgccgaaattgtaatcaaacagaaggtagaactgtatcctaatatgtgtgtgacgaactttaccgaatctgaacaatgggatacggtaatagagggaaacgatgacct

SEQ ID NO: 842 - DNA, 224 bp, Staphylococcus aureus
caagaaaagtatcatgtggagatagaactaaaagagcctacagtcatttatatggagagaccgttaaaaaatgcagaatataccattcacatcgaagtgccgccaaatcctttctgggcttccattggtttatctgtatcaccgcttccgttgggaagtggaatgcagtatgagagctcggtttctcttggatacttaaatcaatcatttcaaaatgcagttat

SEQ ID NO: 843 - DNA, 224 bp, Pseudomonas aeruginosa
gctcggagcgctcgtcgacatgtggcttgggtggcgggctatctttgcgtttctaggtttgggcatgatcgctgcatctgcagcagcgtggcgattctggcctgaaacccgggtgcaacgagttgcgggcttgcaatggtcgcagctgctactccccgttaagtgcctgaacttctggttgtacacgttgtgttacgccgctggaatgggtagcttcttcgtct

SEQ ID NO: 844 - DNA, 224 bp, Pseudomonas aeruginosa
tcgaccatgttgctggaacggtcacggcagtctacttctgcttgggcggtgtactgctaggaagcatcggaacgttgatcatttcgctgttgccgcgcaacacggcttggccggttgtcgtgtactgtttgacccttgcaacagtcgtgctcggtctgtcttgtgtttcccgagtgaagggctctcgcggccagggggagcatgatgtggtcgcgctacaaagt

SEQ ID NO: 845 - DNA, 224 bp, Klebsiella pneumoniae
cagtagtgtcaaagaacggaatttcaagctcaaatgaaaacgtcctagtttttccttcaatagaaaatgctttgaaagagctatcaaaagttacagatcatgtatatgtctctggcgggggtcaaatctataatagccttattgaaaaagcagatataattcatttgtctactgttcacgttgaagtcgaaggtgatatcaaattccctataatgcctgagaat

SEQ ID NO: 846 - DNA, 224 bp, Stenotrophomonas maltophilia
ggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagcca

SEQ ID NO: 847 - DNA, 224 bp, Serratia marcescens
cccgggtggatgccggagacgaacagctggatcggcggatccactaccgccagcaggatctggtggactactccccggtcagcgaaaaacaccttgccgacgggatgaccgttggcgaactctgcgccgccgccatcaccatgagcgacaacagcgccggcaatctgctgttgaagagcgtcggcggccccgcgggattgaccgcttttctgcgccagatcggt

SEQ ID NO: 848 - DNA, 224 bp, Shigella dysenteriae
gcaggcggcttaacgacctcgcagttcgcctggacacaggttccggtgttcggcgcggtgattgtcgcgaatgccatcgtggcgcgtttcgttaaagatccgaccgaaccgcgctttatctggcgtgccgtacccattcaactggtcggcctcgcgctgttgattatcggcaatctgctgtcaccgcacgtctggctgtggtcggtgctgggcaccagtctgta

SEQ ID NO: 849 - DNA, 224 bp, Klebsiella oxytoca
aaaaagtcaccgccttcgcccgcagtatcggcgatgccacctttcgtctcgatcgtacggagcccacgctgaataccgccatcccgggcgatgagcgtgataccagcacgccgctggcgatggctgaaagcctgcgcaagctgacgcttggcgatgcgctgggcgaacagcaacgcgcccagttagtcacctggctgaaaggcaataccaccggcgggcaaagc

SEQ ID NO: 850 - DNA, 224 bp, Escherichia coli
ctggctaggctgttttcttaccggtgccgccttcagtctggtaatgcccttcttacccctctacgttgagcagcttggcgttaccggtcactccgccctgaatatgtggtccggtattgtcttcagcattacatttttattttcggccatcgcctcaccgttttggggtggactcgccgaccgtaaaggccgaaaactcatgctattacgctctgctctcggca

SEQ ID NO: 851 - DNA, 224 bp, Escherichia coli
gcaccacgactcggcaaacttggcgatcgaatcggacccgaaaagatcctgattacagcgctgatcttttctgtactgctgttgatcccaatgtcttacgttcagacgccattgcaacttgggattttacgttttttgctcggtgccgccgatggtgcactactccccgccgtacagacactgttggtttacaactcgagcaaccagatcgccgggcgtatctt

SEQ ID NO: 852 - DNA, 224 bp, Escherichia coli
tgagcatgaccttgcgatgctctatgagtggctaaatcgatctcatatcgtcgagtggtggggcggagaagaagcacgcccgacacttgctgacgtacaggaacagtacttgccaagcgttttagcgcaagagtccgtcactccatacattgcaatgctgaatggagagccgattgggtatgcccagtcgtacgttgctcttggaagcggggacggatggtggg

SEQ ID NO: 853 - DNA, 224 bp, Shigella sonnei
aaactggtgtcataaagaatgagcatcaggttttcaaatgggacggaaagccaagagccatgaagcaatgggaaagagacttgaccttaagaggggcaatacaagtttcagctgttcccgtatttcaacaaatcgccagagaagttggcgaagtaagaatgcagaaataccttaaaaaattttcctatggcaaccagaatatcagtggtggcattgacaaattc

SEQ ID NO: 854 - DNA, 224 bp, Mycobacterium bovis
cactgacctctcgctgcgggtggatccgatctatgagcggatcacgcgtcgctggctggaacaccccgaggaattggccgacgagttcgccaaggcctggtacaagctgatccaccgagacatgggtcccgttgcgagataccttgggccgctggtccccaagcagaccctgctgtggcaggatccggtccctgcggtcagccacgacctcgtcggcgaagccg

SEQ ID NO: 855 - DNA, 224 bp, Shigella flexneri
tacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagt

SEQ ID NO: 856 - DNA, 224 bp, Vibrio splendidus
gcactcctaaatgcgggtttcaggtggcacgaaacccgccctctggatcaagtcaagacgcgccgatctgtcttgccgatcttcgcgagtccggctttttgggtttacactgtcggctttagcgccggtatgggcaccttcttcgtcttcttctcgacggctccccgtgtgctcataggccaagcggaatattccgagatcggattcagctttgccttcgccac

SEQ ID NO: 857 - DNA, 224 bp, Escherichia coli
ggctttactaagcttgccccttccgccgttgtcataatcggttatggcatcgcattttattttctttctctggttctgaaatccatccctgtcggtgttgcttatgcagtctggtcgggactcggcgtcgtcataattacagccattgcctggttgcttcatgggcaaaagcttgatgcgtggggctttgtaggtatggggctcataattgctgcctttttgct

SEQ ID NO: 858 - DNA, 224 bp, Serratia marcescens
cctcacatttccatagcgacagcacgggtggaatagagtggcttaattctcaatctattcccacgtatgcatctgaattaacaaatgaacttcttaaaaaagacggtaaggtacaagctaaaaactcatttagcggagttagctattggctagttaaaaataaaattgaagttttttatcctggcccagggcacactcaagataacgtagtggtttggttacct

SEQ ID NO: 859 - DNA, 224 bp, Neisseria meningitidis
tgtaatttcgtaactgccattgaaatagaccataaattatgcaaaactacagaaaataaacttgttgatcacgataatttccaagttttaaacaaggatatattgcagtttaaatttcctaaaaaccaatcctataaaatatttggtaatataccttataacataagtacggatataatacgcaaaattgtttttgatagtatagctgatgagatttatttaat

SEQ ID NO: 860 - DNA, 224 bp, Klebsiella pneumoniae
ccgggtgagcagaagatttttcgcagactcactgagggaaaagtcgttgtcatggggcgaaagacctttgagtctatcggcaagcctctaccgaaccgtcacacattggtaatctcacgccaagctaactaccgcgccactggctgcgtagttgtttcaacgctgtcgcacgctatcgctttggcatccgaactcggcaatgaactctacgtcgcgggcggagc

SEQ ID NO: 861 - DNA, 224 bp, Ruminococcus sp.
tgcatggacaaatcactggcctacattcttacaaaaaatgcgggcatcgccgttcccgaatttcaaatgattgataaaggtgacaagccggaggcgggtgcgcttacctaccctgtctttgtgaagccggcacggtcaggttcgtcctttggcgtaaccaaagtaaacggtacggaagaacttaacgctgcgatagaagcggcaggacaatatgatggaaaaat

SEQ ID NO: 862 - DNA, 224 bp, Shigella flexneri
tccgtatggcgcagccattctcgctgcgttacatgctggtagacggtcagggtaacttcggttccatcgacggcgactctgcggcggcaatgcgttatacggaaatccgtctggcgaaaattgcccatgaactgatggccgatctcgaaaaagagacggtcgatttcgttgataactatgacggcacggaaaaaattcccgacgtcatgccaaccaaaattcct

SEQ ID NO: 863 - DNA, 224 bp, Pseudomonas aeruginosa
ccgttcttgatactcaaaatggagaatattgggattacaatggcaatcagcgcttcccgttaacaagtacttttaaaacaatagcttgcgctaaattactatatgatgctgagcaaggaaaagttaatcccaatagtacagtcgagattaagaaagcagatcttgtgacctattcccctgtaatagaaaagcaagtagggcaggcaatcacactcgatgatgcg

SEQ ID NO: 864 - DNA, 224 bp, Artificial Sequence
  Description of Artificial Sequence Synthetic plasmid pB10-2 polynucleotide
cagcaatgcggaattctactgtttgggtgtatgagctatttgcaaaggaaattggtgatgacaaagctcggcgctatttgaagaaaatcgactatggcaacgccgatccttcgacaagtaatggcgattactggatagaaggcagccttgcaatctcggcgcaggagcaaattgcatttctcaggaagctctatcgtaacgagctgccctttcgggtagaacat

SEQ ID NO: 865 - DNA, 224 bp, Enterobacter hormaechei
aacccgttacgcctcagaccctgttcgagctgggttctataagtaaaaccttcaccggcgttttaggtggggatgccattgctcgcggtgaaatttcgctggacgatccggtgaccagatactggccacagctgacgggcaagcagtggcagggtattcgtatgctggatctcgccacctacaccgctggcggcctgccgctacaggtaccggatgaggtcacg

SEQ ID NO: 866 - DNA, 224 bp, Klebsiella oxytoca
ccgccgcggtgttaaaacagagcgaaagcaatccagaggtggtgaataaaaggctggagattaaaaaatcggatttagtggtctggagcccgattaccgaaaaacatctgcaaagcggaatgaccctggcggaactcagcgcggcggcgctgcagtacagcgacaataccgcgatgaataagatgattagctaccttggcggaccggaaaaggtgaccgcattc

SEQ ID NO: 867 - DNA, 224 bp, Shigella sonnei
tcttcaatcaccaccggcggcataacatgggcattttccggtaaggtgatcaaagccttacctgcacggttacgcgccaccagatcgttaaaggtgcagacgaaaccgtaacccgcatcggaagccatcagcagtttctgatcgtcgctttccatcagcatatggtcaacggtcgccccaggcggcaacgttaatttgccggtgagcggttcgccctgaccacg

SEQ ID NO: 868 - DNA, 224 bp, Shigella sonnei
ctcgctcaatagcagctcggaatatttcgacaaccgggattcggtgtaacgcattgccgcgaacgatttcggatcgtccggcgcgccccagttcccctgaccatcaaccagcggataacggtaagagaacggctgcgccatcaggaccatcgcttcataacaggcgctatcgccgtgcggatggtatttacccagtacgtcaccgacggtacgggccgattttt

SEQ ID NO: 869 - DNA, 224 bp, Unknown
  Description of Unknown blaIMP=metallo beta- lactamase polynucleotide
cgggcggaatagagtggcttaattctcgatctatccccacgtatgcatctgaattaacaaatgaactgcttaaaaaagacggtaaggttcaagccacaaattcatttagcggagttaactattggctagttaaaaataaaattgaagttttttatccaggcccgggacacactccagataacgtagtggtttggttgcctgaaaggaaaatattattcggtggt

SEQ ID NO: 870 - DNA, 224 bp, Artificial Sequence
  Description of Artificial Sequence Synthetic cloning vector polynucleotide
ttgggataagttagagccactttatacaatttttgatggtgtatctaaaacattctctggtatttggactcctgtaaagaatgacttcaaagagttttatgatttatacctttctgatgtagagaaatataatggttcggggaaattgtttcccaaaacacctatacctgaaaatgctttttctctttctattattccatggacttcatttactgggtttaact

SEQ ID NO: 871 - DNA, 224 bp, Burkholderia multivorans
gcccgagccggtgcggcgcgacggcatccaggtcgagaaggccgccgacaacatccagctcgtcgtgtcgctgacctcggacgacggccggatgaccgacgtgcagctcggcgaatacgcgtcggcgaacgtcgtgcaggcgctgcgccgcgtcgacggcgtcggcaaggtgcagttctggggcgccgaatacgcgatgcggatctggcccgacccggtgaagc

SEQ ID NO: 872 - DNA, 224 bp, Leptothrix cholodnii
atgaacaaggtcgtcacgacgctgatcgaggccggcgtgctggtgttcctcgtgatgttcctgttcatgcagaacctgcgcgcgacgctgattccgacgctcgtcgtgccggtcgcgctcgcgggcacgttcggcgtgatgtatgcggccggcttctcgatcaacgtgctgacgatgttcgggatggtgctcgcgatcggcatcctcgtcgacgatgcgatcgt

SEQ ID NO: 873 - DNA, 224 bp, Burkholderia ambifaria
ggcgagatggtgccgctcgcggcgttcgcgacgctgcactggacgctcgggccgccgcagctcacgcgctacaacggctatccgtcgttcacgatcaacggctcggccgcgcccggccacagcagcggcgaagcgatggccgcgatcgaacggatcgccgcgaagctgccggccggcatcggccatgcgtggtcggggcagtcgttcgaggagcggctgtcggg

SEQ ID NO: 874 - DNA, 224 bp, Staphylococcus epidermidis
ttaaagtatgtcaaagtaagatgcagttctacaaaaagcatataacaattagttatagttcaaacattgataatccgatggtatttggtttagtgaaatcccaaattgtactaccaactgtcgtagtcgaaaccatgaatgacaaagaaattgaatatattattctacatgaactatcacatgtgaaaagtcatgacttaatattcaaccagctttatgttgtt

SEQ ID NO: 875 - DNA, 224 bp, Oligella urethralis
aacagatcaacaagttttaacttttttcaaagactggaaacctaaaaacccaatcggtgaatacagacaatattcaaatccaagtattggcttatttggaaaagttgtagctttgtctatgaataaacctttcgaccaagtcttagaaaaaacaatttttccggcccttggcttaaaacatagctatgtaaatgtacctaagacccagatgcaaaactatgctt

SEQ ID NO: 876 - DNA, 224 bp, Klebsiella pneumoniae
gggcgccaacttttcccgggcccgcttctacaatcaagtcagccataagatgtacttctgctcggcttatatctcaggttgcaacctggcctataccaacttgagtggccaatgcctggaaaaatgcgagctgtttgaaaacaactggagcaatgccaatctcagcggcgcttccttgatgggctcagatctcagccgcggcaccttctcccgcgactgttggc

SEQ ID NO: 877 - DNA, 224 bp, Klebsiella pneumoniae
tggcatttgatgcggaaataatagatcagaaaaccatattcaaatgggataaaacccccaaaggaatggagatctggaacagcaatcatacaccaaagacgtggatgcaattttctgttgtttgggtttcgcaagaaataacccaaaaaattggattaaataaaatcaagaattatctcaaagattttgattatggaaatcaagacttctctggagataaagaa

SEQ ID NO: 878 - DNA, 224 bp, Neisseria gonorrhoeae
agagatgaaggaaatgccgtctgccgcacaattggaacgcctgtccgagcttgtcgatgtgccggttgatgttttgaggaacaagctcgaacagaaaggcaagtcgtttatctggattaagcggcagctcgatcccaaggttgccgaagaggtcaaagccttgggtttggaaaactttgtatttgaaaaagaattaaaacgccattacccgatgggcaacctgt

SEQ ID NO: 879 - DNA, 224 bp, Neisseria meningitidis
atgaagagttgaacaaggcggtcgaataccatcaggcaaaagccggaacggtggtggttttggatgcccgcacgggggaaatcctcgccttggccaatacgcccgcctacgatcccaacaggcccggccgggcagacagcgaacagcggcgcaaccgtgccgtaaccgatatgatcgaacccggttcggcaatcaaaccgtttgtgattgcgaaggcattggat

SEQ ID NO: 880 - DNA, 224 bp, Neisseria gonorrhoeae
gcagacggtaacgtataactttttgaaagaacagggcgacaaccggattgtgcggactcaaacattgccggctacacgcggtacggtttcggaccggaacggtgcggttttggcgttgagtgcgccgacggagtccctgtttgccgtgcctaaagagatgaaggaaatgccgtctgccgcacaattggaacgcctgtccgagcttgtcgatgtgccggttgatg

SEQ ID NO: 881 - DNA, 224 bp, Neisseria gonorrhoeae
gttttgcgggaccggcagggcaatattgtggacagcttggactccccgcgcaataaagccccgaaaaacggcaaagacatcatcctttccctcgatcagaggattcagaccttggcctatgaagagttgaacaaggcggtcgaataccatcaggcaaaagccggaacggtggtggttttggatgcccgcacgggggaaatcctcgccttggccaatacgcccgc

SEQ ID NO: 882 - DNA, 224 bp, Neisseria meningitidis
ggactgtatctgcagacggtaacgtataactttttgaaagaacagggcgacaaccggattgtgcggactcaaacattgccggctacacgcggtacggtttcggaccggaacggtgcggttttggcgttgagtgcgccgacggagtccctgtttgccgtgcctaaagagatgaaggaaatgccgtctgccgcacaattggaacgcctgtccgagcttgtcgatgt

SEQ ID NO: 883 - DNA, 224 bp, Neisseria meningitidis
ggtacggcgcgtaagttggttaacggtcgttacgtcgattacaaacacgttgccactttcatcggttttgccccggctaaaaatccgcgtgtgattgtggcggtaaccattgacgagccgactgcaaacggttactacggcggcgtagtgacaggtccggtcttcaaacaagttatgggcggtagcctgaacatcttgggcgtttctccgaccaaacctctgac

SEQ ID NO: 884 - DNA, 224 bp, Neisseria meningitidis
ttgacggcaaaggtcaggaaggtttggaactttcgcttgaagacagtttgcatgctgaagaaggtgcggaagtcgtattgcgggatcgggaaggcaatattgtggacagtttggattctccgcgtaataaagccccgcaaaacggcaaagacattattctttctctggatcagaggattcagactttggcttatgaagagttgaataaggcggtcgaataccat

SEQ ID NO: 885 - DNA, 224 bp, Acinetobacter baumannii
tttaagtgggacgggcaaaaaaggctattcccagaatgggaaaaggacatgaccctaggcgatgctatgaaagcttccgctattccggtttatcaagatttagctcgtcgtattggacttgaactcatgtctaaggaagtgaagcgtgttggttatggcaatgcagatatcggtacccaagtcgataatttttggctggtgggtcctttaaaaattactcctca

SEQ ID NO: 886 - DNA, 224 bp, Escherichia coli
atgactgcgcacggtatctgtttccggaccgtgatgacgttgataagttgatcatcggtagtttctgctctatcgggagtggggcttcctttatcatggctggcaatcaggggcatcggtacgactgggcatcatctttcccgttcttttatatgcaggaagaacctgcattctcaagcgcactcgatgccttccaaaaagcaggtaatactgtcattggcaat

SEQ ID NO: 887 - DNA, 224 bp, Staphylococcus epidermidis
gaagttacggttataaataggagtgaagttgtcccttggcaatatcctccaaaaagagaatttatatacggtgagtggctcaggggtgaatttgagaatggacaaattcaggaaccaagctatgatcctgatttggctattgttttagcacaagcaagaaagaatagtatttctctatttggtcctgattcttcaagtatacttgtctccgtacctttgacaga

SEQ ID NO: 888 - DNA, 224 bp, Staphylococcus rostri
acatcatatagcatttcttttcttatcgttagcgtgctgtcattcctgatatttgtaaaacatatcaggaaagtaacagatccttttgttgatcccggattagggaaaaatataccttttatgattggagttctttgtgggggaattatatttggaacagtagcagggtttgtctctatggttccttatatgatgaaagatgttcaccagctaagtactgccga

SEQ ID NO: 889 - DNA, 224 bp, Burkholderia cepacia
acgcagttcgtgcgttcgtcgatcaaggccgtcgtgcacacgctgctcgaagcgatcgcgctggtcgtgatcgtcgtgatcgtgttcctgcagacctggcgcgcgtcgctgattccgctgatcgcggtgccggtgtcgatcatcggcacgttctcgctgctgctcgccttcgggtattcgatcaacgcgttgtcgctgttcgggatggtgctcgcgatcgggat

SEQ ID NO: 890 - DNA, 224 bp, Burkholderia cepacia
cctgatcgtgccgatgagcattctgtcggcgctgacgggcgtgtggctcacgcagggcgacaacaacatcttcacgcagatcggcttgatggtgctggtggggctgtcggcgaagaacgcgatcctgatcgtcgaattcgcgcgcgagctcgaacacgacggcaggacgccgctcgaggccgcgatcgaggcgagccggctgcggctgcgcccgatcctgatga

SEQ ID NO: 891 - DNA, 224 bp, Yersinia pestis
gggggatatctcaccgcatctgccgtttgtcattgcggcaatactgaatgcctgcacctttctgatggtcttttttatctttaaaccggcggtacagacagaagaaaaaccggcggagcagaaacaagaaagcgcaggtatcagctttatcacactgcttaaacctctggcgctgttgctgtttgtcttttttaccgcgcagcttatcgggcagatcccggcca

SEQ ID NO: 892 - DNA, 224 bp, Stenotrophomonas maltophilia
agagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaat

SEQ ID NO: 893 - DNA, 224 bp, Artificial Sequence
  Description of Artificial Sequence Synthetic integration vector polynucleotide
cttgtcactactttctcttatggtgttcaatgcttttcaagatacccagatcatatgaaacggcatgactttttcaagagtgccatgcctgaaggttatgtacaggaaagaactatatttttcaaagatgacgggaactacaagacacgtgctgaagtcaagtttgaaggtgatacccttgttaatagaatcgagttaaaaggtattgattttaaagaagatgg

SEQ ID NO: 894 - DNA, 224 bp, Klebsiella pneumoniae
agtgaaacgcaaaagcagctgcttaatcagcctgtcgagatcaagcctgccgatctggttaactacaatccgattgccgaaaaacacgtcaacggcacaatgacgctggcagaactgagcgcggccgcgttgcagtacagcgacaataccgccatgaacaaattgattgcccagctcggtggcccgggaggcgtgacggcttttgcccgcgcgatcggcgatga

SEQ ID NO: 895 - DNA, 224 bp, Serratia marcescens
ccacgttccgtctggaccgctgggagctggagctgaactccgccatcccaggcgatgcgcgcgatacctcatcgccgcgcgccgtgacggaaagcttacaaaaactgacactgggctctgcactggctgcgccgcagcggcagcagtttgttgattggctaaagggaaacacgaccggcaaccaccgcatccgcgcggcggtgccggcagactgggcagtcgga

SEQ ID NO: 896 - DNA, 224 bp, Unknown
  Description of Unknown Polynucleotide from a pathogenic organism
ctgctcgacgccaccacgccgcgctgggattacaagccggaattcaacggctacaaatcgcagcagaagccgaccgatccgaccatctggctgaaggattccatcgtctggtattcgcaggagctgacgcgccgcctcggcgaaagccgcttttccgattacgtgcagcgcttcgattacggcaacaaggatgtttccggcgatcccggcaagcataacggcct

SEQ ID NO: 897 - DNA, 224 bp, Unknown
  Description of Unknown Polynucleotide from a pathogenic organism
acccgcgtatacaccactaccccgataaaaccaaactccgcactgcgtaaagtttgccgtgtgcgtttgaccaacggtttcgaagtgacttcctacatcggtggcgaaggccacaacctgcaggaacactccgtaatcctgatccgtggcggtcgtgtaaaagacttgccaggtgtgcgttaccacaccgtccgtggtgcacttgactgctccggtgttaaaga

SEQ ID NO: 898 - DNA, 224 bp, Unknown
  Description of Unknown Polynucleotide from a pathogenic organism
aaattcaaaagaagggtcataaatggaccaaggcagaagtgaagtcagtaatccagttgctggccagtttgcgttcccttcaaacgccgcgttcggaatgggagatcgcgtgcgcaagaaatctggcgccgcttggcaaggccagattgtcgggtggtactgcacaaaattgacccctgaagggtacgctgtcgagtctgaggctcaccctggctcggtacaga

SEQ ID NO: 899 - DNA, 224 bp, Unknown
  Description of Unknown Polynucleotide from a pathogenic organism
tcatgctgttgatcggtatcgtgaagaaaaatgccatcatgatggtcgacttcgcgctcgaagcgcagcgcaatggcggcatcagcgcccgcgaggcgattttccaggccagcctgctgcgtttccggccgatcatgatgaccacgctggcggcgttgttcggcgcgctgccgctggtgctgacccgcggcgacggcgcggagctgcgccagccgctcggcatc

SEQ ID NO: 900 - DNA, 224 bp, Unknown
  Description of Unknown Polynucleotide from a pathogenic organism
ttcgacacgccgtgcgcactgaagatcacggtgttgccggccggcacttcctcgagttcctcgacgaagatcgcgcccttctttttcagatcctcgaccacgtacttgttgtggacgatttcgtgacgcacgtagatcggcgcaccgtgcatcgcgatggcgcgctcgacgatctcgatcgcgcgatcgacacccgcgcagaaaccgcgcggctgggccagcag

SEQ ID NO: 901 - DNA, 224 bp, Unknown
  Description of Unknown Polynucleotide from a pathogenic organism
cacgaacggtatggtaacgtacacccggtaagtctttaacacgaccaccacggataagaacaacactgtgctcttgtaagttatggccttcaccaccgatgtagctagatacttcaaaacctgaagttaagcgaacacggcaaaccttacgcatagctgagttaggttttttaggtgtagttgtataaacacgtgtacaaacaccacgacgttgtggacaagcc

SEQ ID NO: 902 - DNA, 224 bp, Unknown
  Description of Unknown Polynucleotide from a pathogenic organism
caacgatgttacgcagcagggcagtcgccctaaaacaaagttaggccgcatggacacaacgcaggtcacattgatacaccaaattctagctgcggcagatgagcgaaatctgccgctctggatcggtgggggctgggcgatcgatgcacggctagggcgtgtaacacgcaagcacgatgatattgatctgacgtttcccggcgagaggcgcggcgagctcgagg

SEQ ID NO: 903 - DNA, 224 bp, Unknown
  Description of Unknown Polynucleotide from a pathogenic organism
ctataccggcgtcaaccacaagctgggcgaagtgcatgacggcgctgcgaccaccgactggatggtgcaggagcaggagcgcggtatcaccattacctccgctgccgttaccaccttctggaagggctcgcgcggtcagtacgacaactatcgcgtaaacgtcatcgacacccccggccacgtagacttcaccatcgaggtggagcgttccctgcgtgtactgg

SEQ ID NO: 904 - DNA, 224 bp, Unknown
  Description of Unknown Polynucleotide from a pathogenic organism
gcacccgcatcgagcgctcgacctcgatggtgaagtccacgtgccccggggtgtcgatgatgttgatgcggtgttcgggataattgccggccatgccgcgccagaacgccgtggtggcggccgacgtgatggtgatgccgcgctcctgctcctgctccatccagtccatggtcgccgcgccttcgtgcacctcgcccagcttgtgggtgatgccggtgtagaaa

SEQ ID NO: 905 - DNA, 224 bp, Unknown
  Description of Unknown Polynucleotide from a pathogenic organism
tcgagcgcggcttcgccgactaccggttcgacaacgtggccaacgcgctgtaccgctacgtgtgggacgaatactgcgactggtacctggagctggccaaggtgcagatccagcagggcacaccggcccagcagctgggcacccgccgtacgctgatccgcgtactggaggcggtgctacgcctggcccatccggtcatcccgttcatcacggaagaactgtgg

SEQ ID NO: 906 - DNA, 205 bp, Unknown
  Description of Unknown Polynucleotide from a pathogenic organism
tccggacgggcgcccggacggtcgaccttgttgaccacgacgatcggcttgaagcccatcgcgaaggccttctgggtcacgaagcgggtctgcggcatcgggccgtccatcgcgtcgaccaggatcagcacggtgtcgaccatcgacagcacgcgctcgacctcaccaccgaagtcggcgtggccgggggtgtcgacgatgttga

SEQ ID NO: 907 - DNA, 201 bp, Unknown
  Description of Unknown Polynucleotide from a pathogenic organism
atgaatttaacatttttcggtctatgtcttgcctgtatgggtgtatctcttggtgagggtatgttgatgaatggtttgttgaaatcagtagctcgccaaccagatattatcgctgagtttcgtagtttgatgtttttaggggttgcctttattgaaggaactttcttcgtaactcttgtcttctcatttattattaaataa

SEQ ID NO: 908 - DNA, 224 bp, Unknown
  Description of Unknown Polynucleotide from a pathogenic organism
gtgcgattttacctttacgcaataatacaatacgatcagacattgtaattgcttcttcttgatcgtgggtgacaaaaataaaggtaatgccaagctgacgttgtaacattttgagttcttgttgcatttgtttacgcaatttgtaatccaatgcagataaagactcatcaagtaacaacacttttggtttatttactacggcacgagcgatggcaatacgttgt

SEQ ID NO: 909 - DNA, 224 bp, Unknown
  Description of Unknown Polynucleotide from a pathogenic organism
ggtatggtaaaactagaacaaaaaatgggtgatgaaaaatggatggaatatttgaaaaaatttggttttggtacgagcacgcattcaggtttaattggtgaatctgctggtaaattgccaggaacaaactttgtagatagagcaatgtcggcatttggtcaagcaattacagttactaatttccaaatgatgaaaggtttctcagttattgcaaatgacggttc

SEQ ID NO: 910 - DNA, 223 bp, Unknown
  Description of Unknown Polynucleotide from a pathogenic organism
ggcggaatccactttcggcgcaggtcgaacgattcgggcggtacgtcaatcagcatttccatatcgaaaaaatattgcagcatcacgcccaagcggccgtagtcgttgcttttcggcgcggcaaccatacgatcgaccacttctttttgcagcataaagtgcatatcgacgacatcgtccgccacctccgccagcttgaacaaaagcggcgtggaaatgttgt

SEQ ID NO: 911 - DNA, 224 bp, Unknown
  Description of Unknown Polynucleotide from a pathogenic organism
gactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggcatcacaaagtacagcatcgtgaccaacagcaacgattccgtcacactgcgcctcatgactgagcatgaccttg

SEQ ID NO: 912 - DNA, 224 bp, Unknown
  Description of Unknown Polynucleotide from a pathogenic organism
ctgttctacaccggcaagagccacaagatcggtgaagtgcatgacggcgccgccaccatggactggatggagcaggagcaggagcgtggcatcacgatccagtccgctgccaccaccgcgttctggaagggcatggacaagtccctgccggagcaccgcttcaacatcatcgacacccccgggcacgttgacttcaccatcgaagtcgagcgttcgctgcgcgt

SEQ ID NO: 913 - DNA, 224 bp, Unknown
  Description of Unknown Polynucleotide from a pathogenic organism
ccgcagcgccgtggcgtctgcacccgtgtctacaccaccaccccgaagaagccgaactcggctctgcgcaaggttgccaaggtccgcctgaccaaccaggaagaagtgatttcctacatcggtggtgaaggccacaacctgcaggagcactccgtggtcctgatccgtggcggtcgcgtcaaggatctgccgggcgtgcgttaccacaccgtccgtggttcgct

SEQ ID NO: 914 - DNA, 224 bp, Unknown
  Description of Unknown Polynucleotide from a pathogenic organism
tcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagc

SEQ ID NO: 915 - DNA, 224 bp, Unknown
  Description of Unknown Polynucleotide from a pathogenic organism
actctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaag

SEQ ID NO: 916 - DNA, 224 bp, Unknown
  Description of Unknown Polynucleotide from a pathogenic organism
gtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcg

SEQ ID NO: 917 - DNA, 224 bp, Unknown
  Description of Unknown Polynucleotide from a pathogenic organism
atcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggta

SEQ ID NO: 918 - DNA, 224 bp, Unknown
  Description of Unknown Polynucleotide from a pathogenic organism
ttagcgcaagagtccgtcactccatacattgcaatgctgaatggagagccgattgggtatgcccagtcgtacgttgctcttggaagcggggacggatggtgggaagaagaaaccgatccaggagtacgcggaatagaccagtcactggcgaatgcatcacaactgggcaaaggcttgggaaccaagctggttcgagctctggttgagttgctgttcaatgatcc

SEQ ID NO: 919 - DNA, 224 bp, Unknown
  Description of Unknown Polynucleotide from a pathogenic organism
atgagtggctaaatcgatctcatatcgtcgagtggtggggcggagaagaagcacgcccgacacttgctgacgtacaggaacagtacttgccaagcgttttagcgcaagagtccgtcactccatacattgcaatgctgaatggagagccgattgggtatgcccagtcgtacgttgctcttggaagcggggacggatggtgggaagaagaaaccgatccaggagta

SEQ ID NO: 920 - DNA, 224 bp, Unknown
  Description of Unknown Polynucleotide from a pathogenic organism
cggccctcggcctggtccttcagccaccatgcccgtgcacgcgacagctgctcgcgcaggctgggtgccaagctctcgggtaacatcaaggcccgatccttggagcccttgccctcccgcacgatgatcgtgccgtgatcgaaatccagatccttgacccgcagttgcaaaccctcactgatccgcatgcccgttccatacagaagctgggcgaacaaacgatg

SEQ ID NO: 921 - DNA, 224 bp, Unknown
  Description of Unknown Polynucleotide from a pathogenic organism
gcatggacacaacgcaggtcacattgatacacaaaattctagctgcggcagatgagcgaaatctgccgctctggatcggtgggggctgggcgatcgatgcacggctagggcgtgtaacacgcaagcacgatgatattgatctgacgtttcccggcgagaggcgcggcgagctcgaggcaatagttgaaatgctcggcgggcgcgtcatggaggagttggactat

SEQ ID NO: 922 - DNA, 224 bp, Unknown
  Description of Unknown Polynucleotide from a pathogenic organism
ccctaaaacaaagttaggccgcatggacacaacgcaggtcacattgatacaccaaattctagctgcggcagatgagcgaaatctgccgctctggatcggtgggggctgggcgatcgatgcacggctagggcgtgtaacacgcaagcacgatgatattgatctgacgtttcccggcgagaggcgcggcgagctcgaggcaatagttgaaatgctcggcgggcgcg

SEQ ID NO: 923 - DNA, 224 bp, Unknown
  Description of Unknown Polynucleotide from a pathogenic organism
tcaatgtccgtttgagcgcaggctgtcgcgaatgttcggacgcgccgttgatgtggtgtcccgcaatgccgtcaatcccgacttcttaccggacgaggacaagagtacgccgcagctcgatcttttggctcgtgtcgaacgagagctaccggtgcggctcgaccaagagcgcaccgatatggttgtttgccatggtgatccctgcatgccgaacttcatggtgg

SEQ ID NO: 924 - DNA, 224 bp, Unknown
  Description of Unknown Polynucleotide from a pathogenic organism
ccctgccttctgcccttctcccgatccgggatcgctttgcagctttgtttcagcgggcgcgcgatgatcaaaacgcaggttgtcaaactgactacgtccacgcggcgattatagccgatcaaatgatgagcaatgcctcggaactgcgtgggctacatggcgatctgcatcatgaaaacatcatgttctccagtcgcggctggctggtgatagatcccgtcggt

SEQ ID NO: 925 - DNA, 224 bp, Unknown
  Description of Unknown Polynucleotide from a pathogenic organism
gccaagctctcgggtaacatcaaggcccgatccttggagcccttgccctcccgcacgatgatcgtgccgtgatcgaaatccagatccttgacccgcagttgcaaaccctcactgatccgcatgcccgttccatacagaagctgggcgaacaaacgatgctcgccttccagaaaaccgaggatgcgaaccacttcatccggggtcagcaccaccggcaagcgccg

SEQ ID NO: 926 - DNA, 224 bp, Unknown
  Description of Unknown Polynucleotide from a pathogenic organism
atctgacaatgagaacgtattgatctttccatcaattaaagatgctttaaccaacctaaagaaaataacggatcatgtcattgtttcaggtggtggggagatatacaaaagcctgatcgatcaagtagatacactacatatatctacaatagacatcgagccggaaggtgatgtttactttcctgaaatccccagcaattttaggccagtttttacccaagact

SEQ ID NO: 927 - DNA, 224 bp, Unknown
  Description of Unknown Polynucleotide from a pathogenic organism
tcgtgcgcttaacgctgtggtgtatcgttgttggcctctatccaaccttgctgatgacgttaatcggcgtgctgtggtttcccagtcgtgccatttcgcaaatgcatcaggcgcttaatgatcggcttgatgatgccattagccacctgacggacagcctcgcaccgctacccgaaacgcggattgaaagagaggcgctggcgctgcaaaaactcaatgtcttt

SEQ ID NO: 928 - DNA, 224 bp, Unknown
  Description of Unknown Polynucleotide from a pathogenic organism
cgccacgctcgaaaacgtttttggcccggtgtacgacctggtggaaattcgcgatcgcgccctgggtatcatcattggtaccgtggtgtccgcggtgatttacacctttgtctggcctgaaagtgaagcgcgcacgctgccgcaaaaactggctggcgcgctgggtatgttaagtaaagtaatgcggatcccacgccagcaggaagtcacggctctgcgcactt

SEQ ID NO: 929 - DNA, 224 bp, Unknown
  Description of Unknown Polynucleotide from a pathogenic organism
ggttcacacaccctcgccgaagcgaaactgcgggaagaaaaagcgcagtcccaggccgatttgttagatgccggttcacaattacaggtcgcagcgttagggatgctcaaccgccaacgcgtctcggcgaacggctttttaagcccttatgcgatggatgcgccagcactgggtatggacgggccgtactatacggaagccacagtcggtttgtttgccggact

SEQ ID NO: 930 - DNA, 224 bp, Unknown
  Description of Unknown Polynucleotide from a pathogenic organism
tctggaagcgcaagtgcctttccacggcgcgcgggcacagattctggcggtcgataaacaaattgctgccgtcaaagggcaaatcaccgaaacgcgagaatctctgcgtgcattgattggcgcgggagccagcgatatgccggagatcaaaccggtggcattaccacgagtccagaccggcattccggcgacactctcttatgagttgctcgccagacgcccgg

SEQ ID NO: 931 - DNA, 224 bp, Unknown
  Description of Unknown Polynucleotide from a pathogenic organism
gcatgaccttgcgatgctctatgagtggctaaatcgatctcatatcgtcgagtggtggggcggagaagaagcacgcccgacacttgctgacgtacaggaacagtacttgccaagcgttttagcgcaagagtccgtcactccatacattgcaatgctgaatggagagccgattgggtatgcccagtcgtacgttgctcttggaagcggggacggatggtgggaag

SEQ ID NO: 932 - DNA, 224 bp, Unknown
  Description of Unknown Polynucleotide from a pathogenic organism
tacaggccgagctaaactccgccaaaggctcgctggcgaaagcgctctctaccgccagcaatgcccgcatcacctttaaccgccaggcatcgttgctgaagaccaactacgttagccgtcaggattacgacaccgcgcgcacccagttgaatgaagcagaagccaatgtcaccgtcgccaaagcggctgttgaacaggcgacgatcaatctgcaatacgcgaat

SEQ ID NO: 933 - DNA, 224 bp, Unknown
  Description of Unknown Polynucleotide from a pathogenic organism
aattctccgacccgacggtggatgaaaccacgggctccgtgacgttacgggcgattttccccaacccaaatggtgacttgctgcctggcatgtatgtcacggcattagtggatgaaggtagccgccagaatgtattactggtgccgcaggaaggcgtcacccacaacgcccagggtaaagcaacggcgctcattctggataaagacgatgtcgtgcagctacgc

SEQ ID NO: 934 - DNA, 224 bp, Unknown
  Description of Unknown Polynucleotide from a pathogenic organism
agcgtcgataagtcgagcagtaatatcctgatggtagcggcgtttatttctgataacggcagcctcaaccagtacgatatcgcggactatgtagcgtctaatatcaaagacccgctaagccgtaccgcgggcgttggtagcgtacagctctttggttccgagtacgccatgcgtatctggctggacccgcaaaaactcaataaatacaatctggtaccttccga

SEQ ID NO: 935 - DNA, 224 bp, Unknown
  Description of Unknown Polynucleotide from a pathogenic organism
cgtaccggtggttattctcgggacgtttgcgatcttgtcggcggtcggtttcaccatcaacacgttgactatgttcgggatggtgctggcgatagggttactggtggatgacgccatcgtggtggtggagaacgtcgagcgtgtcattgcggaagataagctaccaccgaaggaagcgacgcataaatcgatggggcagatccaacgtgcgctggtcggtattg

SEQ ID NO: 936 - DNA, 224 bp, Unknown
  Description of Unknown Polynucleotide from a pathogenic organism
cggtggctgaactgggtaccgcgtcaggttttgatatggaactgctggacaacggtaacctggggcacgaaaaactaacccaggcacgaaacgagctgttatcactggcagcgcaatcaccgaatcaggtcaccggggtacgcccgaacggcctggaagatacgccgatgttcaaagtgaacgtcaacgccgcgaaagctgaggcaatgggtgtggcgctgtct

SEQ ID NO: 937 - DNA, 224 bp, Unknown
  Description of Unknown Polynucleotide from a pathogenic organism
caggaagcgttatcctcaaatcaggctcctgcgctgtatgccatttcactggtcgtggtgttcctcgccctcgccgcactttatgagagctggtcaattccgttctcggtgatgttggttgttccgttaggcgtcgttggcgcattactggccaccgatctgcgcggcttaagtaatgacgtctacttccaggttggtttgctgaccaccatcgggctttccgc

SEQ ID NO: 938 - DNA, 224 bp, Unknown
  Description of Unknown Polynucleotide from a pathogenic organism
gaatgcccggcgcgcggatacttccgctcaagggcgtcgggaagcgcaacgccgctgcggccctcggcctggtccttcagccaccatgcccgggcacgcgccagctgctcgcgcaggccgggcgccaagctctcgggcaacatcaatgcccgatccttggagcccttgccctcccgcacgatgatcgtgccgtgatcgaaatccagatccttgacccgcagttg

SEQ ID NO: 939 - DNA, 224 bp, Unknown
  Description of Unknown Polynucleotide from a pathogenic organism
gtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagggcagtcgccctaaaacaaagttagccatta

SEQ ID NO: 940 - DNA, 224 bp, Unknown
  Description of Unknown Polynucleotide from a pathogenic organism
ttgaattgaaaatttcattgatttctgcaacgtcagaaaatggcgtaatcggtaatggccctgatatcccatggtcagcaaaaggtgagcagttactctttaaagcgctcacatataatcagtggctccttgttggaaggaaaacatttgactctatgggtgttcttccaaatcgaaaatatgcagtagtgtcgaggaaaggaatttcaagctcaaatgaaaat

SEQ ID NO: 941 - DNA, 224 bp, Unknown
  Description of Unknown Polynucleotide from a pathogenic organism
ctccttgttggaaggaaaacatttgactctatgggtgttcttccaaatcgaaaatatgcagtagtgtcgaggaaaggaatttcaagctcaaatgaaaatgtattagtctttccttcaatagaaatcgctttgcaagaactatcgaaaattacagatcatttatatgtctctggtggcggtcaaatctacaatagtcttattgaaaaagcagatataattcattt

SEQ ID NO: 942 - DNA, 224 bp, Unknown
  Description of Unknown Polynucleotide from a pathogenic organism
aattaccaggccgcaccaatgcttaccgtatagccgaagttcgcccacaggttagcgggatcgtactgaatcgcaatttcactgaaggcagcgatgtgcaagcaggccagtccctgtaccagatcgatcccgcgacctatcaggcaaattatgacagcgcgaaaggcgaactggcgaaaagtgaagccgccgccgccatcgcgcatttgacggtaaaacgttac

SEQ ID NO: 943 - DNA, 224 bp, Unknown
  Description of Unknown Polynucleotide from a pathogenic organism
gtccagcagctcgatcctatctacgttgatgtgacccaatccagcaacgattttatgaggctgaagcaatccgtagagcaaggaaatttgcataaggaaaacgccaccagcaacgtagagttggtcatggaaaacggtcaaacctatcccctgaaaggtacgctgcaattctccgatgtgaccgttgatgaaagcaccggctccataaccctacgtgctgtctt

SEQ ID NO: 944 - DNA, 224 bp, Unknown
  Description of Unknown Polynucleotide from a pathogenic organism
cgatcctgatatcgcgcaagtgcaggtgcagaacaaactccagctcgccacgccgttgctgccgcaggaggttcagcagcaggggatcagtgttgaaaagtccagtagcagctatttgatggtggcgggctttgtctctgataacccagacaccacacaggacgatatctcggactatgtggcctctaacgttaaagatacgcttagccgtctgaatggcgtcg

SEQ ID NO: 945 - DNA, 224 bp, Unknown
  Description of Unknown Polynucleotide from a pathogenic organism
aaactataacgttatcgctcgcatcaacggaaaaccggcggcgggcctggggattaagctggcaaccggcgcgaatgctctcgataccgcgaaagccattaaggcaaaactggcggaattacagccattcttcccgcagggaatgaaggttctctacccttatgacaccacgccattcgtccagctttctattcacgaagtggtaaaaacgctgttcgaagcca

SEQ ID NO: 946 - DNA, 224 bp, Unknown
  Description of Unknown Polynucleotide from a pathogenic organism
ttctgttctggtggcattgattcttacccctgcgttatgtgcaacactgcttaaacccgtctctgctgagcatcacgaaaataagggcggtttcttcggctggtttaataccaccttcgatcatagcgttaaccactacaccaacagcgtcggcaaaatcctcggttccacaggacgatatttactgatctatgcgctgattgttgcaggaatggtggtgttgt

SEQ ID NO: 947 - DNA, 224 bp, Unknown
  Description of Unknown Polynucleotide from a pathogenic organism
ggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagt

SEQ ID NO: 948 - DNA, 224 bp, Unknown
  Description of Unknown Polynucleotide from a pathogenic organism
gccgtgtacgacaccattgtacgtatggcgcagccattctccctgcgttacatgctggtagatggccagggtaactttggttcggtcgacggcgactccgccgcagcgatgcgttatacggaaatccgtatgtcgaagatcgcccatgagctgatggccgacctcgaaaaagagacggtggatttcgtcgataactatgacggcacggaaaaaatccctgacgt

SEQ ID NO: 949 - DNA, 224 bp, Unknown
  Description of Unknown Polynucleotide from a pathogenic organism
ggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagggcagtcgccctaaaacaaagttaggcatcatgggtgaattctttccctgcacaagttttcaagcagctgtcccacgctc

SEQ ID NO: 950 - DNA, 224 bp, Unknown
  Description of Unknown Polynucleotide from a pathogenic organism
ttggcgctatccggcgcggcgtgagcttcagttcggtgagtggctccgccacgacatcctttccggaacgttcgagcctgccgttctggatcacgatcttgcgattttgctgaccaaggcgaggcaacacagccttgcgcttctaggcccatccgcagccacgtttttcgagccggtgccgaaggagcatttctccaaggcgcttttcgacactattgcccagt

SEQ ID NO: 951 - DNA, 224 bp, Unknown
  Description of Unknown Polynucleotide from a pathogenic organism
ttttcaatcaataacctgacgctgatggcgttaactatcgccaccggattcgtggtcgatgacgccatcgtggtgatcgaaaacatttcccgctatatcgaaaaaggcgaaaaaccgttggcggcggcgctcaagggcgcaggtgaaatcggctttaccattatctcgctgaccttctcactgattgcggtgttgatcccactgctgtttatgggcgatatcgt

SEQ ID NO: 952 - DNA, 224 bp, Unknown
  Description of Unknown Polynucleotide from a pathogenic organism
cagaggtagataaaattacagggttaacaatcggcgcggatgattatataacgaagccctttcgcccactggagttaattgctcgggtaaaggcccagttgcgccgatacaaaaaattcagtggagtaaaggagcagaacgaaaatgttatcgtccactccggccttgtcattaatgttaacacccatgagtgttatctgaacgagaagcagttatcccttact

SEQ ID NO: 953 - DNA, 224 bp, Unknown
  Description of Unknown Polynucleotide from a pathogenic organism
agataaacaaattgagctttctgcggaaatggatgttatggaacaaaagctcaacacattaaaacggactctggaaaagcgagagcaggatgcaaagctggccgaacaaagaaaaaatgacgttgttatgtacttggcgcacgatattaaaacgccccttacatccattatcggttatttgagcctgcttgacgaggctccagacatgccggtagatcaaaagg

SEQ ID NO: 954 - DNA, 224 bp, Unknown
  Description of Unknown Polynucleotide from a pathogenic organism
acaggcggttattcacgcccccgaggatctgaccgtgtccggcgaccctgataaactcgcgagagtctttaacaacattttgaaaaacgccgctgcatacagtgaggataacagcatcattgacattaccgcgggcctctccggggatgtggtgtcaatcgaattcaagaacactggaagcatcccaaaagataagctagctgccatatttgaaaagttctata

SEQ ID NO: 955 - DNA, 224 bp, Unknown
  Description of Unknown Polynucleotide from a pathogenic organism
aatcagtggcttttggtaggccgaaagactttcgagtcaatgggggctttacccaaccgaaaatatgccgttgtaactcgttcaagcttcacttccagtgatgagaatgtattggtatttccatctatcgatgaagcgctaaatcatctgaagacgataacggatcatgtgattgtgtctggtggtggtgaaatatacaaaagcctgatcgataaagttgatac

SEQ ID NO: 956 - DNA, 224 bp, Unknown
  Description of Unknown Polynucleotide from a pathogenic organism
cacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtcta

SEQ ID NO: 957 - DNA, 224 bp, Unknown
  Description of Unknown Polynucleotide from a pathogenic organism
atgctgccggtcagcgagatgctcgggaagaacgccgcgcgtgccgcaccgatgttggcgttggccgccagcagctgatgttcggcggccatgatgtccgggcgctgcagcaacacgtcgctgggcagaccggccggcggcggtgccagtgccagcagctgcggttcgatgctgtccggcagcagtgccggatccagctggccaccggccagcagcgccagcgc

SEQ ID NO: 958 - DNA, 224 bp, Unknown
  Description of Unknown Polynucleotide from a pathogenic organism
aggctcaactgcgcgttgcggcggttggcggccacggcgaagtactgctgcagcgccgcttcactgagattgcgcacgcggccgaacagatccagctcgaactcggctacgccaacaccggcggtgaactgctcggtgacaccggcgtcggtaccacggcggtccatctggccggtgacggccacgccgggcacacggtcggcgcgctgtacgcggtactggcc

SEQ ID NO: 959 - DNA, 224 bp, Unknown
  Description of Unknown Polynucleotide from a pathogenic organism
gccgtgctcaacgttgagcgcgcgcgcggccagtaccgcgtgcagcgcgccgatcgcgtgcccggcgtggccgttaccggccagatgcagcgccagggcaccgacgccggtgtcaccgagcagttcaccgccggtgtcggcgtggccgagttcgagctggacctgttcggccgcgtgcgcaatctcagtgaagcggcgctgcagcagtacttcgccgtcgccgc

SEQ ID NO: 960 - DNA, 224 bp, Unknown
  Description of Unknown Polynucleotide from a pathogenic organism
atgccgcacgcctgcgtggccagctggcccaggaccgcaacgcgctggcgctgctggccggtggccagctggatccggcactgctgccggacagcatcgaaccgcagctgctggcactggcaccgccgccggccggcctgcccagcgacgtgctgctgcagcgcccggacatcatggccgccgaacaccagctgctggcggccaacgccaacatcggtgcggca

SEQ ID NO: 961 - DNA, 224 bp, Unknown
  Description of Unknown Polynucleotide from a pathogenic organism
gttggtgacgtaatcggtaaatatcacccgcatggtgactcagctgtttatgaaaccattgttcgtatggctcaagactttagcttacgttatttattggttgatggtcagggtaacttcggttcgatcgatggtgatagcgctgcggcaatgcgttataccgaagtccgtatgactaagctggcacatgagcttcttgcagatttagaaaaagacacagttga

SEQ ID NO: 962 - DNA, 224 bp, Unknown
  Description of Unknown Polynucleotide from a pathogenic organism
tccatatcaagtaaacaaagcaagagttattgaacgtattgccgagttagtaaaagagaaaaagcttgaaggtatttcagaacttcgtgatgagtctgataaagaaggtatgcgtattgcaattgacttgaaacgcggtgaaaacgcagaagtcgttgtaaataacttattcttaaatacccagcttgaaaactcattcagcatcaacatggtttgtctagaca

SEQ ID NO: 963 - DNA, 224 bp, Unknown
  Description of Unknown Polynucleotide from a pathogenic organism
actggttatacaattattagtggtaaacatttttattttaatactgatggtattatgcagataggagtgtttaaaggacctgatggatttgaatactttgcacctgctaatacagatgctaacaatatagaaggtcaagctatacgttatcaaaatagattcctatatttacatgacaatatatattattttggtaataattcaaaagcggctactggttgggt

SEQ ID NO: 964 - DNA, 224 bp, Unknown
  Description of Unknown Polynucleotide from a pathogenic organism
ctcgaatatggaagatgcagcatgcgactaaacaaaagaaaatgcatcaaaatattaaggctcttgaaacacgtgttgaaaaactagagcgtgtgaaaaaaccaaaagattatccggctgtcaaaatgaagttgtctaaccaagatcaaatacaggggcgcaatgtacttcgggtaaaagacttatctgtttcctttgggaatcatgtgctttggacagatgct

SEQ ID NO: 965 - DNA, 224 bp, Unknown
  Description of Unknown Polynucleotide from a pathogenic organism
tgccattattggcaataatggggtcggtaaaacaacattgttgaaacaaattttagaaagggtaccagcggtaacaatatcacccgcagcaaaaatcggctattttagccagaatttggatacgcttgatacgcatgtgtcgatcttagaaaatgtcatgtccaccgctattcaagatgaaactactgtacggactgttctcgcaagattacatttctaccggg

SEQ ID NO: 966 - DNA, 224 bp, Unknown
  Description of Unknown Polynucleotide from a pathogenic organism
ttacatggaggttatggtgagaatggtgctatgcagggagtatttgagttattagatattccatatgtaggttgtggtatcggagctgcagcaatctctatgaataaaataatgctccatcaatttgctgaagcaattggtgtaaaaagcactcctagtatgattatagaaaagggacaagacctacaaaaagtcgatgaatttgcgaaaatacatggatttcc

SEQ ID NO: 967 - DNA, 224 bp, Unknown
  Description of Unknown Polynucleotide from a pathogenic organism
aaattattaccattgatggctatcgttcaaaggagacacagcaagcactttgggatgagacgattcaagaaaaagggcttgaatttgcgcacaaatatgtggcaaagcctggatgtagtgaacatgaaattggtttagcagtggatttggggttagctacgaaagaaaatgattttattcgcccaagtttcactgatagtccgattgttgataaatttttaaag

SEQ ID NO: 968 - DNA, 224 bp, Unknown
  Description of Unknown Polynucleotide from a pathogenic organism
tgatcgcgatcccgcaactgccggtcgcgcgctacccgtcggtcgcgccgccgtctgtcacgatcaccgcgagctacccgggcgccacgccgcagacgatgaacgacggcgtgctgagcctcatcgagcgcgaattatccggcgtcaagaacctgctgtacttcgaatcgtcggccgatacctcgggccaggcacagatcaccgtgacgttcaagcccggcact

SEQ ID NO: 969 - DNA, 224 bp, Unknown
  Description of Unknown Polynucleotide from a pathogenic organism
ggacgcgatccgcgcccgcatggccgaactgagcaagtcgatgccgtcgggcatgagctactcgatcccgctcgacacgtcgccgttcgtgaagatctcgatcgagaaagtgctgcacacgctgctcgaggcgatggtgctcgtgttcctcgtgatgtacctgttcctgcagaacgtgcgctatacgctgatcccggccatcgtcgcgccggtcgcgatgctcg

SEQ ID NO: 970 - DNA, 224 bp, Unknown
  Description of Unknown Polynucleotide from a pathogenic organism
tgctcgcgctgacgctcacgccggccctgtgcgcgacgatgctcaagccgatcgcagcgggtcatcacgagaagcgcggcttcttcggctggttcaaccgccgcttcgatcgcctgacgaaatggtacgagacgcgcgtcggccgcctcgtcggtcgcacgggccgcgtgatgctggtgttcgtcgcgatttcgggtgcactggtgctcggtttccgcagcctg

SEQ ID NO: 971 - DNA, 224 bp, Acinetobacter baumannii
ccagctgatgatttcaaagcaattgaacaagtaggttcaatctctgctaactctgatactactgttggtaaacttattgctcaagcaatggaaaaagtaggtaaagaaggcgtaatcactgtagaagaaggctctggcttcgaagacgcattagacgttgtagaaggtatgcagtttgaccgtggttatatctctccgtactttgcaaacaaacaagatacttt

SEQ ID NO: 972 - DNA, 224 bp, Acinetobacter baumannii
ctaccttgaaacttgttatttattattaaatggcgagttaccaactgctgaacaaaaagttgagttcgatgcgaaagttcgtgctcatactatggttcatgatcaagttagccgtttcttcaatggtttccgtcgtgatgctcaccctatggcaatcatggttggtgtagtaggcgcattatctgctttctatcacaacaaccttgacattgaagacatcaacc

SEQ ID NO: 973 - DNA, 224 bp, Acinetobacter baumannii
atcttccatttcacagccgatgctgacaacggcattgctgtagaagttgcattgcaatggaacgatagttaccaagaaaatgttcgctgtttcacaaacaacattccacaaaaagatggtggtacgcacttagcaggtttccgcgcagctttaacacgtggcttaaaccagtatcttgaaaatgaaaatattctcaagaaagaaaaagtgaatgtgactggtga

SEQ ID NO: 974 - DNA, 224 bp, Acinetobacter baumannii
gtgttcttgaaagagaagagcgcagaaatcgatgagaaattcgctgagaagaaacgtaagcttgcaacaggtgatgaattaacaactggcgtattaaaagttgttaaagtttacttagctgttaaacgtcgtattcagcctggtgataagatggctggtcgtcacggtaacaagggtgttgtatctaacatcttacctgttgaagacatgccacatgatgctaa

SEQ ID NO: 975 - DNA, 224 bp, Burkholderia ambifaria
cctcaagattctcgagatcttccagcaaagccccgtgcgtcacctgactgccgaagacgtctaccgcaacctgctcaacgagcagctcgacatcgggctcgccaccgtttaccgcgtgctgacgcagttcgagcaggccggcctgctcacgcgcagcaacttcgaatccggcaaggccgtgttcgaattgaatgaaggttcgcaccacgatcacctcgtctgcc

SEQ ID NO: 976 - DNA, 224 bp, Burkholderia ambifaria
cagatgccggacatgatcgagcgcgggtatgtgtatatcgcgcagccgccgctttacaagatcaaggcgggcaaggacgagcggtatctgaaggacgatacggagctgaacgcgcatatgctgcggttggcgctgcaagggtcggagctggtgcctggcgagaatgcggcggcgatttccggcgatgcgctcggggagctggcgcggtcgtacctgctgtcgaa

SEQ ID NO: 977 - DNA, 224 bp, Burkholderia ambifaria
cgcagacggtgttcgacaacgccgtcgtgtggcgccgcttcctcgacgacaagcacttccagagtcagaaggaactgtccgaacatctcggcctcgacgagtcgacggtcgcggttgcgctgtcgatcggcaagctgccggaaaccgtgatgcaggagatggtcgcgcgccccgatcgcttcggttcgaacatggcgtatcaggtcggccgatatcacaatgcg

SEQ ID NO: 978 - DNA, 224 bp, Burkholderia ambifaria
tcatcaaccagatccggatgaagatcggcgtgatgttcggcaacccggaaaccacgacgggcggcaacgcgctgaagttctattcgtcggtgcgtctcgacatccgccggatcggctcgatcaagaagaacgacgaggtgatcggcaacgaaacccgcgtgaaggtcgtcaagaacaaggtgtcgccgccgttccgcgaagcgatcttcgacatcctgtatggc

SEQ ID NO: 979 - DNA, 224 bp, Burkholderia cenocepacia
gctcaacgagcagctcgacatcgggctcgccaccgtctatcgcgtgctcacgcagttcgagcaggccggcctgctgtcgcgcagcaacttcgagtccggcaaggcggtgttcgaactgaacgaaggttcgcaccacgaccacctcgtgtgcctcgattgcggccgcgtcgaggaattcttcgacgccgagatcgagagccgccagcaggcgatcgcgaaggaac

SEQ ID NO: 980 - DNA, 224 bp, Burkholderia cenocepacia
cgtgttgttcgcgatgcacgaactgaacaacgactggaaccgcgcgtacaagaagtcggcgcgtatcgtcggtgacgtgatcggtaaataccaccctcacggcgataccgcggtctacgacacgatcgtgcggatggcgcaggacttctcgctgcgctacatgctgatcgacgggcagggcaacttcggctcgatcgacggcgacaatgccgcggcgatgcgtt

SEQ ID NO: 981 - DNA, 224 bp, Burkholderia cenocepacia
gagcggatcacgacgatgatcggcgacgagctgacctcggtgaaggccgaattcggtgatgcgcgccgctcgaagatcgaactgaacgcgaccgagctgaacaccgaggatctgatcacgccgcaggacatggtcgtcacgatgtcgcatgcgggctacgtgaagtcgcagccgctgtccgagtaccgtgcgcagaagcgcggcggtcgcggcaagcaggcgac

SEQ ID NO: 982 - DNA, 224 bp, Burkholderia cenocepacia
aaggtctggaagcggtgcgcaagcgccccgggatgtacatcggcgacacgtcggacggcaccggtctgcaccacctcgtgttcgaggtgctcgacaactcgatcgacgaagcgttggccgggtactgcaacgacatccacgtgacgattcacgccgacaactcgatttctgtgaccgacaacggccgcgggattccgaccgacgtgaagatgaacgacaagcac

SEQ ID NO: 983 - DNA, 224 bp, Burkholderia cenocepacia
atgatcgagcgcgggtacgtgtatatcgcgcagccgccgctttacaagatcaaggcgggcaaggacgagcggtatctgaaggacgacgtggagctcaacgcgcacatgctgcggttggcgctgcaagggtcggagctggtgccgggtgagaatgcggcggcgatttcgggtgatgcgcttggggagctggcaaggtcgtatctgctgtcgcagagcgtgatcga

SEQ ID NO: 984 - DNA, 224 bp, Burkholderia cenocepacia
gtcgtgtggcgccgcttcctcgacgacaagcatttccagagccagaaggaactctccgagcatctcggcctcgacgagtcgacggtggccgtcgcgctgtcgatcggcaagttgccggaagcgatcatgcaggaaatggtcgcgcgtcccgatcgcttcgggtcgaacatggcgtatcaggtcggccgctatcacaatgcgcgcggcaccgaggccacgctgcg

SEQ ID NO: 985 - DNA, 224 bp, Burkholderia cenocepacia
tggtgcgctcgggctcgatcgacatgatcgtcatcgactcggtcgcggcgctcgtgccgaaggccgaaatcgaaggcgagatgggcgattcgctgccgggtctgcaggctcgcctgatgtcgcaggcgctgcgcaagctgaccggcacgatcaagcgcacgaactgcctcgtgatcttcatcaaccagatccggatgaagatcggcgtgatgttcggcaacccg

SEQ ID NO: 986 - DNA, 224 bp, Burkholderia cepacia
accaatccgaccgatctcaagaatatcgggctaaaggccaccctaccgcgcctcaagattctcgagatcttccagcaaagcccggtgcgccacctgaccgcagaagacgtctaccggaacctgctcaacgagcagctcgacatcgggcttgccaccgtctaccgcgtgctgacgcagttcgagcaggccggcctgctgtcgcgcagcaacttcgaatccggcaa

SEQ ID NO: 987 - DNA, 224 bp, Burkholderia cepacia
catcatgaccgacgcggacgtcgacggtgcgcacatccggacgctgctgctcacgttcctgtatcgccagatgccggacatgatcgagcgcgggtacgtgtatatcgcgcagccgccgctttacaagatcaaggcgggcaaggacgagcggtatctgaaggatgacgtggagctcaacgcgcacatgctgcggttggcgctgcaagggtcggagctggtgccgg

SEQ ID NO: 988 - DNA, 224 bp, Burkholderia cepacia
agacggtgttcgacaacgccgtcgtgtggcgccgcttcctcgacgacaagcatttccagagccagaaggaactctccgagcacctcggcctcgacgaatcgacggtggccgtcgccctgtcgatcggcaaactgccggaagcgatcatgcaggaaatggtcgcgcgccccgatcgcttcggttcaaacatggcgtatcaggtcggccgctatcacaacgcgcgc

SEQ ID NO: 989 - DNA, 224 bp, Burkholderia cepacia
gccgaaatcgaaggcgagatgggcgattcgctgccgggcctgcaggcccgcctgatgtcgcaggcgctgcgcaagctgaccggcacgatcaagcgcacgaactgcctcgtgatcttcatcaaccagatccggatgaagatcggcgtgatgttcggcaacccggaaaccacgacgggcggcaacgcgctgaagttctattcgtcggtgcgtctcgacatccgccg

SEQ ID NO: 990 - DNA, 224 bp, Burkholderia dolosa
caccctaccgcggctcaagattctcgaaatcttccagcaaagccccgtgcgtcacctgacggccgaagacgtctaccgcaatctgctcagcgaacagctcgacatcgggctcgctaccgtctatcgcgtgctcacgcagttcgagcaggccggcctgctcacgcgcagcaacttcgaatccggcaaggccgtgttcgagctgaacgaaggttcgcaccacgatc

SEQ ID NO: 991 - DNA, 224 bp, Burkholderia dolosa
tacatcaacaagacgaagagcaacctgcacccgacgatcttccacgtgatcggcgagaaggacggcgtgggcgtcgaagtcgcgatgcagtggaacgacagctacaacgagaacgtgctgtgcttcacgaacaacattccgcagcgcgacggcggcacgcacctgaccggcctgcgggccgcgatgacgcgcgtgatcaacaagtacatcgccgacaacgaaat

SEQ ID NO: 992 - DNA, 224 bp, Burkholderia dolosa
aagcaggggcgcgaccgcaagttccaggcgatcctgccgctgcgcggcaaggtgctgaacgtcgagaaggcgcgctacgacaagctgctgtcgtccgagcagatcgtcacgctcgtgaccgcgctcggctgcgggatcggcaaggacgattacaacctcgacaagctccgctatcaccggatcatcatcatgaccgacgcggacgtcgacggtgcgcacatccg

SEQ ID NO: 993 - DNA, 224 bp, Burkholderia dolosa
ccggcgagcaggcgctcgaaatcaccgacgcgctggtgcgctcgggttcgatcgacatgatcgtcatcgactcggtcgcggcgctcgtgccgaaggccgaaatcgaaggcgaaatgggcgattcgctgccgggcctgcaggctcgcctgatgtcgcaggcgctgcgcaagctgaccggcacgatcaagcgcacgaattgcctcgtgatcttcatcaaccagatc

SEQ ID NO: 994 - DNA, 224 bp, Burkholderia multivorans
cctgctcaacgagcagctcgacatcgggctcgccaccgtctatcgcgtgctcacgcagttcgagcaggccgggctgctgtcgcgcagcaacttcgaatcgggcaaggccgtgttcgagctgaacgaaggctcgcaccacgaccacctcgtctgcctcgactgcgggcgcgtcgaggaattcttcgacgccgaaatcgaaagccgccagcaggcgatcgcgaagg

SEQ ID NO: 995 - DNA, 224 bp, Burkholderia multivorans
aaccgggcctacaagaagtcggcgcgtatcgtcggcgacgtgatcggtaaataccacccgcacggcgacagcgcggtctacgaaacgatcgtgcggatggcgcaggacttctcgctgcgctacatgctggtcgacgggcaaggcaacttcggctcgatcgacggcgacaacgccgccgcgatgcgttacaccgaaattcgcatggcgaagatcggtcacgagct

SEQ ID NO: 996 - DNA, 224 bp, Burkholderia multivorans
tcacgatgtcgcacgcgggctacgtgaagtcgcagccgctgtccgagtaccgcgcgcagaagcgcggcggtcgcggcaagcaggcgacgcagatgaaggaagacgactggatcgagacgctgttcatcgcgaacacgcacgattacatgctctgcttctcgaaccgcggccgcgtgtactggatcaaggtctacgaggtgccgcagggctcgcgcaactcgcgc

SEQ ID NO: 997 - DNA, 224 bp, Burkholderia multivorans
acccgacgatcttccacgtgatcggcgagaaggacggcgtgggcgtcgaagtcgcgatgcagtggaacgacagctacaacgagaacgtgctgtgcttcacgaacaacattccgcagcgcgacggcggcacgcacctgaccggcctgcgcgccgcgatgacgcgcgtgatcaacaagtacatcgccgataacgaaatcgcgaagaaggcgaaggtcgagacgacc

SEQ ID NO: 998 - DNA, 224 bp, Burkholderia multivorans
gcaagttccaggcgatcctgccgctgcgcggcaaggtgctgaacgtcgagaaggcgcgctacgacaagctgctgtcgtcggagcagatcgtcacgctcgtgaccgcgctcggctgcggcatcggcaaggacgattacaacctcgacaagctgcgctatcaccggatcatcatcatgaccgacgcggacgtcgacggtgcgcacatccggacgctgctgctcacg

SEQ ID NO: 999 - DNA, 224 bp, Burkholderia multivorans
agacggtcttcgacaatgcagtcgtgtggcgtcgcttcctcgacgacaagcacttccagagtcagaaggagctcgccgagcacctcggtctcgacgaatcgacggtcgccgttgccctgtcgatcggcaagctgcccgaagcagtgatgcaggagatggttgcgcggcccgatcgcttcggttcgaacatggcatatcaggtcggccgctatcacgctgcgcgc

SEQ ID NO: 1000 - DNA, 224 bp, Burkholderia multivorans
tctgctgatctcgcagccggacaccggcgagcaggcgctcgaaatcaccgacgcgctggtgcgctcgggctcgatcgacatgatcgtcatcgactcggtcgcggcgctcgtgccgaaggccgaaatcgaaggcgagatgggcgattcgctgccgggcctgcaggcgcgcctgatgtcgcaggcgctgcgcaagctgacgggcacgatcaagcgcacgaactgcc

SEQ ID NO: 1001 - DNA, 224 bp, Bordetella parapertussis
cgaaagcgtggggagcaaacaggattagataccctggtagtccacgccctaaacgatgtcaactagctgttggggccttcgggccttggtagcgcagctaacgcgtgaagttgaccgcctggggagtacggtcgcaagattaaaactcaaaggaattgacggggacccgcacaagcggtggatgatgtggattaattcgatgcaacgcgaaaaaccttacctac

SEQ ID NO: 1002 - DNA, 224 bp, Bordetella parapertussis
acgcaggcgccaacgtcctcaatggcctggcgggcaacgacgtgttgtccggcggcgccggcgacgatgtgctgctgggcgacgagggctcggacctgctcagcggcgatgcgggcaacgacgatctgttcggcgggcagggcgatgatacctatctgttcggggccgggtacggacatgacacgatctacgaatcgggcggcggccatgacaccatccgtatc

SEQ ID NO: 1003 - DNA, 224 bp, Bordetella pertussis
gctagagtgtgtcagagggaggtggaattccgcgtgtagcagtgaaatgcgtagatatgcggaggaacaccgatggcgaaggcagcctcctgggataacactgacgctcatgcacgaaagcgtggggagcaaacaggattagataccctggtagtccacgccctaaacgatgtcaactagctgttggggccttcgggccttggtagcgcagctaacgcgtgaag

SEQ ID NO: 1004 - DNA, 224 bp, Bordetella pertussis
gggcgacgagggctcggacctgctcagcggcgatgcgggcaacgacgatctgttcggcgggcagggcgatgatacttatctgttcggggtcgggtacgggcacgacacgatctacgaatcgggcggcggccatgacaccatccgcatcaacgcgggggcggaccagctgtggttcgcgcgccagggcaacgacctggagatccgcattctcggcaccgacgatg

SEQ ID NO: 1005 - DNA, 224 bp, Bordetella pertussis
gctttctgggcaccgtcaccgtcaaggccggcaagctggtcgccgatcacgccacgctggccaacgttggcgacacctgggacgacgacggcatcgcgctctatgtggccggcgaacaggcccaggccagcatcgccgacagcaccctgcagggcgctggcggcgtgcagatcgagcgcggcgccaatgtcacggtccaacgcagcgccatcgtcgacgggggc

SEQ ID NO: 1006 - DNA, 224 bp, Bordetella pertussis
aggtctatctcgaacatcgcatgcaggaagcggtcgaggccgaacgcgccggcaggggcaccggccacttcatcggctacatctacgaagtccgcgccgacaacaatttctacggcgccgccagctcgtacttcgaatacgtcgacacttatggcgacaatgccggccgtatcctcgccggcgcgctggccacctaccagagcgaatatctggcacaccggcgc

SEQ ID NO: 1007 - DNA, 224 bp, Bordetella pertussis
ggtttctatatggatggcatcgtgcgggtcagccgcattcgccacgcgttcaaggtggacgacgccaagggccggcgcgtgcgcggccagtaccgcggcaatggcgtgggcgcgtcgctggaactgggcaagcgcttcacgtggcccggcgcctggtacgtggagccgcagctggaggtggccgccttccatgcgcaaggggccgactacaccgccagcaacgg

SEQ ID NO: 1008 - DNA, 224 bp, Burkholderia pyrrocinia
ggctcgccaccgtctaccgcgtgctgacgcagttcgagcaggccggcctgctcacgcgcagcaacttcgaatccggcaaggccgtgttcgaactgaacgaaggctcgcaccacgaccacctcgtgtgcctcgattgcggccgcgtcgaggaattcttcgacgccgagatcgaaggccgccagcaggcgatcgcgaaggagcgcggcttccggctccaggagcac

SEQ ID NO: 1009 - DNA, 224 bp, Burkholderia pyrrocinia
tcgccggcggtgtgaagggcttcgtcgaatacatcaacaagacgaagaccaacctgcacccgacgattttccacgccaacggcgagaaggatggtgtgggcgtcgaagtcgcgatgcagtggaacgacagctacaacgagaacgtgctgtgcttcacgaacaacattccgcagcgcgacggcggcacgcacctgaccggcctgcgggccgcgatgacgcgcgtc

SEQ ID NO: 1010 - DNA, 224 bp, Burkholderia pyrrocinia
tcggcaaatgccggacatgatcgagcgcgggtatgtgtatatcgcgcagccgccgctttacaagatcaaggcgggcaaggacgagcggtatctgaaggatgacgtggagctcaacgcgcacatgctgcggttggcgctgcaaggctcggagctggtgcctggcgagaatgcggccgtgatttccggcgatgcgcttggggagctggcgcggtcgtatctgctgt

SEQ ID NO: 1011 - DNA, 224 bp, Burkholderia pyrrocinia
cgcgattcgcagacggtattcgacaacgccgtcgtgtggcgccgctttctcgacgacaagcatttccagagccagaaggagctctccgagcatctcggcctcgacgagtcgacggtggccgtcgcgttgtcgatcggcaagttgccggaagcgatcatgcaggaaatggtcgcgcgtcccgatcgcttcggttcgaacatggcgtatcaggtcggccgctatca

SEQ ID NO: 1012 - DNA, 224 bp, Burkholderia pyrrocinia
gtgccggaactgctgatctcgcagccggacaccggcgagcaggcgctggaaatcaccgatgcgctggtgcgctcgggctcgatcgacatgatcgtcatcgactcggtcgcggcgctcgtgccgaaggccgaaatcgaaggcgagatgggcgattcgctgccgggcctgcaggcccgcctgatgtcgcaggcgctgcgcaagctgaccggcacgatcaagcgcac

SEQ ID NO: 1013 - DNA, 224 bp, Burkholderia stabilis
cgtctaccgcaatctgctcaacgagcagctcgacatcgggctcgccaccgtctaccgcgtgctcacgcagttcgagcaggccggcctgctctcgcgcagcaacttcgaatccggcaaggcggtgttcgaactgaacgaaggttcgcaccacgaccacctcgtgtgcctcgattgcggccgcgtcgaggaattcttcgacgccgagatcgaaagccgccagcagg

SEQ ID NO: 1014 - DNA, 224 bp, Burkholderia stabilis
ggcgtgggcgtcgaagtcgcgatgcagtggaacgacagctacaacgagaacgtgctgtgcttcaccaacaacattccgcagcgcgacggcggcacgcacctgaccggcctgcgggccgcgatgacgcgcgtcatcaacaagtacatcaccgacaacgaaatcgcgaagaaggcgaaggtcgagacgaccggcgacgacatgcgcgaagggctgtcgtgcgtgct

SEQ ID NO: 1015 - DNA, 224 bp, Burkholderia stabilis
gatgccagacatgatcgagcgcgggtacgtgtatatcgcgcagccgccgctttacaagatcaaggcgggcaaggacgagcggtatctgaaggatgacgtggagctcaacgcgcacatgctgcggttggcgctgcaagggtcggagctggtgccgggtgagaatgcggccgtgatttccggcgatgcgctcggggagctggcgcggtcgtatctgctgtcgcgaa

SEQ ID NO: 1016 - DNA, 224 bp, Burkholderia stabilis
cgtgtggcgccgcttcctcgacgacaagcatttccagagccagaaggaactgtccgagcatctcggcctcgacgagtcgacggtggccgtcgcgttgtcgatcggcaagttgccggaagcgatcatgcaggaaatggtcgcgcgccctgatcgcttcggttcgaacatggcgtatcaggtcggtcgctatcacaatgcgcgcggcaccgaggccacgttgcggc

SEQ ID NO: 1017 - DNA, 224 bp, Burkholderia stabilis
cagccggacaccggcgagcaggcgcttgaaatcaccgacgcgctggtgcgctcgggctcgatcgacatgatcgtcatcgactcggtcgcggcgctcgtgccgaaggccgaaatcgaaggcgagatgggcgattcgctgccgggcctgcaggcgcgcctgatgtcgcaggcgctgcgcaagctgaccggcacgatcaagcgcacgaactgcctcgtgatcttcat

SEQ ID NO: 1018 - DNA, 224 bp, Burkholderia vietnamiensis
ctaccgcgcctcaagattctcgagatcttccagcaaagcccggtgcgtcacctgaccgccgaagacgtctaccgcaacctgctcaacgagcagctggacatcgggctcgccaccgtttaccgcgtgctgacgcaattcgagcaggccggcctgctgtcgcgcagcaacttcgaatcgggcaaggccgtgttcgagctcaacgaaggctcccaccacgatcacct

SEQ ID NO: 1019 - DNA, 224 bp, Burkholderia vietnamiensis
atcggcgacacgtcggacggcaccggtctgcaccacctcgtgttcgaggtgctcgacaactcgatcgacgaagcgctggccgggtactgcaacgacatccacgtgacgattcacgccgacaactcgatttccgtgaccgacaacggccgcgggattccgaccgacgtgaagatgaacgacaagcacgagccgaagcgcagcgccgccgagatcgtgatgaccga

SEQ ID NO: 1020 - DNA, 224 bp, Burkholderia vietnamiensis
atccggacgctgctgctcacgttcttctatcggcaaatgccggagatggtcgagcgcgggtacgtgtatatcgcgcagccgccgctttacaagatcaaggccggcaaggacgagcggtatctgaaggacgacgcggagctcaacgcgcatatgctgcggttggcgctgcaagggtcggagttggtgcctagcgagaacggcgcggcgatttcaggcgatgcgct

SEQ ID NO: 1021 - DNA, 224 bp, Burkholderia vietnamiensis
gcagacggtgttcgacaatgccgtcgtgtggcgacgcttcctcgacgacaaacatttccagagccagaaggaactgtccgaacatctcggcctcgatgaatcgacggtcgcggttgcgctgtcgatcggcaaactgccggagtcggtcatgcaggaaatggtcgcgcgtccggatcgcttcggctcgaacatggcgtatcaggtcggccgctatcacaatgcac

SEQ ID NO: 1022 - DNA, 224 bp, Burkholderia vietnamiensis
caccggcgaacaggcgctcgaaatcaccgacgcgctcgtgcgctcgggctcgatcgacatgatcgtcatcgactcggtcgcggcactggtgccgaaggccgaaatcgaaggcgagatgggcgattcgctgccgggtctgcaggcgcgtctgatgtcgcaggcgctgcgcaagctgacgggcacgatcaagcgcacgaactgcctcgtgatcttcatcaaccaga

SEQ ID NO: 1023 - DNA, 224 bp, Clostridium difficile
aggaatacaaggagtaaatgttactgtaccatataaggagcgtattatgaaatatcttgattttatatcaccagaggcaaaacgtataggagcagttaatacaattttactcagagaaaatatgttatatggttacaatacagattactttggtcttgatagtatgtttaaaatggctaatattgatgttcaaggaaaagtagcagttatattaggaactggag

SEQ ID NO: 1024 - DNA, 224 bp, Clostridium difficile
atatacaaatactaacaatgtaagtacattaatagagcaaggatatagtataaaaatagataaaattgttcgtatagttatagatgggaagcactatattaaagcagaagcatctgttgtaagtagtcttgattttaaagatgatgtaagtaagggggactcttggggtaaagcaaattataatgattggagtaataaattaacacctaatgaacttgctgatg

SEQ ID NO: 1025 - DNA, 224 bp, Clostridium difficile
aaagttgatgtctgattgggaagacgaagatttggatacagataatgataatataccagattcatatgaacgaaatggatatactattaaggacttaattgcagttaagtgggaagatagttttgcagaacaaggctataagaaatatgtatcaaattatttagagtcaaatactgctggagatccatatacagattatgaaaaagcttcaggttcttttgaca

SEQ ID NO: 1026 - DNA, 224 bp, Clostridium difficile
attatggatagcacaaaaaattagagaatcagattatattagtccaatcatatttcttacaaatcatatagaaatgattttagggatcttcgattataggttagaagttatggactttatactaaaacatgatatggaaattgctgaaagtaaaataaaagcttgtattaaaattgctcataaaagatacattaaagaaaaaaattatcgctctaattttttta

SEQ ID NO: 1027 - DNA, 224 bp, Clostridium difficile
gaagaaaagaaatgcttcaagatatagcaatacttacaggtgctcaagtaatatcagaagaattaggttatgatttaaaagaagctgatttatctatgctaggtagagcttcatctgttaaggtaactaaagaaagtactacaatagtagatggttctggagataaaaaagctatagaagatagagtaactcaaattaaacatcaagtagagcaaactacttca

SEQ ID NO: 1028 - DNA, 224 bp, Clostridium difficile
tggggacgttttaggtaagtaccatcctcatggagatactgctgtttattatgctatggtaagaatggcacaagatttttcaactagagcacttttagtagatggtcatggtaactttggttctgttgatggggattcaccagctgctatgcgttatacagaagctaaaatgagtaaattatcattagaactactgagagatattgaaaaggaaactgtagact

SEQ ID NO: 1029 - DNA, 224 bp, Clostridium difficile
gctgcaaaaaaagcaagagaattaacaaggagaaaaagtgtattagaaagtacatctttacctggaaaacttgcagattgtgcagaaaaagatccatctaaaagtgaaatattcttagtcgaaggggattcagcgggaggttcagctaaacaaggtagagatagaaatagtcaagctatacttccattaagagggaaaatacttaatgttgagaaatcaagact

SEQ ID NO: 1030 - DNA, 224 bp, Citrobacter freundii
cggtaccggtctgcaggacgaactggacgtggttgaaggtatgcagttcgaccgtggttacctgtctccttacttcatcaacaagccggaaactggcgcagtagaactggaaagcccgttcatcctgctggctgacaagaaaatctccaacatccgcgaaatgctgccggtgctggaagcagtagcaaaagcaggtaaaccactgctgatcatcgctgaagatg

SEQ ID NO: 1031 - DNA, 224 bp, Citrobacter freundii
atgtcgaaaatcgcccatgagctgatggctgacctggaaaaagaaacggttgatttcgtcgataactacgacggcaccgaacaaattcctgacgtcatgccaaccaaaattcctaacctgctggtgaacggttcgtccggtatcgcggtaggtatggcgaccaacattccgccgcacaacctgacggaagtgatcaacggctgtctggcatacattgacgatga

SEQ ID NO: 1032 - DNA, 224 bp, Citrobacter freundii
ctcgtggtgctcaggcaacggatatcgttgttctggttgttgcagcagacgacggcgtgatgccacagaccatcgaagctatccagcacgcgaaagcggcgggtgtgccggtggtggttgcagtcaacaaaatcgataagccggaagcggatccggatcgcgttaaaaacgaactgtcccagtacggcatcatgccggaagagtggggcggcgagagccaattc

SEQ ID NO: 1033 - DNA, 224 bp, Citrobacter freundii
gtggaccaacaacatcggtgacgcgaacaccgttggcggtcgtccggataacggtctgttgagcgttggtgtttcctaccgtttcggtcagcaggaagaagcagctccagtagttgttgctccggctccggctccagaagtacagaccaaacacttcactctgaagtctgacgttctgttcaacttcaacaaagcaactctgaaaccagaaggccagcaggctc

SEQ ID NO: 1034 - DNA, 224 bp, Chlamydophila pneumoniae
ttaaaaactctgccgtgactgtaattgctgatgggggaatccgctattctggagatgtggtaaaagcattagcagcaggagcagactgtgtcatgctaggaagtttgcttgcagggactgatgaagctcctggggatatcgtttctatcgatgagaagctttttaaaaggtaccgcggcatgggatctttaggcgctatgaaacaaggaagtgctgaccggtat

SEQ ID NO: 1035 - DNA, 224 bp, Chlamydophila pneumoniae
aaggtactactgtaaatgcaaatgaactaccaaacgtttctttaagtaacggagttgttgaactttacacagacacctctttctcttggagcgtaggcgctcgtggagccttatgggaatgcggttgtgcaactttgggagctgaattccaatatgcacagtccaaacctaaagttgaagaacttaatgtgatctgtaacgtatcgcaattctctgtaaacaaa

SEQ ID NO: 1036 - DNA, 224 bp, Chlamydophila pneumoniae
acttgtccgtaaaaataaacaaccagttgaacaaaaaagccgtggtgctttttgtgataaagaattttatccctgtgaagagggacgatgtcaacctgtagaggctcagcaagagtcttgctacggaagattgtattctgtaaaagtaaacgatgattgcaacgtagaaatttgccagtccgttccagaatacgctactgtaggatctccttaccctattgaaa

SEQ ID NO: 1037 - DNA, 224 bp, Chlamydophila pneumoniae
cctgaaagaactggaagagagaaaaaaacctcgaccagagcgtagaactactgccgatgtaaaaagatccccacgcttcttaccaaaacaagaagtcgaagagcctgtacctgccgcctctaaagaaaaattagatagtatacaggtttgggaagaaaaacaaaattatgctcgcagagccgtaaatgctatcaatctgagtataaaaaaacaactcgaagagc

SEQ ID NO: 1038 - DNA, 224 bp, Chlamydia trachomatis
gcacagaaaatgcgcaaaaatttgcggtgatacttccggagattatcacccccatggagaaagggtcatttatcctactttagtaaggatggcacaggattgggccatgcgataccctcttgttgatggtcaagggaattttggatccatcgacggggatccagctgctgccatgcgatatacagaggctcgcctgactcacagcgctatctttttgttagagg

SEQ ID NO: 1039 - DNA, 224 bp, Chlamydia trachomatis
acggagcttatcctttgtattactatcctccagaaagtgggaagcagggaggcagttatctctattctcatgaagagaaggaagcagctattgctgccaatgaagaatctgcttcgcggattttagagttatataaaggttctgttctcgaagaacttcagcgcgatttaggtgattacggttacgatatccggaattacctccatccaaaaggatctggtatt

SEQ ID NO: 1040 - DNA, 224 bp, Chlamydia trachomatis
gcttttgatggcattgatgcggcattccttatagggtcagttcctagaggcccaggaatggagagaagagatcttctaaagaaaaatggggagatttttgctacgcaaggaaaagctttgaacacaacagccaagcgggatgcaaagatttttgttgttgggaaccctgtgaataccaattgctggatagcaatgaatcatgctcccagattattgagaaagaa

SEQ ID NO: 1041 - DNA, 224 bp, Chlamydia trachomatis
tgtatcactgcaacgcaaatgctcgagtcaatgattcgcaacccccttcctacacgagccgaagtctctgacgttgccaacgccatttacgatggaacctctgcagtcatgttgtctggagaaactgccttaggagcccatcctgtacatgcagtaaaaacaatgcgttccattatccaagagactgagaagactttcgattaccacgcttttttccagctgaa

SEQ ID NO: 1042 - DNA, 224 bp, Enterobacter aerogenes
ggtaccaccaccgcaaccgtactggctcaggctatcgttaacgaaggtctgaaagccgttgctgcgggcatgaacccgatggatctgaaacgtggtatcgacaaagctgtcgttgccgctgtggaagaactgaaagcgctgtccgtaccgtgctctgactctaaagccattgctcaggttggtactatctccgctaactccgacgaaaccgtaggtaaactgat

SEQ ID NO: 1043 - DNA, 224 bp, Enterobacter aerogenes
ggcgttatcaccgttgaagacggtaccggtctggaagacgaactggacgtcgttgaaggtatgcagttcgaccgcggctacctgtctccttacttcatcaacaagccggataccggcgctgttgaactggaaagcccgttcatcctgctggctgacaaaaaaatctccaacatccgcgaaatgctgccggttctggaagccgtagcgaaagctggcaaaccgct

SEQ ID NO: 1044 - DNA, 224 bp, Enterobacter aerogenes
gctcgtggtgcgcaggcgacggatatcgtggttctggtggtggcggcagacgacggcgtgatgccgcagactgtggaagctatccagcacgccaaagcggcgcaggtgccggtggtggttgcagtgaacaagatcgataagccggaagcggatatggatcgcgtgaagaacgaactctcccagtacggcgtgatgccggaagagtggggcggcgaagcgcagtt

SEQ ID NO: 1045 - DNA, 224 bp, Enterobacter aerogenes
cggtttcagcaccgcggcaacctacgcgtttgataacggtatcgcgctgtctgcaggttacgctaactccaaccgtagcgttgaccagaaacgtgacggcaacggtgacaaagctgaagcatgggcaacctctgccaagtacgatgctaacaacatctatgccgcagttatgtactcccagacttacaacatgacgccggaagaagatgaccacttcgcgggca

SEQ ID NO: 1046 - DNA, 224 bp, Enterobacter aerogenes
cgttgtttacgacgtaacttcctggaccgacgttctgccggaattcggtggcgacacctacggttccgacaacttcctgcagtcccgtgctaacggtgttgccacctaccgtaactctgacttcttcggtctggttgacggcctgaactttgctctgcagtaccagggtaaaaacggcagcgtgagcggcgaagatcagaccaacaacggtcgcggcttccaga

SEQ ID NO: 1047 - DNA, 224 bp, Enterobacter cloacae
ctttggttctatcgacggcgactccgccgcggcaatgcgttatacggaaatccgtctggcgaaaattgcccatgagctgatggctgacctggaaaaagagacggttgatttcgttgataactatgacggcacggaacgcattccggacgttatgccaacgaaaatccctaacctgctggtgaacggttcgtccggtatcgccgtcggtatggcaaccaacattc

SEQ ID NO: 1048 - DNA, 224 bp, Enterobacter cloacae
tacttctctactgaaaaagacggtatcggcgtggaagtggccttgcagtggaacgacggtttccaggaaaacatctactgcttcaccaacaacattccacagcgcgacggcggtacgcaccttgcgggcttccgcgcggcgatgacccgtaccctgaacgcctacatggacaaagaaggctacagcaaaaaagcgaaagtcagcgccaccggtgacgatgcccg

SEQ ID NO: 1049 - DNA, 224 bp, Enterobacter cloacae
ccaacctggatgacgaaggccactttgtagaagatctggttacctgccgtagcaaaggcgaatccagcttgttcagccgcgaccaggttgactacatggacgtatccacccagcaggtggtatccgtcggtgcgtccctgatcccgttcctggaacacgatgacgccaaccgtgcattgatgggtgcgaacatgcaacgtcaggccgttccaactctgcgtgct

SEQ ID NO: 1050 - DNA, 224 bp, Escherichia coli
ccgatgaatgagtatatctggaataccggaaccaatagtcgtccacaggagttgtttatatatttctcacgtgttgatgcattcgctaacagagtaaatcttgcgattgtttcaaacagaagagctgatgaggtgattgtattacctcctccaactgttgtatcacgaccgatcatcggcattagaattggtaatgatgttttcttctcaacccatgcattggc

SEQ ID NO: 1051 - DNA, 224 bp, Escherichia coli
tctccgccatcaacggctgtagatacaggtagagttattccttccccaggaattcctgtccgagagcttatctggaacttgtcgacaaatagcaggccacagcaagtatatatatatttttctgctgttgatgcccttggtggaagagtcaatcttgctctggttagcaatcggcaggccgatgaagtgtttgttcttcgcccggtaagacaaggtggacgacc

SEQ ID NO: 1052 - DNA, 224 bp, Escherichia coli
ttaacatcgacaactccaggaaaatcactcgttagtgcccgtgttagcgatgtcgccgttgatgtcaaagcacctgaagttgaattttttacaacgcttacaattgatgacggtaatattgaaattgttggaaccggagttaaagggaagttacccactgtatggttgcaatatggtcaagttaatctgaaagccagcggaggtaacggaaaatatacatggcg

SEQ ID NO: 1053 - DNA, 224 bp, Escherichia coli
tgagtattgatggtgataaagtgaccgtaattggtactggtatcacgggggctctgccaaagaactggttacagtatggtcaggttaagctacaggcaacagggggcaatggaaaatacacatggaaatccagtaatactaaaattgcttctgttgataactcgggagtgataaccttaaatgaaaaagggagtgccacaattactgtagtatctggtgataat

SEQ ID NO: 1054 - DNA, 224 bp, Escherichia coli
ggtggagccataacatgattactcatggttgttatacccggacccggcacaagcataagctaaaaaaaacatttattatgcttagtgctggtttaggattgtttttttatgttaaccagaattcatttgcaaatggtgaaaattattttaaattgagttcagattcaaaactgttaactcaaaatgccgctcaggatcgccttttttatacgttaaaaacaggt

SEQ ID NO: 1055 - DNA, 224 bp, Escherichia coli
ccaaaacggatggtaacggtaaggcaactgtaacgttgaagtcgggtacgccagggcaggtcgtcgtgtctgctaaaaccgcggagatgacttcagctacttaatgccagtgcggttatatttgttgatcaaaccaaggccagtattactgagattaatgctgataaaacaacagcgaaggcaaatggttctgatgcgattacctatactgttaaagtaatgaa

SEQ ID NO: 1056 - DNA, 224 bp, Escherichia coli
gttgttagtgcaaaagttagtgaagctggctcagtggttaatgcagatgcagttaacttcttcgctacacttagtattgataataatgtcgagatcgttggtacaaaagttcgtggtgaattaccaaatatctggctacgatatggccaagttaagttgaatgcaaatggcggtaatggtggatactcatggagttccgataatccagatattgcatcgataga

SEQ ID NO: 1057 - DNA, 224 bp, Escherichia coli
agttcttcacagaactgagtatcaataaaaatgtagaggttcttggtacaaaggctagtggtgagttacctgatgtctggctacaatatggtcagattaaattaaatgttaatggtggcaatgataaatactcatggagctccagtaatcctaatattgcgtcgatagatgcctcttccggtattattaccttaaaggaaaagggggaggctgttattaaggtt

SEQ ID NO: 1058 - DNA, 224 bp, Escherichia coli
attttttacacagcttacaattgataacggtaatgttgaaattgttggaaccggagctaaggggaaactacctaatgtatggttgcaatatggccaagttaatctgaaagctagcggaggtaacgggaaatatacatggtactcagcaaatccggcaattgcttcggtggatccctcttctggtcaagtcacattaaaagataagggggaaacaacaattaccg

SEQ ID NO: 1059 - DNA, 224 bp, Escherichia coli
tggtgggactgatgttaaagcgactgaggttactttttttgatgaactgaaaattgacaacaaggttgatattattggtaacaatgtcagtggcgacttgcctaatatctggttgcaatatggtcagtttaaactgaaggcaagcggtggtaatggtacatattcatggcattcagagaataccaatattgcgactgttgatgaatcagggaaagtaaccttga

SEQ ID NO: 1060 - DNA, 224 bp, Escherichia coli
gggcttggtgaggcaaaactgaatgctaaaccggcacgcgtagtcatggagaaaccgctggggacgtcgctggcgacctcgcaggaaatcaacgatcaggttggcgaatacttcgaggagtgccaggtttaccgtatcgaccactatcttggtaaagaaacagtgctgaacctgttggcgctgcgttttgctaactctctgtttgtgaacaactgggacaatcg

SEQ ID NO: 1061 - DNA, 224 bp, Escherichia coli
tatctcaggaaatatcaatcaaaagttaagaggcagatattttcagactatcagtcagaggttgacatatataacagaattcgggatgaattatgaataaagtaaaatgttatgttttatttacggcgttactatcctctctatatgcacacggagctccccagactattacagaactatgttcggaatatcgcaacacacaaatatatacgataaatgacaag

SEQ ID NO: 1062 - DNA, 224 bp, Escherichia coli
gcatgtccagtaatggggtaaatgataataggtgttggggaaatccaactgcattaagtatgcattgttatttttttgtgattaacaccacaaaaacagtcattaaacataaatatatcatttagcttaaacttttctgtttttttcggtcgccgaaaaagataatattactatgctcttcgtagcggagagtatagtatgatgttcatcacaaaaaaaataaa

SEQ ID NO: 1063 - DNA, 224 bp, Escherichia coli
aactcacaaaaaaaaataaaaaaattgcaaaatccgtttaactaatctcaaatatccgtgaaacaacatgacgggaggtaacatgaaaaagctaatgttggcaatttttatttctgtattatctttcccctcttttagtcagtcaactgaatcacttgactcttcaaaagagaaaattacattagagactaaaaagtgtgatgttgtaaaaaacaacagtgaaa

SEQ ID NO: 1064 - DNA, 224 bp, Escherichia coli
gtataagttttattgcttatagcaataaggttgaggtgattttatgaaaaagaatatcgcatttcttcttgcatctatgttcgttttttctattgctacaaatgcctatgcatctacacaatcaaataaaaaagatctgtgtgaacattatagacaaatagccaaggaaagttgtaaaaaaggttttttaggggttagagatggtactgctggagcatgctttg

SEQ ID NO: 1065 - DNA, 224 bp, Escherichia coli
gatcagtcgtacggggatgcagataaatcgccattcgttgactacttcttatctggatttaatgtcgcatagtggaacctcactgacgcagtctgtggcaagagcgatgttacggtttgttactgtgacagctgaagctttacgttttcggcaaatacagaggggatttcgtacaacactggatgatctcagtgggcgttcttatgtaatgactgctgaagatg

SEQ ID NO: 1066 - DNA, 224 bp, Escherichia coli
tccatgacaacggacagcagttataccactctgcaacgtgtcgcagcgctggaacgttccggaatgcaaatcagtcgtcactcactggtttcatcatatctggcgttaatggagttcagtggtaatacaatgaccagagatgcatccagagcagttctgcgttttgtcactgtcacagcagaagccttacgcttcaggcagatacagagagaatttcgtcaggc

SEQ ID NO: 1067 - DNA, 224 bp, Escherichia coli
attaaagtaaataatgttttgtgggaagcgaatacaatcgctgctttattaaatcgcaagcctcaggatcttactgaaccaaaccaataacagggggtgaatatgaagaagatgattattgcagttttattcggtctcttttctgctaattccatggcggcggattgtgctgtaggaaaaattgagttttccaagtataatgaggatgatacctttactgtgaa

SEQ ID NO: 1068 - DNA, 224 bp, Escherichia coli
accgaatcgacgccgactttctcgcactgggcataaaactcatcaatgcctttgttaaacaccagattggcatacatcaacaggccaatgggaatggtcgggtgtttctggcgaatcagtgccagcatttcaaaacattgtgccggagtcacacctgccgcaaaggcgcgcagagtggcgttttgaatcgtcgggccatccgccagtgggtcggagaaggggat

SEQ ID NO: 1069 - DNA, 224 bp, Enterococcus faecalis
gaaccaatgcaaactggcttaaaagccattgatgccctagtaccaattggtcgtggacaacgggaattagttattggagaccgtaaaacagggaaaacatcgattgcaattgatacaattattaaccaaaaaggacaagatgtaatttgtatttatgtagcgattggtcaaaaagaatctactgttcgtaaccaagtagaaacacttcgtaaatttggtgcctt

SEQ ID NO: 1070 - DNA, 224 bp, Enterococcus faecalis
gcggatgatgttgatggggaagctctaccaacattagtattgaacaaaatccgtggtacatttaatgttgtcgcagtaaaagcgccaggatttggtgaccgccgcaaagcgatgcttgaagatattgctattttaacaggtggtacagtaatcactgacgacttagggttagagttaaaagacacaactattgaaaacttaggaaatgctagcaaagtagttgt

SEQ ID NO: 1071 - DNA, 224 bp, Enterococcus faecalis
gtgcattacgaatgatttctcctgggaaagtattccgtcgtgatacagatgatgcgacacatagtcaccaattccatcagattgaaggccttgttgtcgataaaaatgtcacaatgggtgatttaaaagggacgttagaagtcatgatgaaaaaaatgtttggtgaagatcgtaaaattcgtttacgtcctagctatttcccttttacagaaccttcagtcgaa

SEQ ID NO: 1072 - DNA, 224 bp, Enterococcus faecalis
aaacaggtgttgcgacgtgaatacctaattggcgagcagccatacctaagtgtaattcaagtacttgtccgatattcatacgagaaggtacccctaatgggttcaacatgatatcaacaggtgttccgtcaggtaagaatggcatatcttcttccggcataatacgggaaacaacccctttatttccgtgacgtcccgccattttatctccttcgtgaatttta

SEQ ID NO: 1073 - DNA, 224 bp, Enterococcus faecium
accaggtgttatgcaacgtaaatctgttaacgaaccaatgcaaacagggctaaaagcgatcgacgcccttgtgccaatcggacgcggacaacgtgaattagtcatcggtgaccgtaaaacagggaaaacttctattgcgatcgatacgatcatcaaccaaaaaggtcaagatatgatctgtatctatgtagcaatcggacaaaaagattctacagttcgtacac

SEQ ID NO: 1074 - DNA, 224 bp, Enterococcus faecium
ggaagctgttctagaaaacccatatatcttgattacagacaaaaaaatctctaacatccaagacattctgccattgttagaacaaatcttgcaacaatcacgtccattattgatcattgctgacgatgtagatggtgaagcattgccaactcttgtattgaacaaaatccgtggaacattcaacgtagtagctgtaaaagctcctggtttcggcgatcgtcgta

SEQ ID NO: 1075 - DNA, 224 bp, Enterococcus faecium
ttttccgcagagatacagatgatgcgacccacagccatcagttccatcaaattgaagggctagttgttgataaaaacatcacgatgggcgaccttaaaggaacattagaagtcgtaatgaaaaaaatgtttggggaagatcgtgaaatccgtttgcgtccaagttatttcccatttacggagccatcagtagaagtagatgtcagctgtttcaaatgtggtggt

SEQ ID NO: 1076 - DNA, 224 bp, Enterococcus faecium
agttgaaaaaggcgatactttagcagatggaccttcaatggaacaaggtgaaatggcgctaggacaaaacgttttagttggtttcatgacttgggaaggttacaactatgaagatgcgatcatcatgagccgtcgtttggtaaaagatgatgtctatacttctatccatattgaagaatatgaatcagaagctcgtgatacaaaattaggacctgaagaaatca

SEQ ID NO: 1077 - DNA, 224 bp, Enterococcus faecium
gttcacgatgtgaaaatctttactcgtgaagctggcgatgaattgtcaccaggcgtaaacatgttagttcgtgtttatatcgttcaaaaacgtaaaatccatgaaggggataaaatggccggtcgtcacggtaataaaggggttgtatcccgtattatgccggaagaagatatgccattcttaccagatggaacaccaatcgatatcatgttgaatcctttagg

SEQ ID NO: 1078 - DNA, 224 bp, Haemophilus influenzae
actgcgcaaatggtttcttgttagatggtttccctcgtactattccacaagcggatgcactgaaagattcaggtgttaaaattgactttgttttagaatttgatgtgccagacgaagtgattgttgaacgtatgagtggccgtcgcgtacaccaagcgtctggtcgttcttaccacatcgtttataatccaccaaaagtggaaggtaaagatgatgtaacaggc

SEQ ID NO: 1079 - DNA, 224 bp, Haemophilus influenzae
actccaggacacgcagcgtttacttctatgcgtgcacgtggtgcaaaagcaacagatatcgttgttcttgttgttgcggccgatgatggtgtgatgcctcaaactatcgaagcgattcaacacgcaaaagcagcgggtgcacctttagtggttgcagtaaataaaatcgataaaccagaagcaaatctagatcgtgttgagcaagaattattacaacatgatgt

SEQ ID NO: 1080 - DNA, 224 bp, Haemophilus influenzae
gttcagatttattcaatattaatgcaggtatcgtgcgtggtttaattgaaaaagtcgcggttacttgcccgaaagcttgtgttggtatcatcactaacccagtaaatactactgttgcgattgcggctgaagtgttgaaaaaagcaggtgtttacgacaaacgtaaattatttggtgtgacaactttagacgtgttacgttctgaaacctttgtggctgaatta

SEQ ID NO: 1081 - DNA, 224 bp, Haemophilus influenzae
atgcgttatacggaatctcgcctatctaaaatctctgaaatcttgttgaatgaactcggacaaggaacagtagattatcaaccaaactttgatggaaccttggctgaaccacaatatttacctgctcgtttaccgcatattttattgaacggcacaacaggtattgcggtggggatggcgacagatattccaccacacaatattaacgaaattgctgatgcggc

SEQ ID NO: 1082 - DNA, 224 bp, Haemophilus influenzae
ttctgtcagcgtattatttctggcgaatggaaaggctatacaggtaaagccattacggatgttgtgaatattggtattggtggctctgacttaggcccttatatggtaaccgaagcacttcgcccgtataaaaatcatctaaatatgcactttgtttcaaatgtcgatggtacacatattgcggaaaccttaaaaaaagtcaatccagaaaccactcttttctt

SEQ ID NO: 1083 - DNA, 224 bp, Haemophilus parainfluenzae
gatcgtatttctcaaccagattgtgcaaatggtttcttattagatggtttcccacgtacgattccacaagcggatgcattaaaagattcaggcgtgaaaattgactacgttttagagtttgatgtaccagatgaagtgatcgttgaacgtatgagtggtcgtcgtgtacaccaagcgtctggtcgttcttatcacatcgtttacaacccaccaaaagtggaagg

SEQ ID NO: 1084 - DNA, 224 bp, Haemophilus parainfluenzae
gacacgccgcatttacctcaatgcgtgcacgtggtgcgaaagcaacggatatcgtcgttcttgtggttgcggcagatgatggtgtgatgcctcaaactattgaagcgattcaacacgcgaaagcagcgggtgcgcctttagtggttgcggtgaacaaaattgataaaccagaagcaaatccagatcgcgtagaacaagaattacttcaacacgaagtgatttct

SEQ ID NO: 1085 - DNA, 224 bp, Haemophilus parainfluenzae
gggtcgtattgttgaaatttttggacctgaatcttctggtaaaacaactttaactttatctgttatcgctcaagcacaaaaagtaggtaaaacctgtgcgtttatcgatgcggagcatgcacttgatcctatttatgcagcaaaacttggtgttgatgttaaagaacttttagtctcccagccagataatggtgaacaagcacttgaaatttgtgatgcattag

SEQ ID NO: 1086 - DNA, 224 bp, Haemophilus parainfluenzae
gaaactttacctgctgaattttctgaaatgtgtgcaggtcaattaatcagccaattagataaaatcccagctgaaaaacgtactgcattacgtaacaacgcaggcggtcacgcaaaccacagcttattctggaaatcattgaaaaaaggcactactttacaaggtgatttaaaagcagctatcgaacgtgatttcggttctgtagaaaacttcaaagctgaatt

SEQ ID NO: 1087 - DNA, 224 bp, Klebsiella oxytoca
agcaaccggtatcttcctgaccgacgaaaccgctcgtaaacacatcaccgctggcgcgaaaaaagtcgtactgactggcccgtccaaagacaacactccgatgtttgttaaaggcgctaactttgacaaatacgaaggccaggacatcgtttccaacgcatcctgcaccaccaactgcctggctccgctggcaaaagttatcaacgacaacttcggcatcatcg

SEQ ID NO: 1088 - DNA, 224 bp, Klebsiella oxytoca
cgtaggtaaactgatcgctgaagcgatggacaaagtcggtaaagaaggcgtgatcaccgttgaagacggtactggtctggaagacgaactggacgtggttgaaggtatgcagtttgaccgcggctacctgtccccgtacttcatcaacaagccggaaaccggcgctgttgaactggaaagcccgttcatcctgctggctgacaaaaaaatctccaacatccgcg

SEQ ID NO: 1089 - DNA, 224 bp, Klebsiella oxytoca
gtgcgtaccacgttgaaaccgacaacggtatgatcaccttcctggataccccgggtcacgccgcgtttacctctatgcgtgctcgtggtgcgcaggcgacggatatcgtggttctggttgtggcggcagacgacggcgtaatgccgcagactatcgaagctatccagcacgcgaaagcggcgcaggtacctctggtggttgcggtgaacaaaatcgataagccg

SEQ ID NO: 1090 - DNA, 224 bp, Klebsiella oxytoca
ctggaataccagtgggttaacaacatcggcgacgcaggcaccgttggtacccgtcctgataacggcatgctgagcctgggtgtttcctaccgtttcggtcaggaagatgttgctccggttgtagctccggctccggctccggctccggaagtgactaccaagcacttcaccctgaagtctgacgttctgttcaacttcaacaaatctactctgaaaccagaagg

SEQ ID NO: 1091 - DNA, 224 bp, Klebsiella oxytoca
cgagccgggcaccaacggtcagcacgcgttctatcagctgattcaccaggggaccaaaatggtgccgtgcgattttatcgctccggcgattacgcataacccgctgtctgaccatcatccgaagctgctgtctaacttctttgcgcagaccgaagcgctggcgtttggtaaatcccgcgaagtggttgaacaggaatatcgcgatcagggtaaagatcccgcga

SEQ ID NO: 1092 - DNA, 224 bp, Klebsiella oxytoca
ggcccgggcggtctgacccgtgaacgcgcaggctttgaagttcgagacgtacacccgacccactacggtcgcgtatgtccaatcgaaacgcctgaaggtccgaacatcggtctgatcaactccctgtccgtgtacgcacagacgaacgaatacggcttccttgagactccgtatcgtaaagtgaccgacggtgttgtaaccgacgaaattcattacctgtctgc

SEQ ID NO: 1093 - DNA, 224 bp, Klebsiella pneumoniae
cgcgccagtgaactgctcggcggagaacgggggatggcgcgaaaaattcatcccaatgacgatgtgaataagagccagagctcaaatgatgtattccccaccgccatgcacgtcgcggcgctgattgccctgcgggaaaaagtgatcccgtcgctgcaggcgctgcgggcgacgctcaatgaaaaagcggtggccttcagggatatcgtcaagatcggccgcac

SEQ ID NO: 1094 - DNA, 224 bp, Klebsiella pneumoniae
cgcggaagcgatggataaagttggtaaagaaggcgtgatcaccgttgaagacggtaccggtctggaagacgaactggacgtagttgaaggtatgcagttcgaccgcggctacctgtctccgtacttcatcaacaagccggacaccggtgccgttgagctggaaagcccgttcatcctgctggctgacaaaaaaatctccaacatccgcgaaatgctgccggttc

SEQ ID NO: 1095 - DNA, 224 bp, Klebsiella pneumoniae
tcgctgcgttacatgctggtggacggccagggtaactttggttccatcgacggcgactccgccgcggcgatgcgttataccgaaattcgtctggcgaaaatcgctcatgagctgatggccgatcttgaaaaagagacggtcgatttcgtcgacaactatgacggtacggagcgtattccggacgtcatgccgaccaaaattcctaacctgctggtgaacggcgc

SEQ ID NO: 1096 - DNA, 224 bp, Klebsiella pneumoniae
ggcttccaggaaaacatctactgctttaccaacaacattccgcagcgtgacggcggtactcacctcgccggcttccgcgcggcgatgacccgtacccttaacgcctatatggacaaagagggctacagcaaaaaagcgaaagtcagcgccaccggcgacgatgcgcgtgaaggcctgattgccgtggtatccgtgaaggtgccggatccgaaattctcctcgca

SEQ ID NO: 1097 - DNA, 224 bp, Klebsiella pneumoniae
cgggcgtggcgcgtaagcccggcatggatcgttccgacctgtttaatgtgaatgcgggtatcgtgaagaacctcgtgcagcagattgccaaaacctgcccgcaggcctgcatcggcattatcaccaacccggtgaataccaccgtggctatcgccgccgaagtactgaaaaaagccggcgtgtacgataaaaacaaactgttcggcgttaccacgctggacatc

SEQ ID NO: 1098 - DNA, 224 bp, Klebsiella pneumoniae
accaacgaatatggcttccttgagacgccgtatcgtaaagtgaccgacggtgtggttactgacgaaattcactacctgtctgctatcgaagaaggcaactacgttatcgctcaggcgaactccaacctggatgaaaacggccacttcgtagaagatctggttacctgccgtagcaaaggcgaatccagcttgttcagccgcgaccaggttgactacatggacgt

SEQ ID NO: 1099 - DNA, 224 bp, Klebsiella pneumoniae
gaaccgccgaaagaggcgccggtggtgatccataaaccggaacctaagccgaagcccaaacctaaacccaagcctaagccggagaaaaaggttgaacagccgaagcgggaagtgaagccggcagcagagccgcgtccggcctcgccgtttgaaaacaacaatacggcgccggcgcgtacagcgccaagtacctcgaccgcagcggctaaacccaccgttactgc

SEQ ID NO: 1100 - DNA, 224 bp, Legionella pneumophilia
gacaatggttatacccgagaagagatgaagatggtctgggaaacccgcaagattatggaagatgacagcattatggttaaccctacagccgtcagggttcctgttatttatgggcattctgaagcggttcatctggaattaaaaaagcctttgacggctgacgatgctcgcgcgcttttggcaaaggcacccggcgttactgtagtggataatctttctaaagc

SEQ ID NO: 1101 - DNA, 224 bp, Legionella pneumophilia
tcaagttggaactatttctgctaattccgatgaagcgattggtgctatcattgctgaagcaatggaaaaagttggtaaagagggtgttattaccgttgaagatggtaatggattggaaaatgagctttctgttgttgaaggtatgcaatttgatcgcggttacatttctccatacttcatcaacaaccagcaaaacatgagctgtgaacttgagcatccattca

SEQ ID NO: 1102 - DNA, 224 bp, Legionella pneumophilia
ttattggtagtatctttgactatcaggtttatacatcttacgagttcatgaaaattgagcaggaactttatccatgctaattatgatgctactctactgattagaggcggctcaatctcttgagtaaaacctggtggtcaataacaaatagtacaacatttgagtgaatcatatctaatcactatgctattgattatttcaatgactaaatttttcattaaaat

SEQ ID NO: 1103 - DNA, 224 bp, Legionella pneumophilia
gatgcttatgtgcgtgcaattctaaatgatttggatgcccttaatgtgatagctaagttgcttgaggtacacgatgcagtacatgccattcgcatgacaggtgatcctgattatactgcagatgattggcgcgcaactttacctggagataaaatacctgttcgcgagctgaataattttgagggggatccttctgatttattatggccgccgttgtccaggca

SEQ ID NO: 1104 - DNA, 224 bp, Legionella pneumophilia
agtagtgtctcttgatttggatgaagtagcgaaaagtggtggtgatgcggcagatagacaaacagcggttatgtatatgttagccagatatgtattggcaaggcactattatttaacagaagaaagtttgaataatgtaccggaacaatataaagagtatcacaaacagagagtattagagattcgagaagatcataagcgtattgtttatgatgaattccatc

SEQ ID NO: 1105 - DNA, 224 bp, Legionella pneumophilia
ttactggaggcgcttatttgattgggtgtgcttttatatttaaggcaatttatagcttaaaggtttatggagaagccaggacaatgatgtcgagtaatacgagtatcaaagagccggtgatgtatttgatggtaggtgcattacttatctatttcccgtcattggtttcctccgtactgcaaacgacttttggttatagcaatccattagcctactcaggaggt

SEQ ID NO: 1106 - DNA, 224 bp, Legionella pneumophilia
atgttaggagactcaaaatatttttcatcccaacccattctggattggtttcaacaacaatggcagggacgtgcaccaggcactacgcaaaaacagttggctcttttaaaacaaactcttagcaatcctcctcaaaatatacccatcaaatcacaaataatcagtgatgcgaggaattatcttaatgcacttccaacaagctatttatattactctattgcgaa

SEQ ID NO: 1107 - DNA, 224 bp, Legionella pneumophilia
tgaagatgaccccgatctaaagaagaaagtttcagcaatagaaatgacacagcagagtcttcgttctgaagtcaatgcccttggtgagcaaatcaatgcagtaaataataatatcaaaaatttaaatgctcaaattgtaaatctgaatcaaattattggtaatatgtcaaatcaaatagctagacaatcagaagtgattaatgtcttaatggcacgtacaacac

SEQ ID NO: 1108 - DNA, 224 bp, Legionella pneumophilia
atctggatggtgattatacaaacaataggttatcaaatttagttacagcttgttgtttttgtgcgcaatgcttttttgttgagtcagtaggagttggtggttatggtggaggtactttgatctacctgccagaattaacccaagctgaactaaatagcttatgccatgttcttttttgtgctattaccaatgataccggatacaaatccagtgcacagaatatt

SEQ ID NO: 1109 - DNA, 224 bp, Legionella pneumophilia
taaacctacagcgacatcacaatttgttaatctgtctccaggctcaacaccgccggtaataagattatcacaaggttttgtgtcttccttagtgttcctggattcaacaggagccccttggcctattgctgcatatgatttgggtgatccctcttcgtttaatattcaatgggataaaaccagtaatactttaatgattcaagctacgaagctatataactatg

SEQ ID NO: 1110 - DNA, 224 bp, Legionella pneumophilia
ggatgaaccaaaccaatcggactccgctgtactacaatgggctaatcaggcagcaattgccgcatttacctataatttcgtaaattatcgcgatgagctacaggcttcttccggcttttttactgcggaaggctgggaccaatttttaggtgcattagagcaatccaataaccttgatgcggttaaagcgaaaaaattagttgtgtcagcagtagcaacccgag

SEQ ID NO: 1111 - DNA, 224 bp, Legionella pneumophilia
ctagcgctctgtttgctgatcaatctcctcggttaaattgggcttcctatagccttttaaatgagatcgcggcgtttcttaaacaatttcgtaaaattgctataacggtaaccagttacagtagtaaatatgtgtcggtaaaaagagaacgagcattgacattggcgagatcaagagtagtaagtgaatacctatggtctcagggggttgatagcagaatcatc

SEQ ID NO: 1112 - DNA, 224 bp, Legionella pneumophilia
ggaacaatttaattggcctgccatcatgcctatagttaaggaggatttggttagtcaagacgtcaataaagggccttgggcaatggctttaacacccatggaatttgcgcgaaaatataatttattgagaaaggacgacgcgttattggataatcctgtgcctggccaagagatgacagcggggattcggcgtggcgatgccaaacgagtatttaccatgcaat

SEQ ID NO: 1113 - DNA, 224 bp, Legionella pneumophilia
aattgccttaaggagtggtcagcaggaaatatatgtttcattatattcctcagatggaagcaatcttcagtcctgggagaaaatagtggggagtttgccccgacaaatgatttcacgacctatttacgcagatgaagaagacataaaagcgatattaaaaacaaaagaaaataaacaaaatgaagcgtatgtggcaatttacatcagtcaatcagatatcttgc

SEQ ID NO: 1114 - DNA, 224 bp, Legionella pneumophilia
caacccatgtggaatcatttgatgaagcagtcaagaaactaaaattgaatgaggctgacttgataatcaaacaaaaagccttgttccggctaagtgtgattatggttatcgctgcttttatgctactcatttacacgggttatcaacttttttatggttcatggaaagctacaataataagtttggttgtggttatgatcgcattggtactagcctttcgttat

SEQ ID NO: 1115 - DNA, 224 bp, Legionella pneumophilia
ttacagggtcatcacttttatggaaagtgtggaaaactggactcttgatggaaatccggaactggaagaggcaatgaaacagttagggcaagaacttgatgatattgaaaaaattgacttgggcttgctagcagaagaagacaaatttattcgtatcgttggaaacatcaaatcaggaagaggcttacgcttgcttcaggcaatagatacagtccatcctggta

SEQ ID NO: 1116 - DNA, 224 bp, Legionella pneumophilia
gggagcttgggcagtggttgttgattggtctattatgccgttagctgtaattttcccatccgcgccggaggatggggagctgaaattttgatatttaaaggtgctgtttgccaagttatttattaatgcatttccttgcacattattggtgacaaaattcattaagctctgtgacatggcatcgacaggaatagcgcctaaaaaggtattgtaagcgtaacttt

SEQ ID NO: 1117 - DNA, 224 bp, Legionella pneumophilia
tttaaccgaacagcaaatgaaagacgttcttaacaagtttcagaaagatttgatggcaaagcgtactgctgaattcaataagaaagcggatgaaaataaagtaaaaggggaagcctttttaactgaaaacaaaaacaagccaggcgttgttgtattgccaagtggtttgcaatacaaagtaatcaattctggaaatggtgttaaacccggaaaatcggatacag

SEQ ID NO: 1118 - DNA, 224 bp, Legionella pneumophilia
tatttcaacaactttgcctttaacggattcaactctaagttcaatggctttggtcctcgcactggtttagacatgaactacgtatttggcaatggctttggtgtttatgctaaaggcgctgctgctattctggttggtaccagcgatttctacgatggaatcaacttcattactggttctaaaaatgctatcgttcctgagttagaagctaagcttggtgctga

SEQ ID NO: 1119 - DNA, 224 bp, Legionella pneumophilia
aaaattttctttatgatatacttaaactggcgcgagttcgatataaaaagcatggaaatacctcttatatctggttaagctggcattatccagtaaaacacaacacatcaggatacgggttccactgcaaatgaagtcgagcatagtatcaaatgagtaagtgaatcacctgaaatattggttttacaagatttatccggataataaaacatttcaacatcaac

SEQ ID NO: 1120 - DNA, 224 bp, Legionella pneumophilia
atgatgtcactgcttagaataaaccgttaataacttgatttttccacaaagtacaagagcttacaattaaatctgtaaatattatttggactaatttattccctgttttcatattgcattttttgatttattatagaacaaaaattactcatcataatgactctcaataccttggagacaggtaactttaatgtttttaacgagggagtttcgataaactaaat

SEQ ID NO: 1121 - DNA, 224 bp, Legionella pneumophilia
catgatgtatcctttggtttctctaggtgtaggtggtcatgaagttagccatggattcactgaacaacattctggacttgaatatttcggtcagtctggtggcatgaatgagtcgttttcagatatggctgctcaagctgctgaatattattcagttggaaaaaacagctggcaaatcggcccggaaataatgaaagaagacagtggttatgacgcattgcgtt

SEQ ID NO: 1122 - DNA, 224 bp, Legionella pneumophilia
tgctttgagctaactgaagagatctcattgcagttattagcattgctatcttcaaagttttttgaggagttctggaacttgcctgtgctcaattcctgtaaatacgaacaccgtattgaaatctatctggacttgcgcccctaatctctttgttaaataatgttttaaatttagtcatttagtttatataaacttcctcattcaaaacattttttgtctcgacc

SEQ ID NO: 1123 - DNA, 224 bp, Legionella pneumophilia
taagcagacttgttttatttatgtcaaagaaatctatctagaatgcattagttaattaattgactgttgcatgaattcaaaaattactctcctgttgggtacggcagggaggataatttgtgatttttaatctaggctttaagtaaggattgttgacatttcatttatcttggcttaaaacccgcgattttctacgcttggctgcttttatcatttcagagcag

SEQ ID NO: 1124 - DNA, 224 bp, Mycobacterium avium
tgcccggcggcccggccgagtcctaccggtcgctgggcccgctgctcgaggagatctccgcgcacgtcgacggggtgccgtgctgcacgcacatcggccccgacggggccgggcacttcgtcaagatggtgcacaacggcatcgagtactccgacatgcagctgatcggtgaggcctaccagctgctgcgcgacgcgctgggcaagaccgccgagcagatcgcc

SEQ ID NO: 1125 - DNA, 224 bp, Mycobacterium avium
tcgaagacgctgtcggagaaggggattcggctgctggattgcggcaccagcggcggcgtctggggtcgggaacgcggttactgcctgatgatcggcggcgacgaggaggcgttcgcgcacgccgagccgatcttcgccaccgtcgcacccggggtggacgccgcgccgcggacgccgggccgcgacggcgaggtcacccggtcggagcaggggtatctgcattg

SEQ ID NO: 1126 - DNA, 224 bp, Mycobacterium avium
gaccaagaccaacgacgtcgccggcgacggcaccaccaccgcgaccgtgctggcgcaggcgctggtgaagggcggcctgcggctggtggcggcgggcgccaaccccatcgagctcggcgccggaatctccaaggccgccgacgcggtgtccgaggcgctgctggcctcggccaccccggtgtccggcaaggacgcgatcgcccaggtggcgaccgtgtcgtcgc

SEQ ID NO: 1127 - DNA, 224 bp, Mycobacterium avium
atcgtcgagggcgccggtgactccgacgccatcgccggccgggtggcccagatccgcaccgagatcgagaacagcgactccgactacgaccgcgagaagctgcaggagcggctggccaagctggccggcggcgtggcggtcatcaaggccggcgccgcgaccgaggtcgagctcaaggagcgcaagcaccgcatcgaggacgcggtccgcaacgccaaggcggc

SEQ ID NO: 1128 - DNA, 224 bp, Mycobacterium avium
tcggggagagcgccggcgcctgcatcaccgccacgctgctggccgtcccggccgccaaaggcctgttcgctcaagcgatttcggaaagcccggcctcgggactggtgcgttcccaggaggtcgccgccgaattcgcgaaccgcttcgccaacctgctcggtgtgcgccgccaggacgcggccaacgcgctgatgcaggcgtcggcggcccaactggtcaagacc

SEQ ID NO: 1129 - DNA, 224 bp, Mycobacterium avium
gcggctggtgcggctgatccaccgcggttcgcggctggccaagaaatccaagcaggcgaaaaaggtggcgctggtcggcaagggcgtcaccttcgacaccggcggcatctcgatcaagccggcggcctcgatgcatcacatgacctcggacatgggcggggccgccgccgtcatcgccaccgtggcgctggccgcgcagcttcagctgccgatcgacgtgatcg

SEQ ID NO: 1130 - DNA, 224 bp, Mycobacterium avium
acgtgctggcggcgctgaagtgggtgcgcgacaacatcaccgcattcggcggcgacccgcggcgggtcaccctgttcggcgagtcggcgggggcggggatcgtcaccacgctgttggccagccccgccgccgcgggcctgttcgccgctgccatcgcgcagagttcgccggccacctcggtgtacgaccgtgaccgcgccggccgtgtcgccgaagccttcctg

SEQ ID NO: 1131 - DNA, 224 bp, Mycobacterium avium
ccaagaagaccgacgacgtcgccggtgacggcacgacgacggccacggtgctcgcccaggcgttggtccgcgagggcctgcgcaacgtcgcggccggcgccaacccgctgggtctcaagcgcggcatcgagaaggccgtcgagaaggtcaccgagaccctgctcaagtcggccaaggaggtcgagaccaaggaccagatcgctgccaccgcggccatctccgcg

SEQ ID NO: 1132 - DNA, 224 bp, Mycobacterium avium
ccacgtcaaccactcgatctggtggaagaacctgtcgccggacggcggtgacaagcccaccggcgagctggccgccgcgatcgacgacgcgttcggctccttcgacaagttccgggcgcaattcagcgccgccgccaacggcctgcagggctccggctgggcggtgctgggttatgacaccgtgggcagccggttgctgaccttccagctctacgaccagcagg

SEQ ID NO: 1133 - DNA, 224 bp, Mycobacterium avium
ccgccgccaatgtggctgagtactacgacattccgttggcatcgctgcaccacttcccgatgcgcgccaacgggcagctggtccccaacatgccgtcaccgttggtgcgctcgacgatgacggccatcgagtggctgttctggcgttcgacgaagaaggtcgacaacgcgcagcgccgccaactcgggctgcccaaggcgacgcgacgttcgcagcgacggatc

SEQ ID NO: 1134 - DNA, 224 bp, Mycobacterium avium
actacggccggatgtgcccgatcgagaccccggagggtcccaacatcggtctgatcggctcgctgtcggtgtatgcgcgggtcaacccgttcgggttcatcgagacgccgtaccgcaaggtggtcgacggcgtggtcaccgacgagatccactacctgaccgccgacgaggaggaccgccacgtggtggcgcaggccaactcgccgatcgacgacaagggccgg

SEQ ID NO: 1135 - DNA, 224 bp, Moraxella catarrhalis
acccccagatggtcaaagtcgtttcggtacaaaaaggggcaggctttgccagtgcaggcattggggcaaccaatggtgcgattgttaccaaaaccttagatgctgatgagcttttaagaaacagcgacaaagattatggctttaaagttggtgcaggcttatcaaccaacaaaggtcattcttatcatggcagtgcctttggtaaagcacagacaggatttggt

SEQ ID NO: 1136 - DNA, 224 bp, Moraxella catarrhalis
ttcaaccacagcctattaaaaggctttggcgttgaccatactttattaaaatatggtatcaactatcgccatcaagaagccgtaccgcccagaagtctaaaacctggtgtggtgcatcaagaaaaaaccgatgctggcatttatctagaagcggttaaccaaatcaatgactttaccatcaatacaggtgtgcgtgttgaccgttttgactttaaagccatgga

SEQ ID NO: 1137 - DNA, 224 bp, Moraxella catarrhalis
caaaaccaaaaacgccgcagtaactgtgggtgatttaaatgccgttgcccaaacaccgctgacctttgcaggggatacaggcacaacggctaaaaaactgggcgagactttgaccatcaaaggtgggcaaacagacaccaataagctaaccgataataacatcggtgtggtagcaggtactgatggcttcactgtcaaacttgccaaagacctaaccaatctta

SEQ ID NO: 1138 - DNA, 224 bp, Moraxella catarrhalis
tgatgatggcaatgtgcttatcaaagccaaagatggtaagttctacaaagcagacgacctcatgccaaacggctcactaaaagcaggcaaatcagccagtgatgccaaaactccaactggtctaagccttgttaaccccaatgctgataaaggcagtacaggcgatgcagtggctcttaataacttatcaaaagcggtatttaaatccaaagatggtacaacta

SEQ ID NO: 1139 - DNA, 224 bp, Moraxella catarrhalis
aaacttttcatacaggcactgcaggttatgtcatttctcaacaaggtgcaaaaataatacttgattatttatcaacactagatgcttttgaattttttccaattgaccatgtgatttttgatgccatcattagtaagatgagcgtcttacaagtcaatcctgccatggtcattcaagcccatttagtatcagaagatgacacatttaaaagtttgatagaaact

SEQ ID NO: 1140 - DNA, 224 bp, Moraxella catarrhalis
atatttaacctaccaacaacaagattatcatctaagcgattctataaccgctgatgtcaaacaaacaggcattggtttgtatcatcgccatgattttgatcacttacgcatcagtgccaaccttggggttgaccatttaagtacacaaagcttacgccaaatcatgtgggacggtgagaatcgcaaccatcaagcacatgccacaggtaagcgtcgatatgcaa

SEQ ID NO: 1141 - DNA, 224 bp, Moraxella catarrhalis
caaaataaagccatcaatcaagagcttgaagggtttgcggctcatgcagatgttcaagataagcaaattttacaaaaccaagctgatatcactaccaataagaccgctattgaacaaaatatcaatagaactgttgccaatgggtttgagattgagaaaaataaagctggtattgctaccaataagcaagagcttattcttcaaaatgatcgattaaatcgaat

SEQ ID NO: 1142 - DNA, 224 bp, Mycobacterium kansaii
gcccggaccgcagccacgccaagtcggcccggtcggttgccgagaccatgggcaactaccacccgcacggcgacgcgtcgatctacgacaccctggtgcgcatggcccagccctggtcgctgcgctacccgctggtggacggccagggcaacttcggctcgccaggcaatgacccaccggcggcgatgaggtacaccgaagcccggctgaccccgttggcgatg

SEQ ID NO: 1143 - DNA, 224 bp, Mycobacterium kansaii
ggagatcgcgatgcagtggaatgccggctattcggagtcggtgcataccttcgccaacaccatcaacacccacgagggtgggacccacgaagaggggttccgcagcgcgctgacctcggtggtgaacaagtacgccaaggaccgcaaactgctcaaggaaaaggaccccaacctcaccggcgacgacatccgggaagggttggccgcggtgatttcggtcaagg

SEQ ID NO: 1144 - DNA, 224 bp, Mycobacterium kansaii
ggaagtgcgtgacgtgcacccgtcgcactacggccgcatgtgcccgatcgagaccccggagggtcccaacatcggcctgatcggctcgctgtcggtgtacgcacgggtcaacccgttcggcttcatcgagacgccgtaccgcaaggtgatcgacggtctcgttactgatgagatccactacttgacggccgacgaggaggaccgccacgtcgtggcacaggcca

SEQ ID NO: 1145 - DNA, 224 bp, Mycobacterium kansaii
tcttcacgatcaccggccgcggcaccgtggtcaccggtcgtgtggagcgcggcgtggtcaacgtcaacgaggaagtcgagatcgtcggcatccgcccgacgaccaccaagaccaccgtcaccggtgtggagatgttccgcaagctgctcgaccaggggcaggccggtgacaacgtcgggctgttgctgcgtggcgtcaagcgtgaggacgtcgagcgcggccag

SEQ ID NO: 1146 - DNA, 224 bp, Mycoplasma pneumoniae
agcagtagggaatttttcacaatgagcgaaagcttgatggagcaatgccgcgtgaacgatgaaggtctttaagattgtaaagttcttttatttgggaagaatgactttagcaggtaatggctagagtttgactgtaccattttgaataagtgacgactaactatgtgccagcagtcgcggtaatacataggtcgcaagcgttatccggatttattgggcgtaaa

SEQ ID NO: 1147 - DNA, 224 bp, Mycoplasma pneumoniae
gtccaagccaccctcgggggcagtcagacgatgattacaggcggttcgcctcgaagaaccctcgaccaagccaacctccagctctgaacgggggcggggtgaaggaatgataaggcttcaagtggacaaagtgacgaaaaccacaccaagttcacgagcgctacggggatggaccagcagggacaatcaggtacctccgcggggaatcccgactcgttaaagca

SEQ ID NO: 1148 - DNA, 224 bp, Mycoplasma pneumoniae
tccccctacctgcacttggtgaagcctaagaaagttacccaatccgacaagttagacgacgatcttaaaaacctgttggaccccaaccaggttcgcaccaagctgcgccaaagctttggtacagaccattccacccagccccagccccaatcgctcaaaacaacgacaccggtatttgggacgagtagtggtaacctcagtagtgtgcttagtggtgggggtgc

SEQ ID NO: 1149 - DNA, 224 bp, Mycoplasma pneumoniae
taagagtgaattgagtccccttaacgttgttttacacgcagaagaggatacagtacaaattcagggcaagccgattactgagcaagcatggtttattcctacagttgctggttgctttggttttagtgccctagccatcatcttgggccttgctataggactgccaattgtgaagcgcaaggaaaaacgcttattggaggaaaaggaacgccaagaacagttag

SEQ ID NO: 1150 - DNA, 224 bp, Mycoplasma pneumoniae
taaaacgcaaaaaacctttatgttacaagcagatccgcaaaataacaatgtctttttggtcgaagtaaaccccaaacaaaagtccagctttccccaaaccatcttcttttgggatgtttatcaacgaatttgtctcaaggatttaactggtgcacaaatcagtctttcgcttactgcctttactactcagtatgctggtcagctcaaagtgcaccttagtgtta

SEQ ID NO: 1151 - DNA, 224 bp, Mycobacterium tuberculosis
ccactgcaccgcacaaagttaagagccgcacctttcactatccgggtggcctggtggacttcgtgaaacacatcaaccgcaccaagaacgcgattcatagcagcatcgtggacttttccggcaagggcaccgggcacgaggtggagatcgcgatgcaatggaacgccgggtattcggagtcggtgcacaccttcgccaacaccatcaacacccacgagggcggc

SEQ ID NO: 1152 - DNA, 224 bp, Mycobacterium tuberculosis
ggccagggcgctccccgacgatgctggccactgacctctcgctgcgggtggatccgatctatgagcggatcacgcgtcgctggctggaacaccccgaggaattggccgacgagttcgccaaggcctggtacaagctgatccaccgagacatgggtcccgttgcgagataccttgggccgctggtccccaagcagaccctgctgtggcaggatccggtccctgcg

SEQ ID NO: 1153 - DNA, 224 bp, Mycobacterium tuberculosis
cgtggccaccgcattgcgtcagcggtactcccggcgcggacttccatcccagtctggacacgtcggcaatcgaggcggtgttctacaagggtgcctacaccggagcgtacagcggcttcgaaggagtcgacgagaacggcacgccactgctgaattggctgcggcaacgcggcgtcgatgaggtcgatgtggtcggtattgccaccgatcattgtgtgcgccag

SEQ ID NO: 1154 - DNA, 224 bp, Mycobacterium tuberculosis
tccgggcggcgtcgaggtgccggtggaaaccgacgacatcgaccacttcggcaaccgccgcctgcgtacggtcggcgagctgatccaaaaccagatccgggtcggcatgtcgcggatggagcgggtggtccgggagcggatgaccacccaggacgtggaggcgatcacaccgcagacgttgatcaacatccggccggtggtcgccgcgatcaaggagttcttcg

SEQ ID NO: 1155 - DNA, 224 bp, Neisseria meningitidis
gatatgggggacagccacgtcggactgcaggcgcgcctgatgagtcaggctttgcgcaaactgaccggacacatcaaaaaaaccaatacgctggttgtgttcatcaaccaaatccgaatgaagatcggcgtaatgttcggcagccccgagaccacgaccggcggcaacgcgctgaaattctattcttccgtccgcctcgacatccgccgcaccggatccatcaa

SEQ ID NO: 1156 - DNA, 224 bp, Neisseria meningitidis
gggcgtggaagaacgcggcgagttttacgacattaccggcgcgttgcgcgatatggtgcaaaaccacttgatgcaaatgctgtgcatgactgcgatggaagcccccgccagcttggatgccgacgcggtgcgcgatgaaaaagtcaaagtcatcaagtcattgaagccgctgaccgtcgaatctgtcaatgaaaatgtcgtgcgcggacaatataccgccgcca

SEQ ID NO: 1157 - DNA, 224 bp, Neisseria meningitidis
cgaattgaaaaacaactggaatgccgcctacaaaaaatcggcgcgcattgtcggcgacgtcatcggtaaataccacccccacggcgataccgccgtatacgacaccatcgtccgtatggcgcaaaatttcgctatgcgttatgtgctgatagacggacagggcaacttcggatcggtggacgggcttgccgccgcagccatgcgctacaccgaaatccgcatgg

SEQ ID NO: 1158 - DNA, 224 bp, Neisseria meningitidis
gatatgggggacagccatgtcggactgcaggcgcgcctgatgagccaggctttgcgcaaactgaccggacacatcaaaaaaaccaacacgctggttgtgttcatcaaccaaatccggatgaagatcggcgtaatgttcggcagccccgaaaccaccaccggcggcaacgcgctgaaattctattcttccgtccgcctcgacatccgccgcaccggatccatcaa

SEQ ID NO: 1159 - DNA, 224 bp, Pseudomonas aeruginosa
gggtgtggaagtcgccttgcagtggaacgacagcttcaacgagaacctgctctgcttcaccaacaacatcccgcagcgtgacggcggcacccacctggccggtttccgttcggcgctgacgcgtaacctgaacaactacatcgaggccgaaggcctggcgaagaagttcaagatcgccaccaccggcgacgatgcccgcgaaggcctcaccgcgatcatctcgg

SEQ ID NO: 1160 - DNA, 224 bp, Pseudomonas aeruginosa
cgggtgatgagcctcggcttccgtggcgaggcgctggcctcgatcagctcggtagcgcgcctgaccatgacctcgcgtaccgccgacgccggcgaagcctggcaggtggaaaccgagggccgcgacatgcagccgcgggtacagccggcggcgcacccggtggggaccagcgtcgaggttcgcgacctgttcttcaacaccccggcgcggcgcaagttcctgcg

SEQ ID NO: 1161 - DNA, 224 bp, Pseudomonas aeruginosa
acggccgcgtgtgcccgatcgaaacccctgaaggtccgaacatcggtctgatcaactccctggcgacctacgcccgcaccaacaagtacggcttcctcgagagcccgtaccgcgtggtcaaggacagcctggtaaccgacgagatcgtgttcctgtcggcgatcgaagaagccgaccacgtcatcgcccaggcttcggcgaccctcaacgagaagggtcaactg

SEQ ID NO: 1162 - DNA, 224 bp, Pseudomonas aeruginosa
gtgatgtcgccccagggcggtggccgtcccgatggcgacctggggcgggccatcgatgagcagctcgggggcttcgaggcgttcaaggatgcgttcaccaaggctgcgctgacccgcttcggcagcggctgggcctggctcagcgtgactccccagggcagcttgctggtggagagcagcggcaaccaggacagcccgctgatgaacggcaatacgccgatcct

SEQ ID NO: 1163 - DNA, 224 bp, Pseudomonas aeruginosa
gcgcggtttctatatcgccggcgatccggcgctggcctacggctacgcccaggaccaggaacccgacgcgcgcggccggatccgcaacggtgccctgctgcgggtctatgtgccgcgctcgagcctgccgggcttctaccgcaccggcctgaccctggccgcgccggaggcggcgggcgaggtcgaacggctgatcggccatccgctgccgctgcgcctggacg

SEQ ID NO: 1164 - DNA, 224 bp, Proteus mirabilis
aggaacctttgttcgtttctggccaagtcttgatacctttaaaggtgagactgaattccaatatgatattttagcgaaacgtctgcgtgagctctccttcttaaactcaggagtctctattcgtttaattgataaacgtgataaccgcgaagatcatttccactatgaaggtggtatcaaagcattcgttgaatacttaagccgtaataaaacgcctattcacc

SEQ ID NO: 1165 - DNA, 224 bp, Proteus mirabilis
cttctgaagttaaaacagcggtagaaacgctgatgaatgagaagctggtggaatatctattagaaaatccgactgacgcaaaaattgttgtcggtaaaattattgatgctgctcgtgcccgtgaagctgcgcgtaaagcacgtgagatgacgcgtcgtaaaggcgcactcgatttaggtggtttaccgggtaaattagcggactgctctgagcgtgatcctgct

SEQ ID NO: 1166 - DNA, 224 bp, Proteus mirabilis
tagatactccaggtcacgccgcatttacttcaatgcgtgcccgtggtgctcaggtaacggatatcgttgttctggttgttgctgctgatgatggtgtgatgccacaaactatcgaagctatccaacatgccaaagcagcaaatgtgcctgttgttgttgccgtgaacaaaattgataaaccagaagctgatcctgatcgtgttaaaactgagctgtctcaatat

SEQ ID NO: 1167 - DNA, 224 bp, Proteus mirabilis
actcattatctgtttatgcacagactaacgagtacggattcttagaaacgccttaccgtgttgtagaaaacaacgctgtaacagatgaaatccactatctgtctgcaattgaagaaggtaacttcatcattgctcaggcgaactccgtattagatgatgatggccacttcgtagaagaattagtaacttgccgtcataaaggtgagtcaagtttatttagtcgt

SEQ ID NO: 1168 - DNA, 224 bp, Pseudomonas pseudoalcaligenes
ggtaccggcacgcagatccacttcaatccgtccgaagagaccttagccaacatccacttcagatgggacatcctggtcaagcgtctgcgcgaaatgtctttcatcaaatccggcgtgggcatcgtgctgcgtggcgagcgcaccggcaaggaagagctgttcaagtacgaaggtggcctgaaacccttcgttgagtacctgaacaccaacaagacagtggtcaa

SEQ ID NO: 1169 - DNA, 224 bp, Pseudomonas pseudoalcaligenes
tcgaaacgccggaaggtccgaacatcggtctgatcaactccctggcggcctacgcccgcaccaaccagtacggcttcctggagagcccgtaccgcgtggtcaaggaaggcaaggtcaccgacgagatcgtgttcctgtccgccatcgaagaagccgatcacgtgatcgcgcaggcttccgccacccttaacgacaagagagcgagctggtcgactgagctggta

SEQ ID NO: 1170 - DNA, 224 bp, Proteus vulgaris
tcacggcggtaacaggggttcgaatcccctaggggacgccaattgcgcggtatgagtgaaaggcgtaccacactatagtctgatgcaaatcagagaatagttaagataattttagcaagttattttaactattatgctctttaacaatctggaacaagctgaaaaattgaaaacaaatcaatatatcaccgaggtatattgatgagtctctcaaaatctcaaac

SEQ ID NO: 1171 - DNA, 224 bp, Proteus vulgaris
ttccaataagaaagaaacctgagaaataaaaacggtattaaagaatgcattatggggctatagctcagctgggagagcgcctgccttgcacgcaggaggtcagcggttcgatcccgcttagctccaccataatctcttgaatataaaataataattcagagtatattagcaatagtatactgcgaattattttgctctttaacaatctggaacaagctgaaaaa

SEQ ID NO: 1172 - DNA, 224 bp, Proteus vulgaris
tttatgcacagactaatgagtacggtttccttgaaacgccttaccgtgttgttgaaaacaacgctgtaacagatgaaattcactatctgtctgcaatcgaagaaggtaacttcatcattgctcaggctaactccgtattagacgatgacaatcacttcgttgaagaattagtaacttgccgtcataaaggtgagtcaagcttatttagtcgtgaccaagttcag

SEQ ID NO: 1173 - DNA, 224 bp, Staphylococcus aureus
aaatatctgattactatccaagaaattcgattgatacaaaagagtatatgagtactttaacttatggattcaacggtaatgttactggtgatgatacaggaaaaattggcggccttattggtgcaaatgtttcgattggtcatacactgaaatatgttcaacctgatttcaaaacaattttagagagcccaactgataaaaaagtaggctggaaagtgatattt

SEQ ID NO: 1174 - DNA, 224 bp, Staphylococcus aureus
ttgatgaacaagcgattaatgcaggtgcagttaacactgttttgataaaagatggcaagtggatagggtataatacagatggtattggttatgttaaaggattgcacagcgtttatccagatttagaaaatgcatacattttaattttgggcgcaggtggtgcaagtaaaggtattgcttatgaattagcaaaatttgtaaagcccaaattaactgttgcgaat

SEQ ID NO: 1175 - DNA, 224 bp, Staphylococcus aureus
tggtataacgatttattagtagattttgattcaaaggatattgttgataaatataaagggaaaaaagtagacttgtatggtgcttattatggttatcaatgtgcgggtggtacaccaaacaaaacagcttgtatgtatggtggtgtaacgttacatgataataatcgattgaccgaagagaaaaaagtgccgatcaatttatggctagacggtaaacaaaatac

SEQ ID NO: 1176 - DNA, 224 bp, Staphylococcus aureus
gcaataaacgttaaatctatagatcaatttctatactttgacttaatatattctattaaggacactaagttagggaattatgataatgttcgagtcgaatttaaaaacaaagatttagctgataaatacaaagataaatacgtagatgtgtttggagctaattattattatcaatgttatttttctaaaaaaacgaatgatattaattcacatcaaactgacaa

SEQ ID NO: 1177 - DNA, 224 bp, Staphylococcus aureus
atgacaaagtgaaaacagagttattaaatgaagatttagcaaagaagtacaaagatgaagtagttgatgtgtatggatcaaattactatgtaaactgctatttttcatccaaagataatgtaggtaaagttacaggtggtaaaacttgtatgtatggaggaataacaaaacatgaaggaaaccactttgataatgggaacttacaaaatgtacttataagagtt

SEQ ID NO: 1178 - DNA, 224 bp, Staphylococcus aureus
caattttcaaatgaatcttatgatttaatttcagaatcaaaagattttaataaatttagtaatttcaagggaaaaaaacttgatgtttttggtattagttataatggccagtgtaacactaaatacatatatggtggagtcacagctactaacgaatatctagataaatctagaaatatacctataaatatatggatcaatggaaatcacaaaactatttctac

SEQ ID NO: 1179 - DNA, 224 bp, Staphylococcus aureus
acctgaaaagcttcaaggagttagttctggaaatttttctacttctcatcaattagagtatattgatggaaaatacactttatattcacagtttcataatgaatatgaagcgaagagattaaaagatcataaagtagatatctttggaataagttactcaggtctttgtaatacaaaatatatgtatggtggaattacgttggcgaatcaaaatttagataaac

SEQ ID NO: 1180 - DNA, 224 bp, Staphylococcus aureus
tggaaaaagtaggtaacgatggtgtcattacaattgaagaatcaaatggactaaacactgaactagaagtggttgaaggtatgcaatttgatcgtggttatcaatcaccgtatatggttactgattcagataaaatggttgctgaattagaacgcccatacattttagtaacagataagaaaatctcgtctttccaagatatcttacctttattagaacaagtg

SEQ ID NO: 1181 - DNA, 224 bp, Staphylococcus aureus
atataaaaaatcagcacgtatcgttggtgacgtaatgggtaaatatcaccctcatggtgacttatctatttatgaagcaatggtacgtatggctcaagatttcagttatcgttatccgcttgttgatggccaaggtaactttggttcaatggatggagatggcgcagcagcaatgcgttatactgaagcgcgtatgactaaaatcacacttgaactgttacgtg

SEQ ID NO: 1182 - DNA, 224 bp, Staphylococcus aureus
tctacctaaaaataaaatagattcagtaaatgttagtcaaacattaggttataacataggtggtaattttaatagtggtccatcaacaggaggtaatggttcatttaattattcaaaaacaattagttataatcaacaaaactatatcagtgaagtagaacatcaaaattcaaaaagtgttcaatggggaataaaagctaattcatttatcacatcattaggta

SEQ ID NO: 1183 - DNA, 224 bp, Staphylococcus aureus
ttctcagattttaacttttaattttattaaagataaaagttatgataaagatacattaatactcaaagctgctggaaacatttattctggctatacaaagccaaatccaaaagacactattagttctcaattttattggggttctaagtacaacatttcaattaattcagattctaatgactcagtaaacgttgtagattatgcacctaaaaatcaaaatgaag

SEQ ID NO: 1184 - DNA, 224 bp, Staphylococcus aureus
tgtggaagttagattgggatcatagcgtcattattccaggaatgcagaaagaccaaagcatacatattgaaaatttaaaatcagaacgtggtaaaattttagaccgaaacaatgtggaattggccaatacaggaacagcatatgagataggcatcgttccaaagaatgtatctaaaaaagattataaagcaatcgctaaagaactaagtatttctgaagactat

SEQ ID NO: 1185 - DNA, 224 bp, Staphylococcus aureus
cagattacaacttcaccaggttcaactcaaaaaatattaacagcaatgattgggttaaataacaaaacattagacgataaaacaagttataaaatcgatggtaaaggttggcaaaaagataaatcttggggtggttacaacgttacaagatatgaagtggtaaatggtaatatcgacttaaaacaagcaatagaatcatcagataacattttctttgctagagt

SEQ ID NO: 1186 - DNA, 224 bp, Staphylococcus aureus
attgaaacacctgagggaccaaacattggattgattaactcattatcaagttatgcacgtgtaaatgaattcggctttattgaaacaccatatcgtaaagttgatttagatacacatgctatcactgatcaaattgactatttaacagctgacgaagaagatagctatgttgtagcacaagcaaactctaaattagatgaaaatggtcgtttcatggatgatga

SEQ ID NO: 1187 - DNA, 224 bp, Staphylococcus aureus
ggaaaaagttgacttaaacacaaaaagaactaaaaaaagccaacatactagcgaaggaacttatatccatttccaaataagtggcgttacaaatactgaaaaattacctactccaatagaactacctttaaaagttaaggttcatggtaaagatagccccttaaagtattggccaaagttcgataaaaaacaattagctatatcaactttagactttgaaattc

SEQ ID NO: 1188 - DNA, 224 bp, Staphylococcus epidermidis
cattaaaaaaataatttcagaaaagagcattgatggattcaatgttactattccacataaagaacgtattattccgtacctagatgatattaatgaacaagcgaaatctgttggggcggtaaatacagttttagttaaagatggtaagtggattggttataatactgatggaattggttatgttaatggtttaaaacaaatatatgaaggtatagaagacgctt

SEQ ID NO: 1189 - DNA, 224 bp, Staphylococcus epidermidis
ggtgattataatactggtacaaattatggttgggtaaaacaagatgaggtcatttacaacacagctaaatcacctgtaaaaatcaatcaaacatacaacgtcaaacctggtgttaaattccacacagtaccttggggcacatataatcaagtggctggaacagtttcaggtaaaggcgatcaaacttttaaagcaactaaacaacaacaaattgataaagcaac

SEQ ID NO: 1190 - DNA, 224 bp, Staphylococcus epidermidis
gttggagctatttcagcagcagatgaagaaatcggtcgctacatttctgaagcaatggataaagtaggtaacgatggcgttatcactattgaagaatcaaatgggtttaatacagaattagaagtagttgaaggaatgcaatttgatcgcggttatcaatcaccatatatggtaactgactcagataaaatgatagctgaattagaacgtccatatatattagt

SEQ ID NO: 1191 - DNA, 224 bp, Staphylococcus epidermidis
gcgatgctgaatacgaacaaaaaatcttagacttaatgcaagcagttgatgattacattccaactccagaacgtgattctgacaaaccattcatgatgccagttgaggacgtattctcaatcactggtcgtggtactgttgctacaggccgtgttgaacgtggtcaaatcaaagttggtgaagaagttgaaatcatcggtatgcacgaaacttctaaaacaact

SEQ ID NO: 1192 - DNA, 224 bp, Stenotrophomonas maltophilia
gacctgcgtgttcgtggataccggcctgctgcgctggcaggaaggcgaccaggtgatggcgatgttcgccgagcacatgggcgtgaaggtggtgcgcgtgaatgccgccgaccgttacttcgccgcgctggaaggcgtgagcgacccggaagccaagcgcaagatcatcggcaacctgttcgttgagatcttcgacgaagagtcgaacaagctgaagaacgcca

SEQ ID NO: 1193 - DNA, 224 bp, Stenotrophomonas maltophilia
cgcgaactgtcgttcctgaactccggcgtcaagatcgtccttgccgacgagcgtggcgatggccgtcgcgatgacttccactacgaaggcggcatccgcagcttcgtggagcatctggcccagctgaagaccccgctgcacccgaacgtcatctcggtcaccggcgagcacaacggcatcgtcgtggatgtggcgctgcagtggaccgactcctaccaggagac

SEQ ID NO: 1194 - DNA, 224 bp, Stenotrophomonas maltophilia
cgtcgcgccaagtacctgctgctggacaccgccatcggcagcgccgtgctgcacctgggcatgtccggcagcctgcgcgtgctgcccggcgataccccgctgcgcgcgcacgaccatgtggatatcagcctggacaacggccgcctgctgcgcttcaacgacccgcgccgcttcggcagcctgctctggcagccggccggcgaagtccacccgctgctgcaggg

SEQ ID NO: 1195 - DNA, 224 bp, Stenotrophomonas maltophilia
accctgcaggccatcgccgaatgccagaagatgggcggcaccgcggccttcatcgacgccgagcacgcgctggacccgatctacgccgccaagctgggcgtgaacgtggacgacctgctgctgtcgcagccggataccggtgagcaggcgctggaaatcgccgacatgctggtccgttcgggttcggtcgacatcctggtgatcgactcggttgccgcgctgac

SEQ ID NO: 1196 - DNA, 224 bp, Serratia marcescens
agcatactatattgtgttttttctactcccatatatcgtgcaaatgctttaattcaagtcgagcaaaaacagggtaatgcgattttagacaacttaagtcagatgctacctgacagacaacctcaatctgcacccgaaatctctttgttgcagtcacgaatgattcttggaaaaactgtcagtgatctgaatttacaaatagttgcagaacagaactactttcc

SEQ ID NO: 1197 - DNA, 224 bp, Serratia marcescens
tgaggacaaactaaattcttatcgacgtcagaaagattctgttgatttaaatttggaagctcagtctgtattaaatcgcatagtcaatgtagataaccagttaaatgaactgacatttaaagaagcagagatctctcaactttatacaaaggatcacccgacatataatgcactaatgcagaaaagggaaacgttacagcaagagaagtccaagcttaacaagc

SEQ ID NO: 1198 - DNA, 224 bp, Serratia marcescens
atgacgctcgcgtgaaaatgctccgcggcgtgaatgttctcgctgatgcagtaaaagtgaccctgggcccgaaaggccgcaacgtagtgctggataaatctttcggcgcgccgaccatcaccaaagacggcgtttccgttgcgcgtgaaatcgaactggaagacaagttcgagaacatgggcgcacagatggtgaaagaagtggcctccaaagcgaacgacgcg

SEQ ID NO: 1199 - DNA, 224 bp, Serratia marcescens
gcgtttgtcgagtacctgaacaagaacaaaaccccgatccacccgaacgtgttctacttctccaccgtgaaagacgacatcggcgtggaagtggcgctgcagtggaacgacggtttccaggaaaacatctactgcttcaccaacaacattccgcagcgcgacggcggcacgcacctggtcggtttccgtaccgcgatgacccgtacgctgaacagctacatgga

SEQ ID NO: 1200 - DNA, 224 bp, Streptococcus mitis
tggtgataaccaaccagatgttgtctttatgacacttggtactggtgttggtggcggtatcgtcgcagaaggcaaattgcttcacggtgttgctggtgcagcaggtgagcttggccacatcactgttgactttgaccaaccaatcgcatgtacttgcggtaagaaaggttgccttgagacagttgcttcagcaacagggattgtcaacttgactcgtcgctatg

SEQ ID NO: 1201 - DNA, 224 bp, Streptococcus mitis
ggtcaataatgtcccagctagaatgcagtatgaaaaaatcacggctcacagcatggaacaactcaaggtcaagtttggttctgactttgaaaagacagggaattctcttgatattgattttaactctgtccattcgggcgaaaagcagattcagattgttaattttaagcagatttattatacagtcagcgtagatgctgttaaaaatccaggagatgtgtttc

SEQ ID NO: 1202 - DNA, 224 bp, Streptococcus mitis
gtgggttcaacatgatatcgactggagttccgtctggaaggtaaggcatgtcttctacaggaacgatacgagagacaacccctttatttccgtgacgtccggccatcttatctccgaccttgatcttacgtttttgagcgatgtagacgcgaaccagcatgttaacacctgattgcaactcatctccatttgcacgtgtaaagatcttaacatcacgaacgaca

SEQ ID NO: 1203 - DNA, 224 bp, Streptococcus mitis
gaaggtgacactaaatacgaagacatcgttatggaattgatgaacacagttgatgagtacatcccagaaccagaacgtgacactgacaaaccattgcttcttccagtcgaagacgtattctcaatcactggacgtggtacagttgcttcaggacgtatcgaccgtggtatcgttaaagtcaacgacgaaatcgaaatcgttggtatcaaagaagaaactcaaaa

SEQ ID NO: 1204 - DNA, 224 bp, Streptococcus mutans
ctattgttaatcgtgatggcagattgatcggttacaatacagatggttttggtttctttaagagtttgggaacctttgcagatttcgatgttgcagataaggtcataaccatacttggtggcggaggagctgcaactgccattattgcacaagcagctatcaatggtgctaagaaaattaatatttttaatcaaacagcctttcttgaagaaacaaaagaaaaa

SEQ ID NO: 1205 - DNA, 224 bp, Streptococcus mutans
ttcggatggcacaatggtggtcttaccgtcacatgttggttgatggtcatggaaattttggttctatggacggagatggcgctgcagcgcagcgttatacagaagcgcgtatgagcaaaattgctatggaaatgcttcgtgatattaataaaaatacagttgattttgcagataactatgatggttctgaacgtgaacctttagttttaccgtcacgttttcca

SEQ ID NO: 1206 - DNA, 224 bp, Streptococcus mutans
gtcgtcaggacaacttctatcaaacattttgacattgttgttgctgctgaaggcaataaaaatattgtcaaacgtgtgattggcatgcccggtgataccattacctatgaaaatgatatgctttctattaatgggaaaaaagtcaatgaaacttatctcaagcaatacaaggataaatttgccaaggacaaactccaaaagacttatgcctacaatcagtattt

SEQ ID NO: 1207 - DNA, 224 bp, Streptococcus mutans
tgttttgtcagctggtcatggctcaatgcttttatatgctctcttgcacttgtcgggatttgaagatatttctattgatgaaatcaaaaatttccgtcagtggggctctaaaacaccggggcaccctgagtatggccataccgtaggtgttgatgtgacaacaggccctcttggccaagggatttcaatggctgttggtttggctcaagctgaacgtttcttag

SEQ ID NO: 1208 - DNA, 224 bp, Streptococcus pneumoniae
ttctttgtagacagcgatcgattccgtggtgttcctttctttttccgtacaggtaaacgactgactgaaaaaggaactcatgtcaacatcgtctttaaacaaatggattctatctttggagaaccacttgctccaaatattttgaccatctatattcaaccaacagaaggcttctctcttagcctaaatgggaagcaagtaggagaagaatttaacttggctcc

SEQ ID NO: 1209 - DNA, 224 bp, Streptococcus pneumoniae
gtgttggtggcggtatcgtcgcagaaggcaaattgcttcacggtgttgctggtgcagcaggtgagcttggtcacatcactgttgactttgaccaaccaatctcatgtacttgtggtaaaaaaggctgccttgagacagttgcttcagcaacagggattgtcaacttgactcgtcgctatgccgatgaatacgaaggcgatgcagccttgaaacgcttgattgat

SEQ ID NO: 1210 - DNA, 224 bp, Streptococcus pneumoniae
ggaaaaacgcattgagggtatcacagcagtacgtgatgagtcaaaccgtgaaggtgttcgatttgttattgaagtcaagcgcgacgcctcagccaatgttattctcaataacctcttcaaaatgacccaaatgcaaaccaattttggtttcaatatgctcgctatccaaaatggtataccgaaaattttgtctcttcgtcagattttggatgcttatatcgagc

SEQ ID NO: 1211 - DNA, 224 bp, Streptococcus pneumoniae
atcatctgctcttcttcacaaacaagggacgtgtctatcgtcttaaaggttatgaaattcctgagtatggtcggactgccaaagggctaccagtagtcaatctcttgaaattggatgaagacgaaagtattcagacggttatcaatgttgagtctgatcgcagtgatgatgcttatctcttctttacaacccgtcacggtattgtgaagagaaccagtgttaag

SEQ ID NO: 1212 - DNA, 224 bp, Streptococcus pneumoniae
atcttcactgaaacaacaatctttgattttgataaattaaataaacggattcaagagttggcctttctaaatcgcggtcttcaaatttcaattacagataagcgccaaggtttggaacaaaccaagcattatcattatgaaggtgggattgctagttacgttgaatatatcaacgagaacaaggatgtaatctttgatacaccaatctatacagacggtgagat

SEQ ID NO: 1213 - DNA, 224 bp, Streptococcus pneumoniae
taagattctagctaacgaagaaattcgtagtcttttcacagccatgggaacaggatttggcgcagaatttgatgtttcgaaagcccgttaccaaaaactcgttttgatgaccgatgccgatgtcgatggagcccacattcgtacccttcttttaaccttgatttatcgttatatgaaaccaatcctagaagctggttatgtttatattgcccaaccaccaatct

SEQ ID NO: 1214 - DNA, 224 bp, Streptococcus pneumoniae
cacagcatggaacaactcaaggtcaagtttggttctgactttgaaaagacagggaattctcttgatattgattttaactctgtccattcaggcgaaaagcagattcagattgttaattttaagcagatttattatacagtcagcgtagatgctgttaaaaatccaggagatgtgtttcaagatactgtaacggtagaggatttaaaacagagaggaatttctgc

SEQ ID NO: 1215 - DNA, 224 bp, Streptococcus pyogenes
tatgataaatcgcatcaatttgcttatgtcaaagagtcaactgatgcgggttataacgcacaagacgttaaaggtaaaattgctttaattgaacgtgatcccaataaaacctatgacgaaatgattgctttggctaagaaacatggagccctgggagtacttatttttaataacaagcctggtcaatcaaaccgctcaatgcgtctaacagctaatgggatggg

SEQ ID NO: 1216 - DNA, 224 bp, Streptococcus pyogenes
tagtcacaaatcaagcccagctagcagtggtgcaccgcaatcagccgcaaagccagctaacaaagatgaatcaggatttctttatctcaccaaacgaagatgggaataaagacttcgtggcctttaaaggcttgaaaaataacgtgtataatgacttaacggttaacgtatacgctaaagatgaccaccaaaaacaaacccctatctggtctagtcaagcaggc

SEQ ID NO: 1217 - DNA, 224 bp, Streptococcus pyogenes
atgactattttagaaaatatcactttatcgccaatcaaaactaagggaatggctaaagcagaggctgacaaaacagccttgagcttgttggacaaagttggattatcagaaaaagccaaggcttatcctgctagtctttctggtgggcaacagcagcggattgcgattgcgcgtggactggctatggatccagatgttttactctttgatgaaccaacttcagc

SEQ ID NO: 1218 - DNA, 224 bp, Streptococcus pyogenes
ggtagtgacaaagagtatcgtatcactagaagagactcttatatgggagatatttctgatccagaagttattttagagtatggggtttaccctgctttcataaaaggctatacccaattgaaagctaacatcgaagaagcattattagaaatgtcaaatagcggtcaagcattagacatttaccaagcggttcaaaccctaaacgctgaaaacatgttattaaa

SEQ ID NO: 1219 - DNA, 224 bp, Streptococcus pyogenes
ttagatgttgatcgtattgatattgaggtaacagaggtccatcagggacaaaacaaggatattttagccttgtcagaggtcacttttggcaatatagaacgcgatatttttgaaaaaaagtttaaagaaattaaagataaatgggtaacagataaacaagcagatgagtttattgaaactgccgacaagtatgctgataaagctgttcagatgtctgctgttgc

SEQ ID NO: 1220 - DNA, 224 bp, Streptococcus pyogenes
aaaaagagtttgttgaaaaactaaaacaagtctacaaagaaagcggagcttcccacgttacaagcaaaaaagatttgatgttagcttttgacgatgctaaaaaaaagtcaacgattggtcgacaagaaaacggtctttttgtgacaagttttgctgaagatatggcattgctctttactgatcaaggtaagttaaaatcagctgaccaaattgaaaatataaaa

SEQ ID NO: 1221 - DNA, 224 bp, Streptococcus pyogenes
caactgatggggttgaaatcgttcttgaagcaactggtttctttgctaaaaaagaagcagctgaaaaacacttacatgctaacggtgctaaaaaagttgttatcacagctcctggtggaaacgatgttaaaacagttgttttcaacactaaccacgacattcttgacggtactgaaacagttatctcaggtgcttcatgtactacaaactgtttagctcctatg

SEQ ID NO: 1222 - DNA, 224 bp, Streptococcus pyogenes
aaatagctgtgttctccgtcccatttgattggaactatttatcaaaaggaaaagtcacagcatatacgtacggcggaataacaccctaccaaaaaacttcaatacctaaaaatatccctgttaatttatggattaatggaaagcagatctctgttccttacaacgaaatatcaactaacaaaacaacagttacagctcaagaaattgatctaaaggttagaaaa

SEQ ID NO: 1223 - DNA, 224 bp, Streptococcus pyogenes
agttaaaaaccttcaaaatatatattttctttatgagggtgaccctgttactcacgagaatgtgaaatctgttgatcaacttttatctcacgatttaatatataatgtttcagggccaaattatgataaattaaaaactgaacttaagaaccaagagatggcaactttatttaaggataaaaacgttgatatttatggtgtagaatattaccatctctgttatt

SEQ ID NO: 1224 - DNA, 224 bp, Streptococcus pyogenes
ttttgaaagaggtataaaaaaaatgaataaaaagaaattaggtatcagattattaagtcttttagcattaggtggatttgttcttgctaacccagtatttgccgatcaaaactttgctcgtaacgaaaaagaagcaaaagatagcgctatcacatttatccaaaaatcagcagctatcaaagcaggtgcacgaagcgcagaagatattaagcttgacaaagtta

SEQ ID NO: 1225 - DNA, 224 bp, Streptococcus pyogenes
aattatgaaatatcataattaccctaacaaagggttgaaagactacacttacacactaagctcaaataacccatatttcaaccatcctaagaacttgtttgcagctatctctactagacaatacaactggaacaacatcctacctacttatagcggaagagaatctaacgttcaaaaaatggcgatttcagaattgatggctgatgttggtatttcagtagaca

SEQ ID NO: 1226 - DNA, 224 bp, Streptococcus pyogenes
agatgatcatgtagatgtttttggattattttatattcttaattctcacaccggtgagtacatctatggaggaattacgcctgctcaaaataataaagtaaatcataaattattgggaaatctatttatttcgggagaatctcaacagaacttaaataacaaaattattctagaaaaagatatcgtaactttccaggaaattgactttaaaatcagaaaatacc

SEQ ID NO: 1227 - DNA, 224 bp, Streptococcus pyogenes
atttgttactacaaatagcatacatattaatactaaacaaaaaagatcggaatgtattctttatgttgattctattgtatctttaggcattactgatcagtttataaaaggggataaggtcgatgtttttggtctcccttataatttttccccaccttatgtagataatatttatggtggtattgtaaaacattcgaatcaaggaaataaatcattacagtttg

SEQ ID NO: 1228 - DNA, 224 bp, Streptococcus pyogenes
tgctctaataaaatcctacagtgtgatatgtaaggaacaatataattatgttgatggaggtttgataagaacaagtgacagagagaaacttgattcaacaatatatatgaatatttttggggaacagataccgctaaaagagcaatcaaaatataaaatcacatttcagaataagtttgtaacttttcaagaaattgatgttagacttaggaaaagtttgatga

SEQ ID NO: 1229 - DNA, 224 bp, Streptococcus pyogenes
caccagccgtatacgctgcagaagcaatggatgttcctatgatttttgcgaaaaaacataaaaacattaccatgacagaaggcattttgacagcagaagtttattctttcactaaacaagtgacgagcacggtgtctatcgctggtaaattcctatctaaagaagacaaggttttgattattgatgactttttagctaatggtcaggcagccaaaggcttgatc

SEQ ID NO: 1230 - DNA, 224 bp, Streptococcus viridans
aaaaaattgcttcagatattgacagagctgaagaagcacgtcaaaaagcggaagtattggctcaaaaacgcgaagatgaattggctggtagccgtaaagaagctaagacaatcattgagaatgcgaaagaaacagctgagaaaagtaaagctagtattttagcagatgctaaactagaagcaggacgcttaaaagaaaaagcaaaccaagaaattgctcaaaac

SEQ ID NO: 1231 - DNA, 224 bp, Streptococcus viridans
tggtcaattttttgctcttgagcaggcacttcagcaggctcgtaaattgatagcagaaggggctagtatgctagatatcggcggagaatcgactaggccgggaagtagctatgttgctagatagaaatagaagaggaaatccagcgtgttgttccagtgatcaaagcgattcgcaaggaaagtgatgtcctcatttctatcgatacttggaaaagtcaggtggc

SEQ ID NO: 1232 - DNA, 224 bp, Streptococcus viridans
tggcagctcttggtgagcgttggatgggtgctggtgaaaaccaaccagacgttgtctttatgacacttggtacaggtgttggtggcggtatcgtggcagaaggaaaattacttcacggtgttgctggtgctgcaggtgaacttggacacatcacagttgactttgatcaaccaatcgcatgtacgtgtggtaaaaaaggctgtcttgagacagtagcttcagca

SEQ ID NO: 1233 - DNA, 123 bp, Escherichia coli
tgggttgcaaaagaagtaggtagcttaaccttcgggagggcgcttaccactttgtgattcatgactggggtgaagtcgtaacaaggtaaccgtaggggaacctgcggttggatcacctcctta

SEQ ID NO: 1234 - DNA, 51 bp, Escherichia coli
gcaccggctaactccgtgccagcagccgcggtaatacggagggtgcaagcg

SEQ ID NO: 1235 - DNA, 361 bp, Escherichia coli
agctaacgcgttaagtcgaccgcctggggagtacggccgcaaggttaaaactcaaatgaattgacgggggcccgcacaagcggtggagcatgtggtttaattcgatgcaacgcgaagaaccttacctggtcttgacatccacggaagttttcagagatgagaatgtgccttcgggaaccgtgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgaccag

SEQ ID NO: 1236 - DNA, 118 bp, Mycobacterium tuberculosis
cacccgaagccagtggcctaaccctcgggagggagctgtcgaaggtgggatcggcgattgggacgaagtcgtaacaaggtagccgtaccggaaggtgcggctggatcacctcctttct

SEQ ID NO: 1237 - DNA, 51 bp, Mycobacterium tuberculosis
agcaccggccaactacgtgccagcagccgcggtaatacgtagggtgcgagc

SEQ ID NO: 1238 - DNA, 361 bp, Mycobacterium tuberculosis
cgtagctaacgcattaagtaccccgcctggggagtacggccgcaaggctaaaactcaaaggaattgacgggggcccgcacaagcggcggagcatgtggattaattcgatgcaacgcgaagaaccttacctgggtttgacatgcacaggacgcgtctagagataggcgttcccttgtggcctgtgtgcaggtggtgcatggctgtcgtcagctcgtgtcgtgagatgttgggttaagtcccgcaacgagcgcaacccttgtctcatgttgccagcacgtaatggtggggactcgtgagagactgccggggtcaactcggaggaaggtggggatgacgtcaagtcatcatgccccttatgtcc

SEQ ID NO: 1239 - DNA, 125 bp, Neisseria meningitidis
tgggggataccagaagtaggtagggtaaccgcaaggagcccgcttaccacggtatgcttcatgactggggtgaagtcgtaacaaggtagccgtaggggaacctgcggctggatcacctcctttct

SEQ ID NO: 1240 - DNA, 51 bp, Neisseria meningitidis
agcaccggctaactacgtgccagcagccgcggtaatacgtagggtgcgagc

SEQ ID NO: 1241 - DNA, 361 bp, Neisseria meningitidis
tagctaacgcgtgaaattgaccgcctggggagtacggtcgcaagattaaaactcaaaggaattgacggggacccgcacaagcggtggatgatgtggattaattcgatgcaacgcgaagaaccttacctggtcttgacatgtacggaatcctccggagacggaggagtgccttcgggagccgtaacacaggtgctgcatggctgtcgtcagctcgtgtcgtgagatgttgggttaagtcccgcaacgagcgcaacccttgtcattagttgccatcattcagttgggcactctaatgagactgccggtgacaagccggaggaaggtggggatgacgtcaagtcctcatggcccttatgaccag

SEQ ID NO: 1242 - DNA, 691 bp, Bordetella pertussis
aacgatggccacactgtctcctcctgagactcagcgaagttgaagtgtttgtgatgatgcaatctacccgcggctagacggaaagaccccatgaacctttactgtagctttgcattggactgtgaaccggcctgtgtaggataggtgggaggcgcagaactcgagtcgccagattcgagggagccatccttgaaataccaccctggtttgtttgcggttctaaccttggtccgttatccggatcggggacagtgcatggtaggcagtttgactggggcggtctcctcccaaagcgtaacggaggagttcgaaggtacgctaggtacggtcggaaatcgtgctgatagtgcaatggcataagcgtgcttgactgtgagactgacagtgcgaacaggtgcgaacgggacatagtgatccggtggttctgatggaagggccatcgctcaacggataaaggtactctgggataacaggctgataccgcccaagagttcatatcgacggcggtgtttggcacctcgatgtcggctcatctcatcctgggctgtagccggtccaagggtatgctgttcgccatttaaagaggtacgtgagctgggtttaaaacgtcgtgaagacagtttggtccctatctgccgtgggcgttggatacttgaacaggagcctgctcctagtacgagaggaccggagt

SEQ ID NO: 1243 - DNA, 701 bp, Enterococcus faecium
taacgatttgggcactgtctcaacgagagactcggtgaaattttagtacctgtgaagatgcaggttacccgcgacaggacggaaagaccccatggagctttactgtagtttgatattgagtgtctgtaccgcatgtacaggataggtaggagccgtagaaatcggaacgctagtttcgatggaggcgctggtgggatactacccctgcgttatggccactctaacccgcaccactaatcgtggtgggagacagtgtcagatgggcagtttgactggggcggtcgcctcctaaaaggtaacggaggcgcccaaaggttccctcagaatggttggaaatcattcgaagagtgtaaaggcagaagggagcttgactgcgagaccaacaagtcgagcagggacgaaagtcgggcttagtgatccggtggttccgcatggaagggccatcgctcaacggataaaagctaccctggggataacaggcttatctcccccaagagtccacatcgacggggaggtttggcacctcgatgtcggctcgtcgcatcctggggctgtagtcggtcccaagggttgggctgttcgcccattaaagcggcacgcgagctgggttcagaacgtcgtgagacagttcggtccctatccgtcgcgggcgttggaaatttgagaggagctgtccttagtacgagaggaccgggatggac

SEQ ID NO: 1244 - DNA, 701 bp, Enterococcus faecalis
aacgatttgggcactgtctcaacgagagactcggtgaaattttagtacctgtgaagatgcaggttacccgcgacaggacggaaagaccccatggagctttactgtagtttgatattgagtgtttgtaccacatgtacaggataggtaggagccgatgagaccggaacgctagtttcggaggaggcgctggtgggatactacccttgtgttatgaaccctctaacccgcaccactaatcgtggtgggagacagtgtcagatgggcagtttgactggggcggtcgcctcctaaaaggtaacggaggcgcccaaaggttccctcagaatggttggaaatcattcgaagagtgtaaaggcagaagggagcttgactgcgagacctacaagtcgagcagggacgaaagtcgggcttagtgatccggtggttccgcatggaagggccatcgctcaacggtaaaagctaccctggggataacaggcttatctcccccaagagtccacatcgacggggaggtttggcacctcgatgtcggctcgtcgcatcctggggctgtagtcggtcccaagggttgggctgttcgcccattaaagcggcacgcgagctgggttcagaacgtcgtgagacagttcggtccctatccgtcgcgggcgttggaaatttgagaggagctgtccttagtacgagaggaccgggatggactt

SEQ ID NO: 1245 - DNA, 691 bp, Escherichia coli
gatggccaggctgtctccacccgagactcagtgaaattgaactcgctgtgaagatgcagtgtacccgcggcaagacggaaagaccccgtgaacctttactatagcttgacactgaacattgagccttgatgtgtaggataggtgggaggctttgaagtgtggacgccagtctgcatggagccgaccttgaaataccaccctttaatgtttgatgttctaacgttgacccgtaatccgggttgcggacagtgtctggtgggtagtttgactggggcggtctcctcctaaagagtaacggaggagcacgaaggttggctaatcctggtcggacatcaggaggttagtgcaatggcataagccagcttgactgcgagcgtgacggcgcgagcaggtgcgaaagcaggtcatagtgatccggtggttctgaatggaagggccatcgctcaacggataaaaggtactccggggataacaggctgataccgcccaagagttcatatcgacggcggtgtttggcacctcgatgtcggctcatcacatcctggggctgaagtaggtcccaagggtatggctgttcgccatttaaagtggtacgcgagctgggtttagaacgtcgtgagacagttcggtccctatctgccgtgggcgctggagaactgaggggggctgctcctagtacgagaggaccgga

SEQ ID NO: 1246 - DNA, 911 bp, Haemophilus influenzae
tggctgcaactgtttattaaaaacacagcactctgcaaacacgaaagtggacgtatagggtgtgatgcctgcccggtgctggaaggttaattgatggtgtcatcgcaagagaagcacctgatcgaagccccagtaaacggcggccgtaactataacggtcctaaggtagcgaaattccttgtcgggtaagttccgacctgcacgaatggcataatgatggccaggctgtctccacccgagactcagtgaaattgaaatcgccgtgaagatgcggtgtacccgcggctagacggaaagaccccgtgaacctttactatagcttgacactgaacattgaattttgatgtgtaggataggtgggagcctttgaagcagtgacgccagtcattgtggaggcgaccttgaaataccaccctttaacgtttgatgttctaacgaagatgacgaaacgtggtctcggacagtgtctggtgggtagtttgactggggcggtctcctcccaaagcgtaacggaggagcacgaaggtttgctaatcacggtcggacatcgtgaggttagtgcaatggtataagcaagcttaactgcgagacagacaagtcgagcaggtacgaaagtaggtcatagtgatccggtggttctgaatggaagggccatcgctcaacggataaaaggtactccggggataacaggctgataccgcccaagagttcatatcgacggcggtgtttggcacctcgatgtcggctcatcacatcctggggctgaagtaggtcccaagggtatggctgttcgccatttaaagtggtacgcgagctgggtttagaacgtcgtgagacagttcggtccctatctgccgtgggcgttggatgattgattggggctgctcctagtacgagaggaccggagtgga

SEQ ID NO: 1247 - DNA, 881 bp, Mycobacterium tuberculosis
cgtgcgaagtcgcaagacgatgtatacggactgacgcctgcccggtgctggaaggttaagaggacccgttaacccgcaagggtgaagcggagaatttaagccccagtaaacggcggtggtaactataaccatcctaaggtagcgaaattccttgtcgggtaagttccgacctgcacgaatggcgtaacgacttctcaactgtctcaaccatagactcggcgaaattgcactacgagtaaagatgctcgttacgcgcggcaggacgaaaagaccccgggaccttcactacaacttggtattgatgttcggtacggtttgtgtaggataggtgggagactgtgaaacctcgacgccagttggggcggagtcgttgttgaaataccactctgatcgtattgggcatctaacctcgaaccctgaatcgggtttagggacagtgcctggcgggtagtttaactggggcggttgcctcctaaaatgtaacggaggcgcccaaaggttccctcaacctggacggcaatcaggtggcgagtgtaaatgcacaagggagcttgactgcgagacttacaagtcaagcagggacgaaagtcgggattagtgatccggcacccccgagtggaaggggtgtcgctcaacggataaaaggtaccccggggataacaggctgatcttccccaagagtccatatcgacgggatggtttggcacctcgatgtcggctcgtcgcatcctggggctggagcaggtcccaagggttgggctgttcgcccattaaagcggcacgcgagctgggtttagaacgtcgtgagacagttcggtctctatccgccgcgcgcgtcagaaacttgaggaaacctgtccctagtacgagaggaccgggac

SEQ ID NO: 1248 - DNA, 701 bp, Neisseria meningitidis
ggcgtaacgatggccacactgtctcctcctgagactcagcgaagttgaagtggttgtgaagatgcaatctacccgctgctagacggaaagaccccgtgaacctttactgtagctttgcattggactttgaagtcacttgtgtaggataggtgggaggcttagaagcagagacgccagtctctgtggagccgtccttgaaataccaccctggtgtctttgaggttctaacccagacccgtcatccgggtcggggaccgtgcatggtaggcagtttgactggggcggtctcctcccaaagcgtaacggaggagttcgaaggttacctaggtccggtcggaaatcggactgatagtgcaatggcaaaaggtagcttaactgcgagaccgacaagtcgagcaggtgcgaaagcaggacatagtgatccggtggttctgtatggaagggccatcgctcaacggataaaaggtactccggggataacaggctgattccgcccaagagttcatatcgacggcggagtttggcacctcgatgtcggctcatcacatcctggggctgtagtcggtcccaagggtatggctgttcgccatttaaagtggtacgtgagctgggtttaaaacgtcgtgagacagtttggtccctatctgcagtgggcgttggaagtttgacgggggctgctcctagtacgagaggaccggagt

SEQ ID NO: 1249 - DNA, 701 bp, Staphylococcus aureus
gcgtaacgatttgggcactgtctcaacgagagactcggtgaaatcatagtacctgtgaagatgcaggttacccgcgacaggacggaaagaccccgtggagctttactgtagcctgatattgaaattcggcacagcttgtacaggataggtaggagcctttgaaacgtgagcgctagcttacgtggaggcgctggtgggatactaccctagctgtgttggctttctaacccgcaccacttatcgtggtgggagacagtgtcaggcgggcagtttgactggggcggtcgcctcctaaaaggtaacggaggcgctcaaaggttccctcagaatggttggaaatcattcatagagtgtaaaggcataagggagcttgactgcgagacctacaagtcgagcagggtcgaaagacggacttagtgatccggtggttccgcatggaagggccatcgctcaacggataaaagctaccccggggataacaggcttatctcccccaagagttcacatcgacggggaggtttggcacctcgatgtcggctcatcgcatcctggggctgtagtcggtcccaagggttgggctgttcgcccattaaagcggtacgcgagctgggttcagaacgtcgtgagacagttcggtccctatccgtcgtgggcgtaggaaatttgagaggagctgtccttagtacgagaggaccgggatg

SEQ ID NO: 1250 - DNA, 701 bp, Staphylococcus epidermidis
cgtaacgatttgggcactgtctcaacgagagactcggtgaaatcatagtacctgtgaagatgcaggttacccgcgacaggacggaaagaccccgtggagctttactgtagcctgatattgaaattcggcacagcttgtacaggataggtaggagcctttgaaacgtgagcgctagcttacgtggaggcgttggtgggatactaccctagctgtgttggctttctaacccgcaccacttatcgtggtgggagacagtgtcaggcgggcagtttgactggggcggtcgcctcctaaaaggtaacggaggcgctcaaaggttccctcagaatggttggaaatcattcatagagtgtaaaggcataagggagcttgactgcgagacctacaagtcgagcagggtcgaaagacggacttagtgatccggtggttccgcatggaagggccatcgctcaacggataaaagctaccccggggataacaggcttatctcccccaagagttcacatcgacggggaggtttggcacctcgatgtcggctcatcgcatcctggggctgtagtcggtcccaagggttgggctgttcgcccattaaagcggtacgcgagctgggttcagaacgtcgtgagacagttcggtccctatccgtcgtgggcgtaggaaatttgagaggagctgtccttagtacgagaggaccgggatgg

SEQ ID NO: 1251 - DNA, 701 bp, Streptococcus pneumoniae
atgatttgggcactgtctcaacgagagactcggtgaaattttagtacctgtgaagatgcaggttacccgcgacaggacggaaagaccccatggagctttactgcagtttgatattgagtgtctgtaccacatgtacaggataggtaggagtctaagagatcgggacgccagtttcgaaggagacgctgttgggatactacccttgtgttatggccactctaacccagataggtgatccctatcggagacagtgtctgacgggcagtttgactggggcggtcgcctcctaaaaggtaacggaggcgcccaaaggttccctcagaatggttggaaatcattcgcagagtgtaaaggtataagggagcttgactgcgagagctacaactcgagcagggacgaaagtcgggcttagtgatccggtggttccgtatggaagggccatcgctcaacggataaaagctaccctggggataacaggcttatctcccccaagagttcacatcgacggggaggtttggcacctcgatgtcggctcgtcgcatcctggggctgtagtcggtcccaagggttgggctgttcgcccattaaagcggcacgcgagctgggttcagaacgtcgtgagacagttcggtccctatccgtcgcgggcgtaggaaatttgagaggatctgctcctagtacgagaggaccagagtggactt

SEQ ID NO: 1252 - DNA, 711 bp, Streptococcus pyogenes
aatgatttgggcactgtctcaacgagagactcggtgaaattttagtacctgtgaagatgcaggttacccgcgacaggacggaaagaccccatggagctttactgcagtttgatattgagtatcttgtaccacatgtacaggataggtaggagccattgacttcgggacgccagtttcgaatgaggcgttgttgggatactacccttgtgttatggctactctaacccagataggttatccctatcggagacagtgtctgacgggcagtttgactggggcggtcgcctcctaaagagtaacggaggcgcccaaaggttccctcagattggttggaaatcaatcgcagagtgtaaaggtataagggagcttgactgcgagagctacaactcgagcagggacgaaagtcgggcttagtgatccggtggtaccgaatggaagggccatcgctcaacggataaaagctaccctggggataacaggcttatctcccccaagagttcacatcgacggggaggtttggcacctcgatgtcggctcgtcgcatcctggggctgtagttcggtcccaaagggttgggctgttcgcccattaaagcggccaccgcgagctgggttcagaacgttcgtgagacagttcgggtcccttatcccgtcgcgggcgtaggaaatttgagaggatctgctcctagtacgagaggaccagagtggactt

SEQ ID NO: 1253 - DNA, 191 bp, Mycobacterium tuberculosis
acctcttggcggcgatgccgataaatatggtgtgatatatcacctttgcctgacagcgacttcacggcacgatggaatgtcgcaaccaaatgcattgtccgctttgatgatgaggagagtcatgccactgctaaccattggcgatcaattccccgcctaccagctcaccgctctcatcggcggtgacctgt

SEQ ID NO: 1254 - DNA, 191 bp, Mycobacterium tuberculosis
atctgagccaaacaccgggtgatcggcaaccaaccaccgagccacaggacagtcacgccgacacggctgcagcccagcaccgcgacccggggcaatcgggcacaggccggaatactgctcagtgaaacggttctgggagaatgtcggaaagccaaacgacacgacagatgggcggggcacgacttcgttgg

SEQ ID NO: 1255 - DNA, 61 bp, Mycobacterium tuberculosis
catcaacgggtggttctacgtcggcaactacggggtgccgtggtatgacatccagcccgtc

SEQ ID NO: 1256 - DNA, 261 bp, Mycobacterium tuberculosis
cttgttgctctggcatgtcatcggcgccacctcgtccgatgacggctaccttctgaccgtcgcccgggtcgccccgaaggccggctatgtagccaactactaccggtatttcggcacgacggaggcgccgttcgactggtatacatcggtgcttgcccagctggcggcggtgagcaccgccggcgtctggatgcgcctgcccgccaccctggccggaatcgcctgctggctgatcgtcagccgtttcgtgctgcggcggct

SEQ ID NO: 1257 - DNA, 61 bp, Mycobacterium tuberculosis
ctggtcgcgcaaccggatggcgttcctggcggcgttattcttcctgctggcgttgtgttgg

SEQ ID NO: 1258 - DNA, 261 bp, Mycobacterium tuberculosis
gtcgccgggcggcatgcgccggctgattccggcaagctggcgcaccttcaccctgaccgacgccgtggtgatattcggcttcctgctctggcatgtcatcggcgcgaattcgtcggacgacggctacatcctgggcatggcccgagtcgccgaccacgccggctacatgtccaactatttccgctggttcggcagcccggaggatcccttcggctggtattacaacctgctggcgctgatgacccatgtcagcgacgccag

SEQ ID NO: 1259 - DNA, 61 bp, Mycobacterium tuberculosis
cgtgttcgccgccgtggtggtcttcgtgttggccctgtcgttcgccagtgtcaacggctgg

SEQ ID NO: 1260 - DNA, 261 bp, Mycobacterium tuberculosis
gcggttcctgcccgcgcgctggtggtcgaccggcggtctggacaccctggttatcgccgtgctggtgtggtggcatttcgtcggggccaacacctccgacgacggctacatcctgaccatggcccgggtgtccgagcatgcgggctatatggccaactactaccgctggttcggcacacccgaggcgcctttcggctggtactacgacctgctggcgctgtgggctcatgtcagcacggccagtatctggatgcgcctacc

SEQ ID NO: 1261 - DNA, 161 bp, Mycobacterium tuberculosis
gccggcccctaggcagcgaagcctgactggccgcggaggtggtcaccctggcagcttactacgtgtcgatagtgtcgacatctcgttgacggcctcgacattacgttgatagcgtggatccatgaccgagcacctcgacgttgtcatcgtgggcgctggaa

SEQ ID NO: 1262 - DNA, 91 bp, Mycobacterium tuberculosis
catgggcggcacctgggatttgttccgttatcccggaattcgctccgactccgacatgtacacgctaggtttccgattccgtccctggacc

SEQ ID NO: 1263 - DNA, 161 bp, Mycobacterium tuberculosis
acaccgacaaacgtcacgagcgtaaccccagtgcgaaagttcccgccggaaatcgcagccacgttacgctcgtggacataccgatttcggcccggccgcggcgagacgataggttgtcggggtgactgccacagccactgaaggggccaaacccccattcg

SEQ ID NO: 1264 - DNA, 51 bp, Escherichia coli
cctcaacgtcacgcctgattccttttcggatggtggcacgcataactcgct

SEQ ID NO: 1265 - DNA, 231 bp, Neisseria meningitidis
cgaaatatcaagatgttgtcggtgaagtagcgcggtatttgaaagcgcgctcagcggaatgtatcgcggcaggcatcgcaccgcaacgcatcatactcgaccccggcttcggctccggcttcggcaaacccttgcaacacaatatcgcgctgatgcggcatttgcccgaattgatggcggaaacaggttttccgctgctcatcggcgtatcgcgcaaaagcacgataggcg

SEQ ID NO: 1266 - DNA, 161 bp, Neisseria meningitidis
gtgaatctcacacccgattctttttccgacggcggcgtgtattcgcaaaacgcccaaacagccttggcacatgccgaacagcttctaaaagaaggcgcagacattctcgacatcggcggcgaatcgacgcggtcgggtgcggattatgtttcgcctgaaga

SEQ ID NO: 1267 - DNA, 81 bp, Streptococcus pneumoniae
agcaggctcgtaaattgatagcagaaggagccagcatgctagatatcggcggtgaatcgactcggcctcggccgggaagta

SEQ ID NO: 1268 - DNA, 161 bp, Streptococcus pyogenes
gactccagattctttttcagatggggggtcttacactactgtgcaaaaagcattagatcacgttgagcaaatgattgctgatggggctaaaatcatcgacgttggtggagaatcaacacgtccaggttgccaatttgtaagcgctaccgatgaaattgacc

SEQ ID NO: 1269 - DNA, 241 bp, Acinetobacter baumannii
gatgtgagagacggtcttaaacctgttcaccgtcgtgtgctttatgccatgcacgaattgggcaatgactataacaaagcctacaagaaatctgctcgtgtcgttggggacgtaatcggtaaatatcacccgcatggtgacttagctgtttatgaaaccattgttcgtatggctcaagactttagcttacgttatttattggttgatggtcagggtaacttcggttcgatcgatggtgata

SEQ ID NO: 1270 - DNA, 241 bp, Bordetella pertussis
ctgccagacgtgcgtgacggtctcaagcctgtgcatcgccgcgtgctgtacgcgatgcacgaactgaacaacgattggaaccgtgcctataagaagtccgcgcgtatcgtcggggacgtcatcggtaagtaccacccgcacggcgaccagtcggtatacgacaccatcgtccgcatggcgcaggacttctccatgcgctacatgctggtcgacggccagggcaacttcggctccatcgacg

SEQ ID NO: 1271 - DNA, 241 bp, Citrobacter freundii
caccgtcgcgtactttacgccatgaacgtattgggcaacgactggaataaagcctataaaaaatctgcccgtgtcgttggtgacgtaatcggtaaataccaccctcatggtgataccgccgtttacgacaccattgttcgtatggcgcagccattctccttgcgttacatgctggtagatggtcagggtaactttggttctgtcgatggcgactccgcagcggcgatgcgttatacggaaa

SEQ ID NO: 1272 - DNA, 241 bp, Clostridium difficile
cctgatgttagagatggtttaaagccagttcatagaagaatattatattcaatgagtgagttaaatttaactccagataaaccatacaggaagtcagctcgtattgttggggacgttttaggtaagtaccatcctcatggagatactgctgtttattatgctatggtaagaatggcacaagatttttcaactagagcacttttagtagatggtcatggtaactttggttctgttgatgggg

SEQ ID NO: 1273 - DNA, 241 bp, Enterobacter cloacae
ctgccggacgtccgcgatggcctgaagccggtacaccgtcgcgtactatacgccatgaacgtattgggcaatgactggaataaagcctacaaaaaatctgcccgtgtcgttggtgacgtaatcggtaaataccatccccatggtgattccgcggtgtacgacaccatcgttcgtatggcgcagcctttctcgctgcgttacatgctggtagatggtcagggtaactttggttctatcgacg

SEQ ID NO: 1274 - DNA, 241 bp, Enterococcus faecium
gcactgcctgatgtcagagatggtctgaaacccgttcatcgccgtattttatacggcatgaacgaattaggtgtgacaccggataaagcccataaaaaatctgccagaatcgtcggagatgtcatgggtaaataccatcctcatggggatagtgcgatctatgagtcaatggtacggatggcgcagccatttagctaccgttatatgctagtggatggccatggaaactttggttctgttg

SEQ ID NO: 1275 - DNA, 241 bp, Enterococcus faecalis
gcgttacctgacgttcgtgacggtttaaaacctgttcatcgccgaatcttatatggaatgaacgaattaggggtaacccctgataaaccacacaaaaaatcagcccggattgttggggatgttatgggtaaatatcacccccatggggacagtgcgatttacgaatcaatggtgcggatggcacaaccttttagttatcgggctatgttagttgacggccacggaaacttcggttccgtcg

SEQ ID NO: 1276 - DNA, 241 bp, Escherichia coli
ctgccagatgtccgagatggcctgaagccggtacaccgtcgcgtactttacgccatgaacgtactaggcaatgactggaacaaagcctataaaaaatctgcccgtgtcgttggtgacgtaatcggtaaataccatccccatggtgactcggcggtctatgacacgatcgtccgcatggcgcagccattctcgctgcgttatatgctggtagacggtcagggtaacttcggttctatcgacg

SEQ ID NO: 1277 - DNA, 241 bp, Haemophilus influenzae
gcattacctgacgttcgagatggtttaaaacctgttcaccgccgcgtactattctcaatggatcgcgaaggcaataccgccaataaaaaatacgtaaaatcagcgcgtgttgtgggtgatgtaatcggtaaatatcacccgcatggtgactccgccgtgtacgataccatcgttcgtatggcacaacccttctcacttcgctatatgttggttgatgggcaaggtaactttggttcaattg

SEQ ID NO: 1278 - DNA, 241 bp, Klebsiella pneumoniae
ctgccggatgtccgagatggcctgaagccggtacaccgtcgcgtactttacgccatgaacgtattgggcaatgactggaacaaagcctataaaaaatcagcccgtgtcgttggtgacgtaatcggtaaataccacccgcacggcgactccgcggtatacgacaccatcgttcgtatggcgcagccgttctcgctgcgttacatgctggtggacggccagggtaacttcggttccatcgacg

SEQ ID NO: 1279 - DNA, 241 bp, Mycobacterium tuberculosis
agcgtgatcgtcggccgcgcgctgccggaggtgcgcgacgggctcaagcccgtgcatcgccgggtgctctatgcaatgttcgattccggcttccgcccggaccgcagccacgccaagtcggcccggtcggttgccgagaccatgggcaactaccacccgcacggcgacgcgtcgatctacgacaccctggtgcgcatggcccagccctggtcgctgcgctacccgctggtggacggccagg

SEQ ID NO: 1280 - DNA, 241 bp, Staphylococcus aureus
gcattgccagatgttcgtgacggtttaaaaccagtacatcgtcgtatactatatggattaaatgaacaaggtatgacaccggataaatcatataaaaaatcagcacgtatcgttggtgacgtaatgggtaaatatcaccctcatggtgacttatctatttatgaagcaatggtacgtatggctcaagatttcagttatcgttatccgcttgttgatggccaaggtaactttggttcaatgg

SEQ ID NO: 1281 - DNA, 241 bp, Streptococcus pneumoniae
gatgttcgagatggcttaaaacctgttcaccgtcgcattctctacggaatgaatgaattgggtgtgaccccagacaaaccccataaaaaatctgctcgtattacaggggatgtcatgggtaaatatcacccacacggggattcctctatttatgaagccatggtccgtatggctcaatggtggagctaccgttacatgcttgtagatggtcacgggaattttggttccatggatggagata

SEQ ID NO: 1282 - DNA, 241 bp, Streptococcus pyogenes
gatgtgcgtgatggtctgaaacctgtgcatcgtcgtattctctatgggatgaatgaactcggtgtgacacctgataaaccgcataaaaaatctgctcgtattacgggtgatgtcatgggtaaatatcacccgcatggggattcttctatttatgaagctatggttcgtatggctcagtggtggagttatcgccatatgcttgttgatggccatggcaatttttgttctatggatggtgatg

SEQ ID NO: 1283 - DNA, 221 bp, Escherichia coli
ccaggaacgcgatccggcgctttccgaactgtacctggtggaaggggactccgcgggcggctctgcgaagcaggggcgtaaccgcaagaaccaggcgattctgccgctgaagggtaaaatcctcaacgtcgagaaagcgcgcttcgataagatgctctcttctcaggaagtggcgacgcttatcaccgcgcttggctgtggtatcggtcgtgacgagtaca

SEQ ID NO: 1284 - DNA, 221 bp, Mycobacterium tuberculosis
gttccacggatccgcgcaagtccgaactgtatgtcgtagaaggtgactcggccggcggttctgcaaaaagcggtcgcgattcgatgttccaggcgatacttccgctgcgcggcaagatcatcaatgtggagaaagcgcgcatcgaccgggtgctaaagaacaccgaagttcaggcgatcatcacggcgctgggcaccgggatccacgacgagttcgatatc

SEQ ID NO: 1285 - DNA, 51 bp, Mycobacterium tuberculosis
cgactcggacgcgtatgcgatatctggtggtctgcacggcgtcggcgtgtc

SEQ ID NO: 1286 - DNA, 221 bp, Mycobacterium tuberculosis
agaaggcccaagacgaatacggcgctgcgtctatcaccattctcgaagggctggaggccgtccgcaaacgtcccggcatgtacattggctcgaccggtgagcgcggtttacaccatctcatttgggaggtggtcgacaacgcggtcgacgaggcgatggccggttatgcaaccacagtgaacgtagtgctgcttgaggatggcggtgtcgaggtcgccgac

SEQ ID NO: 1287 - DNA, 221 bp, Pseudomonas aeruginosa
gaaaaggacccggcgctctccgaactgtacatcgtggagggtgactccgcgggcggttccgccaagcagggccgcaatcgccggacccaggcgatcctgccgctcaagggcaagatcctcaacgtcgaaaaggcgcgcttcgacaagatgctctcctcccaggaggtcggtacgctgatcaccgccctgggctgtggcatcggccgcgaggaatacaacat

SEQ ID NO: 1288 - DNA, 231 bp, Staphylococcus aureus
ttgctctagtaaaagtcctgaagaatgtgagattttcttagtcgaaggggactctgccggggggtctacaaaatctggtcgtgactctagaacgcaggcgattttaccattacgaggtaagatattaaatgttgaaaaagcacgattagatagaattttgaataacaatgaaattcgtcaaatgatcacagcatttggtacaggaatcggtggcgactttgatctagcgaa

SEQ ID NO: 1289 - DNA, 132 bp, Mycobacterium tuberculosis
gagacacccgatcccgagatcgaccttgacgtcgtcgccggcgaaccgcgctatggcgattaccgctacgcagtcaacaactcgttcgggttcggcggccacaatgtggcgcttgccttcgggcgttactga

SEQ ID NO: 1290 - DNA, 231 bp, Mycobacterium tuberculosis
cggacatcgagagcacgtggaagggtctgttggccggcgagagcggcatccacgcactcgaagacgagttcgtcaccaagtgggatctagcggtcaagatcggcggtcacctcaaggatccggtcgacagccacatgggccgactcgacatgcgacgcatgtcgtacgtccagcggatgggcaagttgctgggcggacagctatgggagtccgccggcagcccggaggtcg

SEQ ID NO: 1291 - DNA, 181 bp, Mycobacterium tuberculosis
gccgttggcccgattgctgggtgccggtatcacctcggacgcctttcatatggtggcgcccgcggccgatggtgttcgtgccggtagggcgatgactcgctcgctggagctggccgggttgtcgccggcggacatcgaccacgtcaacgcgcacggcacggcgacgcctatcggcgacgcc

SEQ ID NO: 1292 - DNA, 140 bp, Escherichia coli
tatgtaagatctcaactatcgcatccgtggattaattcaattataacttctctctaacgctgtgtatcgtaacggtaacactgtagaggggagcacattgatgagcacgtcagacgatatccataacaccacagccactg

SEQ ID NO: 1293 - DNA, 151 bp, Escherichia coli
ccggtctgtttattcgtatggcctggcacggcgcggggacttaccgttcaatcgatggacgcggtggcgcgggtcgtggtcagcaacgttttgcaccgctgaactcctggccggataacgtaagcctcgataaagcgcgtcgcctgttgtg

SEQ ID NO: 1294 - DNA, 171 bp, Escherichia coli
atacgctgggtaaaacccacggtgccggtccgacatcaaatgtaggtcctgatccagaagctgcaccgattgaagaacaaggtttaggttgggcgagcacttacggcagcggcgttggcgcagatgccattacctctggtctggaagtagtctggacccagacgccgaccc

SEQ ID NO: 1295 - DNA, 140 bp, Klebsiella pneumoniae
tgaacggaattagctattaaatctgtagactcattcaaatatcgcttattggtagcagtgtatatcgtaatggcattcacaaaagcaaacggagcctgatatgagcacgtctaacgacccatccaacaacgcatccgccg

SEQ ID NO: 1296 - DNA, 151 bp, Klebsiella pneumoniae
ttggcttgtttattcgcatggcctggcacggcgccggcacctaccgcaccgttgacggtcgcggcggcgcaggccgtgggcagcagcgctttgcgccgctgaactcctggccagacaacgtcagtctcgataaagcccgtcgcctgctgtg

SEQ ID NO: 1297 - DNA, 171 bp, Klebsiella pneumoniae
atacgctgggcaaaacccacggcgccgccgaaaccagccacgtcggcgccgagccggaagccgccccactggaagcgcaaggcctgggctggcactccagctacggcagcggcgcaggtgcggatgccatcacctccggtctggaagtggtctggacgcagacgcccaccc

SEQ ID NO: 1298 - DNA, 140 bp, Mycobacterium tuberculosis
gtcatctactggggtctatgtcctgattgttcgatatccgacacttcgcgatcacatccgtgatcacagcccgataacaccaactcctggaaggaatgctgtgcccgagcaacacccacccattacagaaaccaccaccg

SEQ ID NO: 1299 - DNA, 151 bp, Mycobacterium tuberculosis
actacgggccgctgtttatccggatggcgtggcacgctgccggcacctaccgcatccacgacggccgcggcggcgccgggggcggcatgcagcggttcgcgccgcttaacagctggcccgacaacgccagcttggacaaggcgcgccggct

SEQ ID NO: 1300 - DNA, 171 bp, Mycobacterium tuberculosis
tcggcggtcacactttcggtaagacccatggcgccggcccggccgatctggtcggccccgaacccgaggctgctccgctggagcagatgggcttgggctggaagagctcgtatggcaccggaaccggtaaggacgcgatcaccagcggcatcgaggtcgtatggacgaaca

SEQ ID NO: 1301 - DNA, 521 bp, Mycobacterium tuberculosis
ctcggtcacacctaccaaaccccctacgacagcctgatcgtcgccgcgggtgctggccagtcttatttcggcaacgaccatttcgccgaattcgcacccggcatgaagtccatcgacgacgcgttggagttgcgtggccgcatattgagcgctttcgagcaagccgaacggtccagcgatccggaacggcgggccaagctactgacattcaccgttgtcggggctggccccaccggtgttgaaatggccggacagatcgccgagctggccgagcacacgttgaagggcgcattccggcacatcgactcgaccaaggcgcgggtgattctgcttgacgccgccccggcggtgctgccaccgatgggcgcaaagctcggtcagcgggcggctgcccggttgcagaagctgggcgtggaaatccagctgggtgcgatggtcaccgacgtcgaccgcaacggcatcaccgtcaaggactccgacggcaccgtccggcgcatcgagtcggcctgcaaggtctggtc

SEQ ID NO: 1302 - DNA, 330 bp, Citrobacter freundii
atgtacgtcatcatggacagggcgttgccatttattggcgatggtttgaaacccgttcagcgtcgcattgtgtatgcaatgtccgaactggggctgaatgccagcgccaaatttaaaaaatccgcccgtaccgtcggtgacgtgctgggtaaataccacccgcacggcgacagcgcctgctatgaagcgatggtgctgatggcgcagccgttctcttaccgttatccgctggttgatggtcagggaaactggggggcgccggacgatcccaaatccttcgcggcgatgcgttataccgaatcccgcttgtctaaatattccgagctgcta

SEQ ID NO: 1303 - DNA, 511 bp, Enterococcus faecalis
gcgatcgttttggaagatattccaaatacatcattcaagaacgggccctaccagatattcgagatgggctaaaaccagtacaacgccgcattctcttttcaatgaataaagacggcaatacctttgataaaggctttcgtaaatcagcaaaatctgtcggaaacattatggggaattatcatccccatggcgacagcagtatttatgaagcgatggtgcgtctaagtcaagactggaaattacgggaagtactaattgaaatgcacggaaacaacggaagtatggatggcgacccaccagcggcgatgcgttatacagaagctcgtttatcacagttaagtggtgagttgttaaaagatatcgacaaaaataccgtcgatttcgtctggaattttgacgatacagaaaaagaacccactgttttaccagccaaatatcctaatttattggtaaacggctcaacggggatttcagcaggctatgcaacagaaattcctacgcataatttagc

SEQ ID NO: 1304 - DNA, 521 bp, Escherichia coli
acggaaaacgcctacttaaactactccatgtacgtgatcatggaccgtgcgttgccgtttattggtgatggtctgaaacctgttcagcgccgcattgtgtatgcgatgtctgaactgggcctgaatgccagcgccaaatttaaaaaatcggcccgtaccgtcggtgacgtactgggtaaataccatccgcacggcgatagcgcctgttatgaagcgatggtcctgatggcgcaaccgttctcttaccgttatccgctggttgatggtcaggggaactggggcgcgccggacgatccgaaatcgttcgcggcaatgcgttacaccgaatcccggttgtcgaaatattccgagctgctattgagcgagctggggcaggggacggctgactgggtgccaaacttcgacggcactttgcaggagccgaaaatgctacctgcccgtctgccaaacattttgcttaacggcaccaccggtattgccgtcggcatggcgaccgatattccaccgcataacctgcgtga

SEQ ID NO: 1305 - DNA, 511 bp, Haemophilus influenzae
ggcttatctcaactattctatgtatgtcatcatggatcgtgcgttgccttttatcggtgatggtttaaaacccgttcaacgtcgtattgtatatgcgatgtctgaacttggcttaaatgccacggcaaaatacaaaaaatctgctcgtaccgtcggtgatgtactcggtaaattccatccacatggtgacagtgcttgttatgaagctatggtgttaatggcacaacccttctcttatcgttatccacttgtagatggtcaaggtaactggggggcaccagatgatccaaaatccttcgcagccatgcgttatacggaatctcgcctatctaaaatctctgaaatcttgttgaatgaactcggacaaggaacagtagattatcaaccaaactttgatggaaccttggctgaaccacaatatttacctgctcgtttaccgcatattttattgaacggcacaacaggtattgcggtggggatggccacagatattccaccacacaatattaac

SEQ ID NO: 1306 - DNA, 521 bp, Klebsiella pneumoniae
acggaaaatgcttacctgaactactccatgtacgtgatcatggacagggcattaccgtttattggcgatggcttaaaaccggtccagcgtcgcatcgtctacgcgatgtccgagctggggctgaacgccagcgcgaaattcaaaaagtccgcccgcaccgtcggcgacgtgttgggtaaatatcacccgcacggcgacagcgcctgctatgaagcgatggtgctgatggcgcagccgttctcttaccgttacccgctggtagacgggcagggaaactggggtgcgccggacgatccgaaatccttcgccgccatgcgttacaccgagtcccgcctgtcgaagtatgctgaactgctgctcagcgagctgggacagggaacggtcgactgggtaccaaactttgacgggacgctgcaggagccgaaaatgctgcctgcgcgtctaccgaacatcctgcttaatggcaccaccggcatcgcggtgggtatggccactgatattccgccgcacaacctgcgtga

SEQ ID NO: 1307 - DNA, 511 bp, Pseudomonas aeruginosa
gcctatctgaactattccatgtacgtgatcatggaccgcgccctgccgcatatcggcgacggcctgaaaccggtgcagcgacgcatcgtctacgccatgagcgaactggggctggatgccgattccaagcacaagaagtcggcgcgcaccgtcggcgacgtgctcggcaagttccacccgcacggcgactcggcctgctacgaggccatggtgctgatggcgcagccgttctcctatcgctatccgctggtggacggccagggcaactggggggctccggacgatcccaagtccttcgccgccatgcgttataccgaggcgcgcctgtcgcgctattccgaggtgctgctcagcgaactgggccagggtaccgtggactgggtaccgaacttcgacggcaccctcgacgagccggccgtgctgccggcccgcctgcccaacctgctgctcaacggcaccaccggcatcgcggtgggcatggccaccgacgtgccgccgcacaacctgcggg

SEQ ID NO: 1308 - DNA, 511 bp, Staphylococcus aureus
ttaggtgatcgctttggaagatatagtaaatatattattcaagagcgtgcattgccagatgttcgtgatggtttaaaaccagtacaacgtcgtattttatatgcaatgtattcaagtggtaatacacacgataaaaatttccgtaaaagtgcgaaaacagtcggtgatgttattggtcaatatcatccacatggagacttctcagtgtacgaagcaatggtccgtttaagtcaagactggaagttacgacatgtcttaatagaaatgcatggtaataatggtagtatcgataatgatccgccagcggcaatgcgttacactgaagctaagttaagcttactagctgaagagttattacgtgatattaataaagagacagtttctttcattccaaactatgatgatacgacactcgaaccaatggtattgccatcaagatttcctaacttactagtgaatggttctacaggtatatctgcaggttacgcgacagatataccaccacataatt

SEQ ID NO: 1309 - DNA, 281 bp, Streptococcus pneumoniae
gtcgggaacatcatggggaatttccacccacacggggattcttctatctatgatgccatggttcgtatgtcacagaactggaaaaatcgtgagattctagttgaaatgcacggtaataacggttctatggacggagatcctcctgcggctatgcgttatactgaggcacgtttgtctgaaattgcaggttaccttcttcaggatatcgagaaaaatacagttccttttgcatggaactttgacgatacggagaaagaaccaacggtcttgccagcagcctt

SEQ ID NO: 1310 - DNA, 81 bp, Streptococcus pyogenes
tatcatggggaatttccacccgcatggggattcctctatttatgatgccatggtacggatgagtcaaaattggaaaaaccg

SEQ ID NO: 1311 - DNA, 281 bp, Escherichia coli
tgcgcaaaaagctgaccagcggcccagcactgcctggcaaactggctgactgtaccgcgcaggatcttaaccgtaccgaactgttccttgtggaaggtgactccgcaggcggatctgccaagcaggcgcgcgatcgcgaatatcaggcgatcatgccactgaaaggtaagatccttaacacctgggaagtttcttccgacgaagtgctggcctcgcaggaagtgcatgatatttcggtagcgatcggtatcgatcctgacagcgacgatctgagccagctt

SEQ ID NO: 1312 - DNA, 371 bp, Escherichia coli
accgggcttgagccggttcgccgccgtccggggatgtataccgataccactcgccctaaccatttggggcaagaagttattgataacagtgtcgatgaagcactggcgggtcacgcaaaacgcgtggatgtaatcttacatgctgaccagtcgttagaagttattgacgatgggcgcgggatgccggtggatattcacccggaagagggtgtaccggcggttgaactgattctttgccgtctgcatgcaggcggtaaattctctaacaaaaattaccagttctctggcggcctgcatggcgtggggatttcggtggttaacgccctgtcgaagcgcgtagaagttaacgtgcgccgcgatggtcaggttta

SEQ ID NO: 1313 - DNA, 281 bp, Pseudomonas aeruginosa
gcaagaagatcacccaggggccggcgctgcccggcaaactggccgactgcgccggacaggaaccgatgcgcgcggaactgttcctggtcgagggcgactccgccggcggctcggcgaagcaggcgcgggacaaggaattccaggcgatcatgccgctgcgcggaaagatcctgaacacctgggaagtggacggcggcgaggtgctcgccagccaggaggtccacgacatcgcggtggccatcggcgtcgatccgggtgccagtgacctggcccagctgcgc

SEQ ID NO: 1314 - DNA, 381 bp, Pseudomonas aeruginosa
agtcctttccggcctcgacccggtgcgcaagcgcccggggatgtacaccgacaccacccgccccaaccatctggcccaggaagtcatcgacaacagcgtcgacgaagccctggccggccatgcgaagagcgtgcaggtgatcctgcaccaggacaactcgctggaagtcatcgacgatggccgcggcatgccggtggacatccacccggaagagggcgtgccgggcgtcgagctgatccttaccaagctgcatgccggcggcaagttctcgaacaagaactaccagttctccggcggcttgcacggggtcggcatctcggtggtgaacgcgctctcgacccgggtcgaggtacgcgtcaagcgcgacgccaacgagtaccg

SEQ ID NO: 1315 - DNA, 281 bp, Staphylococcus aureus
aacaagcgtaaagacactttgctatctggtaaattaacacctgcacaaagtaaaaacactgaaaaaaatgaattgtatttagtcgaaggtgattctgcgggaggttcagcaaaacttggacgagaccgcaaattccaagcgatattaccattacgtggtaaggtaattaatacagagaaagcacgtctagaagatatttttaaaaatgaagaaattaatacaattatccacacaatcggggcaggcgttggtactgactttaaaattgaagatagtaatta

SEQ ID NO: 1316 - DNA, 381 bp, Staphylococcus aureus
tagaggggttagaagcagttcgtaaaagacctggtatgtatattggatcaactgataaacggggattacatcatctagtatatgaaattgtcgataactccgtcgatgaagtattgaatggttacggtaacgaaatagatgtaacaattaataaagatggtagtatttctatagaagataatggacgtggtatgccaacaggtatacataaatcaggtaaaccgacagtcgaagttatctttactgttttacatgcaggaggtaaatttggacaaggcggctataaaacttcaggtggtcttcacggtgttggtgcttcagttgtaaatgcattgagtgaatggcttgaagttgaaatccatcgagatggtagtatatatc

SEQ ID NO: 1317 - DNA, 271 bp, Streptococcus pneumoniae
aaaaacaagaaagataagggcttgttgtctgggaaattgaccccagcccaatctaagaatcctgctaagaatgaactctatctagttgagggggactctgccggtggttctgccaaacaaggtcgtgaccgcaagttccaggctattctacctcttcgtggtaaggttatcaatacagccaaggccaagatggcggatatcctcaaaaatgaagagatcaataccatgatttataccattggtgcgggtgttggagcagacttctctattg

SEQ ID NO: 1318 - DNA, 391 bp, Streptococcus pneumoniae
aggtgctagaagggttggatgcggtccgaaaacgtccagggatgtatattggatcgaccgatggcgctggtcttcatcacctagtttgggaaatcgttgataatgcagtcgatgaagccttgtctgggtttggtgatcgtatcgatgtaaccatcaataaagacggtagtctaacggttcaagaccatggacgtgggatgccgacaggtatgcacgctatgggaattccaactgttgaggttatctttaccattcttcatgccggagggaaattcggtcaaggtggctataagacatcaggtggacttcacggagtgggttcttccgttgttaacgccctttctagctggttagaagttgaaattacccgtgatggcgcagtttacaagca

SEQ ID NO: 1319 - DNA, 131 bp, Mycobacterium tuberculosis
gccaccgatcattgtgtgcgccagacggccgaggacgcggtacgcaatggcttggccaccagggtgctggtggacctgacagcgggtgtgtcggccgataccaccgtcgccgcgctggaggagatgcgcac

SEQ ID NO: 1320 - DNA, 1095 bp, Staphylococcus aureus
atgataactgctgaaaagaaaaagaagaataaatttcttccaaattttgacaagcaatcaatatattcattgcgatttgacgaaatgcaaaactggctcgttgaacaaggtcaacaaaaatttcgagcgaaacagatttttgaatggttatatcaaaaaagagtagactcgattgatgaaatgacgaacttatcgaaagacttacgacagcttttaaaagataactttactgttacaactttaacaactgtagtaaaacaagaaagtaaagacggtacaattaaattcttatttgaattacaagatggctatacaattgaaactgttttaatgagacatgattatggaaattcagtatgtgtaacgacacaagtaggttgtcgtatcggatgtacgttttgtgcttctacacttggcggtttaaaaagaaaccttgaagctggcgaaattgtttcacaagttttaacagttcaaaaagcccttgatgctacagaagagcgcgtatctcaaattgtcataatgggtatcggtgaaccatttgaaaattatgatgaaatgatggactttttaagaatcgtcaatgatgataacagtttaaatattggtgcacgtcacattacagtatcaacatcaggtatcattcctagaatatatgactttgcggatgaagatattcaaattaatttcgctgtaagtttacacgccgcaaaagatgaagtgcgatcacgcttgatgccaattaaccgtgcatataatgttgagaagttaatcgaagcaattcaatattatcaagaaaaaacaaatcgtcgtgttacttttgaatatggtctgtttggtggtgtgaatgaccaactagaacatgcaagagaattagcacatttaataaaaggcttaaactgccatgttaacttaattcctgtcaaccatgttccagaaagaaattatgtaaaaacggctaaaaatgatatctttaaatttgaaaaagaattaaagagactaggaattaatgccacaatacgtcgtgaacaaggttcggatattgacgcagcttgtggtcaattaagagcaaaggaacgacaagtagaaacgaggtaa

SEQ ID NO: 1321 - DNA, 111 bp, Mycobacterium tuberculosis
gcgcgggtcaacccgttcgggttcatcgaaacgccgtaccgcaaggtggtcgacggcgtggttagcgacgagatcgtgtacctgaccgccgacgaggaggaccgccacgtg

SEQ ID NO: 1322 - DNA, 231 bp, Mycobacterium tuberculosis
tctctgaccctcgtttcgacgatgtcaaggcacccgtcgacgagtgcaaagacaaggacatgacgtacgcggctccactgttcgtcaccgccgagttcatcaacaacaacaccggtgagatcaagagtcagacggtgttcatgggtgacttcccgatgatgaccgagaagggcacgttcatcatcaacgggaccgagcgtgtggtggtcagccagctggtgcggtcgcccg

SEQ ID NO: 1323 - DNA, 191 bp, Mycobacterium tuberculosis
tgcacccgcgtgtacaccaccactccgaagaagccgaactcggcgcttcggaaggttgcccgcgtgaagttgacgagtcaggtcgaggtcacggcgtacattcccggcgagggccacaacctgcaggagcactcgatggtgctggtgcgcggcggccgggtgaaggacctgcctggtgtgcgctacaagat